(19) United States
(12) Patent Application Publication
(10) Pub. No.: US 2009/0226420 A1
(43) Pub. Date: Sep. 10, 2009

(54) Methods of Determining the Risk of Developing Coronary Artery Disease

(76) Inventors: Elizabeth Hauser, Durham, NC (US); Pascal Goldschmidt, Miami, FL (US); Simon Gregory, Durham, NC (US); William Kraus, Hillsborough, NC (US); Jeffery Vance, Coral Gables, FL (US)

Correspondence Address: MYERS BIGEL SIBLEY & SAJOVEC, PO BOX 37428, RALEIGH, NC, 27627, US

(21) Appl. No.: 12/084,759

(22) PCT Filed: Nov. 10, 2006
(86) PCT No.: PCT/US06/43534

§ 371 Date: Mar. 23, 2009

Related U.S. Application Data
(60) Provisional application No. 60/735,694, filed on Nov. 10, 2005.

(51) Int. Cl. C12Q 1/68; A61K 39/395; A61P 9/00

(52) U.S. Cl. 424/130.1; 435/6

(57) ABSTRACT

The invention relates to predicting, or aiding in predicting, which individuals are at risk of developing coronary artery disease. The invention provides a method for identifying an individual who has an altered risk for developing CAD. The invention further relates to methods of reducing the likelihood that a subject will develop CAD. The invention further provides reagents, nucleic acids and kits comprising nucleic acids containing a polymorphism in a CAD-determinative gene.

### CROSS-REFERENCE TO RELATED APPLICATIONS

This application claims the benefit of the filing date of U.S. Application No. 60/735,694, filed Nov. 10, 2005, entitled “METHODS OF DETERMINING THE RISK OF DEVELOPING CORONARY ARTERY DISEASE.” The entire teachings of the referenced application are herein incorporated by reference.

### STATEMENT REGARDING FEDERALLY-SPONSORED RESEARCH OR DEVELOPMENT

The invention described herein was supported, in whole or in part, by the National Institute of Health Grant Nos. P01-HL73042 and R01-HL073389. The United States government has certain rights in the invention.

### FIELD OF THE INVENTION

The present invention is in the field of vascular disease diagnosis and therapy. In particular, the present invention relates to specific single nucleotide polymorphisms (SNPs) in the human genome, and their association with vascular disease and related pathologies, in particular, coronary artery disease (CAD) such as coronary stenosis.

### BACKGROUND OF THE INVENTION

Cardiovascular disorders are a cause of significant morbidity and mortality in the United States. Among the more common cardiovascular disorders are coronary artery diseases (CADs). CADs, sometimes designated coronary heart diseases or ischemic heart diseases, are characterized by insufficiency in blood supply to cardiac muscle. CADs can be manifested as acute cardiac ischemia (e.g., angina pectoris or myocardial infarction) or chronic cardiac ischemia (e.g., coronary arteriosclerosis or coronary atherosclerosis). CADs are a common cause of cardiac failure, cardiac arrhythmias, and sudden death. In patients afflicted with CADs, the cardiac muscle is not sufficiently supplied with oxygen. Severe cardiac ischemia can be manifested as severe pain or cardiac damage. Less severe ischemia can damage cardiac muscle and cause changes to cardiac tissues over the long term that impair cardiac function.

Many disorders, including CADs, develop over time and could be delayed, inhibited, lessened in severity, or prevented altogether by making lifestyle changes or through pharmaceutical treatment. For cardiovascular disorders such as CAD, such changes include increasing exercise, adjusting diet, consuming nutritional or pharmaceutical products known to be effective against cardiovascular disorders, and undergoing heightened medical monitoring. These changes are often not made, due to the expense or inconvenience of the changes to an individual and on her subjective belief that she is not at high risk for cardiovascular disorders. Improved monitoring of cardiovascular health can help to identify individuals at risk for developing cardiovascular disorders, including CAD, and permit for more informed decisions as to whether lifestyle changes are justified.

One way to identify subjects at high risk for developing CAD is by identifying genetic elements that predispose an individual to develop CAD. Polymorphisms conferring higher risks to non-cardiovascular diseases have been identified which aid in their diagnosis. Apolipoprotein E genetic screening aids in identifying genetic carriers of the apoE4 polymorphism in dementia patients for the differential diagnosis of Alzheimer's disease. Factor V Leiden polymorphisms signals a predisposition to deep venous thrombosis. The identification of polymorphisms in disease-associated genes also aids in designing an effective treatment plan for the disorder. For example, in the treatment of cancer, diagnosis of genetic variants in tumor cells is used for the selection of the most appropriate treatment regimen for the individual patient. In breast cancer, genetic variation in estrogen receptor expression or heregulin type 2 (Her2) receptor tyrosine kinase expression determine if anti-estrogenic drugs (e.g. tamoxifen) or anti-Her2 antibody (e.g. Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (ST1571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

Therefore, a need remains for the identification of genomic polymorphisms that predispose an individual to develop cardiovascular diseases such as CAD and that aid in their treatment. The invention provides such CAD-determinative genes and polymorphisms, and related assays, satisfying this need.

### SUMMARY OF THE INVENTION

The invention broadly relates to estimating, and aiding to estimate, the likelihood that a subject will be afflicted with cardiovascular disease, and to identifying subjects with an elevated risk of developing cardiovascular disease and to related kits and reagents. In one embodiment, the cardiovascular disease is coronary artery disease (CAD). The invention also relates, in part, to methods and reagents for identifying, or aiding in the identification of, subjects at high risk of developing CAD or other cardiovascular diseases.

Another aspect of the invention provides a method for identifying an individual who has an altered risk for developing CAD, comprising detecting the presence of a single nucleotide polymorphism (SNP) in said individual's nucleic acids, wherein the presence of the SNP is correlated with an altered risk for coronary stenosis in said individual. In one embodiment, the SNP is selected from SNPs set forth in Tables 1-5. In one embodiment, the SNP is represented by a SEQ ID NOs: selected from 1-575. In one embodiment, the altered risk is an increased risk. In one embodiment, the detection is carried out by a process selected from the group consisting of: allele-specific probe hybridization, allele-specific primer extension, allele-specific amplification, sequencing, 5′ nuclease digestion, molecular beacon assay, oligonucleotide ligation assay, size analysis, and single-stranded conformation polymorphism.

Assessments of genomic polymorphism content in two or more of the CAD-determinative genes can be combined to determine the risk of a subject in developing cardiovascular disease. This assessment of cardiovascular health can be used to predict the likelihood that the human will develop CAD or other cardiovascular disorders such as myocardial infarction and hypertension. Identification of high-risk subjects allows for the early intervention to prevent, delay, or ameliorate the onset of cardiovascular disease.

Another aspect of the invention provides an isolated nucleic acid molecule comprising at least 10, 15, 20, 21 or more contiguous nucleotides, wherein one of the nucleotides is a single nucleotide polymorphism (SNP) selected from any one of the nucleotide sequences in SEQ ID NOS: 1-575, or a complement thereof.

One aspect of the present invention relates to an isolated nucleic acid molecule comprising a nucleotide sequence in which at least one nucleotide is a SNP disclosed in Tables 1-4. In an alternative embodiment, a nucleic acid of the invention is an amplified polynucleotide, which is produced by amplification of a SNP-containing nucleic acid template. In another embodiment, the invention provides for a variant protein which is encoded by a nucleic acid molecule containing a SNP disclosed herein. In yet another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest. In an alternative embodiment, a protein detection reagent is used to detect a variant protein which is encoded by a nucleic acid molecule containing a SNP disclosed herein. A preferred embodiment of a protein detection reagent is an antibody or an antigen-reactive antibody fragment.

Another aspect of the invention provides kits comprising SNP detection reagents, and methods for detecting the SNPs disclosed herein by employing detection reagents. In a specific embodiment, the present invention provides for a method of identifying an individual having an increased or decreased risk of developing coronary artery disease by detecting the presence or absence of one or more SNP alleles disclosed herein. In another embodiment, a method for diagnosis of coronary artery disease by detecting the presence or absence of one or more SNP alleles disclosed herein is provided.

The nucleic acid molecules of the invention can be inserted in an expression vector, such as to produce a variant protein in a host cell. Thus, the present invention also provides for a vector comprising a SNP-containing nucleic acid molecule, genetically-engineered host cells containing the vector, and methods for expressing a recombinant variant protein using such host cells. In another specific embodiment, the host cells, SNP-containing nucleic acid molecules, and/or variant proteins can be used as targets in a method for screening and identifying therapeutic agents or pharmaceutical compounds useful in the treatment of coronary artery disease.

Another aspect of the invention provides a method for treating coronary artery disease in a human subject wherein said human subject harbors a SNP, gene, transcript, and/or encoded protein identified in Tables 1-4, which method comprises administering to said human subject a therapeutically or prophylactically effective amount of one or more agents counteracting the effects of the disease, such as by inhibiting (or stimulating) the activity of the gene, transcript, and/or encoded protein identified in Tables 1-4.

Another aspect of this invention provides a method for treating coronary artery disease in a human subject, which method comprises: (i) determining that said human subject harbors a SNP, gene, transcript, and/or encoded protein identified in Tables 1-4, and (ii) administering to said subject a therapeutically or prophylactically effective amount of one or more agents counteracting the effects of the disease.

Another aspect of this invention provides a method for identifying an agent useful in therapeutically or prophylactically treating coronary artery disease in a human subject wherein said human subject harbors a SNP, gene, transcript, and/or encoded protein identified in Tables 1-2, which method comprises contacting the gene, transcript, or encoded protein with a candidate agent under conditions suitable to allow formation of a binding complex between the gene, transcript, or encoded protein and the candidate agent and detecting the formation of the binding complex, wherein the presence of the complex identifies said agent.

Another aspect of the invention provides a method for stratifying a patient population for treatment of coronary artery disease, wherein said population has an altered risk for developing coronary artery disease due to the presence of a single nucleotide polymorphism (SNP) in any one of the nucleotide sequences of SEQ ID NOS: 1-575 in an individual's nucleic acids from said population, comprising detecting the SNP, wherein the presence of the SNP is correlated with an altered risk for coronary artery disease in said individual thereby indicating said individual should receive treatment for coronary artery disease.

The methods of SNP genotyping provided by the invention are useful for numerous practical applications. Examples of such applications include, but are not limited to, disease predisposition screening, disease diagnosis, disease prognosis, disease progression monitoring, determining therapeutic strategies based on an individual's genotype (“pharmacogenomics”), developing therapeutic agents based on SNP genotypes associated with a disease or likelihood of responding to a drug, stratifying a patient population for clinical trial for a treatment regimen, predicting the likelihood that an individual will experience toxic side effects from a therapeutic agent, and human identification applications such as forensics.

### BRIEF DESCRIPTION OF THE DRAWINGS

FIG. 1 shows SNP selection algorithm for candidate genes from the association with human-disease components of the AGENDA study.

FIG. 2 shows a graphical representation of the largest negative Log (base 10) p-values for 1065 SNPs in 275 genes. This figure is in color.

### DETAILED DESCRIPTION OF THE INVENTION

### I. Overview

The invention provides, in part, novel methods of determining the risk that an individual will develop a cardiovascular disease. The invention also provides methods of identifying subjects having an elevated risk of developing a cardiovascular disease, such as CAD. The invention is based, in part, on the unexpected findings by applicants that polymorphisms in several genes are highly correlated with the susceptibility of the subject to develop CAD.

The methods and compositions described herein can be used in determining the susceptibility to prognosis of various forms of coronary artery disease. Moreover, the methods and compositions of the present invention can also be used to facilitate the prevention of cardiovascular disease in an individuals found to be at an elevated risk for developing the disease.

One aspect of the invention relates to specific single nucleotide polymorphisms (SNPs) in the human genome, and their association with vascular disease and related pathologies, in particular, coronary artery disease (CAD) such as coronary stenosis. Based on differences in allele frequencies in the vascular disease patient population relative to normal individuals, the naturally-occurring SNPs disclosed herein can be used as targets for the design of diagnostic reagents and the development of therapeutic agents, as well as for disease association and linkage analysis. In particular, the SNPs of the present invention are useful for identifying an individual who is at an increased or decreased risk of developing vascular disease and for early detection of the disease, for providing clinically important information for the prevention and/or treatment of vascular disease, and for screening and selecting therapeutic agents. The SNPs disclosed herein are also useful for human identification applications. Methods, assays, kits, and reagents for detecting the presence of these polymorphisms and their encoded products are provided.

The present invention provides novel SNPs associated with coronary artery disease, as well as some SNPs that were previously known in the art, but were not previously known to be associated with coronary stenosis. Accordingly, the present invention provides novel compositions and methods based on the novel SNPs disclosed herein, and also provides novel methods of using the known, but previously unassociated, SNPs in methods relating to coronary stenosis (e.g., for diagnosing coronary stenosis, etc.).

One specific aspect of the invention provides methods of predicting the risk of developing CAD. One aspect of the invention provides a method of diagnosing premature CAD in an individual, including previously undiagnosed individuals or individuals without any type of cardiovascular disease. In one embodiment, the method comprises obtaining a DNA sample from the individual and determining the presence of one or more polymorphisms in at least one CAD-determinative gene. The presence of one or more polymorphisms is an indication that the individual is at high risk of developing a cardiovascular disease, such as CAD. Preferred polymorphisms are listed on Tables 1, 2 and 3. In one embodiment, the polymorphism is a polymorphism from Table 1 showing a p value of less than 0.05, 0.04, 0.03. 0.02. 0.01, 0.05, 0.02, 0.01, 0.005, 0.002 or 0.001. In some embodiments, the polymorphic change is at the same location along the genome as the polymorphisms found in Tables 1, 2 or 3. As an illustrative embodiment, if a given polymorphism in Table 1 consisted of a G to A nucleotide change at a given position on the genome, some embodiments would include screening for the change of G to C or G to T. Accordingly, in some embodiments, the presence of a polymorphism at the genomic position, regardless of the nature of the nucleotide change(s), indicates that the subject is at a higher risk of developing a cardiovascular disease. In one embodiment, the absence of the wild-type sequence in a polymorphic region is indicative of a higher likelihood of developing CAD.

The methods of the present invention may be used with a variety of contexts and maybe be used to assess the status of a variety of individuals. For example, the methods may be used to assess the status of individuals with no previous diagnosis of coronary artery disease, or with no significant cardiovascular risk factors. Cardiovascular risk factors include, but are not limited to, cholesterol, HDL cholesterol, systolic blood pressure, cigarette smoking, exercise, alcohol, race, obesity, family history of premature coronary artery disease, and medication use, including aspirin, statins, B-blockers and hormone replacement therapy in women.

Other indicia predictive of CAD can be detected or monitored in the subject in conjunction with the detection of polymorphisms in CAD-determinative genes. This may be useful to increase the predictive power of the methods described herein. Preferred indicia include the detection of additional CAD-determinative polymorphisms in genes not listed in Tables 1, 2 or 3, medical examination of the subject's cardiovascular system, and detection of gene products or other metabolites in a sample from a patient, such as a blood sample. In some embodiments, additional factors that may be monitored may be administration of pharmaceuticals known or suspected of having cardiovascular effects, such as increasing blood pressure, preferably in at least 5% or 10% of subjects who are administered the pharmaceuticals. In addition, the presence of cardiovascular risk factors, such as those listed in the preceding paragraph, may be also be weighed when assessing the risk of a subject for developing the cardiovascular disease.

### II. Definitions

A “coronary artery disease” (“CAD”) is a pathological state characterized by insufficiency of oxygen delivery to cardiac muscle, wherein the condition is associated with some dysfunction of coronary blood vessels. As used in this disclosure, CADs include both disorders in which symptomatic and/or asymptomatic cardiac ischemia occurs (e.g., angina pectoris and myocardial infarction) and disorders that gradually lead to chronic or acute cardiac ischemia, even at the stage of the disorder at which such ischemia is not yet evident (e.g., coronary arteriosclerosis and atherosclerosis).

An “increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

A “treatment plan” refers to at least one intervention undertaken to modify the effect of a risk factor upon a patient. A treatment plan for a cardiovascular disorder or disease can address those risk factors that pertain to cardiovascular disorders or diseases. A treatment plan can include an intervention that focuses on changing patient behavior, such as stopping smoking. A treatment plan can include an intervention whereby a therapeutic agent is administered to a patient. As examples, cholesterol levels can be lowered with proper medication, and diabetes can be controlled with insulin. Nicotine addiction can be treated by withdrawal medications. A treatment plan can include an intervention that is diagnostic. The presence of the risk factor of hypertension, for example, can give rise to a diagnostic intervention whereby the etiology of the hypertension is determined. After the reason for the hypertension is identified, further treatments may be administered.

The phrase “predicting the likelihood of developing” as used herein refers to methods by which the skilled artisan can predict onset of a cardiovascular condition in an individual. The term “predicting” does not refer to the ability to predict the outcome with 100% accuracy. Instead, the skilled artisan will understand that the term “predicting” refers to forecast of an increased or a decreased probability that a certain outcome will occur; that is, that an outcome is more likely to occur in an individual having one or more CAD-determinative polymorphisms.

A subject at higher risk of developing a cardiovascular disease refers to a subject having at least a 20%, 30%, 40%, 50%, 60%, 70%, 80%, 90%, 100%, 125%, 150%, 200%, 300%, 400%, 500%, 600%, 7000/, 800%, 900% or 1000% greater probability of developing the condition, relative to the general population. In one embodiment, the comparison is not to a general population but rather to a population matched by one or more factors such as age, sex, race, ethnicity, etc. In one embodiment, the population is one existing within a time frame of 5, 6, 7, 8, 9, 10, 15, 20, 25, 30, 35, 40, 45 or 50 years from the time of testing.

The term “polymorphism”, as used herein, refers to a difference in the nucleotide sequence of a given region, such as a region in a chromosome, as compared to a nucleotide sequence in a homologous region of another individual, in particular, a difference in the nucleotide of a given region which differs between individuals of the same species. A polymorphism is generally defined in relation to a reference sequence, usually referred to as the “wild-type” sequence. Polymorphisms include single nucleotide differences, differences in sequence of more than one nucleotide, and single or multiple nucleotide insertions, inversions and deletions. In certain embodiments, the polymorphism is within a non-coding region or in a translated region. In certain embodiments, the polymorphism is a silent polymorphism within a translated region. In some embodiments, the polymorphism results in an amino acid substitution. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Each version of the sequence with respect to the polymorphic site is referred to herein as an “allele” of the polymorphic site. Thus, in the previous example, the SNP allows for both an adenine allele and a thymine allele.

A “haplotype,” as described herein, refers to a combination of genetic markers (“alleles”), such as the SNPs set forth in Tables 1 and 2 and 3.

The nucleotide designation “R” refers to A or G nucleotides, while designation ‘N’ refers to G or A or T or C nucleotides, in accordance with IUPAC designations.

### III. CAD-Determinative Alleles and Polymorohisms

The present invention is based, at least in part, on the identification of alleles, in multiple genes, that are associated (to a statistically-significant extent) with the development of CAD in humans. Detection of these alleles in a subject indicates that the subject is predisposed to the development of a cardiovascular disease and in particular CAD. The identification of individuals predisposed to developing CAD, as identified using the methods described here, may prove useful in allowing the implementation of preventive treatment plans to delay or reduce the incidence of CAD.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position, reference is generally made to the protein-encoding strand, only for the purpose of convenience One aspect of the invention provides a method of estimating, or aiding in the estimation of, the risk of developing a cardiovascular disease, such as CAD, in a subject, the method comprising (i) providing a nucleic acid sample from the subject; (ii) detecting the presence of one or more single nucleotide polymorphisms (SNPs) in a CAD-determinative gene in the nucleic acid sample, wherein the presence of one or more SNPs reflects a higher risk of developing the cardiovascular disease. A related aspect of the invention provides a method of identifying a subject having an elevated risk of developing a cardiovascular disease, such as CAD, the method comprising (i) providing a nucleic acid sample from the subject; (ii) detecting the presence of one or more single nucleotide polymorphisms (SNPs) in a CAD-determinative gene in the genomic sample, wherein a subject having one or more SNPs is identified as a subject having an elevated risk of developing cardiovascular disease. To better characterize the subject's genetic content, occurrence of polymorphisms that are not associated with a disorder can also be assessed, so that one can determine whether the human is 1) homozygous for the CAD-determinative polymorphism at a genomic site, 2) heterozygous for a CAD-determinative and disorder-non-associated polymorphisms at the genomic site, or 3) homozygous for a CAD-non-associated polymorphisms at the site. In one embodiment, both the presence of a SNP polymorphism and of the wild-type sequence is determined.

Tables 1-5 provide a variety of information about SNPs of the present invention that are associated with coronary artery disease. Tables 4 (SEQ ID NOs:1-575) and Table 5 (SEQ ID NOs: 576-1050) disclose genomic SNP sequences. The sequences on Table 4 correspond to genomic sequences containing the SNP, while those on Table 5 have the corresponding genomic sequences without the SNP. Table 3 provides additional information for these sequences, including the chromosome position of the SNP, the gene locus in which the SNP is found, the Genbank accession number (which provides another way of naming the gene locus), a probe number and a genomic location within the chromosomes. Table 3 also provides the SEQ ID NOs for the SNP sequence and the nonSNP sequence for cross-reference with Tables 4-5.

In one embodiment, the CAD-determinative gene containing the SNP is one of the genes listed in Table 1. Table 1 includes the following genes: A1M1L, PLA2G7, OR7E29P, PLN, PTPN6, C1ORF38, GATA2, IL7R, MYLK, ANPEP, PIK3R4, RPLP2, OLR1, PNPLA2, TCF4, ACP5, SELP, BAX, CPNE4, TAL1, KLF15, ABCB1, LHFPL2, ITGAX, LOC389142, PLXNC1, SLA, ELL, NPY, IGSF11, ITPK1, ASB1, SELB, LOC131873, PCCA, HAPIP, PLAUR, SIDT1, RPN1, BPAG1, ROR2, MMP12, GAP43, FSTL1, MAP4, ZNF217, ALOX5, NPHP3, GPNMB, SPP1, ZNF80, MGP, C3ORF15, NEK11, POLQ, ADFP, UBXD1, 38413, FLJ46299, ZBTB20, HLA-DQA2, ZXDC, GRN, PSCD1, GYS1, C14ORF132, CD80, CDGAP, LMOD1, SLC41A3, HOXD1, STAT5A, OPRM1, ITPR2, HIF1A, PKD2, STEAP, AGTR1, NDUFB4, GLRA3, MEF2A, STXBP5L, APOBEC3D, FMNL1, PLXND1, ATP2C1, RUVBL1, CASR, PTPRR, SMPDL3A, APOD, APG3L, FLJ35880, TMCC1, CD96, C1QB, CTSD, FLI1, MMP9, TCIRG1, ITGB5, FLJ25414, NR1H3, HSPBAP1, APOC1, THPO, FTL, HADHSC, ALOX5AP, LAIR1, UPP1, LAPTM5, CSTA, ADCY5, PHLDB2, GM2A, NUDT16, ACSL1, VAMP5, ACP2, HLA-DPA1, TUBA3, MMP7, H41, NR112, FGFR2, OBA, CHAF1A, GSK3B, DOCK2, URB, HCLS1, CD200R1, SLCO2B1, B4GALT4, PLCXD2, FABP7, CAMKK2, FCGR1A, SELL, SELE, HNRPM, MGC45840, F5, SMTN, RAI3, HLA-DRA, CSTB, FLJ12592 and TAGLN3.

In one embodiment, the SNP is one of those listed in Tables 1-4. In another embodiment, the SNP is one that is highly-statistically associated (p<0.1, p<0.05 or p<0.01) with the development of CAD. In another embodiment, the SNP is a SNP in linkage disequilibrium with one of the aforementioned SNPs. The third and fourth columns in Table 1 indicate the chromosome and the location within chromosome where the polymorphism in located.

In one embodiment, the method of estimating the risk of developing coronary artery disease (CAD) in a subject comprises determining the presence of more than one SNP from Tables 1-4 in the genomic sample from the subject, which may be from one gene of from two or more genes.

In addition to the SNPs described in Tables 1-4, one of skill in the art can readily identify other alleles (including polymorphisms and mutations) that are in linkage disequilibrium with one of the SNPs described herein. For example, a nucleic acid sample from a first group of subjects without CAD can be collected, as well as DNA from a second group of subjects with CAD. The nucleic acid sample can then be compared to identify those alleles that are over-represented in the second group as compared with the first group, wherein such alleles are presumably associated with CAD. Alternatively, alleles that are in linkage disequilibrium with a CAD associated-allele can be identified, for example, by genotyping a large population and performing statistical analysis to determine which alleles appear more commonly together than expected.

Preferably the group is chosen to be comprised of genetically-related individuals. Genetically-related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Appropriate probes may be designed to hybridize to one of the alleles listed in Tables 1-3. Alternatively, these probes may incorporate other regions of the relevant genomic locus, including intergenic sequences. Yet other polymorphisms available for use with the immediate invention are obtainable from various public sources. For example, the human genome database collects intragenic SNPs, is searchable by sequence (http://hgbase.interactiva.de). Also available is a human polymorphism database maintained by NCBI (http://www.ncbi.nim.nih.gov/projects/SNP/). From such sources SNPs as well as other human polymorphisms may be found.

### IV. Detection of CAD-Determinative Polymorphisms

Many methods are available for detecting specific alleles at human polymorphic loci. The preferred method for detecting a specific polymorphic allele will depend, in part, upon the molecular nature of the polymorphism. SNPs are most frequently biallelic-occurring in only two different forms (although up to four different forms of an SNP, corresponding to the four different nucleotide bases occurring in DNA, are theoretically possible). Because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Any cell type or tissue may be utilized to obtain nucleic acid samples for use in the diagnostics described herein. In a preferred embodiment, the DNA sample is obtained from a bodily fluid, e.g., blood, obtained by known techniques (e.g. venipuncture), or saliva. Alternatively, nucleic acid tests can be performed on dry samples (e.g. hair or skin). When using RNA or protein, the cells or tissues that may be utilized must express a CAD-determinative gene. In one embodiment, biological samples such as blood, bone, hair, saliva, or semen may be used.

### Exonuclease-Resistant Nucleotide

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

### Solution-Based Method

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

### Genetic Bit Analysis

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

### Primer-Guided Nucleotide Incorporation

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

### Protein Truncation Test (PTT)

For SNPs that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:14). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

### In Situ Tissue Sections

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

### Allele-Specific Hybridization

In one preferred detection method is allele specific hybridization using probes overlapping a region of at least one allele of a CAD-determinative gene having about 5, 10, 20, 25, or 30 nucleotides around the mutation or polymorphic region. In one embodiment of the invention, several probes capable of hybridizing specifically to other allelic variants involved in CAD are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a CAD-determinative gene. The solid phase support is then contacted with a test nucleic acid and hybridization to the specific probes is detected. Accordingly, the identity of numerous allelic variants of one or more genes can be identified in a simple hybridization experiment. The design and use of allele-specific probes for analyzing polymorphisms is known in the art (see, e.g., Dattagupta, EP 235,726, Saiki, WO 89/11548). WO 95/11995 describes subarrays that are optimized for detection of variant forms of a pre-characterized polymorphism.

### DNA-Amplification and PCR-Based Methods

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self-sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197). PCR-based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers. Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

A merely illustrative embodiment of a method using PCR-amplification includes the steps of (i) collecting a sample of cells from a subject, (ii) isolating nucleic acid (e.g., genomic, mRNA or both) from the cells of the sample, (iii) contacting the nucleic acid sample with one or more primers which specifically hybridize 5′ and 3′ to at least one CAD-determinative gene under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an CAD-determinative gene is identified by alterations in restriction enzyme cleavage patterns. For example, sample and control DNA is isolated, amplified (optionally), digested with one or more restriction endonucleases, and fragment length sizes are determined by gel electrophoresis.

Alternatively, allele-specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; WO 93/22456). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### Nucleic Acid Sequencing

In yet another embodiment, any of a variety of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci. USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

### Mismatch Cleavage

In a further embodiment, protection from cleavage agents (such as a nuclease, hydroxylamine or osmium tetraoxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of a CAD-determinative gene locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

### Mobility of Nucleic Acids

In other embodiments, alterations in electrophoretic mobility will be used to identify a CAD-determinative allele. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control CAD-terminative alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5). In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

### Oligonucleotide Ligation Assay

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an CAD-determinative haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci. USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA. Other methods of detecting polymorphisms, e.g., SNPs, are known, e.g., as described in U.S. Pat. Nos. 6,410,231; 6,361,947; 6,322,980; 6,316,196; 6,258,539; and U.S. Publication Nos. 2004/0137464 and 2004/0072156.

### V. Subjects

The subjects to be tested for characterizing its risk of CAD in the foregoing methods may be any human or other animal, preferably a mammal. In certain embodiments, the subject does not otherwise have an elevated risk of cardiovascular disease according to the traditional risk factors. Subjects having an elevated risk of cardiovascular disease include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.—hereinafter “Harrison's”).

In certain embodiments the subject is an apparently healthy nonsmoker. “Apparently healthy”, as used herein, means individuals who have not previously being diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. Apparently healthy individuals also do not otherwise exhibit symptoms of disease. In other words, such individuals, if examined by a medical professional, would be characterized as healthy and free of symptoms of disease. “Nonsmoker” means an individual who, at the time of the evaluation, is not a smoker. This includes individuals who have never smoked as well as individuals who in the past have smoked but presently no longer smoke.

In certain embodiments, the test subjects are apparently healthy subjects otherwise free of current need for treatment for a cardiovascular disease. In some embodiments, the subject is otherwise free of symptoms calling for treatment with any one of any combination of or all of the foregoing categories of agents. For example, with respect to anti-inflammatory agents, the subject is free of symptoms of rheumatoid arthritis, chronic back pain, autoimmune diseases, vascular diseases, viral diseases, malignancies, and the like. In another embodiment, the subject is not at an elevated risk of an adverse cardiovascular event (e.g., subject with no family history of such events, subjects who are nonsmokers, subjects who are nonhyperlipidemic, subjects who do not have elevated levels of a systemic inflammatory marker), other than having an elevated level of one or more oxidized apoA-I related biomolecules.

In some embodiments, the subject is a nonhyperlipidemic subject. A “nonhyperlipidemic” is a subject that is a nonhypercholesterolemic and/or a nonhypertriglyceridemic subject. A “nonhypercholesterolemic” subject is one that does not fit the current criteria established for a hypercholesterolemic subject. A nonhypertriglyceridemic subject is one that does not fit the current criteria established for a hypertriglyceridemic subject (See, e.g., Harrison's Principles of Experimental Medicine, 15th Edition, McGraw-Hill, Inc., N.Y.—hereinafter “Harrison's”). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL (<35 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TO) level of >250 mg/dL. Thus, a nonhyperlipidemic subject is defined as one whose cholesterol and triglyceride levels are below the limits set as described above for both the hypercholesterolemic and hypertriglyceridemic subjects.

### VI. Pharmacogenomics

Knowledge of CAD-determinative alleles, such as those described in Tables 1-4, alone or in conjunction with information on other genetic defects contributing to CAD, al lows customization of a therapy to the individual's genetic profile. For example, subjects having an CAD-determinative allele of AIM1L, PLA2G7, OR7E29P, PLN, PTPN6, C1ORF38, GATA2, IL7R or MYLK, or any polymorphic nucleic acid sequence in linkage disequilibrium with any of these alleles, may be predisposed to developing CAD and may respond better to particular therapeutics that address the particular molecular basis of the disease in the subject. Thus, comparison of an individual's CAD-determinative allele profile to the population profile for CAD, permits the selection or design of drugs or other therapeutic regimens that are expected to be safe and efficacious for a particular subject or subject population (i.e., a group of subjects having the same genetic alteration).

In addition, the ability to target populations expected to show the highest clinical benefit, based on genetic profile can enable: 1) the repositioning of marketed drugs with disappointing market results; 2) the rescue of drug candidates whose clinical development has been discontinued as a result of safety or efficacy limitations, which are subject subgroup-specific; and 3) an accelerated and less costly development for drug candidates and more optimal drug labeling (e.g. since measuring the effect of various doses of an agent on a CAD causative mutation is useful for optimizing effective dose).

The treatment of an individual with a particular therapeutic can be monitored by determining protein, mRNA and/or transcriptional level of a CAD-determinative gene. Depending on the level detected, the therapeutic regimen can then be maintained or adjusted (increased or decreased in dose). In a preferred embodiment, the effectiveness of treating a subject with an agent comprises the steps of: (i) obtaining a preadministration sample from a subject prior to administration of the agent; (ii) detecting the level or amount of a protein, mRNA or genomic DNA in the preadministration sample of a CAD-determinative gene; (iii) obtaining one or more post-administration samples from the subject; (iv) detecting the level of expression or activity of the protein, mRNA or genomic DNA in the post-administration sample of the CAD-determinative gene; (v) comparing the level of expression or activity of the protein, mRNA or genomic DNA of the CAD-determinative gene in the preadministration sample with the corresponding one in the postadministration sample, respectively; and (vi) altering the administration of the agent to the subject accordingly.

Cells of a subject may also be obtained before and after administration of a therapeutic to detect the level of expression of genes other than an CAD-determinative gene to verify that the therapeutic does not increase or decrease the expression of genes which could be deleterious. This can be done, e.g., by using the method of transcriptional profiling. Thus, mRNA from cells exposed in vivo to a therapeutic and mRNA from the same type of cells that were not exposed to the therapeutic could be reverse transcribed and hybridized to a chip containing DNA from numerous genes, to thereby compare the expression of genes in cells treated and not treated with the therapeutic.

In still another aspect, the invention relates to a method of selecting a dose of a cardiovascular protective agent for administration to a subject. The method comprises assessing occurrence in the human's genome of a CAD-determinative allele. Occurrence of any of the polymorphisms is an indication that a greater dose of the agent should be administered to the human. The dose of the agent can be selected based on occurrence of the polymorphisms. A greater number of CAD-determinative polymorphisms indicates a greater dosage.

### VII. Additional Diagnostic/Predictive Markers

In certain embodiments, assessment of one or more markers are combined to increase the predictive value of the analysis in comparison to that obtained from the identification of polymorphisms in CAD-determinative allele(s) alone. Such markers may be assessed, for example, by detecting genetic changes in the genes (e.g. mutations or polymorphisms) or by detecting the level of gene products, metabolites or other molecules level in a biological sample obtained from the subject, such as a serum or blood sample. In one embodiment, the levels of one or more markers for myocardial injury, coagulation, or atherosclerotic plaque rupture are measured from a sample from the subject to increase the predictive value of the described methods

In one embodiment, assessment of one or more additional markers indicative of atherosclerotic plaque rupture is combined with detection of polymorphism(s) in CAD-determinative gene(s). Markers of atherosclerotic plaque rupture that may be useful include human neutrophil elastase, inducible nitric oxide synthase, lysophosphatidic acid, malondialdehyde-modified low-density lipoprotein, matrix metalloproteinase-1, matrix metalloproteinase-2, matrix metalloproteinase-3, and matrix metalloproteinase-9. In one embodiment, assessment of one or more additional markers indicative of coagulation is combined with detection of polymorphism(s) in CAD-determinative gene(s). Coagulation markers include β-thromboglobulin, D-dimer, fibrinopeptide A, platelet-derived growth factor, plasmin-α-2-anti-plasmin complex, platelet factor 4, prothrombin fragment 1+2, P-selectin, thrombin-antithrombin III complex, thrombus precursor protein, tissue factor and von Willebrand factor.

In one embodiment, the marker(s) that may be tested in conjunction with the detection of polymorphism(s) in CAD-determinative gene(s) includes soluble tumor necrosis factor-α receptor-2, interleukin-6, lipoprotein-associated phospholipase A2, C-reactive protein (CRP), Creatine Kinase with Muscle and/or Brain subunits (CKMB), thrombin anti-thrombin (TAT), soluble fibrin monomer (SFM), fibrin peptide A (FPA), myoglobin, thrombin precursor protein (TPP), platelet monocyte aggregate (PMA) troponin and homocysteine. In another embodiment, the additional markers can be Annexin V, B-type natriuretic peptide (BNP) which is also called brain-type natriuretic peptide, enolase, Troponin I (TnI), cardiac-troponin T, Creatine kinase (CK), Glycogen phosphorylase (GP), Heart-type fatty acid binding protein (H-FABP), Phosphoglyceric acid mutase (PGAM) and S-100.

In embodiments where one or more markers are used in combination with detection of polymorphism(s) in CAD-determinative gene(s) to increase the predictive value of the analysis, the patient sample from which the level of the additional marker(s) is to be measured may be the same or different from one used to detect polymorphism(s) in CAD-determinative gene(s). In one embodiment, the biological sample from which the level of additional marker is determined is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions, employing one of a variety of buffers, such as phosphate, Tris, or the like, at physiological pH can be used.

In certain embodiments, the subject's risk profile for CAD is determined by combining a first risk value, which is obtained by determining the presence of one or more CAD-determinative polymorphisms, with one or more additional risk values to provide a final risk value. Such additional risk values may be obtained by procedures including, but not limited to, determining the subject's blood pressure, assessing the subject's response to a stress test, determining levels of myeloperoxidase, C-reactive protein, low density lipoprotein, or cholesterol in a bodily sample from the subject, or assessing the subject's atherosclerotic plaque burden.

In some embodiments, genetic variations in additional marker genes are combined with detection of polymorphism(s) in a gene not listed in Tables 1 or 2. In specific embodiments, the additional marker gene is selected from apolipoprotein B, apolipoprotein E, paraoxonase 1, type I angiotensin II receptor, cytochrome b-245(alpha), prothrombin, coagulation factor VII, platelet glycoprotein 1b alpha, platelet glycoprotein IIIa, endothelial nitric oxide synthase, 5,10-methylene tetrahydrofolate reductase, angiotensinogen, plasminogen activator inhibitor 1, coagulation factor V, alpha adducin I, cytochrome P450, G-protein beta, polypeptide 3, methionine synthase reductase, endothelial adhesion molecule 1 and cholesteryl ester transferase. Polymorphisms in these genes are described, for example, in U.S. Patent Publication No. 2004/0005566.

In one embodiment, the methods to assess the test subject's risk of developing CAD comprise performing a medical examination of the subject's cardiovascular systems. Such examinations may be useful to increase the predictive power of the methods. Types of medical examinations include, for example, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA).

### VIII. Nucleic Acids

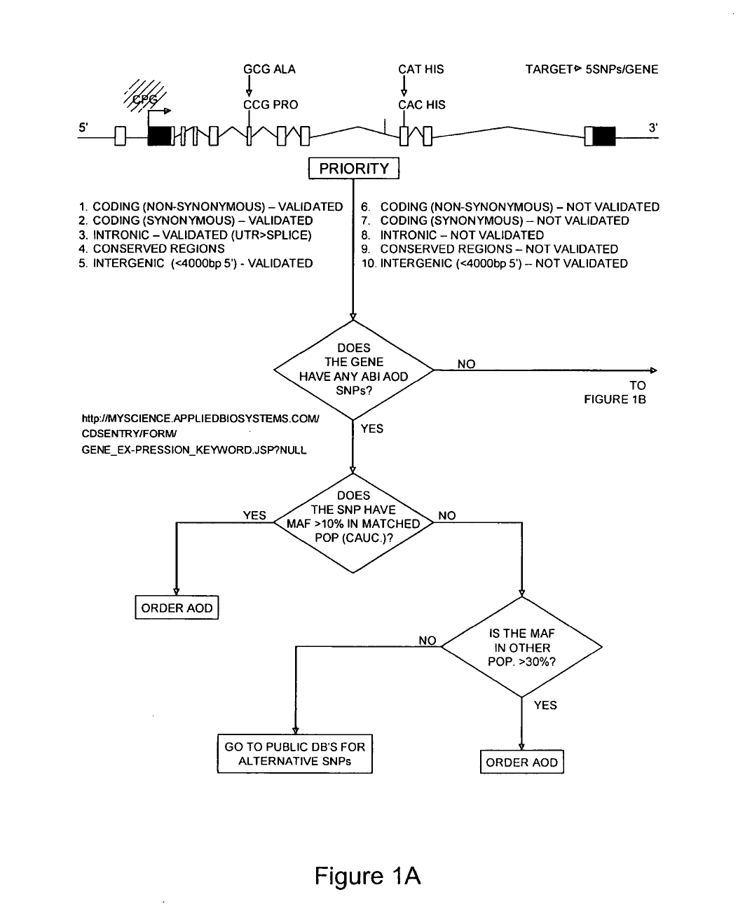

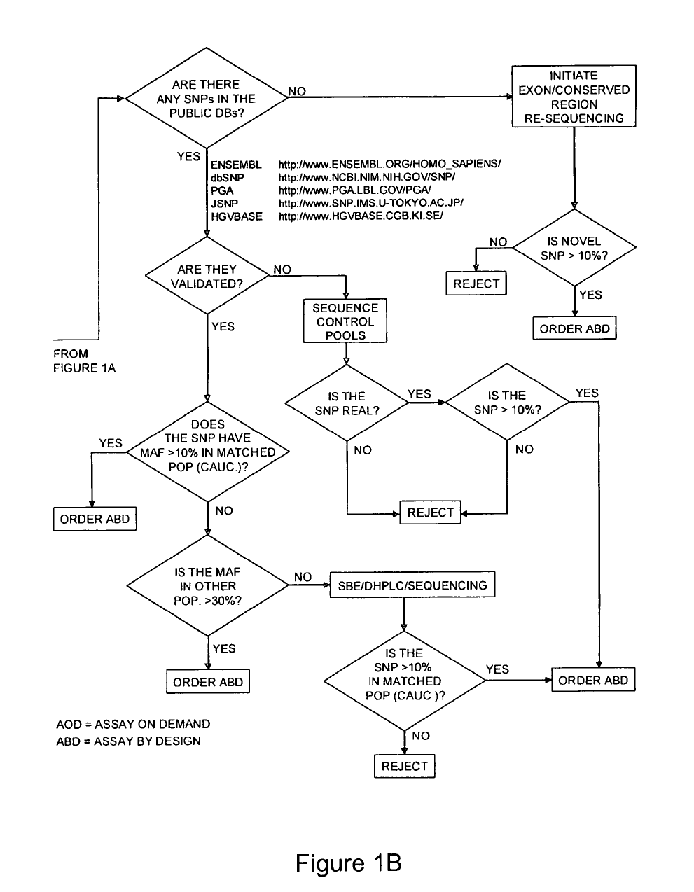

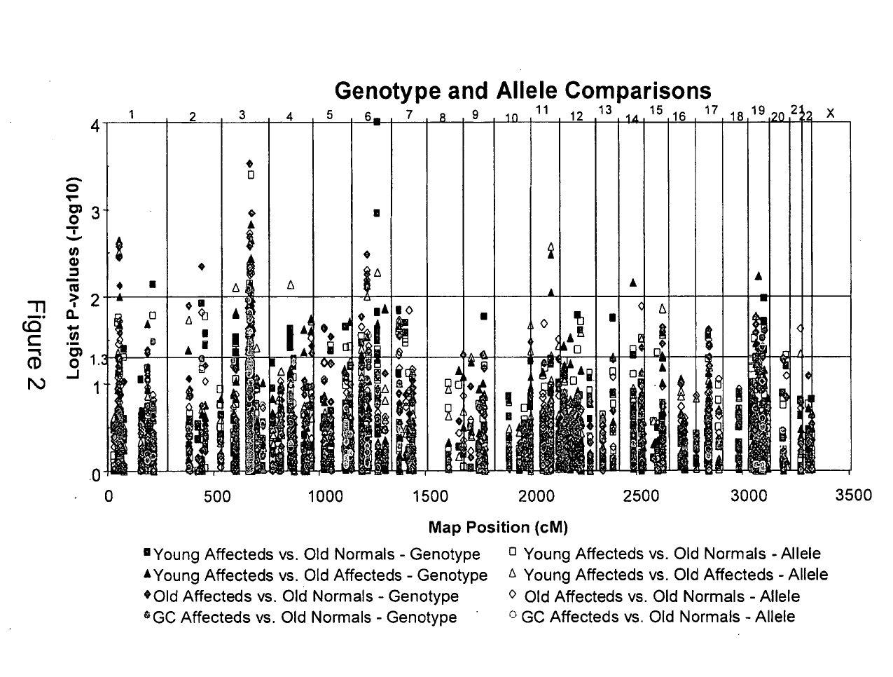

The present invention provides isolated polynucleotides comprising one or more CAD-determinative polymorphic nucleic acid sequences. In some embodiments, the polymorphism is one that is described in FIG. 1 or Tables 1-5. The isolated polynucleotides are useful in a variety of diagnostic methods. Isolated polymorphic nucleic acid molecules of the invention can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

An isolated polymorphic nucleic acid molecule comprises one or more polymorphisms listed in Tables 1-5. Preferred polymorphism are those found in any one of the following genes: A1M1L, PLA2G7, OR7E29P, PLN, PTPN6, C1ORF38, GATA2, IL7R, MYLK, ANPEP, PIK3R4, RPLP2, OLR1, PNPLA2, TCF4, ACP5, SELP, BAX, CPNE4, TALI, KLF15, ABCB1, LHFPL2, ITGAX, LOC389142, PLXNC1, SLA, ELL, NPY, IGSF11, ITPK1, ASB1, SELB, LOC131873, PCCA, HAPIP, PLAUR, SIDT1, RPN1, BPAG1, ROR2, MMP12, GAP43, FSTL1, MAP4, ZNF217, ALOX5, NPHP3, GPNMB, SPP1, ZNF80, MGP, C3ORF15, NEK11, POLQ, ADFP, UBXD1, 38413, FLJ46299, ZBTB20, HLA-DQA2, ZXDC, GRN, PSCD1, GYS1, C14ORF132, CD80, CDGAP, LMOD1, SLC41A3, HOXD1, STAT5A, OPRM1, 1TPR2, HIF1A, PKD2, STEAP, AGTR1, NDUFB4, GLRA3, MEF2A, STXBP5L, APOBEC3D, FMNL1, PLXND1, ATP2Cl, RUVBL1, CASR, PTPRR, SMPDL3A, APOD, APG3L, FLJ35880, TMCC1, CD96, C1QB, CTSD, FLI1, MMP9, TCIRG1, ITGB5, FLJ25414, NR1H3, HSPBAP1, APOC1, THPO, FTL, HADHSC, ALOX5AP, LAIR1, UPP1, LAPTM5, CSTA, ADCY5, PHLDB2, GM2A, NUDT16, ACSL1, VAMP5, ACP2, HLA-DPA1, TUBA3, MMP7, H41, NR112, FGFR2, GBA, CHAF1A, GSK3B, DOCK2, URB, HCLS1, CD200R1, SLCO2B1, B4GALT4, PLCXD2, FABP7, CAMKK2, FCGR1A, SELL, SELE, HNRPM, MGC45840, F5, SMTN, RAI3, HLA-DRA, CSTB, FLJ2592 and TAGLN3.

In a preferred embodiment, the polymorphism is from A1MIL, PLA2G7, OR7E29P, PLN, PTPN6, C1ORF38, GATA2, IL7R or MYLK. For some uses, e.g., in screening assays, CAD-determinative polymorphic nucleic acid molecules will be of at least about 15 nucleotides (nt), at least about 18 nt, at least about 20 nt, or at least about 25 nt in length, and often at least about 50 nt. Such small DNA fragments are useful as primers for polymerase chain reaction (PCR), hybridization screening, etc. Larger polynucleotide fragments, e.g., at least about 50 nt, at least about 100 nt, at least about 200 nt, at least about 300 nt, at least about 500 nt, at least about 1000 nt, at least about 1500 nt, up to the entire coding region, or up to the entire coding region plus up to about 1000 nt 5′ and/or up to about 1000 nt 3′ flanking sequences from a CAD-determinative gene, are useful for production of the encoded polypeptide, promoter motifs, etc. For use in amplification reactions, such as PCR, a pair of primers will be used. The exact composition of primer sequences is not critical to the invention, but for most applications the primers will hybridize to the subject sequence under stringent conditions, as known in the art.

The present invention also provides isolated nucleic acid molecules that contain one or more SNPs disclosed in Tables 1-4, and in preferred embodiments from Table 4. Preferred isolated nucleic acid molecules contain one or more SNPs identified in Tables 1-1. Isolated nucleic acid molecules containing one or more SNPs disclosed in at least one of Tables 1-4 may be interchangeably referred to throughout the present text as “SNP-containing nucleic, acid molecules.” Isolated nucleic acid molecules may optionally encode a full-length variant protein or fragment thereof. The isolated nucleic acid molecules of the present invention also include probes and primers, which may be used for assaying the disclosed SNPs, and isolated full-length genes, transcripts cDNA molecules, and fragments thereof, which may be used for such purposes as expressing an encoded protein.

As used herein, an “isolated nucleic acid molecule” generally is one that contains a SNP of the present invention or a complement thereof and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated”. Examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. Preferably the flanking sequence is up to about 500, 300, 100, 60, 50, 30, 25, 20, 15, 10, 8, or 4 nucleotides (or any other length in-between) on either side of a SNP position, or as long as the full-length gene or entire protein-coding sequence (or any portion thereof such as an exon), especially if the SNP-containing nucleic acid molecule is to be used to produce a protein or protein fragment.

Table 4 shows SNP-containing nucleic acid molecules having 20 nucleotides flanking the SNP site. In one embodiment, the invention provides an isolated SNP-containing nucleic acid molecule comprises the nucleotide sequence of any one of SEQ ID NOs: 1-575. In another embodiment, the SNP-containing nucleic acid molecule provided by the invention comprises a nucleotide sequence identical to 10, 11, 12, 13, 14, 15, 16, 17, 18, 19, 20, 21, 22, 23, 24, 25, 26, 27, 27, 28, 29, 30, 31, 32, 34, 35, 36, 37, 38, 39, or 40 contiguous nucleotides of any one of SEQ ID NOs: 1-575. In another embodiment, the SNP-containing nucleic acid molecule provided by the invention comprises a nucleotide sequence identical to 10, 11, 12, 13, 14, 15, 16, 17, 18, 19, 20, 21, 22, 23, 24, 25, 26, 27, 27, 28, 29, 30, 31, 32, 34, 35, 36, 37, 38, 39, or 40 contiguous nucleotides of any one of SEQ ID NOs: 1-575 wherein the contiguous nucleotides contain the SNP site (shown in brackets, i.e. “[ ]” in Table 4).

For full-length genes and entire protein-coding sequences, a SNP flanking sequence can be, for example, up to about 5 Kb, 4 Kb, 3 Kb, 2 Kb, 1 Kb on either side of the SNP. Furthermore, in such instances, the isolated nucleic acid molecule comprises exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long as it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In a preferred embodiment of the invention, an amplified polynucleotide is at least about 30 nucleotides in length. In a more preferred embodiment of the invention, an amplified polynucleotide is at least about 32, 40, 45, 50, or, 60 nucleotides in length. In yet another preferred embodiment of the invention, an amplified polynucleotide is at least about 100, 200, 300, 400, or 500 nucleotides in length. While the total length of an amplified polynucleotide of the invention can be as long as an exon, an intron or the entire gene where the SNP of interest resides, an amplified product is typically up to about 1,000 nucleotides in length (although certain amplification methods may generate amplified products greater than 1000 nucleotides in length). More preferably, an amplified polynucleotide is not greater than about 600-700 nucleotides in length. It is understood that irrespective of the length of an amplified polynucleotide, a SNP of interest may be located anywhere along its sequence.

In a specific embodiment of the invention, the amplified product is at least about 21 nucleotides in length, comprises one of the transcript-based context sequences or the genomic-based context sequences shown in Tables 1-4. Such a product may have additional sequences on its 5′ end or 3′ end or both. In another embodiment, the amplified product is about 21 nucleotides in length, and it contains a SNP disclosed herein. Preferably, the SNP is located at the middle of the amplified product (e.g., at position 11 in an amplified product that is 21 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product, (however, as indicated above, the SNP of interest may be located anywhere along the length of the amplified product).

The present invention provides isolated nucleic acid molecules that comprise, consist of, or consist essentially of one or more polynucleotide sequences that contain one or more SNPs disclosed herein, complements thereof, and SNP-containing fragments thereof.

The isolated nucleic acid molecules can encode mature proteins plus additional amino or carboxyl-terminal amino acids or both, or amino acids interior to the mature peptide (when the mature form has more than one peptide chain, for instance). Such sequences may play a role in processing of a protein from precursor to a mature form, facilitate protein trafficking, prolong or shorten protein half-life, or facilitate manipulation of a protein for assay or production. As generally is the case in situ, the additional amino acids may be processed away from the mature protein by cellular enzymes.

Thus, the isolated nucleic acid molecules include, but are not limited to, nucleic acid molecules having a sequence encoding a peptide alone, a sequence encoding a mature peptide and additional coding sequences such as a leader or secretory sequence (e.g., a pre-pro or pro-protein sequence), a sequence encoding a mature peptide with or without additional coding sequences, plus additional non-coding sequences, for example introns and non-coding 5′ and 3′ sequences such as transcribed but untranslated sequences that play a role in, for example, transcription, mRNA processing (including splicing and polyadenylation signals), ribosome binding, and/or stability of mRNA. In addition, the nucleic acid molecules may be fused to heterologous marker sequences encoding, for example, a peptide that facilitates purification.

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof: (Sambrook and Russell, 2000, Molecular Cloning: A Laboratory Manual, Cold Spring Harbor Press, NY). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding; from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well-known in the art (see, e.g., Corey, “Peptide nucleic acids: expanding the scope of nucleic acid recognition”, Trends Biotechnol. June 1997; 15(6):224-9, and Hyrup et al., “Peptide nucleic acids (PNA): synthesis, properties and potential applications”, Bioorg Med. Chem. January 1996; 4(1):5-23). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in Tables 1-4. Furthermore, kits/systems (such as beads, arrays, etc.) that include these analogs are also encompassed by the present invention. For example, PNA oligomers that are based on the polymorphic sequences of the present invention are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994), Petersen et al., Bioorganic & Medicinal Chemistry Letters, 6: 793-796 (1996), Kumar et al., Organic Letters 3(9): 1269-1272:(2001), WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

Additional examples of nucleic acid modifications that improve the binding properties and/or stability of a nucleic acid include the use of base analogs such as U.S. Pat. No. 5,801,115). Thus, references herein to nucleic acid molecules, SNP-containing nucleic acid molecules, SNP detection reagents (e.g., probes and primers), oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

The present invention further provides nucleic acid molecules that encode fragments of the variant polypeptides disclosed herein as well as nucleic acid molecules that encode obvious variants of such variant polypeptides. Such nucleic acid molecules may be naturally occurring, such as paralogs (different locus) and orthologs (different organism), or may be constructed by recombinant DNA methods or by chemical synthesis. Non-naturally occurring variants may be made by mutagenesis techniques, including those applied to nucleic acid molecules, cells, or organisms. Accordingly, the variants can contain nucleotide substitutions, deletions, inversions and insertions (in addition to the SNPs disclosed in Tables 1-4). Variation can occur in either or both the coding and non-coding regions. The variations can produce conservative and/or non-conservative amino acid substitutions.

The nucleic acid molecules of the invention may be used as probes. When used as a probe, an isolated polymorphic CAD-determinative nucleic acid molecule may comprise non-CAD-determinative nucleotide sequences, as long as the additional non-CAD-determinative nucleotide sequences do not interfere with the detection assay. A probe may comprise an isolated polymorphic CAD-determinative sequence, and any number of non-CAD-determinative nucleotide sequences, e.g., from about 1 bp to about 1 kb or more.

For screening purposes, hybridization probes of the polymorphic sequences may be used where both forms are present, either in separate reactions, spatially separated on a solid phase matrix, or labeled such that they can be distinguished from each other. Assays (described below) may utilize nucleic acids that hybridize to one or more of the described polymorphisms. Isolated polymorphic CAD-determinative nucleic acid molecules of the invention may be coupled (e.g., chemically conjugated), directly or indirectly (e.g., through a linker molecule) to a solid substrate. Solid substrates may be any known in the art including, but not limited to, beads, e.g., polystyrene beads; chips, e.g., glass, SiO₂, and the like; plastic surfaces, e.g., polystyrene, polycarbonate plastic multi-well plates; and the like.

Additional CAD-determinative gene polymorphisms may be identified using any of a variety of methods known in the art, including, but not limited to SSCP, denaturing HPLC, and sequencing. SSCP may be used to identify additional CAD-determinative gene polymorphisms. In general, PCR primers and restriction enzymes are chosen so as to generate products in a size range of from about 25 bp to about 500 bp, or from about 100 bp to about 250 bp, or any intermediate or overlapping range therein.

### IX. Kits

The invention further relates to a kit for assessing relative susceptibility of a human to developing CAD. The kit comprises reagents for assessing occurrence in the human's genome of a CAD-determinative polymorphism in at least one, two, three, four or five or more of the CAD-determinative genes. Another aspect of the invention provides kits for detecting a predisposition for developing a CAD.

The kits may contain one or more oligonucleotides, including 5′ and 3′ oligonucleotides that hybridize 5′ and 3′ to at least one allele of a CAD-determinative locus haplotype, such as to any of the SNPs listed in Tables 1 and 2. PCR-amplification oligonucleotides should hybridize between 25 and 2500 base pairs apart, preferably between about 100 and about 500 bases apart, in order to produce a PCR product of convenient size for subsequent analysis.

The design of oligonucleotides for use in the amplification and detection of CAD-determinative polymorphic alleles by the method of the invention is facilitated by the availability of public genomic data for the CAD-determinative genes. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dematol. 103:387-389 (1994)) and the like; DNA purification reagents such as Nucleon™ kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood.

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

One aspect of the invention provides DNA microarrays containing one or more SNP nucleic acid molecules. In one embodiment, the microarray includes 1, 2, 3, 4, 5 or more polymorphic CAD-determinative nucleic acid molecules e.g., probes or primers described herein, that are capable of detecting (e.g., hybridizing to) a polymorphic CAD-determinative nucleic acid molecules. Isolated polymorphic CAD-determinative nucleic acid molecules can be obtained by chemical or biochemical synthesis, by recombinant DNA techniques, or by isolating the nucleic acids from a biological source, or a combination of any of the foregoing. For example, the nucleic acid may be synthesized using solid phase synthesis techniques, as are known in the art. Oligonucleotide synthesis is also described in Edge et al. (1981) Nature 292:756; Duckworth et al. (1981) Nucleic Acids Res. 9:1691 and Beaucage and Caruthers (1981) Tet. Letters 22:1859. Following preparation of the nucleic acid, the nucleic acid is then ligated to other members of the expression system to produce an expression cassette or system comprising a nucleic acid encoding the subject product in operational combination with transcriptional initiation and termination regions, which provide for expression of the nucleic acid into the subject polypeptide products under suitable conditions.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting at least 1; 10; 100; 1000; 10,000; 100,000 (or any other number in-between) or substantially all of the SNPs shown in Tables 1-5.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002; 8:85-101; Sosnowski et al., “Active microelectronic array system for DNA hybridization, genotyping and pharmacogenomic applications”, Psychiatr Genet. December 2002; 12(4): 181-92; Heller, “DNA microarray technology: devices, systems, and applications”; Annu Rev Biomed Eng. 2002; 4: 129-53. Epub Mar. 22, 2002; Kolchirisky et al., “Analysis of SNPs and other genomic variations using gel-based chips”, Hum Mutat. April 2002; 19(4):343-60; and McGall et al., “High-density genechip oligonucleotide probe arrays”, Adv Biochem Eng Biotechnol. 2002; 77:21-42.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a tight-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70-nucleotides in length, more preferably about 5565 nucleotides in length, and most preferably about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular areas along the length of a target gene/transcript sequence, particularly areas corresponding to one or more SNPs disclosed in Table 1 and/or Table 2. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. Published application US2002/0110828 discloses methods and compositions for microarray controls.

In one embodiment of the invention, a nucleic acid array can comprise an array of probes of about 15-25 nucleotides in length. In further embodiments, a nucleic acid array can comprise any number of probes, in which at least one probe is capable of detecting one or more SNPs disclosed in Tables 1-4, and/or at least one probe comprises a fragment of one of the sequences selected from the group consisting of those disclosed in Table 1-4, the Sequence Listing, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a novel SNP allele disclosed in Table 1-4. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other, number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM™ 6700 sample preparation system, and Roche Molecular Systems' COBAS AmpliPrep System.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. Feb. 24, 2003; 55(3):349-77). In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. Nos. 6,153,073, Dubrow et al., and U.S. Pat. No. 6,156,181, Parce et al.

For genotyping SNPs, an exemplary microfluidic system may integrate, for example, nucleic acid amplification, primer extension, capillary electrophoresis, and a detection method such as laser induced fluorescence detection. In a first step of an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

### X. Therapeutic Methods

In another aspect, the invention features methods of treating a subject, e.g., a human, at risk of developing a cardiovascular disease, such as coronary artery disease (CAD). The methods include: identifying a subject having, or at risk of developing, CAD, and administering to the subject an agent that decreases CAD-determinative gene signaling (e.g., decreases CAD-determinative gene expression, levels or activity).

The present invention also relates to methods of treating a subject to reduce the risk of developing CAD or a complication from CAD. In one embodiment, the method comprises determining the presence of one or more CAD-determinative polymorphisms in the subject, and for subjects with one, two, three, four, five or more such polymorphisms, administering an agent expected to reduce the onset of cardiovascular disease. In one embodiment, the agent is selected from an anti-inflammatory agent, an antithrombotic agent, an anti-platelet agent, a fibrinolytic agent, a lipid reducing agent, a direct thrombin inhibitor, a glycoprotein Ilb/IIIa receptor inhibitor, a calcium channel blocker, a beta-adrenergic receptor blocker, a cyclooxygenase-2 inhibitor, an angiotensin system inhibitor, and/or combinations thereof. The agent is administered in an amount effective to lower the risk of the subject developing a the cardiovascular disease.

Anti-inflammatory agents include but are not limited to, Aldlofenac; Aldlometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate, Cornethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include but are not limited to, Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant); rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include but are not limited to, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide.

Lipid-reducing agents include but are not limited to, gemfibrozil, cholystyramine, colestipol, nicotinic acid, probucol lovastatin, fluvastatin, simvastatin, atorvastatin, pravastatin, cerivastatin, and other HMG-CoA reductase inhibitors.

Direct thrombin inhibitors include but are not limited to, hirudin, hirugen, hirulog, agatroban, PPACK, thrombin aptamers.

Glycoprotein IIb/IIIa receptor inhibitors are both antibodies and non-antibodies, and include but are not limited to ReoPro (abcixamab), lamifiban, tirofiban.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogenous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, anrinone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, beftunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hyd-roxypropoxy)-3-pyridenecarbonitrilHCl, 1-butylamino-3-(2,5-dichlorophenoxy-)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

Suitable COX-2 inhibitors include, but are not limited to, COX-2 inhibitors described in U.S. Pat. No. 5,474,995 Phenyl heterocycles as cox-2 inhibitors; U.S. Pat. No. 5,521,213 Diaryl bicyclic heterocycles as inhibitors of cyclooxygenase-2; U.S. Pat. No. 5,536,752 Phenyl heterocycles as COX-2 inhibitors; U.S. Pat. No. 5,550,142 Phenyl heterocycles as COX-2 inhibitors; U.S. Pat. No. 5,552,422 Aryl substituted 5,5 fused aromatic nitrogen compounds as anti-inflammatory agents; U.S. Pat. No. 5,604,253 N-benzylindol-3-yl propanoic acid derivatives as cyclooxygenase inhibitors; U.S. Pat. No. 5,604,260 5-methanesulfonamido-1-indanones as an inhibitor of cyclooxygenase-2; U.S. Pat. No. 5,639,780 N-benzyl indol-3-yl butanoic acid derivatives as cyclooxygenase inhibitors; U.S. Pat. No. 5,677,318 Diphenyl-1, 2-3-thiadiazoles as anti-inflammatory agents; U.S. Pat. No. 5,691,374 Diaryl-5-oxygenated-2-(SH)-furanones as COX-2 inhibitors; U.S. Pat. No. 5,698,584 3,4-diaryl-2-hydroxy-2,5-d-ihydrofurans as prodrugs to COX-2 inhibitors; U.S. Pat. No. 5,710,140 Phenyl heterocycles as COX-2 inhibitors; U.S. Pat. No. 5,733,909 Diphenyl stilbenes as prodrugs to COX-2 inhibitors; U.S. Pat. No. 5,789,413 Alkylated styrenes as prodrugs to COX-2 inhibitors; U.S. Pat. No. 5,817,700 Bisaryl cyclobutenes derivatives as cyclooxygenase inhibitors; U.S. Pat. No. 5,849,943 Stilbene derivatives useful as cyclooxygenase-2 inhibitors; U.S. Pat. No. 5,861,419 Substituted pyridines as selective cyclooxygenase-2 inhibitors; U.S. Pat. No. 5,922,742 Pyridinyl-2-cyclopenten-1-ones as selective cyclooxygenase-2 inhibitors; U.S. Pat. No. 5,925,631 Alkylated styrenes as prodrugs to COX-2 inhibitors; all of which are commonly assigned to Merck Frost Canada, Inc. (Kirkland, Calif.). Additional COX-2 inhibitors are also described in U.S. Pat. No. 5,643,933, assigned to G. D. Searle & Co. (Skokie, Ill.), entitled: Substituted sulfonylphenylheterocycles as cyclooxygenase-2 and 5-lipoxygenase inhibitors.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin (renin-angiotensin) system inhibitors are compounds that act to interfere with the production of angiotensin II from angiotensinogen or angiotensin I or interfere with the activity of angiotensin II. Such inhibitors are well known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include antibodies (e.g., to renin), amino acids and analogs thereof (including those conjugated to larger molecules), peptides (including peptide analogs of angiotensin and angiotensin 1), pro-renin related analogs, etc. Among the most potent and useful renin-angiotensin system inhibitors are renin inhibitors, ACE inhibitors, and angiotensin II antagonists.

Examples of angiotensin II antagonists include: peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alany-1-(4, thiazolyl)-L-alanyl (35, 45)-4-amino-3-hydroxy-5-cyclo-hexapentanoyl-1-N-hexylamide, Sankyo Company, Ltd., Tokyo, Japan); SKF108566 (E-alpha-2-[2-butyl-1-(carboxy phenyl)methyl]1H-imidazole-5-yl[methyl-ane]-2-thiophenepropanoic acid, Smith Kline Beecham Pharmaceuticals, Pa.); Losartan (DUP7531MK954, DuPont Merck Pharmaceutical Company); Remikirin (RO42-5892, F. Hoffman LaRoche AG); A₂ agonists (Marion Merrill Dow) and certain non-peptide heterocycles (G. D. Searle and Company). Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

### XI. Predisposition Screening

Information on association/correlation between genotypes and disease-related phenotypes can be exploited in several ways. For example, in the case of a highly-statistically significant association between one or more SNPs with predisposition to a disease for which treatment is available, detection of such a genotype pattern in an individual may justify immediate administration of treatment, or at least the institution of regular monitoring of the individual. Even if detection of one of the SNPs of the invention did not call for immediate therapeutic intervention or monitoring in a particular individual, the subject can nevertheless be motivated to begin simple life-style changes (e.g., diet, exercise) that can be accomplished at little or no cost to the individual but would confer potential benefits in reducing the risk of developing conditions for which that individual may have an increased risk by virtue of having the CAD-susceptibility allele(s).

The SNPs of the invention may contribute to coronary artery disease in an individual in different ways. Some polymorphisms occur within a protein coding sequence and contribute to disease phenotype by affecting protein structure. Other polymorphisms occur in noncoding regions but may exert phenotypic effects indirectly via influence on, for example, replication, transcription, and/or translation. A single SNP may affect more than one phenotypic trait. Likewise, a single phenotypic trait may be affected by multiple SNPs in different genes.

As used herein, the terms “diagnose”, “diagnosis”, and “diagnostics” include, but are not limited to any of the following: detection of coronary artery disease that an individual may presently have, predisposition/susceptibility screening (i.e., determining the increased risk of an individual in developing coronary artery disease in the future, or determining whether an individual has a decreased risk of developing coronary artery disease in the future), determining a particular type or subclass of coronary artery disease in an individual known to have coronary artery disease, confirming or reinforcing a previously made diagnosis of artery disease, pharmacogenomic evaluation of an individual to determine which therapeutic strategy that individual is most likely to positively respond to or to predict whether a patient is likely to respond to a particular treatment, predicting whether a patient is likely to experience toxic effects from a particular treatment or therapeutic compound, and evaluating the future prognosis of an individual having coronary artery disease. Such diagnostic uses are based on the SNPs individually or in a unique combination or SNP haplotypes of the present invention.

Haplotypes are particularly useful in that, for example, fewer SNPs can be genotyped to determine if a particular genomic region harbors a locus that influences a particular phenotype, such as in linkage disequilibrium-based SNP association analysis.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP-site located nearby. Hence, genotyping one of the SNP sites will give almost the same information as genotyping the other SNP site that is in LD.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For diagnostic purposes and similar uses, if a particular SNP site is found to be useful for diagnosing coronary artery disease (e.g., has a significant statistical association with the condition and/or is recognized as a causative polymorphism for the condition), then the skilled artisan would recognize that other SNP sites which are in LD with this SNP site would also be useful for diagnosing the condition. Thus, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is, predictive of the genotype of the causative polymorphism and, consequently, predictive of the phenotype (e.g., coronary artery disease) that is influenced by the causative SNP(s). Therefore, polymorphic markers that are in LD with causative polymorphisms are useful as diagnostic markers, and are particularly useful when the actual causative polymorphism(s) is/are unknown.

Examples of polymorphisms that can be in LD with one or more causative polymorphisms (and/or in LD with one or more polymorphisms that have a significant statistical association with a condition) and therefore useful for diagnosing the same condition that the causative/associated SNP(s) is used to diagnose, include, for example, other SNPs in the same gene, protein-coding, or mRNA transcript-coding region as the causative/associated SNP, other SNPs in the same exon or same intron as the causative/associated SNP, other SNPs in the same haplotype block as the causative/associated SNP, other SNPs in the same intergenic region as the causative/associated SNP, SNPs that are outside but near a gene (e.g., within 6 kb on either side, 5′ or 3′, of a gene boundary) that harbors a causative/associated SNP, etc.

Linkage disequilibrium in the human genome is reviewed in: Wall et al., “Haplotype blocks and linkage disequilibrium in the human genome”, Nat Rev Genet August 2003; 4(8):587-97; Garner et al., “On selecting markers for association studies: patterns of linkage disequilibrium between two and three diallelic loci”, Genet Epidemiol. January 2003; 24(1):57-67; Ardlie et al., “Patterns of linkage disequilibrium in the human genome”, Nat Rev Genet. April 2002; 3(4):299-309 (erratum in Nat Rev Genet July 2002; 3(7):566); and Remm et al., “High-density genotyping and linkage disequilibrium in the human genome using chromosome 22 as a model”; Curr Opin Chem. Biol. February 2002; 6(1):24-30.

The contribution or association of particular SNP and/or SNP haplotype with disease phenotypes, such as coronary artery disease, enables the SNPs of the present invention to be used to develop superior diagnostic tests capable of identifying individuals who express a detectable trait, such as coronary artery disease, as the result of a specific genotype, or individuals whose genotype places them at an increased or decreased risk of developing a detectable trait at a subsequent time as compared to individuals who do not have that genotype. As described herein, diagnostics may be based on a single SNP or a group of SNPs. Combined detection of a plurality of SNPs (for example, 2, 3, 4, 5, 6, 7, 8, 9, 10, 11, 12, 13, 14, 15, 16, 17, 18, 19, 20, 24, 25, 30, 32, 48, 50, 64, 96, 100, or any other number in-between, or more, of the SNPs provided in Tables 1-4) typically increases the probability of an accurate diagnosis. For example, the presence of a single SNP known to correlate with coronary artery disease might indicate a probability of 20% that an individual has or is at risk of developing coronary artery disease, whereas detection of five SNPs, each of which correlates with coronary artery disease, might indicate a probability of 80% that an individual has or is at risk of developing coronary artery disease. To further increase the accuracy of diagnosis or predisposition screening, analysis of the SNPs of the present invention can be combined with that of other polymorphisms or other risk factors of coronary artery disease, such as disease symptoms, pathological characteristics, family history, diet, environmental factors or lifestyle factors.

It will, of course, be understood by practitioners skilled in the treatment or diagnosis of coronary artery disease that the present invention generally does not intend to provide an absolute identification of individuals who are at risk (or less at risk) of developing coronary artery disease, and/or pathologies related to coronary artery disease, but rather to indicate a certain increased (or decreased) degree or likelihood of developing the disease based on statistically significant association results. However, this information is extremely valuable as it can be used to, for example, initiate preventive treatments or to allow an individual carrying one or more significant SNPs or SNP haplotypes to foresee warning signs such as minor clinical symptoms, or to have regularly scheduled physical exams to monitor for appearance of a condition in order to identify and begin treatment of the condition at an early stage. Particularly with diseases that are extremely debilitating or fatal if not treated on time, the knowledge of a potential predisposition, manner to treatment efficacy.

The diagnostic techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders related to coronary artery disease.

Another aspect of the present invention relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more SNP positions, wherein the assayed nucleotide(s) is/are indicative of an increased or decreased risk of developing the trait or indicative that the individual expresses the trait as a result of possessing a particular trait-causing or trait-influencing allele.

### EXEMPLIFICATION

The invention now being generally described, it will be more readily understood by reference to the following examples, which are included merely for purposes of illustration of certain aspects and embodiments of the present invention and are not intended to be limiting in any way.

The contents of any patents, patent applications, patent publications, or scientific articles referenced anywhere in this application are herein incorporated by reference in their entirety.

### Example 1

### Identification of Human Alleles and SNPs Determinative of CAD

Cardiovascular disease (CVD) is the leading cause of morbidity and mortality in the United States. Among the risk factors for cardiovascular disease are behavioral (e.g.) smoking, sedentary lifestyle, or poor diet), age and health-related (e.g. diabetes, hyperlipidemia or hypertension), and genetic factors. Family history as a general marker for genetic risk is one of the most consistently identified risk factors for CVD, yet there are no examples of genes known to increase risk in even a fraction of individuals with CVD. One of the reasons that these genes are so difficult to find is that the genetic effects of any given gene are likely to be small and are likely to interact with other genes. In addition, these effects are likely to manifest themselves at different ages and stages along the CVD continuum.

The Approaches for Genomic Discovery in Atherosclerosis (AGENDA) study was initiated to discover genes for CVD among a large number of genes implicated in a study of gene expression in human aortas. The goal of the human disease association components of the AGENDA study is to evaluate these genes in a clinic-based sample of individuals presenting to the Duke Diagnostic Catheterization Laboratory (DDLC). Patients presenting to the DDCL have been offered the opportunity to contribute to the CATHGEN study blood bank, which houses blood, plasma and RNA samples. These samples are later matched to the diagnostic and outcome information stored in the DISSC database maintained at the Duke Clinical Research Institute. The CATHGEN subjects have consented and the samples have been collected under the appropriate authorizations from the Duke University Medical Center IRB.

Two sets of samples have been obtained from the CATHGEN study for analysis in the AGENDA study. These samples have been selected on the basis of CAD index (CADi, an angiographically-defined measure of disease risk) and age. The first set of samples includes 468 young affected (YA) subjects (age ≦55, CADi>32), 260 older affected (OA) subjects (age >55, CADi>74) and 320 unaffected elderly (ON) subjects (age >60, CADi<23). The OA vs. ON and YA vs. ON comparisons are performed to identify genetic polymorphisms that increase susceptibility to CVD per se. The OA vs. YA comparison is performed to identify genetic polymorphisms that modify risk resulting in disease that presents at a young age, under the assumption that all individuals are at risk for CVD.

Over 1050 single nucleotide polymorphisms in 275 genes have been genotyped. These genes have been selected on the basis of location in the genome relative to a genetic linkage analysis of early onset coronary artery disease in families (the GENECARD study), ability to predict aortic atherosclerosis using gene expression in the human aorta, ability to predict aortic atherosclerosis in APO-E knockout mice, and published reports of genes identified through linkage analysis of CAD.

SNP candidates were selected using an algorithm to identify high-quality SNPs from public resources. FIG. 1 graphically describes the algorithm used. In some cases, high-quality SNPs could not be identified from public sources, in which case, exon re-sequencing of a limited number of individuals was performed to identify de novo SNPs in target genes.

The statistical analysis of these variants was performed in a two-step process. First the genotypes were analyzed to evaluate the quality of the genotyping experiment. The CHG quality control protocol includes error analysis of duplicated samples arranged throughout the SNP analysis plates, evaluation of genotyping efficiency, analysis of allele frequencies and consistency with Hardy-Weinberg equilibrium. Once the SNPs were shown to meet error rate and consistency standards, the second part of the analysis was performed to evaluate association of SNP alleles and genotypes with disease status. Logistic regression was performed of diseased vs. normal or young vs. old disease adjusting for ethnicity and gender. Indicators for SNP alleles or SNP genotypes were included in the model. SNPs with model coefficients providing p-values less than 0.10 were considered interesting and worthy of additional analysis.

Table 1 provides an overview of the lowest p-values for each SNP. The x-axis represents location in the genome and the y-axis shows the negative log(base 10) of the lowest p-value for that SNP. Thus log p-values greater than 1.3 represent p-values less than 0.05 and log p-values greater than 2 represent p-values less than 0.01. Abbreviated gene names are included on the plot for all significant SNPs. Detailed results of this analysis are shown in Table 1:

Most Significant Logist P values Per SNP

| | | | | | Marshfield | | | |
|---|---|---|---|---|---|---|---|---|
| | | | Location | genotype | (cM) | | y axis | |
| PROBE | LOCUS | Chrom | (bp) | logist p-value | | x axis | (−log 10) | model |
| RS976881 | TNFRSF1B | 1 | 11943300 | 0.2413 | 29.93 | 29.93 | 0.6174 | affec |
| RS235251 | TNFRSF1B | 1 | 11966936 | 0.316 | 29.93 | 29.93 | 0.5003 | affec |
| RS3397 | TNFRSF1B | 1 | 11976838 | 0.8109 | 29.93 | 29.93 | 0.0910 | young |
| RS1892345 | PINK1 | 1 | 20426528 | 0.2157 | 48.53 | 48.53 | 0.6661 | affec |
| RS7517909 | PINK1 | 1 | 20430479 | 0.5168 | 48.53 | 48.53 | 0.2867 | affec |
| RS2298298 | PINK1 | 1 | 20433803 | 0.2843 | 48.53 | 48.53 | 0.5462 | young |
| RS3121394 | PINK1 | 1 | 20436355 | 0.2797 | 48.53 | 48.53 | 0.5533 | affec |
| RS879086 | PINK1 | 1 | 20439165 | 0.4876 | 48.53 | 48.53 | 0.3119 | affec |
| RS1043424 | PINK1 | 1 | 20446475 | 0.1911 | 48.53 | 48.53 | 0.7187 | affec |
| RS607254 | DDOST | 1 | 20450355 | 0.4739 | 48.53 | 48.53 | 0.3243 | affec |
| RS640742 | PINK1 | 1 | 20454029 | 0.489 | 48.53 | 48.53 | 0.3107 | affec |
| RS291988 | C1QB | 1 | 22449533 | 0.05 | 50.93 | 50.93 | 1.3028 | old |
| RS631090 | C1QB | 1 | 22455878 | 0.021 | 50.97 | 50.97 | 1.6882 | young |
| RS623607 | C1QB | 1 | 22456191 | 0.0861 | 50.98 | 50.98 | 1.0650 | old |
| RS10580 | C1QB | 1 | 22457433 | 0.2066 | 50.98 | 50.98 | 0.6849 | young |
| RS292007 | C1QB | 1 | 22460987 | 0.045 | 51.00 | 51.00 | 1.3458 | affec |
| RS4659371 | AIM1L | 1 | 26262249 | 0.1168 | 55.10 | 55.10 | 0.9326 | old |
| RS4659431 | AIM1L | 1 | 26263079 | 0.0646 | 55.10 | 55.10 | 1.1898 | old |
| RS7517559 | AIM1L | 1 | 26267462 | 0.036 | 55.10 | 55.10 | 1.4473 | old |
| RS4454539 | AIM1L | 1 | 26284951 | 0.018 | 55.10 | 55.10 | 1.7520 | young |
| HCV1271113 | C1ORF38 | 1 | 27810572 | 0.008 | 56.50 | 56.50 | 2.1192 | old |
| RS3766398 | C1ORF38 | 1 | 27813993 | 0.002 | 56.50 | 56.50 | 2.6383 | affec |
| RS1467465 | C1ORF38 | 1 | 27816091 | 0.0502 | 56.50 | 56.50 | 1.2993 | affec |
| RS1467464 | C1ORF38 | 1 | 27816338 | 0.004 | 56.50 | 56.50 | 2.4437 | old |
| RS6564 | C1ORF38 | 1 | 27817663 | 0.003 | 56.50 | 56.50 | 2.5686 | old |
| 1P0259 | LAM5_HUMAN | 1 | 30709045 | 0.0584 | 59.29 | 59.29 | 1.2336 | affec |
| RS3795438 | LAM5_HUMAN | 1 | 30709109 | 0.0522 | 59.29 | 59.29 | 1.2823 | old |
| 1P0258 | LAM5_HUMAN | 1 | 30710514 | 0.0635 | 59.30 | 59.30 | 1.1972 | affec |
| RS1188360 | LAPTM5 | 1 | 30714848 | 0.2397 | 59.32 | 59.32 | 0.6203 | young |
| 1P0257 | LAM5_HUMAN | 1 | 30717443 | 0.1338 | 59.34 | 59.34 | 0.8735 | old |
| HCV9635468 | LAPTM5 | 1 | 30717836 | 0.3572 | 59.34 | 59.34 | 0.4471 | old |
| RS1188349 | LAM5_HUMAN | 1 | 30726129 | 0.1056 | 59.39 | 59.39 | 0.9763 | old |
| 1P0260 | LAM5_HUMAN | 1 | 30732520 | 0.3152 | 59.42 | 59.42 | 0.5014 | young |
| RS1407882 | LAM5_HUMAN | 1 | 30732667 | 0.2735 | 59.42 | 59.42 | 0.5630 | old |
| RS2273979 | LAPTM5 | 1 | 30733140 | 0.5827 | 59.43 | 59.43 | 0.2346 | young |
| RS2070929 | TAL1 | 1 | 47053524 | 0.2556 | 75.66 | 75.66 | 0.5924 | young |
| RS2249665 | TAL1 | 1 | 47057001 | 0.3905 | 75.66 | 75.66 | 0.4084 | old |
| RS2250495 | TAL1 | 1 | 47063294 | 0.04 | 75.66 | 75.66 | 1.3958 | young |
| RS1050204 | FCGR1A | 1 | 146979500 | 0.0908 | 155.89 | 155.89 | 1.0419 | young |
| RS1050208 | FCGR1A | 1 | 146979543 | 0.9719 | 155.89 | 155.89 | 0.0124 | affec |
| HCV2596598 | GBA | 1 | 152417982 | 0.2076 | 161.05 | 161.05 | 0.6828 | affec |
| HCV9632667 | GBA | 1 | 152418027 | 0.2939 | 161.05 | 161.05 | 0.5318 | affec |
| RS2075569 | GBA | 1 | 152426152 | 0.2321 | 161.05 | 161.05 | 0.6343 | affec |
| RS734073 | GBA | 1 | 152435157 | 0.2729 | 161.05 | 161.05 | 0.5640 | young |
| RS1417938 | CRP | 1 | 156900978 | 0.205 | 165.97 | 165.97 | 0.6882 | young |
| RS6027 | F5 | 1 | 166670938 | 0.281 | 187.43 | 187.43 | 0.5513 | young |
| RS4524 | F5 | 1 | 166699132 | 0.1895 | 187.46 | 187.46 | 0.7224 | old |
| RS2040442 | F5 | 1 | 166722265 | 0.4052 | 187.48 | 187.48 | 0.3923 | young |
| HCV341182 | F5 | 1 | 166732964 | 0.5683 | 187.49 | 187.49 | 0.2454 | young |
| RS6128 | SELP | 1 | 166750281 | 0.1835 | 187.51 | 187.51 | 0.7364 | old |
| RS6136 | SELP | 1 | 166751328 | 0.065 | 187.51 | 187.51 | 1.1871 | affec |
| RS6133 | SELP | 1 | 166752723 | 0.021 | 187.51 | 187.51 | 1.6778 | affec |
| RS6132 | SELP | 1 | 166753685 | 0.021 | 187.52 | 187.52 | 1.6819 | affec |
| RS6131 | SELP | 1 | 166768262 | 0.1962 | 187.53 | 187.53 | 0.7073 | old |
| RS732314 | SELP | 1 | 166786631 | 0.4003 | 187.55 | 187.55 | 0.3976 | old |
| RS909628 | SELL | 1 | 166848042 | 0.114 | 187.62 | 187.62 | 0.9431 | affec |
| RS4987286 | SELL | 1 | 166865056 | 0.2802 | 187.63 | 187.63 | 0.5525 | affec |
| RS1051091 | SELL | 1 | 166865086 | 0.1843 | 187.63 | 187.63 | 0.7345 | young |
| RS689470 | PTGS2 | 1 | 183880050 | 0.2675 | 201.28 | 201.28 | 0.5727 | affec |
| RS2820312 | LMOD1 | 1 | 199157514 | 0.2171 | 215.99 | 215.99 | 0.6633 | affec |
| HCV1467674 | LMOD1 | 1 | 199162969 | 0.1764 | 215.99 | 215.99 | 0.7535 | young |
| RS2819346 | LMOD1 | 1 | 199170344 | 0.1355 | 215.99 | 215.99 | 0.8681 | old |
| RS2819366 | LMOD1 | 1 | 199196238 | 0.007 | 215.99 | 215.99 | 2.1367 | young |
| RS903357 | CHI3L1 | 1 | 200435867 | 0.4242 | 216.82 | 216.82 | 0.3724 | young |
| RS4950927 | CHI3L1 | 1 | 200436890 | 0.9798 | 216.82 | 216.82 | 0.0089 | old |
| RS946259 | CHI3L1 | 1 | 200440434 | 0.154 | 216.82 | 216.82 | 0.8125 | old |
| RS880633 | CHI3L1 | 1 | 200441058 | 0.3597 | 216.82 | 216.82 | 0.4441 | old |
| RS7515776 | CHI3L1 | 1 | 200443960 | 0.5349 | 216.82 | 216.82 | 0.2717 | old |
| RS2271627 | CAPG | 2 | 85596599 | 0.013 | 106.17 | 388.17 | 1.8928 | old |
| RS2271625 | CAPG | 2 | 85600053 | 0.2977 | 106.18 | 388.18 | 0.5262 | young |
| HCV2763587 | CAPG | 2 | 85612863 | 0.1336 | 106.23 | 388.23 | 0.8742 | affec |
| RS1877954 | CAPG | 2 | 85628839 | 0.3883 | 106.28 | 388.28 | 0.4108 | old |
| RS1877955 | CAPG | 2 | 85629066 | 0.089 | 106.28 | 388.28 | 1.0506 | old |
| RS12888 | VAMP5 | 2 | 85783277 | 0.1183 | 106.82 | 388.82 | 0.9270 | old |
| HCV2091655 | VAMP5 | 2 | 85793028 | 0.3619 | 106.86 | 388.86 | 0.4414 | affec |
| RS14976 | VAMP5 | 2 | 85793426 | 0.1344 | 106.86 | 388.86 | 0.8716 | old |
| RS2228014 | CXCR4 | 2 | 137083853 | 0.2998 | 146.11 | 428.11 | 0.5232 | affec |
| RS6706557 | CXCR4 | 2 | 137095930 | 0.2892 | 146.14 | 428.14 | 0.5388 | young |
| RS3764917 | FAP | 2 | 163232880 | 0.1794 | 166.17 | 448.17 | 0.7462 | old |
| RS2300750 | FAP | 2 | 163271853 | 0.5851 | 166.20 | 448.20 | 0.2328 | old |
| HCV2780261 | c | 2 | 163284939 | 0.005 | 166.20 | 448.20 | 2.3372 | old |
| RS3788968 | FAP | 2 | 163300144 | 0.5832 | 166.21 | 448.21 | 0.2342 | old |
| RS1562315 | HOXD1 | 2 | 177248026 | 0.017 | 182.39 | 464.39 | 1.7670 | young |
| RS1446575 | HOXD1 | 2 | 177250345 | 0.0624 | 182.39 | 464.39 | 1.2048 | old |
| RS1374326 | HOXD1 | 2 | 177260860 | 0.2378 | 182.40 | 464.40 | 0.6238 | affec |
| RS1026032 | HOXD1 | 2 | 177267367 | 0.028 | 182.41 | 464.41 | 1.5607 | young |
| RS501333 | ASB1 | 2 | 239622795 | 0.4175 | 254.82 | 536.82 | 0.3793 | young |
| RS489244 | ASB1 | 2 | 239627940 | 0.1784 | 254.83 | 536.83 | 0.7486 | young |
| RS507812 | ASB1 | 2 | 239638400 | 0.1181 | 254.86 | 536.86 | 0.9278 | young |
| RS477041 | ASB1 | 2 | 239642745 | 0.3191 | 254.87 | 536.87 | 0.4961 | young |
| HCV320258 | GLB1 | 3 | 33009951 | 0.0566 | 60.40 | 598.40 | 1.2472 | affec |
| HCV440839 | GLB1 | 3 | 33018248 | 0.1269 | 60.42 | 598.42 | 0.8965 | affec |
| HCV143637 | GLB1 | 3 | 33034652 | 0.0638 | 60.47 | 598.47 | 1.1952 | old |
| HCV78337 | GLB1 | 3 | 33047463 | 0.2526 | 60.50 | 598.50 | 0.5976 | affec |
| HCV223628 | GLB1 | 3 | 33103912 | 0.1368 | 60.66 | 598.66 | 0.8639 | affec |
| RS3774634 | CXCR6 | 3 | 45947034 | 0.3307 | 69.46 | 607.46 | 0.4806 | old |
| RS936939 | CXCR6 | 3 | 45947215 | 0.465 | 69.46 | 607.46 | 0.3325 | affec |
| HCV1929536 | CXCR6 | 3 | 45949636 | 0.4349 | 69.46 | 607.46 | 0.3616 | young |
| RS2234358 | CXCR6 | 3 | 45949636 | 0.4886 | 69.46 | 607.46 | 0.3110 | young |
| RS319689 | MAP4 | 3 | 47888759 | 0.016 | 70.56 | 608.56 | 1.8069 | affec |
| RS6442089 | MAP4 | 3 | 47917016 | 0.036 | 70.58 | 608.58 | 1.4473 | young |
| RS2230169 | MAP4 | 3 | 47918588 | 0.0562 | 70.58 | 608.58 | 1.2503 | young |
| RS1060407 | MAP4 | 3 | 47918629 | 0.034 | 70.58 | 608.58 | 1.4698 | young |
| RS2166770 | MAP4 | 3 | 47966265 | 0.008 | 70.61 | 608.61 | 2.1024 | affec |
| RS1009316 | BAX | 3 | 54150382 | 0.0558 | 70.81 | 608.81 | 1.2534 | young |
| RS905238 | FTL | 3 | 54157196 | 0.0955 | 70.82 | 608.82 | 1.0200 | affec |
| HCV1845492 | PVRL3 | 3 | 112123926 | 0.0928 | 126.83 | 664.83 | 1.0325 | old |
| RS1477848 | PVRL3 | 3 | 112138883 | 0.0658 | 126.83 | 664.83 | 1.1818 | young |
| RS1477844 | PVRL3 | 3 | 112150732 | 0.3471 | 126.83 | 664.83 | 0.4595 | old |
| RS1351049 | PVRL3 | 3 | 112163177 | 0.008 | 126.83 | 664.83 | 2.0757 | young |
| RS2221065 | CD96 | 3 | 112575294 | 0.3912 | 126.83 | 664.83 | 0.4076 | young |
| RS1553970 | CD96 | 3 | 112644012 | 0.098 | 126.87 | 664.87 | 1.0088 | young |
| RS1877575 | | 3 | 112748356 | 0.1576 | 126.94 | 664.94 | 0.8024 | affec |
| RS1282980 | LL5BETA | 3 | 112893222 | 0.008 | 127.04 | 665.04 | 2.0915 | affec |
| HCV3134278 | LL5BETA | 3 | 112984829 | 0.016 | 127.10 | 665.10 | 1.8097 | old |
| HCV1941929 | NP25 | 3 | 113041230 | 0.0663 | 127.13 | 665.13 | 1.1785 | young |
| HCV9724398 | | 3 | 113128656 | 0.358 | 127.19 | 665.19 | 0.4461 | affec |
| RS1492486 | GCET2 | 3 | 113168430 | 0.1326 | 127.22 | 665.22 | 0.8775 | young |
| RS2029636 | | 3 | 113242673 | 0.4073 | 127.27 | 665.27 | 0.3901 | old |
| RS2272022 | MOX2 | 3 | 113384751 | 0.3569 | 127.37 | 665.37 | 0.4475 | old |
| HCV1195991 | APG3 | 3 | 113573291 | 0.2683 | 127.49 | 665.49 | 0.5714 | old |
| HCV3129378 | URB | 3 | 113729302 | 0.3287 | 127.60 | 665.60 | 0.4832 | young |
| RS717706 | | 3 | 113781096 | 0.096 | 127.63 | 665.63 | 1.0177 | young |
| HCV1483985 | | 3 | 113964461 | 0.0555 | 127.75 | 665.75 | 1.2557 | affec |
| HCV3158985 | | 3 | 114046343 | 0.025 | 127.81 | 665.81 | 1.6073 | affec |
| RS3732812 | | 3 | 114183928 | 0.3191 | 127.89 | 665.89 | 0.4961 | affec |
| RS1875111 | BOC | 3 | 114299665 | 0.1162 | 127.89 | 665.89 | 0.9348 | affec |
| HCV25644981 | | 3 | 114402583 | 0.2345 | 127.89 | 665.89 | 0.6299 | affec |
| HCV3040817 | | 3 | 114609331 | 0.2661 | 127.92 | 665.92 | 0.5750 | affec |
| RS921741 | MAK3P | 3 | 114764952 | 0.1687 | 128.07 | 666.07 | 0.7729 | old |
| RS3773681 | ATP6V1A | 3 | 114844802 | 0.3954 | 128.15 | 666.15 | 0.4030 | old |
| HCV2056002 | DKFZP434C0328 | 3 | 114916170 | 0.014 | 128.22 | 666.22 | 1.8665 | old |
| RS3765114 | MGC42530 | 3 | 114976108 | 0.2955 | 128.28 | 666.28 | 0.5294 | affec |
| HCV9020734 | MGC42530 | 3 | 114994026 | 0.0522 | 128.30 | 666.30 | 1.2823 | affec |
| RS324555 | KIAA1407 | 3 | 115051009 | 0.037 | 128.36 | 666.36 | 1.4306 | affec |
| HCV1941287 | QTRTD1 | 3 | 115122727 | 0.1248 | 128.43 | 666.43 | 0.9038 | affec |
| RS6280 | DRD3 | 3 | 115211716 | 0.1731 | 128.52 | 666.52 | 0.7617 | young |
| RS3732782 | ZNF80 | 3 | 115276065 | 0.043 | 128.58 | 666.58 | 1.3625 | affec |
| RS3732781 | ZNF80 | 3 | 115276088 | 0.3825 | 128.58 | 666.58 | 0.4174 | young |
| HCV1499152 | ZNF80 | 3 | 115276221 | 0.026 | 128.58 | 666.58 | 1.5817 | affec |
| RS3732780 | ZNF80 | 3 | 115276721 | 0.0953 | 128.58 | 666.58 | 1.0209 | old |
| HCV7789260 | ZNF288 | 3 | 115387211 | 0.1086 | 128.69 | 666.69 | 0.9642 | young |
| HCV74522 | ZNF288 | 3 | 115485057 | 0.167 | 128.79 | 666.79 | 0.7773 | affec |
| HCV11231447 | ZNF288 | 3 | 115543280 | 0.1496 | 128.85 | 666.85 | 0.8251 | young |
| HCV11239258 | ZNF288 | 3 | 115662281 | 0.024 | 128.97 | 666.97 | 1.6253 | old |
| RS3732481 | ZNF288 | 3 | 115804314 | 0.1899 | 129.11 | 667.11 | 0.7215 | young |
| RS2033406 | LSAMP | 3 | 117386034 | 0.1443 | 131.83 | 669.83 | 0.8407 | affec |
| RS10934345 | | 3 | 117530516 | 0.1973 | 131.87 | 669.87 | 0.7049 | affec |
| RS2037009 | | 3 | 117948900 | 0.1813 | 132.70 | 670.70 | 0.7416 | old |
| RS1133603 | | 3 | 117971681 | 0.2437 | 132.74 | 670.74 | 0.6131 | young |
| RS4855909 | | 3 | 118050719 | 0.3101 | 132.90 | 670.90 | 0.5085 | old |
| RS938115 | | 3 | 118096175 | 0.1376 | 132.99 | 670.99 | 0.8614 | young |
| RS6788787 | | 3 | 118191749 | 0.058 | 133.18 | 671.18 | 1.2366 | affec |
| RS1915585 | | 3 | 118229733 | 0.049 | 133.25 | 671.25 | 1.3089 | old |
| RS1462845 | | 3 | 118263911 | 0.2095 | 133.32 | 671.32 | 0.6788 | young |
| RS4855900 | | 3 | 118316168 | 0.233 | 133.42 | 671.42 | 0.6326 | affec |
| RS1513162 | | 3 | 118455987 | 0.042 | 133.70 | 671.70 | 1.3726 | young |
| RS7427839 | | 3 | 118486224 | 0.2652 | 133.76 | 671.76 | 0.5764 | old |
| RS4643716 | | 3 | 118488473 | 0.4683 | 133.77 | 671.77 | 0.3295 | young |
| RS6790819 | | 3 | 118497691 | 0.1693 | 133.78 | 671.78 | 0.7713 | affec |
| RS4356827 | | 3 | 118499645 | 0.1035 | 133.79 | 671.79 | 0.9851 | old |
| RS2927275 | | 3 | 118504970 | 0.2647 | 133.80 | 671.80 | 0.5772 | old |
| RS1698042 | | 3 | 118506049 | 0.005 | 133.80 | 671.80 | 2.3468 | old |
| RS1501881 | | 3 | 118510741 | 0.023 | 133.81 | 671.81 | 1.6326 | affec |
| RS1698041 | | 3 | 118520652 | 0.01 | 133.83 | 671.83 | 1.9872 | old |
| RS2055426 | | 3 | 118541245 | 0.003 | 133.87 | 671.87 | 2.5686 | old |
| RS2937675 | | 3 | 118544791 | 0.002 | 133.88 | 671.88 | 2.6778 | old |
| 3ID0340 | | 3 | 118549524 | 0.005 | 133.89 | 671.89 | 2.2757 | old |
| RS1875518 | | 3 | 118550681 | 0.004 | 133.89 | 671.89 | 2.4089 | affec |
| RS2937673 | | 3 | 118553288 | 0.009 | 133.89 | 671.89 | 2.0362 | old |
| RS1676232 | | 3 | 118555740 | 3E−04 | 133.90 | 671.90 | 3.5229 | old |
| 3I0311 | | 3 | 118557351 | 0.1187 | 133.90 | 671.90 | 0.9255 | affec |
| RS1381801 | | 3 | 118561796 | 0.1099 | 133.91 | 671.91 | 0.9590 | young |
| RS2937666 | | 3 | 118567599 | 0.007 | 133.92 | 671.92 | 2.1308 | young |
| RS1910044 | | 3 | 118571620 | 0.3167 | 133.93 | 671.93 | 0.4994 | young |
| RS6778437 | | 3 | 118584839 | 0.0714 | 133.99 | 671.99 | 1.1463 | young |
| RS6795971 | | 3 | 118589894 | 0.1006 | 134.01 | 672.01 | 0.9974 | affec |
| RS1466416 | | 3 | 118591707 | 0.1335 | 134.02 | 672.02 | 0.8745 | old |
| RS1456186 | | 3 | 118948306 | 0.215 | 134.64 | 672.64 | 0.6676 | young |
| RS843855 | | 3 | 119077436 | 0.3914 | 135.01 | 673.01 | 0.4074 | young |
| RS1486336 | | 3 | 119224904 | 0.048 | 135.49 | 673.49 | 1.3188 | affec |
| RS1499989 | | 3 | 119322105 | 0.0984 | 135.81 | 673.81 | 1.0070 | young |
| RS1968010 | | 3 | 119390121 | 0.1945 | 136.03 | 674.03 | 0.7111 | affec |
| RS553070 | | 3 | 119475838 | 0.1997 | 136.31 | 674.31 | 0.6996 | affec |
| RS1401951 | | 3 | 119546927 | 0.368 | 136.32 | 674.32 | 0.4342 | young |
| RS705233 | | 3 | 119790824 | 0.2371 | 136.32 | 674.32 | 0.6251 | young |
| R5812824 | | 3 | 119875547 | 0.1559 | 136.36 | 674.36 | 0.8072 | young |
| RS1521299 | | 3 | 119931630 | 0.0874 | 136.45 | 674.45 | 1.0585 | young |
| RS4687959 | IGSF11 | 3 | 119944315 | 0.0924 | 136.47 | 674.47 | 1.0343 | affec |
| RS6779428 | IGSF11 | 3 | 119960525 | 0.6104 | 136.50 | 674.50 | 0.2144 | young |
| RS2160052 | IGSF11 | 3 | 119962780 | 0.011 | 136.50 | 674.50 | 1.9469 | old |
| RS39688 | | 3 | 120063749 | 0.243 | 136.68 | 674.68 | 0.6144 | affec |
| HCV106740 | UPK1B | 3 | 120213944 | 0.1641 | 136.93 | 674.93 | 0.7849 | old |
| HCV394161 | B4GALT4 | 3 | 120274470 | 0.0836 | 137.03 | 675.03 | 1.0778 | young |
| HCV1291178 | | 3 | 120351861 | 0.0888 | 137.16 | 675.16 | 1.0516 | old |
| HCV392638 | FLJ10902 | 3 | 120471737 | 0.6869 | 137.37 | 675.37 | 0.1631 | old |
| RS1060569 | C3ORF1 | 3 | 120557788 | 0.1157 | 137.45 | 675.45 | 0.9367 | young |
| RS25676 | ADPRH | 3 | 120626280 | 0.4085 | 137.48 | 675.48 | 0.3888 | young |
| RS1723969 | PLA1A | 3 | 120648554 | 0.2437 | 137.48 | 675.48 | 0.6131 | affec |
| RS2272269 | PLA1A | 3 | 120652793 | 0.3243 | 137.49 | 675.49 | 0.4891 | affec |
| RS2692622 | PLA1A | 3 | 120657863 | 0.382 | 137.49 | 675.49 | 0.4179 | young |
| RS2873788 | COX17 | 3 | 120699878 | 0.2143 | 137.50 | 675.50 | 0.6690 | affec |
| HCV9152783 | NR1I2 | 3 | 120820408 | 0.2415 | 137.54 | 675.54 | 0.6171 | old |
| HCV148571 | GSK3B | 3 | 120931466 | 0.149 | 137.58 | 675.58 | 0.8268 | affec |
| HCV1849042 | GSK3B | 3 | 121005728 | 0.3652 | 137.60 | 675.60 | 0.4375 | affec |
| RS2199503 | GSK3B | 3 | 121099390 | 0.1349 | 137.63 | 675.63 | 0.8700 | young |
| RS787204 | | 3 | 121338849 | 0.1206 | 137.71 | 675.71 | 0.9187 | old |
| HCV1545736 | FSTL1 | 3 | 121435029 | 0.092 | 137.74 | 675.74 | 1.0362 | old |
| RS1147707 | FSTL1 | 3 | 121490149 | 0.033 | 137.76 | 675.76 | 1.4763 | old |
| HCV11236738 | NDUFB4 | 3 | 121635292 | 0.265 | 137.81 | 675.81 | 0.5768 | young |
| RS2298958 | HGD | 3 | 121708192 | 0.4476 | 137.83 | 675.83 | 0.3491 | affec |
| RS2229308 | GTF2E1 | 3 | 121821347 | 0.4869 | 137.87 | 675.87 | 0.3126 | young |
| RS470931 | | 3 | 121947330 | 0.2619 | 137.91 | 675.91 | 0.5819 | old |
| HCV123952 | | 3 | 122111233 | 0.4231 | 137.97 | 675.97 | 0.3736 | affec |
| RS1191299 | | 3 | 122243458 | 0.2253 | 138.00 | 676.00 | 0.6472 | old |
| RS2030531 | POLQ | 3 | 122476099 | 0.3214 | 138.00 | 676.00 | 0.4930 | young |
| RS2877516 | POLQ | 3 | 122554774 | 0.1859 | 138.00 | 676.00 | 0.7307 | affec |
| RS1873645 | HCLS1 | 3 | 122669400 | 0.2481 | 138.00 | 676.00 | 0.6054 | old |
| RS1128158 | HCLS1 | 3 | 122671494 | 0.048 | 138.00 | 676.00 | 1.3170 | young |
| RS2070180 | HCLS1 | 3 | 122672239 | 0.2809 | 138.00 | 676.00 | 0.5514 | affec |
| RS6807963 | HCLS1 | 3 | 122674155 | 0.1739 | 138.00 | 676.00 | 0.7597 | old |
| RS3772126 | HCLS1 | 3 | 122675484 | 0.026 | 138.00 | 676.00 | 1.5850 | young |
| RS3772127 | HCLS1 | 3 | 122675826 | 0.2036 | 138.00 | 676.00 | 0.6912 | old |
| HCV1986471 | HCLS1 | 3 | 122683308 | 0.4187 | 138.00 | 676.00 | 0.3781 | young |
| HCV1986466 | HCLS1 | 3 | 122694194 | 0.0695 | 138.00 | 676.00 | 1.1580 | young |
| HCV11236049 | GOLGB1 | 3 | 122703105 | 0.2023 | 138.00 | 676.00 | 0.6940 | young |
| RS1574115 | GOLGB1 | 3 | 122790126 | 0.0989 | 138.00 | 676.00 | 1.0048 | young |
| HCV173175 | TRAITS | 3 | 122885890 | 0.142 | 138.00 | 676.00 | 0.8477 | old |
| HCV510429 | SLC15A2 | 3 | 122935076 | 0.3797 | 138.00 | 676.00 | 0.4206 | affec |
| RS1920309 | SLC15A2 | 3 | 122986380 | 0.039 | 138.00 | 676.00 | 1.4089 | affec |
| HCV180867 | MGC50831 | 3 | 123061205 | 0.1797 | 138.00 | 676.00 | 0.7455 | old |
| RS2681416 | | 3 | 123138514 | 0.1102 | 138.00 | 676.00 | 0.9578 | young |
| RS1501899 | CASR | 3 | 123228229 | 0.1182 | 138.00 | 676.00 | 0.9274 | affec |
| HCV1412358 | CASR | 3 | 123237587 | 0.075 | 138.00 | 676.00 | 1.1249 | old |
| HCV1412289 | CASR | 3 | 123311158 | 0.0583 | 138.00 | 676.00 | 1.2343 | old |
| RS2270917 | CASR | 3 | 123322147 | 0.0949 | 138.00 | 676.00 | 1.0227 | affec |
| NCV1412273 | CASR | 3 | 123329454 | 0.047 | 138.00 | 676.00 | 1.3307 | old |
| HCV1844609 | CSTA | 3 | 123377333 | 0.5897 | 138.00 | 676.00 | 0.2294 | affec |
| RS3749213 | WDR5B | 3 | 123454731 | 0.1845 | 138.00 | 676.00 | 0.7340 | affec |
| HCV23715 | KPNA1 | 3 | 123489016 | 0.1981 | 138.00 | 676.00 | 0.7031 | affec |
| HCV58011 | BAL | 3 | 123592571 | 0.4893 | 138.04 | 676.04 | 0.3104 | affec |
| RS1256196 | | 3 | 123680823 | 0.2218 | 138.17 | 676.17 | 0.6540 | young |
| HCV1402346 | HSPBAP1 | 3 | 123780378 | 0.1366 | 138.32 | 676.32 | 0.8645 | affec |
| RS2288677 | DIRC2 | 3 | 123919192 | 0.4153 | 138.53 | 676.53 | 0.3816 | young |
| HCV8993037 | | 3 | 124034412 | 0.131 | 138.71 | 676.71 | 0.8827 | young |
| HCV1541693 | PDIR | 3 | 124131457 | 0.4211 | 138.85 | 676.85 | 0.3756 | old |
| RS3749286 | PDIR | 3 | 124201092 | 0.2756 | 138.96 | 676.96 | 0.5597 | young |
| HCV1541690 | SEC22A | 3 | 124298953 | 0.1375 | 139.10 | 677.10 | 0.8617 | young |
| HCV426990 | | 3 | 124327354 | 0.0862 | 139.14 | 677.14 | 1.0645 | young |
| HCV11231121 | | 3 | 124365956 | 0.6655 | 139.17 | 677.17 | 0.1769 | old |
| HCV3035758 | | 3 | 124497648 | 0.0578 | 139.30 | 677.30 | 1.2381 | affec |
| RS2697519 | | 3 | 124612520 | 0.029 | 139.41 | 677.41 | 1.5421 | young |
| HCV1602661 | MYLK | 3 | 124729452 | 0.035 | 139.47 | 677.47 | 1.4572 | old |
| HCV1602707 | MYLK | 3 | 124824325 | 0.1483 | 139.49 | 677.49 | 0.8289 | old |
| HCV1720000 | HAPIP | 3 | 125143188 | 0.044 | 139.57 | 677.57 | 1.3565 | old |
| HCV9532700 | HAPIP | 3 | 125386334 | 0.6823 | 139.62 | 677.62 | 0.1660 | affec |
| RS333349 | HAPIP | 3 | 125717239 | 0.2766 | 139.83 | 677.83 | 0.5581 | young |
| RS1846892 | HAPIP | 3 | 125720194 | 0.1978 | 139.83 | 677.83 | 0.7038 | affec |
| HCV1485549 | HAPIP | 3 | 125733054 | 0.042 | 139.84 | 677.84 | 1.3809 | old |
| HCV11792770 | HAPIP | 3 | 125742906 | 0.0557 | 139.85 | 677.85 | 1.2541 | young |
| HCV1901477 | UMPS | 3 | 125777643 | 0.4462 | 139.88 | 677.88 | 0.3505 | affec |
| HCV1901488 | UMPS | 3 | 125783709 | 0.4369 | 139.89 | 677.89 | 0.3596 | affec |
| RS674165 | ITGB5 | 3 | 125798581 | 0.3427 | 139.90 | 677.90 | 0.4651 | affec |
| RS585021 | ITGB5 | 3 | 125803770 | 0.3179 | 139.90 | 677.90 | 0.4977 | old |
| HCV11792629 | ITGB5 | 3 | 125831953 | 0.3481 | 139.93 | 677.93 | 0.4583 | affec |
| HCV3113140 | ITGB5 | 3 | 125841936 | 0.584 | 139.94 | 677.94 | 0.2336 | affec |
| RS3772840 | ITGB5 | 3 | 125871265 | 0.2416 | 139.96 | 677.96 | 0.6169 | affec |
| HCV1901570 | ITGB5 | 3 | 125884474 | 0.9204 | 139.97 | 677.97 | 0.0360 | young |
| HCV108358 | MUC13 | 3 | 125977596 | 0.5084 | 140.05 | 678.05 | 0.2938 | old |
| RS2981534 | | 3 | 126085011 | 0.2116 | 140.15 | 678.15 | 0.6745 | old |
| RS1574340 | SLC12A8 | 3 | 126123578 | 0.6703 | 140.18 | 678.18 | 0.1737 | young |
| HCV1514189 | SLC12A8 | 3 | 126183586 | 0.3824 | 140.19 | 678.19 | 0.4175 | affec |
| HCV1514244 | ZNF148 | 3 | 126272722 | 0.257 | 140.23 | 678.23 | 0.5901 | old |
| HCV11230314 | OSBPL11 | 3 | 126585122 | 0.6525 | 140.72 | 678.72 | 0.1854 | affec |
| RS2979310 | | 3 | 126709410 | 0.013 | 140.92 | 678.92 | 1.8928 | young |
| HCV1477490 | | 3 | 127000905 | 0.2088 | 141.37 | 679.37 | 0.6803 | young |
| HCV11237732 | | 3 | 127019853 | 0.3247 | 141.40 | 679.40 | 0.4885 | affec |
| HCV123667 | FLJ20473 | 3 | 127124162 | 0.4552 | 141.57 | 679.57 | 0.3418 | old |
| RS1868121 | FTHFD | 3 | 127226562 | 0.1462 | 141.73 | 679.73 | 0.8351 | affec |
| HCV9474551 | KLF15 | 3 | 127378591 | 4E−04 | 141.99 | 679.99 | 3.3979 | young |
| RS777513 | FLJ31300 | 3 | 127521055 | 0.4505 | 142.24 | 680.24 | 0.3463 | young |
| RS1056523 | C4ST3 | 3 | 127582116 | 0.4658 | 142.34 | 680.34 | 0.3318 | old |
| RS1056522 | C4ST3 | 3 | 127582254 | 0.1436 | 142.34 | 680.34 | 0.8428 | young |
| HCV1935770 | | 3 | 127673585 | 0.5224 | 142.50 | 680.50 | 0.2820 | young |
| RS2053820 | MGC13016 | 3 | 127816215 | 0.081 | 142.75 | 680.75 | 1.0915 | young |
| HCV1290372 | MGC13016 | 3 | 127929507 | 0.7683 | 142.95 | 680.95 | 0.1145 | affec |
| HCV2067961 | PLXNA1 | 3 | 128034908 | 0.0734 | 143.13 | 681.13 | 1.1343 | affec |
| RS900422 | | 3 | 128188468 | 0.2725 | 143.40 | 681.40 | 0.5646 | young |
| RS1001942 | | 3 | 128452430 | 0.5701 | 143.86 | 681.86 | 0.2440 | young |
| RS2720240 | | 3 | 128568283 | 0.1387 | 143.94 | 681.94 | 0.8579 | old |
| RS920233 | TPRA40 | 3 | 128615993 | 0.0618 | 143.94 | 681.94 | 1.2090 | affec |
| HCV7468669 | PODLX2 | 3 | 128700179 | 0.6324 | 143.94 | 681.94 | 0.1990 | old |
| RS664910 | MGLL | 3 | 128794939 | 0.1381 | 143.94 | 681.94 | 0.8598 | affec |
| RS874546 | MGLL | 3 | 128859321 | 0.7188 | 143.94 | 681.94 | 0.1434 | affec |
| RS2217628 | | 3 | 128972538 | 0.1182 | 143.94 | 681.94 | 0.9274 | affec |
| HCV177600 | RUVBL1 | 3 | 129138417 | 0.009 | 143.94 | 681.94 | 2.0362 | young |
| RS2687720 | SELB | 3 | 129239864 | 0.2306 | 143.94 | 681.94 | 0.6371 | old |
| RS2955103 | SELB | 3 | 129336145 | 0.015 | 143.94 | 681.94 | 1.8386 | young |
| RS760383 | SELB | 3 | 129440474 | 0.2085 | 143.94 | 681.94 | 0.6809 | affec |
| HCV375170 | GATA2 | 3 | 129521443 | 0.005 | 143.94 | 681.94 | 2.3188 | affec |
| RS1573858 | GATA2 | 3 | 129526769 | 0.002 | 143.94 | 681.94 | 2.8239 | affec |
| RS6439129 | GATA2 | 3 | 129533682 | 0.002 | 143.94 | 681.94 | 2.6383 | affec |
| HCV1842067 | GR6 | 3 | 129618478 | 0.1485 | 143.94 | 681.94 | 0.8283 | affec |
| RS1127030 | RPN1 | 3 | 129659862 | 0.004 | 143.94 | 681.94 | 2.3872 | affec |
| RS2712418 | RPN1 | 3 | 129664545 | 0.2584 | 143.94 | 681.94 | 0.5877 | affec |
| RS2712371 | RPN1 | 3 | 129667123 | 0.004 | 143.94 | 681.94 | 2.4318 | affec |
| RS4857914 | RPN1 | 3 | 129671593 | 0.045 | 143.94 | 681.94 | 1.3458 | affec |
| HCV115673 | RAB7 | 3 | 129767321 | 0.4451 | 143.94 | 681.94 | 0.3515 | young |
| HCV11237369 | RAB7 | 3 | 129852779 | 0.2276 | 143.94 | 681.94 | 0.6428 | young |
| RS1683804 | NPD002 | 3 | 129937234 | 0.1769 | 143.94 | 681.94 | 0.7523 | old |
| HCV1861453 | | 3 | 130029463 | 0.3279 | 143.94 | 681.94 | 0.4843 | affec |
| RS395020 | FLJ12057 | 3 | 130065644 | 0.3972 | 143.94 | 681.94 | 0.4010 | affec |
| HCV1862760 | ZNF9 | 3 | 130227866 | 0.431 | 143.94 | 681.94 | 0.3655 | young |
| HCV11231355 | H1FX | 3 | 130356394 | 0.087 | 143.94 | 681.94 | 1.0605 | old |
| HCV11909732 | MBD4 | 3 | 130472576 | 0.02 | 143.94 | 681.94 | 1.7033 | old |
| HCV8765854 | PLXND1 | 3 | 130588168 | 0.0613 | 143.97 | 681.97 | 1.2125 | affec |
| RS2245285 | PLXND1 | 3 | 130607322 | 0.012 | 144.00 | 682.00 | 1.9245 | affec |
| RS2245278 | PLXND1 | 3 | 130607544 | 0.019 | 144.00 | 682.00 | 1.7190 | affec |
| RS2285368 | PLXND1 | 3 | 130612408 | 0.214 | 144.01 | 682.01 | 0.6696 | old |
| RS2255703 | PLXND1 | 3 | 130614165 | 0.0731 | 144.02 | 682.02 | 1.1361 | old |
| RS2255226 | PLXND1 | 3 | 130618132 | 0.018 | 144.02 | 682.02 | 1.7447 | affec |
| RS2285370 | PLXND1 | 3 | 130623364 | 0.2378 | 144.03 | 682.03 | 0.6238 | young |
| RS2285373 | PLXND1 | 3 | 130629118 | 0.3172 | 144.04 | 682.04 | 0.4987 | young |
| HCV8765558 | | 3 | 130687872 | 0.3718 | 144.14 | 682.14 | 0.4297 | young |
| RS2811343 | | 3 | 130871486 | 0.004 | 144.46 | 682.46 | 2.3565 | affec |
| RS938194 | | 3 | 130994409 | 0.0506 | 144.67 | 682.67 | 1.2958 | young |
| HCV8290655 | | 3 | 131296957 | 0.026 | 145.19 | 683.19 | 1.5884 | affec |
| HCV8291996 | | 3 | 131387466 | 0.1991 | 145.34 | 683.34 | 0.7009 | affec |
| RS322115 | FLJ35880 | 3 | 131510935 | 0.1305 | 145.55 | 683.55 | 0.8844 | old |
| RS1508520 | | 3 | 131618568 | 0.001 | 145.74 | 683.74 | 2.9586 | old |
| HCV3134777 | AGTR1 | 3 | 149741425 | 0.1038 | 165.32 | 703.32 | 0.9838 | affec |
| RS2640543 | AGTR1 | 3 | 149753278 | 0.1184 | 165.32 | 703.32 | 0.9266 | old |
| RS389566 | AGTR1 | 3 | 149767291 | 0.3337 | 165.32 | 703.32 | 0.4766 | affec |
| HCV8758668 | AGTR1 | 3 | 149780304 | 0.039 | 165.32 | 703.32 | 1.4056 | affec |
| RS275645 | AGTR1 | 3 | 149785363 | 0.3124 | 165.32 | 703.32 | 0.5053 | affec |
| HCV11803100 | AGTR1 | 3 | 149861063 | 0.0879 | 165.32 | 703.32 | 1.0560 | old |
| RS3772587 | AGTR1 | 3 | 149897825 | 0.1834 | 165.32 | 703.32 | 0.7366 | old |
| RS6141 | THPO | 3 | 185411179 | 0.0969 | 195.60 | 733.60 | 1.0137 | affec |
| RS6142 | THPO | 3 | 185412062 | 0.1926 | 195.60 | 733.60 | 0.7153 | old |
| RS1801212 | WFS1 | 4 | 6367061 | 0.2393 | 12.14 | 779.14 | 0.6211 | affec |
| RS1801214 | WFS1 | 4 | 6367564 | 0.0583 | 12.14 | 779.14 | 1.2343 | young |
| RS734312 | WFS1 | 4 | 6367896 | 0.242 | 12.14 | 779.14 | 0.6162 | old |
| RS1046314 | WFS1 | 4 | 6368497 | 0.1159 | 12.14 | 779.14 | 0.9359 | young |
| RS1046316 | WFS1 | 4 | 6368629 | 0.2628 | 12.14 | 779.14 | 0.5804 | young |
| HCV2674568 | SLA/LP | 4 | 24892583 | 0.5865 | 38.77 | 805.77 | 0.2317 | young |
| RS1035091 | SLA/LP | 4 | 24902757 | 0.1528 | 38.77 | 805.77 | 0.8159 | affec |
| RS5743618 | TLR1 | 4 | 38695828 | 0.1168 | 53.45 | 820.45 | 0.9326 | affec |
| RS5743614 | TLR1 | 4 | 38696115 | 0.0734 | 53.45 | 820.45 | 1.1343 | affec |
| RS3923647 | TLR1 | 4 | 38696719 | 0.1077 | 53.45 | 820.45 | 0.9678 | affec |
| RS4833095 | TLR1 | 4 | 38696890 | 0.1076 | 53.45 | 820.45 | 0.9682 | affec |
| RS5743596 | TLR1 | 4 | 38699708 | 0.1226 | 53.46 | 820.46 | 0.9115 | young |
| RS5743565 | TLR1 | 4 | 38703163 | 0.1994 | 53.47 | 820.47 | 0.7003 | affec |
| HCV151279 | SPP1 | 4 | 89349139 | 0.031 | 96.16 | 863.16 | 1.5100 | young |
| RS2853744 | SPP1 | 4 | 89354643 | 0.1428 | 96.16 | 863.16 | 0.8453 | young |
| HCV1840808 | SPP1 | 4 | 89354816 | 0.1346 | 96.16 | 863.16 | 0.8710 | young |
| RS2853749 | SPP1 | 4 | 89356209 | 0.025 | 96.16 | 863.16 | 1.6038 | affec |
| RS4754 | SPP1 | 4 | 89361087 | 0.024 | 96.16 | 863.16 | 1.6234 | young |
| RS1126616 | SPP1 | 4 | 89362248 | 0.027 | 96.16 | 863.16 | 1.5751 | young |
| RS1126772 | SPP1 | 4 | 89362581 | 0.1167 | 96.16 | 863.16 | 0.9329 | young |
| RS9138 | SPP1 | 4 | 89362737 | 0.03 | 96.16 | 863.16 | 1.5302 | young |
| RS2728116 | PKD2 | 4 | 89389445 | 0.038 | 96.16 | 863.16 | 1.4260 | affec |
| HCV258916 | PKD2 | 4 | 89390859 | 0.0803 | 96.16 | 863.16 | 1.0953 | affec |
| RS2728110 | PKD2 | 4 | 89411278 | 0.007 | 96.16 | 863.16 | 2.1367 | affec |
| RS2725218 | PKD2 | 4 | 89418363 | 0.1703 | 96.16 | 863.16 | 0.7688 | affec |
| RS2728105 | PKD2 | 4 | 89430462 | 0.1943 | 96.16 | 863.16 | 0.7115 | affec |
| RS221330 | HADHSC | 4 | 109380187 | 0.053 | 112.87 | 879.87 | 1.2757 | young |
| RS141066 | HADHSC | 4 | 109390371 | 0.0647 | 112.89 | 879.89 | 1.1891 | young |
| RS763432 | HADHSC | 4 | 109390457 | 0.2931 | 112.89 | 879.89 | 0.5330 | old |
| RS732941 | HADHSC | 4 | 109403924 | 0.4668 | 112.90 | 879.90 | 0.3309 | old |
| RS221347 | HADHSC | 4 | 109414442 | 0.6811 | 112.92 | 879.92 | 0.1668 | affec |
| RS1574637 | NPY1R | 4 | 164819719 | 0.044 | 163.24 | 930.24 | 1.3575 | affec |
| RS9764 | NPY1R | 4 | 164823032 | 0.3101 | 163.25 | 930.25 | 0.5085 | old |
| RS5577 | NPY1R | 4 | 164825370 | 0.4758 | 163.25 | 930.25 | 0.3226 | affec |
| RS4518200 | NPY1R | 4 | 164832049 | 0.025 | 163.26 | 930.26 | 1.6091 | affec |
| HCV385214 | GLRA3 | 4 | 176263722 | 0.1128 | 176.19 | 943.19 | 0.9477 | young |
| HCV9539364 | GLRA3 | 4 | 176274606 | 0.235 | 176.19 | 943.19 | 0.6289 | old |
| HCV8299063 | GLRA3 | 4 | 176303082 | 0.2026 | 176.19 | 943.19 | 0.6934 | affec |
| RS2046485 | GLRA3 | 4 | 176355509 | 0.6248 | 176.19 | 943.19 | 0.2043 | young |
| RS3749233 | ACSL1 | 4 | 186383123 | 0.037 | 198.10 | 965.10 | 1.4318 | old |
| HCV1170089 | ACSL1 | 4 | 186391214 | 0.025 | 198.18 | 965.18 | 1.6003 | affec |
| HCV1170063 | ACSL1 | 4 | 186419601 | 0.018 | 198.43 | 965.43 | 1.7375 | affec |
| RS2280297 | ACSL1 | 4 | 186432003 | 0.03 | 198.54 | 965.54 | 1.5229 | old |
| HCV2390582 | MATP | 5 | 34018032 | 0.1542 | 49.98 | 1022.98 | 0.8119 | young |
| RS2228140 | IL7R | 5 | 35871330 | 0.024 | 52.55 | 1025.55 | 1.6253 | old |
| RS1494555 | IL7R | 5 | 35916691 | 0.023 | 52.55 | 1025.55 | 1.6308 | old |
| RS2270555 | IL7R | 5 | 35916774 | 0.2835 | 52.55 | 1025.55 | 0.5474 | affec |
| RS987106 | IL7R | 5 | 35921094 | 0.0903 | 52.55 | 1025.55 | 1.0443 | old |
| RS3194051 | IL7R | 5 | 35921775 | 0.4191 | 52.55 | 1025.55 | 0.3777 | affec |
| RS1050674 | LHFPL2 | 5 | 77867162 | 0.037 | 83.09 | 1056.09 | 1.4306 | young |
| HCV3263440 | LHFPL2 | 5 | 77899327 | 0.042 | 83.12 | 1056.12 | 1.3726 | young |
| HCV3263427 | LHFPL2 | 5 | 77913885 | 0.1637 | 83.14 | 1056.14 | 0.7860 | old |
| HCV3263409 | LHFPL2 | 5 | 77926977 | 0.2523 | 83.16 | 1056.16 | 0.5981 | old |
| RS1561735 | LHFPL2 | 5 | 77950301 | 0.029 | 83.18 | 1056.18 | 1.5331 | old |
| HCV2084766 | ADRB2 | 5 | 148235156 | 0.0686 | 150.34 | 1123.34 | 1.1637 | affec |
| RS2277028 | GM2A | 5 | 150661186 | 0.0828 | 154.43 | 1127.43 | 1.0820 | old |
| 5P0001GM2A | GM2A | 5 | 150667919 | 0.0948 | 154.45 | 1127.45 | 1.0232 | old |
| RS153478 | GM2A | 5 | 150667949 | 0.1278 | 154.45 | 1127.45 | 0.8935 | old |
| RS248465 | GM2A | 5 | 150671563 | 0.0875 | 154.45 | 1127.45 | 1.0580 | young |
| 5P0002GM2A | GM2A | 5 | 150675246 | 0.5203 | 154.46 | 1127.46 | 0.2837 | old |
| RS2075783 | GM2A | 5 | 150675290 | 0.1292 | 154.46 | 1127.46 | 0.8887 | old |
| RS1048723 | GM2A | 5 | 150675522 | 0.4374 | 154.46 | 1127.46 | 0.3591 | affec |
| RS153450 | GM2A | 5 | 150676967 | 0.022 | 154.47 | 1127.47 | 1.6517 | young |
| RS264834 | DOCK2 | 5 | 169063385 | 0.1693 | 175.34 | 1148.34 | 0.7713 | affec |
| RS2279318 | DOCK2 | 5 | 169111769 | 0.02 | 175.34 | 1148.34 | 1.7011 | affec |
| HCV3138900 | DOCK2 | 5 | 169129475 | 0.039 | 175.34 | 1148.34 | 1.4145 | young |
| HCV1991155 | DOCK2 | 5 | 169285820 | 0.0643 | 175.34 | 1148.34 | 1.1918 | young |
| RS259894 | DOCK2 | 5 | 169339778 | 0.2832 | 175.34 | 1148.34 | 0.5479 | young |
| RS3776754 | STK10 | 5 | 171553499 | 0.2856 | 179.15 | 1152.15 | 0.5442 | young |
| HCV1191601 | STK10 | 5 | 171570835 | 0.4158 | 179.16 | 1152.16 | 0.3811 | young |
| RS2009658 | LTA | 6 | 31642549 | 0.1112 | 44.91 | 1200.91 | 0.9539 | affec |
| RS1800683 | LTA | 6 | 31644375 | 0.2059 | 44.91 | 1200.91 | 0.6863 | affec |
| RS2239704 | LTA | 6 | 31644445 | 0.1247 | 44.91 | 1200.91 | 0.9041 | affec |
| RS2857713 | LTA | 6 | 31644860 | 0.03 | 44.91 | 1200.91 | 1.5258 | young |
| HCV2451908 | LTA | 6 | 31644860 | 0.1574 | 44.91 | 1200.91 | 0.8030 | young |
| RS1041981 | LTA | 6 | 31645088 | 0.162 | 44.91 | 1200.91 | 0.7905 | affec |
| RS3093665 | LTA | 6 | 31649695 | 0.6647 | 44.91 | 1200.91 | 0.1774 | old |
| HCV2455646 | HLA- | 6 | 32480465 | 0.034 | 45.50 | 1201.50 | 1.4647 | young |
| | DRA | | | | | | | |
| RS8084 | HLA- | 6 | 32482258 | 0.0982 | 45.50 | 1201.50 | 1.0079 | affec |
| | DRA | | | | | | | |
| RS3134994 | HLA- | 6 | 32684102 | 0.04 | 45.56 | 1201.56 | 1.3947 | affec |
| | DQB1 | | | | | | | |
| RS2051600 | HLA- | 6 | 32756316 | 0.048 | 45.79 | 1201.79 | 1.3170 | affec |
| | DQA2 | | | | | | | |
| RS5018343 | HLA- | 6 | 32757126 | 0.017 | 45.79 | 1201.79 | 1.7620 | old |
| | DQA2 | | | | | | | |
| RS2395252 | HLA- | 6 | 32758327 | 0.2093 | 45.79 | 1201.79 | 0.6792 | old |
| | DQA2 | | | | | | | |
| RS2213566 | HLA- | 6 | 32760040 | 0.1537 | 45.80 | 1201.80 | 0.8133 | old |
| | DQA2 | | | | | | | |
| RS1042434 | HLA- | 6 | 33083392 | 0.3749 | 46.84 | 1202.84 | 0.4261 | young |
| | DPA1 | | | | | | | |
| RS1042174 | HLA- | 6 | 33084513 | 0.1988 | 46.84 | 1202.84 | 0.7016 | old |
| | DPA1 | | | | | | | |
| RS3135021 | HLA- | 6 | 33092445 | 0.0536 | 46.87 | 1202.87 | 1.2708 | affec |
| | DPA1 | | | | | | | |
| RS1367730 | HLA- | 6 | 33105001 | 0.3394 | 46.91 | 1202.91 | 0.4693 | young |
| | DPA1 | | | | | | | |
| RS1051931 | PLA2G7 | 6 | 46719779 | 0.1224 | 73.13 | 1229.13 | 0.9122 | old |
| RS1805018 | PLA2G7 | 6 | 46726139 | 0.0938 | 73.13 | 1229.13 | 1.0278 | young |
| RS1805017 | PLA2G7 | 6 | 46731058 | 0.006 | 73.13 | 1229.13 | 2.2518 | old |
| HCV2032816 | PLA2G7 | 6 | 46746128 | 0.003 | 73.13 | 1229.13 | 2.4815 | old |
| RS1862008 | PLA2G7 | 6 | 46757115 | 0.007 | 73.13 | 1229.13 | 2.1739 | affec |
| RS1014310 | BPAG1 | 6 | 56484911 | 0.029 | 80.01 | 1236.01 | 1.5406 | old |
| RS2024751 | BPAG1 | 6 | 56491004 | 0.0659 | 80.01 | 1236.01 | 1.1811 | affec |
| RS1024196 | BPAG1 | 6 | 56554325 | 0.2547 | 80.02 | 1236.02 | 0.5940 | old |
| RS2613118 | BPAG1 | 6 | 56775479 | 0.042 | 80.06 | 1236.06 | 1.3737 | affec |
| RS3752581 | PLN | 6 | 118915300 | 0.015 | 121.97 | 1277.97 | 1.8125 | young |
| 6P0325 | PLN | 6 | 118915300 | 0.025 | 121.97 | 1277.97 | 1.6073 | young |
| RS503031 | PLN | 6 | 118922380 | 0.3777 | 121.97 | 1277.97 | 0.4229 | young |
| 6P0324 | PLN | 6 | 118926210 | 0.027 | 121.97 | 1277.97 | 1.5768 | young |
| 6P0326 | PLN | 6 | 118927230 | 0.046 | 121.97 | 1277.97 | 1.3335 | affec |
| RS1051429 | PLN | 6 | 118927392 | 1E−04 | 121.97 | 1277.97 | 4.0000 | young |
| RS1998482 | PLN | 6 | 118931682 | 0.3632 | 121.97 | 1277.97 | 0.4399 | affec |
| RS3734382 | PLN | 6 | 118932531 | 0.001 | 121.97 | 1277.97 | 2.9586 | young |
| RS1385681 | SMPDL3A | 6 | 123099762 | 0.033 | 123.04 | 1279.04 | 1.4881 | young |
| HCV375819 | SMPDL3A | 6 | 123103040 | 0.0761 | 123.05 | 1279.05 | 1.1186 | old |
| RS869478 | SMPDL3A | 6 | 123104550 | 0.046 | 123.05 | 1279.05 | 1.3344 | young |
| HCV11639376 | SMPDL3A | 6 | 123109735 | 0.151 | 123.05 | 1279.05 | 0.8210 | young |
| RS1799971 | OPRM1 | 6 | 154391788 | 0.4823 | 155.08 | 1311.08 | 0.3167 | affec |
| RS524731 | OPRM1 | 6 | 154406083 | 0.2537 | 155.11 | 1311.11 | 0.5957 | affec |
| RS495491 | OPRM1 | 6 | 154413533 | 0.3433 | 155.12 | 1311.12 | 0.4643 | young |
| RS2075572 | OPRM1 | 6 | 154442995 | 0.1601 | 155.17 | 1311.17 | 0.7956 | affec |
| RS609148 | OPRM1 | 6 | 154462005 | 0.014 | 155.17 | 1311.17 | 1.8570 | affec |
| HCV11233252 | STEAP | 7 | 22355367 | 0.026 | 36.03 | 1379.03 | 1.5850 | young |
| RS199348 | GPNMB | 7 | 23035370 | 0.023 | 37.81 | 1380.81 | 1.6383 | affec |
| HCV963057 | GPNMB | 7 | 23036018 | 0.029 | 37.81 | 1380.81 | 1.5452 | old |
| HCV3148292 | GPNMB | 7 | 23038805 | 0.019 | 37.81 | 1380.81 | 1.7122 | old |
| RS199354 | GPNMB | 7 | 23038844 | 0.2886 | 37.81 | 1380.81 | 0.5397 | old |
| RS2268748 | GPNMB | 7 | 23055443 | 0.3093 | 37.85 | 1380.85 | 0.5096 | affec |
| RS5574 | NPY | 7 | 24071405 | 0.023 | 38.94 | 1381.94 | 1.6440 | young |
| RS5573 | NPY | 7 | 24325077 | 0.015 | 39.02 | 1382.02 | 1.8327 | young |
| RS1554494 | UPP1 | 7 | 47868969 | 0.021 | 69.82 | 1412.82 | 1.6882 | young |
| HCV406653 | UPP1 | 7 | 47872326 | 0.027 | 69.82 | 1412.82 | 1.5751 | young |
| RS1178970 | FKBP6 | 7 | 71817507 | 0.2217 | 84.52 | 1427.52 | 0.6542 | old |
| RS757941 | FKBP6 | 7 | 71823497 | 0.015 | 84.52 | 1427.52 | 1.8268 | old |
| RS374890 | FKBP6 | 7 | 71852334 | 0.1216 | 84.52 | 1427.52 | 0.9151 | old |
| RS1178968 | FKBP6 | 7 | 72179548 | 0.3068 | 87.08 | 1430.08 | 0.5131 | old |
| RS1045642 | ABCB1 | 7 | 86099488 | 0.0746 | 98.27 | 1441.27 | 1.1273 | affec |
| RS1128503 | ABCB1 | 7 | 86791630 | 0.2757 | 98.44 | 1441.44 | 0.5596 | affec |
| HCV2614970 | ABCB1 | 7 | 86841469 | 0.0693 | 98.44 | 1441.44 | 1.1593 | old |
| RS2214102 | ABCB1 | 7 | 86841530 | 0.1155 | 98.44 | 1441.44 | 0.9374 | old |
| RS2158746 | STEAP | 7 | 89396798 | 0.0928 | 100.23 | 1443.23 | 1.0325 | old |
| RS39283 | STEAP | 7 | 89400835 | 0.3351 | 100.24 | 1443.24 | 0.4748 | affec |
| RS39286 | STEAP | 7 | 89404279 | 0.1618 | 100.24 | 1443.24 | 0.7910 | old |
| RS2286254 | STEAP | 7 | 89405670 | 0.5045 | 100.24 | 1443.24 | 0.2971 | affec |
| RS437831 | FABP5 | 8 | 82240584 | 0.5983 | 97.93 | 1610.43 | 0.2231 | young |
| RS202275 | FABP5 | 8 | 82250480 | 0.1955 | 97.97 | 1610.47 | 0.7089 | young |
| RS202281 | FABP5 | 8 | 82253950 | 0.0997 | 97.99 | 1610.49 | 1.0013 | young |
| RS2252807 | SLA | 8 | 134019501 | 0.0719 | 147.49 | 1659.99 | 1.1433 | affec |
| HCV1190217 | ANKRD15 | 9 | 522762 | 0.3159 | 0.00 | 1684.00 | 0.5005 | affec |
| RS2641989 | ANKRD15 | 9 | 542379 | 0.047 | 0.00 | 1684.00 | 1.3242 | old |
| HCV1182387 | ADFP | 9 | 19105720 | 0.2863 | 33.60 | 1717.60 | 0.5432 | young |
| RS3824369 | ADFP | 9 | 19116565 | 0.0508 | 33.62 | 1717.62 | 1.2941 | affec |
| RS1969980 | GCNT1 | 9 | 74573227 | 0.4972 | 73.03 | 1757.03 | 0.3035 | young |
| RS1057406 | GCNT1 | 9 | 74575448 | 0.1741 | 73.03 | 1757.03 | 0.7592 | young |
| RS707739 | GCNT1 | 9 | 74576022 | 0.3716 | 73.03 | 1757.03 | 0.4299 | affec |
| HCV11763416 | GCNT1 | 9 | 74582459 | 0.1179 | 73.03 | 1757.03 | 0.9285 | affec |
| HCV2704852 | CTSL | 9 | 85790518 | 0.047 | 92.00 | 1776.00 | 1.3242 | old |
| RS2274611 | CTSL | 9 | 85799794 | 0.101 | 92.02 | 1776.02 | 0.9957 | affec |
| RS2378757 | CTSL | 9 | 85800899 | 0.1833 | 92.03 | 1776.03 | 0.7368 | affec |
| RS3128510 | CTSL | 9 | 85802727 | 0.1418 | 92.03 | 1776.03 | 0.8483 | affec |
| RS1027268 | ROR2 | 9 | 89712759 | 0.4149 | 99.32 | 1783.32 | 0.3821 | old |
| HCV11889939 | ROR2 | 9 | 89823450 | 0.0676 | 99.45 | 1783.45 | 1.1701 | old |
| RS4744098 | ROR2 | 9 | 89885691 | 0.1741 | 99.51 | 1783.51 | 0.7592 | affec |
| RS1881385 | ROR2 | 9 | 89938190 | 0.1356 | 99.57 | 1783.57 | 0.8677 | affec |
| HCV203542 | ROR2 | 9 | 89993111 | 0.017 | 99.62 | 1783.62 | 1.7670 | young |
| HCV1435528 | FBP1 | 9 | 92725520 | 0.5689 | 102.25 | 1786.25 | 0.2450 | old |
| HCV11380659 | ALOX5 | 10 | 45175490 | 0.5012 | 66.97 | 1896.97 | 0.3000 | young |
| RS2115819 | ALOX5 | 10 | 45185095 | 0.1601 | 66.98 | 1896.98 | 0.7956 | young |
| RS892691 | ALOX5 | 10 | 45201098 | 0.5357 | 66.99 | 1896.99 | 0.2711 | young |
| RS3740107 | ALOX5 | 10 | 45207776 | 0.1431 | 66.99 | 1896.99 | 0.8444 | young |
| RS2242332 | ALOX5 | 10 | 45222245 | 0.1535 | 66.99 | 1896.99 | 0.8139 | young |
| RS2255174 | SLIT1 | 10 | 98480960 | 0.319 | 118.58 | 1948.58 | 0.4962 | affec |
| RS2784920 | SLIT1 | 10 | 98578215 | 0.3611 | 118.86 | 1948.86 | 0.4424 | affec |
| RS1565495 | SLIT1 | 10 | 98603394 | 0.567 | 118.93 | 1948.93 | 0.2464 | old |
| RS2071616 | FGFR2 | 10 | 122944382 | 0.4336 | 142.78 | 1972.78 | 0.3629 | affec |
| RS1047100 | FGFR2 | 10 | 122962745 | 0.1942 | 142.82 | 1972.82 | 0.7118 | young |
| HCV8899692 | FGFR2 | 10 | 122962745 | 0.319 | 142.82 | 1972.82 | 0.4962 | affec |
| RS1078806 | FGFR2 | 10 | 123003562 | 0.2664 | 142.93 | 1972.93 | 0.5745 | young |
| HCV438264 | RPLP2 | 11 | 803830 | 0.043 | 0.00 | 2000.00 | 1.3655 | affec |
| RS4131364 | TTS-2.2 | 11 | 803830 | 0.021 | 0.00 | 2000.00 | 1.6696 | affec |
| RS1135628 | TTS-2.2 | 11 | 815456 | 0.1877 | 0.00 | 2000.00 | 0.7265 | young |
| HCV113313 | CD151 | 11 | 816753 | 0.1094 | 0.00 | 2000.00 | 0.9610 | affec |
| RS1138714 | TTS-2.2 | 11 | 816753 | 0.1086 | 0.00 | 2000.00 | 0.9642 | affec |
| RS4075289 | TTS-2.2 | 11 | 822313 | 0.2992 | 0.00 | 2000.00 | 0.5240 | old |
| RS2292962 | CTSD | 11 | 1742630 | 0.1193 | 2.44 | 2002.44 | 0.9234 | affec |
| RS1317356 | CTSD | 11 | 1743447 | 0.1309 | 2.44 | 2002.44 | 0.8831 | old |
| RS17571 | CTSD | 11 | 1746903 | 0.034 | 2.44 | 2002.44 | 1.4750 | old |
| RS830083 | ACP2 | 11 | 47218360 | 0.2321 | 58.40 | 2058.40 | 0.6343 | affec |
| RS11988 | ACP2 | 11 | 47225569 | 0.2032 | 58.40 | 2058.40 | 0.6921 | affec |
| RS2242261 | ACP2 | 11 | 47231117 | 0.2201 | 58.40 | 2058.40 | 0.6574 | affec |
| HCV1301047 | ACP2 | 11 | 47234245 | 0.2999 | 58.40 | 2058.40 | 0.5230 | affec |
| RS3758673 | NR1H3 | 11 | 47243226 | 0.1476 | 58.40 | 2058.40 | 0.8309 | old |
| RS2279238 | NR1H3 | 11 | 47246333 | 0.4009 | 58.40 | 2058.40 | 0.3970 | affec |
| RS1449627 | NR1H3 | 11 | 47255293 | 0.3559 | 58.40 | 2058.40 | 0.4487 | young |
| RS2291119 | NR1H3 | 11 | 47262510 | 0.0758 | 58.40 | 2058.40 | 1.1203 | affec |
| RS326214 | NR1H3 | 11 | 47262669 | 0.3146 | 58.40 | 2058.40 | 0.5022 | old |
| HCV25595878 | TCIRG1 | 11 | 64676766 | 0.1957 | 66.50 | 2066.50 | 0.7084 | affec |
| RS906713 | TCIRG1 | 11 | 67589290 | 0.0679 | 67.48 | 2067.48 | 1.1681 | young |
| RS2075609 | TCIRG1 | 11 | 67592296 | 0.021 | 67.48 | 2067.48 | 1.6861 | old |
| RS11481 | TCIRG1 | 11 | 67595695 | 0.2304 | 67.48 | 2067.48 | 0.6375 | old |
| RS2851069 | SLC21A9 | 11 | 74588664 | 0.6426 | 77.78 | 2077.78 | 0.1921 | old |
| HCV1786352 | SLC21A9 | 11 | 74601797 | 0.4833 | 77.78 | 2077.78 | 0.3158 | affec |
| RS2851109 | SLC21A9 | 11 | 74604384 | 0.555 | 77.78 | 2077.78 | 0.2557 | affec |
| RS609887 | MMP7 | 11 | 101922284 | 0.0613 | 98.98 | 2098.98 | 1.2125 | young |
| 11P0321 | MMP7 | 11 | 101929004 | 0.1658 | 98.98 | 2098.98 | 0.7804 | old |
| HCV12088722 | MMP7 | 11 | 101929142 | 0.009 | 98.98 | 2098.98 | 2.0362 | affec |
| RS1996352 | MMP7 | 11 | 101933964 | 0.0932 | 98.98 | 2098.98 | 1.0306 | affec |
| HCV3210838 | MMP7 | 11 | 101936310 | 0.003 | 98.98 | 2098.98 | 2.5686 | affec |
| RS1943779 | MMP7 | 11 | 101944908 | 0.2188 | 98.98 | 2098.98 | 0.6600 | young |
| RS674546 | MMP12 | 11 | 102268356 | 0.049 | 99.11 | 2099.11 | 1.3098 | affec |
| RS505770 | MMP12 | 11 | 102271927 | 0.1518 | 99.11 | 2099.11 | 0.8187 | affec |
| HCV785907 | MMP12 | 11 | 102274359 | 0.2482 | 99.11 | 2099.11 | 0.6052 | young |
| RS2276109 | MMP12 | 11 | 102283508 | 0.1997 | 99.12 | 2099.12 | 0.6996 | young |
| RS1277718 | MMP12 | 11 | 102285268 | 0.1288 | 99.12 | 2099.12 | 0.8901 | young |
| RS660407 | FLI1 | 11 | 128175388 | 0.032 | 131.26 | 2131.26 | 1.5003 | old |
| RS497714 | FLI1 | 11 | 128181205 | 0.0531 | 131.26 | 2131.26 | 1.2749 | old |
| RS526091 | FLI1 | 11 | 128188604 | 0.231 | 131.26 | 2131.26 | 0.6364 | affec |
| RS656972 | FLI1 | 11 | 128198398 | 0.2855 | 131.27 | 2131.27 | 0.5444 | affec |
| RS687326 | FLI1 | 11 | 128210233 | 0.1014 | 131.47 | 2131.47 | 0.9940 | young |
| RS2301262 | PTPN6 | 12 | 6926121 | 0.1455 | 16.22 | 2157.22 | 0.8371 | old |
| HCV3266450 | PTPN6 | 12 | 6927395 | 0.038 | 16.23 | 2157.23 | 1.4214 | affec |
| RS7978658 | PTPN6 | 12 | 6928231 | 0.039 | 16.23 | 2157.23 | 1.4101 | affec |
| RS7966756 | PTPN6 | 12 | 6932652 | 0.2119 | 16.25 | 2157.25 | 0.6739 | affec |
| RS2110072 | PTPN6 | 12 | 6935896 | 0.1585 | 16.26 | 2157.26 | 0.8000 | old |
| RS253147 | CLECSF2 | 12 | 9906349 | 0.2628 | 20.27 | 2161.27 | 0.5804 | affec |
| RS1050286 | OLR1 | 12 | 10202830 | 0.0555 | 20.27 | 2161.27 | 1.2557 | young |
| 12P0322 | OLR1 | 12 | 10203915 | 0.0737 | 20.27 | 2161.27 | 1.1325 | affec |
| HCV3130874 | OLR1 | 12 | 10204532 | 0.0635 | 20.27 | 2161.27 | 1.1972 | affec |
| RS3736232 | OLR1 | 12 | 10204625 | 0.0819 | 20.27 | 2161.27 | 1.0867 | affec |
| RS3741860 | OLR1 | 12 | 10211711 | 0.0952 | 20.27 | 2161.27 | 1.0214 | affec |
| RS2742113 | OLR1 | 12 | 10214149 | 0.063 | 20.27 | 2161.27 | 1.2007 | affec |
| RS1548836 | RAI3 | 12 | 12945501 | 0.2949 | 29.49 | 2170.49 | 0.5303 | young |
| RS2075288 | RAI3 | 12 | 12952561 | 0.4566 | 29.55 | 2170.55 | 0.3405 | young |
| RS1061047 | RAI3 | 12 | 12957487 | 0.1835 | 29.59 | 2170.59 | 0.7364 | young |
| RS1800801 | MGP | 12 | 14930055 | 0.2413 | 31.67 | 2172.67 | 0.6174 | old |
| RS3741552 | ITPR2 | 12 | 26624254 | 0.1641 | 46.84 | 2187.84 | 0.7849 | affec |
| RS2230372 | ITPR2 | 12 | 26676117 | 0.3387 | 46.95 | 2187.95 | 0.4702 | old |
| RS2291264 | ITPR2 | 12 | 26702044 | 0.1558 | 47.01 | 2188.01 | 0.8074 | affec |
| RS1900941 | ITPR2 | 12 | 26759589 | 0.0604 | 47.13 | 2188.13 | 1.2190 | affec |
| RS1449568 | ITPR2 | 12 | 26802972 | 0.03 | 47.23 | 2188.23 | 1.5229 | affec |
| RS2016107 | TUBA3 | 12 | 47863524 | 0.1658 | 64.43 | 2205.43 | 0.7804 | old |
| RS6580704 | TUBA3 | 12 | 47866447 | 0.2749 | 64.43 | 2205.43 | 0.5608 | old |
| RS1039225 | TUBA3 | 12 | 47868959 | 0.0957 | 64.43 | 2205.43 | 1.0191 | young |
| RS1874908 | TUBA3 | 12 | 47870297 | 0.3594 | 64.43 | 2205.43 | 0.4444 | old |
| HCV48424 | PTPRR | 12 | 69334002 | 0.6934 | 82.12 | 2223.12 | 0.1590 | affec |
| RS2137537 | PTPRR | 12 | 69399354 | 0.017 | 82.12 | 2223.12 | 1.7773 | young |
| RS972769 | PTPRR | 12 | 69419738 | 0.125 | 82.14 | 2223.14 | 0.9031 | affec |
| HCV155408 | PTPRR | 12 | 69525275 | 0.1261 | 82.33 | 2223.33 | 0.8993 | affec |
| HCV93800 | PTPRR | 12 | 69570924 | 0.042 | 82.41 | 2223.41 | 1.3809 | young |
| RS2300588 | LUM | 12 | 90002365 | 0.1377 | 96.09 | 2237.09 | 0.8611 | young |
| RS2230754 | PLXNC1 | 12 | 93045974 | 0.3762 | 97.16 | 2238.16 | 0.4246 | affec |
| RS3858609 | PLXNC1 | 12 | 93067143 | 0.2971 | 97.16 | 2238.16 | 0.5271 | old |
| RS2305971 | PLXNC1 | 12 | 93105768 | 0.02 | 97.16 | 2238.16 | 1.7011 | young |
| RS2242498 | PLXNC1 | 12 | 93152063 | 0.3119 | 97.30 | 2238.30 | 0.5060 | young |
| RS1681866 | PLXNC1 | 12 | 93178913 | 0.2017 | 97.41 | 2238.41 | 0.6953 | old |
| RS25642 | | 12 | 120071870 | 0.2587 | 139.61 | 2280.61 | 0.5872 | young |
| RS25643 | P2RX4 | 12 | 120072740 | 0.146 | 139.61 | 2280.61 | 0.8356 | young |
| RS25644 | CAMKK2 | 12 | 120078599 | 0.0763 | 139.61 | 2280.61 | 1.1175 | young |
| RS2071272 | P2RX4 | 12 | 120082745 | 0.1241 | 139.61 | 2280.61 | 0.9062 | young |
| RS6750 | OSF-2 | 13 | 35934832 | 0.3582 | 31.37 | 2342.37 | 0.4459 | old |
| HCV227836 | OSF-2 | 13 | 35944998 | 0.2274 | 31.39 | 2342.39 | 0.6432 | young |
| HCV11170344 | OSF-2 | 13 | 35952189 | 0.2378 | 31.41 | 2342.41 | 0.6238 | young |
| HCV1909050 | OSF-2 | 13 | 35952905 | 0.5775 | 31.41 | 2342.41 | 0.2384 | old |
| HCV1909043 | OSF-2 | 13 | 35961093 | 0.3062 | 31.43 | 2342.43 | 0.5140 | old |
| HCV1909039 | OSF-2 | 13 | 35969741 | 0.2654 | 31.45 | 2342.45 | 0.5761 | young |
| RS1890139 | PCCA | 13 | 98480093 | 0.3662 | 81.06 | 2392.06 | 0.4363 | young |
| HCV1823453 | PCCA | 13 | 98526614 | 0.018 | 81.29 | 2392.29 | 1.7423 | young |
| HCV2747127 | PCCA | 13 | 98616632 | 0.0531 | 81.64 | 2392.64 | 1.2749 | old |
| RS1296332 | PCCA | 13 | 98811747 | 0.2807 | 81.64 | 2392.64 | 0.5518 | affec |
| HCV2786590 | RTN1 | 14 | 58099812 | 0.2634 | 66.81 | 2486.81 | 0.5794 | affec |
| HCV1964266 | RTN1 | 14 | 58169316 | 0.1343 | 66.81 | 2486.81 | 0.8719 | affec |
| HCV1964289 | RTN1 | 14 | 58199104 | 0.116 | 66.81 | 2486.81 | 0.9355 | affec |
| HCV2141342 | RTN1 | 14 | 58293242 | 0.0786 | 66.81 | 2486.81 | 1.1046 | young |
| RS1951795 | HIF1A | 14 | 60161467 | 0.1871 | 67.51 | 2487.51 | 0.7279 | young |
| RS3783752 | HIF1A | 14 | 60175733 | 0.007 | 67.52 | 2487.52 | 2.1612 | affec |
| RS2301111 | HIF1A | 14 | 60190242 | 0.3087 | 67.54 | 2487.54 | 0.5105 | young |
| RS2301113 | HIF1A | 14 | 60196589 | 0.1522 | 67.54 | 2487.54 | 0.8176 | affec |
| RS2057482 | HIF1A | 14 | 60203889 | 0.2456 | 67.55 | 2487.55 | 0.6098 | young |
| RS875395 | ITPK1 | 14 | 91392134 | 0.031 | 107.41 | 2527.41 | 1.5072 | young |
| HCV1259613 | ITPK1 | 14 | 91399119 | 0.039 | 107.43 | 2527.43 | 1.4067 | old |
| RS2295394 | ITPK1 | 14 | 91402784 | 0.4938 | 107.44 | 2527.44 | 0.3064 | young |
| RS2402226 | ITPK1 | 14 | 91409576 | 0.013 | 107.45 | 2527.45 | 1.8729 | old |
| RS1614269 | ITPK1 | 14 | 91493546 | 0.1583 | 107.63 | 2527.63 | 0.8005 | old |
| RS1740595 | ITPK1 | 14 | 91502571 | 0.1124 | 107.65 | 2527.65 | 0.9492 | old |
| RS4905043 | ITPK1 | 14 | 91540050 | 0.0743 | 107.73 | 2527.73 | 1.1290 | affec |
| HCV1258994 | ITPK1 | 14 | 91544259 | 0.659 | 107.74 | 2527.74 | 0.1811 | affec |
| HCV1882714 | C14ORF132 | 14 | 94541644 | 0.2806 | 114.81 | 2534.81 | 0.5519 | affec |
| HCV1882697 | C14ORF132 | 14 | 94549500 | 0.1478 | 114.81 | 2534.81 | 0.8303 | young |
| HCV9706786 | PP9099 | 15 | 43808547 | 0.2693 | 42.89 | 2583.89 | 0.5698 | affec |
| HCV1977407 | PP9099 | 15 | 62851357 | 0.3227 | 60.14 | 2601.14 | 0.4912 | young |
| HCV497654 | PP9099 | 15 | 62869949 | 0.046 | 60.15 | 2601.15 | 1.3420 | young |
| HGV497653 | PP9099 | 15 | 62870401 | 0.4805 | 60.15 | 2601.15 | 0.3183 | young |
| RS293379 | ABHD2 | 15 | 87363708 | 0.3178 | 85.64 | 2626.64 | 0.4978 | affec |
| HCV1597898 | ABHD2 | 15 | 87381022 | 0.3416 | 85.64 | 2626.64 | 0.4665 | affec |
| RS4327024 | ABHD2 | 15 | 87430443 | 0.4509 | 85.64 | 2626.64 | 0.3459 | young |
| RS1005398 | ABHD2 | 15 | 87449143 | 0.023 | 85.64 | 2626.64 | 1.6345 | old |
| RS2239288 | ABHD2 | 15 | 87461115 | 0.4109 | 85.64 | 2626.64 | 0.3863 | old |
| RS10584 | ANPEP | 15 | 88058319 | 0.026 | 85.64 | 2626.64 | 1.5850 | affec |
| RS1992250 | ANPEP | 15 | 88063748 | 0.014 | 85.64 | 2626.64 | 1.8508 | affec |
| RS7168793 | ANPEP | 15 | 88064008 | 0.028 | 85.64 | 2626.64 | 1.5575 | affec |
| RS1439119 | ANPEP | 15 | 88068014 | 0.049 | 85.64 | 2626.64 | 1.3089 | affec |
| HCV1576494 | CIB1 | 15 | 88516119 | 0.2975 | 85.64 | 2626.64 | 0.5265 | affec |
| HCV12104474 | CIB1 | 15 | 88524613 | 0.3159 | 85.64 | 2626.64 | 0.5005 | affec |
| RS1105702 | CIB1 | 15 | 88525621 | 0.5284 | 85.64 | 2626.64 | 0.2770 | young |
| RS2048707 | CIB1 | 15 | 88531352 | 0.2061 | 85.65 | 2626.65 | 0.6859 | affec |
| HCV1576445 | CIB1 | 15 | 88538275 | 0.1703 | 85.69 | 2626.69 | 0.7688 | young |
| RS4378630 | ITGAX | 16 | 31406512 | 0.1847 | 57.79 | 2712.79 | 0.7335 | old |
| RS4264407 | ITGAX | 16 | 31407253 | 0.0903 | 57.79 | 2712.79 | 1.0443 | affec |
| RS2070896 | ITGAX | 16 | 31420614 | 0.1255 | 57.79 | 2712.79 | 0.9014 | affec |
| RS2929 | ITGAX | 16 | 31429368 | 0.3302 | 57.80 | 2712.80 | 0.4812 | old |
| RS1140195 | ITGAX | 16 | 31430239 | 0.1047 | 57.80 | 2712.80 | 0.9801 | old |
| RS1030868 | MMP2 | 16 | 55295369 | 0.3025 | 73.77 | 2728.77 | 0.5193 | old |
| RS1053605 | MMP2 | 16 | 55298209 | 0.2501 | 73.78 | 2728.78 | 0.6019 | old |
| RS243849 | MMP2 | 16 | 55302307 | 0.2651 | 73.80 | 2728.80 | 0.5766 | affec |
| RS2287076 | MMP2 | 16 | 55311060 | 0.3748 | 73.84 | 2728.84 | 0.4262 | young |
| RS7201 | MMP2 | 16 | 55318216 | 0.7635 | 73.87 | 2728.87 | 0.1172 | affec |
| RS3180279 | CYBA | 16 | 88454958 | 0.1388 | 131.44 | 2786.44 | 0.8576 | old |
| RS4987131 | CYBA | 16 | 88457338 | 0.467 | 131.44 | 2786.44 | 0.3307 | old |
| RS3812948 | CYBA | 16 | 88460659 | 0.3852 | 131.45 | 2786.45 | 0.4143 | old |
| RS3817655 | CCL5 | 17 | 34345191 | 0.2133 | 57.76 | 2839.26 | 0.6710 | old |
| RS2107538 | CCL5 | 17 | 34353330 | 0.2404 | 57.77 | 2839.27 | 0.6191 | old |
| RS1634481 | CCL3 | 17 | 34551225 | 0.2031 | 57.91 | 2839.41 | 0.6923 | old |
| RS1719134 | CCL3 | 17 | 34562496 | 0.132 | 57.92 | 2839.42 | 0.8794 | old |
| RS4432296 | CNP | 17 | 40491972 | 0.4999 | 62.01 | 2843.51 | 0.3011 | old |
| HCV11618196 | CNP | 17 | 40496994 | 0.1954 | 62.01 | 2843.51 | 0.7091 | old |
| RS2229931 | CNP | 17 | 40498936 | 0.2762 | 62.01 | 2843.51 | 0.5588 | young |
| RS2070106 | CNP | 17 | 40499029 | 0.4811 | 62.01 | 2843.51 | 0.3178 | affec |
| HCV437993 | CNP | 17 | 40499352 | 0.0818 | 62.01 | 2843.51 | 1.0872 | old |
| RS2272087 | STAT5A | 17 | 40832727 | 0.2716 | 63.09 | 2844.59 | 0.5661 | young |
| RS1135669 | STAT5A | 17 | 40832902 | 0.3946 | 63.09 | 2844.59 | 0.4038 | young |
| RS3198502 | STAT5A | 17 | 40836159 | 0.1796 | 63.09 | 2844.59 | 0.7457 | old |
| HCV2548250 | GRN | 17 | 42898428 | 0.029 | 63.70 | 2845.20 | 1.5421 | young |
| RS3785817 | GRN | 17 | 42898830 | 0.1495 | 63.70 | 2845.20 | 0.8254 | affec |
| RS3815057 | GRN | 17 | 42902795 | 0.024 | 63.71 | 2845.21 | 1.6144 | old |
| RS25647 | GRN | 17 | 42905004 | 0.503 | 63.71 | 2845.21 | 0.2984 | affec |
| RS5848 | GRN | 17 | 42905409 | 0.2219 | 63.71 | 2845.21 | 0.6538 | affec |
| HCV9267947 | FMNL1 | 17 | 43791782 | 0.036 | 64.86 | 2846.36 | 1.4425 | old |
| RS1801353 | FMNL1 | 17 | 43795192 | 0.05 | 64.87 | 2846.37 | 1.3019 | old |
| HCV9267944 | FLJ25414 | 17 | 43808547 | 0.0635 | 64.88 | 2846.38 | 1.1972 | old |
| RS1384367 | PSCD1 | 17 | 77326880 | 0.2378 | 109.57 | 2891.07 | 0.6238 | old |
| RS1871935 | PSCD1 | 17 | 77361268 | 0.1061 | 109.76 | 2891.26 | 0.9743 | young |
| HCV12126963 | PSCD1 | 17 | 77370761 | 0.0904 | 109.81 | 2891.31 | 1.0438 | old |
| RS2276195 | TCF4 | 18 | 51045496 | 0.1176 | 77.12 | 2985.12 | 0.9296 | old |
| RS1261076 | TCF4 | 18 | 51057313 | 0.1318 | 77.13 | 2985.13 | 0.8801 | old |
| HCV11452698 | TCF4 | 18 | 51067704 | 0.133 | 77.14 | 2985.14 | 0.8761 | young |
| RS1440476 | TCF4 | 18 | 51189053 | 0.6782 | 77.25 | 2985.25 | 0.1686 | affec |
| RS2119292 | TCF4 | 18 | 51231961 | 0.1306 | 77.29 | 2985.29 | 0.8841 | affec |
| RS2958182 | TCF4 | 18 | 51298008 | 0.2291 | 77.35 | 2985.35 | 0.6400 | young |
| RS613872 | TCF4 | 18 | 51359289 | 0.2826 | 77.36 | 2985.36 | 0.5488 | old |
| RS243375 | CHAF1A | 19 | 4388089 | 0.1945 | 15.91 | 3044.91 | 0.7111 | young |
| RS932276 | UBXD1 | 19 | 4390525 | 0.0551 | 15.93 | 3044.93 | 1.2588 | young |
| RS9352 | CHAF1A | 19 | 4393336 | 0.1659 | 15.95 | 3044.95 | 0.7802 | young |
| RS243382 | CHAF1A | 19 | 4393529 | 0.1287 | 15.95 | 3044.95 | 0.8904 | affec |
| RS741923 | UBXD1 | 19 | 4399843 | 0.0696 | 16.00 | 3045.00 | 1.1574 | young |
| RS243395 | UBXD1 | 19 | 4403497 | 0.0972 | 16.03 | 3045.03 | 1.0123 | young |
| RS1044510 | UBXD1 | 19 | 4408650 | 0.1256 | 16.07 | 3045.07 | 0.9010 | young |
| RS6510808 | UBXD1 | 19 | 4409911 | 0.1791 | 16.08 | 3045.08 | 0.7469 | young |
| RS2913984 | | 19 | 8379580 | 0.1217 | 28.07 | 3057.07 | 0.9147 | young |
| RS2396141 | | 19 | 8398574 | 0.031 | 28.17 | 3057.17 | 1.5072 | young |
| RS2303180 | | 19 | 8401765 | 0.5057 | 28.18 | 3057.18 | 0.2961 | old |
| RS3815783 | | 19 | 8409025 | 0.1804 | 28.22 | 3057.22 | 0.7438 | young |
| RS6603074 | | 19 | 8411332 | 0.1505 | 28.23 | 3057.23 | 0.8225 | affec |
| RS6603076 | | 19 | 8413177 | 0.2261 | 28.24 | 3057.24 | 0.6457 | affec |
| RS2229531 | ACP5 | 19 | 11548195 | 0.017 | 35.57 | 3064.57 | 1.7645 | affec |
| RS2305799 | ACP5 | 19 | 11548351 | 0.021 | 35.57 | 3064.57 | 1.6737 | old |
| RS2071485 | ACP5 | 19 | 11549200 | 0.0511 | 35.57 | 3064.57 | 1.2916 | young |
| RS2071484 | ACP5 | 19 | 11549460 | 0.027 | 35.57 | 3064.57 | 1.5751 | old |
| RS2071483 | ACP5 | 19 | 11549539 | 0.2532 | 35.57 | 3064.57 | 0.5965 | affec |
| RS2241089 | IFI30 | 19 | 18145651 | 0.5143 | 47.72 | 3076.72 | 0.2888 | affec |
| RS2241090 | IFI30 | 19 | 18146751 | 0.497 | 47.72 | 3076.72 | 0.3036 | affec |
| RS4808756 | IFI30 | 19 | 18149004 | 0.972 | 47.72 | 3076.72 | 0.0123 | affec |
| RS7125 | IFI30 | 19 | 18149069 | 0.0723 | 47.72 | 3076.72 | 1.1409 | affec |
| RS2921 | IFI30 | 19 | 18149810 | 0.5117 | 47.72 | 3076.72 | 0.2910 | young |
| RS2303692 | ELL | 19 | 18418657 | 0.0654 | 47.78 | 3076.78 | 1.1844 | affec |
| HCV1399152 | ELL | 19 | 18421282 | 0.038 | 47.78 | 3076.78 | 1.4202 | affec |
| RS731945 | ELL | 19 | 18428039 | 0.2426 | 47.79 | 3076.79 | 0.6151 | old |
| HCV8161961 | ELL | 19 | 18473351 | 0.037 | 47.80 | 3076.80 | 1.4283 | affec |
| HCV8161938 | ELL | 19 | 18479225 | 0.006 | 47.80 | 3076.80 | 2.2291 | affec |
| RS3786874 | SPINT2 | 19 | 43450782 | 0.4105 | 62.56 | 3091.56 | 0.3867 | young |
| HCV7822158 | SPINT2 | 19 | 43456912 | 0.0743 | 62.59 | 3091.59 | 1.1290 | young |
| RS1006140 | SPINT2 | 19 | 43470755 | 0.1026 | 62.65 | 3091.65 | 0.9889 | young |
| RS4760 | PLAUR | 19 | 48844940 | 0.2575 | 67.37 | 3096.37 | 0.5892 | young |
| RS2283628 | PLAUR | 19 | 48854901 | 0.1747 | 67.37 | 3096.37 | 0.7577 | old |
| RS399145 | PLAUR | 19 | 48861362 | 0.5229 | 67.37 | 3096.37 | 0.2816 | affec |
| RS2286960 | PLAUR | 19 | 48863865 | 0.031 | 67.37 | 3096.37 | 1.5129 | old |
| RS440446 | APOE | 19 | 50101007 | 0.2555 | 69.50 | 3098.50 | 0.5926 | young |
| RS769449 | APOE | 19 | 50101842 | 0.0989 | 69.50 | 3098.50 | 1.0048 | old |
| RS769450 | APOE | 19 | 50102284 | 0.0962 | 69.50 | 3098.50 | 1.0168 | affec |
| RS7412 | APOE | 19 | 50103919 | 0.3126 | 69.50 | 3098.50 | 0.5050 | old |
| RS483082 | APOC1 | 19 | 50108018 | 0.08 | 69.50 | 3098.50 | 1.0969 | affec |
| RS1064725 | APOC1 | 19 | 50114401 | 0.3498 | 69.50 | 3098.50 | 0.4562 | young |
| RS5157 | APOC2 | 19 | 50139001 | 0.5997 | 69.50 | 3098.50 | 0.2221 | young |
| RS5120 | APOC2 | 19 | 50143460 | 0.5398 | 69.50 | 3098.50 | 0.2678 | young |
| RS5126 | APOC2 | 19 | 50144269 | 0.6604 | 69.50 | 3098.50 | 0.1802 | young |
| RS3760627 | APOC2 | 19 | 50149020 | 0.2672 | 69.50 | 3098.50 | 0.5732 | young |
| RS2239375 | APOC2 | 19 | 50151691 | 0.3258 | 69.50 | 3098.50 | 0.4870 | young |
| RS1805419 | FTL | 19 | 54150916 | 0.02 | 74.43 | 3103.43 | 1.7033 | young |
| RS4645887 | FTL | 19 | 54151688 | 0.0859 | 74.44 | 3103.44 | 1.0660 | affec |
| RS2387583 | FTL | 19 | 54153117 | 0.041 | 74.44 | 3103.44 | 1.3851 | young |
| RS1042265 | GYS1 | 19 | 54163632 | 0.011 | 74.46 | 3103.46 | 1.9706 | young |
| RS2270938 | GYS1 | 19 | 54165839 | 0.0706 | 74.46 | 3103.46 | 1.1512 | affec |
| HCV1997111 | LAIR1 | 19 | 59544316 | 0.3295 | 90.99 | 3119.99 | 0.4821 | old |
| RS2287824 | LAIR1 | 19 | 59554712 | 0.1514 | 91.01 | 3120.01 | 0.8199 | affec |
| RS2664538 | MMP9 | 20 | 45325647 | 0.5834 | 64.88 | 3190.88 | 0.2340 | young |
| RS13969 | MMP9 | 20 | 45328255 | 0.0526 | 64.88 | 3190.88 | 1.2790 | old |
| RS2274756 | MMP9 | 20 | 45328533 | 0.3632 | 64.88 | 3190.88 | 0.4399 | young |
| RS13925 | MMP9 | 20 | 45330387 | 0.4266 | 64.88 | 3190.88 | 0.3700 | young |
| RS9509 | MMP9 | 20 | 45330575 | 0.1485 | 64.88 | 3190.88 | 0.8283 | young |
| RS2766669 | ZNF217 | 20 | 52863648 | 0.048 | 80.63 | 3206.63 | 1.3152 | young |
| RS743466 | CSTB | 21 | 44047207 | 0.159 | 52.50 | 3274.50 | 0.7986 | young |
| RS2838363 | CSTB | 21 | 44047341 | 0.404 | 52.50 | 3274.50 | 0.3936 | affec |
| RS6375 | CSTB | 21 | 44051731 | 0.9802 | 52.50 | 3274.50 | 0.0087 | old |
| RS3761385 | CSTB | 21 | 44054557 | 0.024 | 52.50 | 3274.50 | 1.6234 | old |
| HCV11479371 | SMTN | 22 | 29789771 | 0.4881 | 29.03 | 3309.03 | 0.3115 | young |
| HCV2628881 | SMTN | 22 | 29806208 | 0.4063 | 29.04 | 3309.04 | 0.3912 | young |
| HCV2628867 | SMTN | 22 | 29817679 | 0.0834 | 29.05 | 3309.05 | 1.0788 | old |
| HCV2628861 | SMTN | 22 | 29819997 | 0.4669 | 29.05 | 3309.05 | 0.3308 | affec |
| HCV2628858 | SMTN | 22 | 29822433 | 0.4293 | 29.05 | 3309.05 | 0.3672 | young |
| RS5757424 | APOBEC3D | 22 | 37658819 | 0.3269 | 46.12 | 3326.12 | 0.4856 | affec |
| RS5757425 | APOBEC3D | 22 | 37662522 | 0.1552 | 46.13 | 3326.13 | 0.8091 | young |
| RS6001388 | APOBEC3D | 22 | 37662892 | 0.2216 | 46.13 | 3326.13 | 0.6544 | affec |

A detailed list of the top genes and SNPs in those genes ranked by p-value for each gene is included as Table 2 below. Genes identified having at least 1 SNP with a p-value less than 0.10 are shown in bold. Genes were identified from logistic regression analysis of three models only: OA vs. ON, YA vs. ON and YA vs. OA. The column headers represent the following: GENE: Gene name (HUGO ID); Gene alias: Non-HUGO ID gene aliases or previous gene names; Meta Rank: Gene rank from the David Seo/Mike West microarray expression study [PMID:15297278]; Pᵥₐₗ Rank: Gene rank based on lowest Cathgen p-value for any SNP/model in that gene (lowest p-value has rank of 1); Startloc: Gene's base pair start location from NCBI build 35; Chr: Chromosome; # SNPS: Number of SNPs in that gene genotyped in Cathgen individuals; Lowest p-value: Lowest p-value for any SNP/model in that gene from logistic regression analysis of groups C1+C2 (1037 individuals); adjusted for sex and ethnicity; Model: SNP model with the lowest p-value (responsible for that gene's Top Gene p-value ranking); Other models <0.10: All other SNPs, models in that gene with a p-value <0.10. Abbreviations are as follows: A, Allele test; G, Genotype test; YVN, Young Affected v. Old Normal; OVN, Old Affected v. Old Normal; YVO, Young Affected v. Old Affected

TABLE 2

| | Meta | P val | | | Lowest | | |
|---|---|---|---|---|---|---|---|
| GENE | Rank | Rank | Startloc | CH | p-value | Model | Other models <.10 |
| AIM1L | 230 | 1 | 26356066 | 1 | 0.0001 | RS4454539, | RS4454539, G, YVN |
| | | | | | | A, YVN | RS4454539, A, OVN |
| | | | | | | | RS4454539, G, OVN |
| | | | | | | | RS4454539, G, YVO |
| | | | | | | | RS4659431, A, OVN |
| PLA2G7 | 0 | 2 | 46780013 | 6 | 0.0001 | RS1805017, | RS9381475, G, OVN |
| | | | | | | G, OVN | RS9381475, A, OVN |
| | | | | | | | RS1805017, A, OVN |
| | | | | | | | RS1862008, A, OVN |
| | | | | | | | RS1862008, G, OVN |
| | | | | | | | RS1051931, G, YVN |
| | | | | | | | RS9381475, G, YVO |
| | | | | | | | RS1051931, A, YVN |
| | | | | | | | RS1051931, G, OVN |
| | | | | | | | RS9381475, A, YVO |
| | | | | | | | RS1862008, G, YVO |
| | | | | | | | RS1862008, A, YVO |
| | | | | | | | RS1051931, A, OVN |
| | | | | | | | RS1805017, A, YVN |
| | | | | | | | RS1805017, G, YVO |
| | | | | | | | RS1805017, G, YVN |
| | | | | | | | RS1805018, A, YVN |
| | | | | | | | RS1805018, G, YVN |
| OR7E29P | 0 | 4 | 126913676 | 3 | 0.0003 | RS2979310, | RS2979310, G, YVN |
| | | | | | | A, YVN | RS2979310, A, OVN |
| PLN | 108 | 5 | 118986778 | 6 | 0.0003 | RS1051429, | RS1051429, A, YVN |
| | | | | | | G, YVN | RS3734382, G, YVN |
| | | | | | | | RS3734382, A, YVN |
| | | | | | | | RS1051429, A, YVO |
| | | | | | | | RS1051429, G, YVO |
| | | | | | | | 6P0326, G, YVN |
| | | | | | | | RS1051429, G, OVN |
| | | | | | | | RS3734382, G, YVO |
| | | | | | | | 6P0326, A, YVO |
| | | | | | | | 6P0326, G, YVO |
| | | | | | | | RS3734382, A, YVO |
| | | | | | | | 6P0326, A, YVN |
| | | | | | | | RS3752581, A, YVO |
| | | | | | | | 6P0324, A, YVO |
| PTPN6 | 187 | 6 | 6911553 | 12 | 0.0003 | RS7310161, | RS7978658, G, YVO |
| | | | | | | A, YVO | RS7978658, A, YVO |
| | | | | | | | RS7310161, G, YVO |
| | | | | | | | RS7310161, A, YVN |
| CIORF38 | 32 | 7 | 27883228 | 1 | 0.0005 | RS3766398, | RS3766398, G, YVO |
| | | | | | | A, YVO | RS3766398, G, OVN |
| | | | | | | | RS12048235, G, OVN |
| | | | | | | | RS3766398, A, OVN |
| | | | | | | | RS6564, A, OVN |
| | | | | | | | RS6564, G, OVN |
| | | | | | | | RS12048235, A, OVN |
| | | | | | | | RS1467464, G, OVN |
| | | | | | | | RS6564, A, YVO |
| | | | | | | | RS1467464, A, OVN |
| | | | | | | | RS12048235, A, YVO |
| | | | | | | | RS1467465, A, YVO |
| | | | | | | | RS12048235, G, YVO |
| GATA2 | 0 | 8 | 129680970 | 3 | 0.0006 | RS2335052, | RS6439129, G, YVO |
| | | | | | | A, YVO | RS1573858, G, YVO |
| | | | | | | | RS2335052, G, YVO |
| | | | | | | | RS6439129, A, YVO |
| | | | | | | | RS2335052, A, OVN |
| | | | | | | | RS2335052, G, OVN |
| | | | | | | | RS1573858, A, YVO |
| | | | | | | | RS2713603, G, YVO |
| | | | | | | | RS1573858, G, OVN |
| | | | | | | | RS2713603, G, OVN |
| | | | | | | | RS2713603, A, YVO |
| | | | | | | | RS2713603, A, OVN |
| | | | | | | | RS6439129, G, OVN |
| | | | | | | | RS3803, A, OVN |
| | | | | | | | RS3803, A, YVN |
| IL7R | 0 | 9 | 35892748 | 5 | 0.0006 | RS1494555, | RS1494555, A, OVN |
| | | | | | | G, OVN | RS1494555, A, YVN |
| | | | | | | | RS1494555, G, YVN |
| | | | | | | | RS987106, G, OVN |
| | | | | | | | RS2228141, G, YVO |
| MYLK | 0 | 10 | 124813835 | 3 | 0.0007 | RS16834817, | RS16834817, A, YVN |
| | | | | | | G, YVN | HCV1602689, G, YVN |
| | | | | | | | HCV1602689, A, YVN |
| | | | | | | | RS16834817, G, OVN |
| | | | | | | | HCV1602689, G, OVN |
| | | | | | | | RS4118366, A, OVN |
| | | | | | | | RS16834817, A, OVN |
| | | | | | | | RS2682215, A, OVN |
| | | | | | | | RS2682239, A, OVN |
| | | | | | | | RS4118366, G, OVN |
| | | | | | | | RS4461370, A, OVN |
| | | | | | | | HCV1602689, A, OVN |
| | | | | | | | RS4461370, G, OVN |
| | | | | | | | RS2682215, G, OVN |
| | | | | | | | RS2700358, G, OVN |
| | | | | | | | RS2682229, A, OVN |
| | | | | | | | RS2700358, A, OVN |
| | | | | | | | RS2682239, G, OVN |
| | | | | | | | RS2682229, G, OVN |
| | | | | | | | RS11717814, G, OVN |
| | | | | | | | RS16834826, G, YVO |
| | | | | | | | RS2605417, A, OVN |
| | | | | | | | RS1343700, G, YVO |
| | | | | | | | RS820371, G, OVN |
| | | | | | | | RS2682215, G, YVO |
| | | | | | | | RS2700408, G, OVN |
| | | | | | | | RS2605417, G, OVN |
| | | | | | | | RS4118366, A, YVN |
| | | | | | | | RS2700408, A, OVN |
| | | | | | | | RS4461370, G, YVO |
| | | | | | | | RS1343700, A, YVN |
| | | | | | | | RS1343700, G, YVN |
| | | | | | | | RS4118366, G, YVO |
| | | | | | | | RS2700358, G, YVO |
| | | | | | | | RS2682215, A, YVN |
| | | | | | | | RS2682239, A, YVN |
| | | | | | | | RS2682229, G, YVO |
| | | | | | | | RS11717814, A, OVN |
| | | | | | | | RS2700408, G, YVO |
| ANPEP | 146 | 11 | 88129131 | 15 | 0.0008 | RS10584, A, | RS10584, A, OVN |
| | | | | | | YVO | RS25653, A, OVN |
| | | | | | | | RS25653, G, OVN |
| | | | | | | | RS10584, G, OVN |
| | | | | | | | RS10584, G, YVO |
| | | | | | | | RS25653, G, YVO |
| | | | | | | | RS25653, A, YVO |
| | | | | | | | RS1992250, A, YVO |
| | | | | | | | RS1439119, G, YVO |
| | | | | | | | RS1992250, G, YVO |
| | | | | | | | RS1439119, G, OVN |
| | | | | | | | RS7168793, A, YVO |
| | | | | | | | RS1439119, A, YVO |
| | | | | | | | RS7168793, G, YVO |
| PIK3R4 | 0 | 12 | 131880476 | 3 | 0.0013 | RS900989, | RS900989, G, YVN |
| | | | | | | A, YVN | RS10934955, G, YVN |
| | | | | | | | RS10934955, A, YVN |
| | | | | | | | RS11710068, G, YVN |
| | | | | | | | RS11710068, A, YVN |
| | | | | | | | RS10934954, G, YVN |
| | | | | | | | RS10934954, A, YVN |
| | | | | | | | RS4682627, G, YVN |
| | | | | | | | RS4682627, A, YVN |
| | | | | | | | RS900989, G, YVO |
| | | | | | | | RS10934955, G, YVO |
| | | | | | | | RS900989, A, YVO |
| RPLP2 | 0 | 13 | 799965 | 11 | 0.0016 | RS4131364, | RS4131364, G, YVO |
| | | | | | | G, YVN | RS4131364, A, YVN |
| | | | | | | | RS4131364, A, YVO |
| OLR1 | 145 | 14 | 10202171 | 12 | 0.002 | RS2742113, | RS2742113, G, YVO |
| | | | | | | A, YVO | RS3741860, A, YVO |
| | | | | | | | RS3741860, G, YVO |
| | | | | | | | 12P0322, A, YVN |
| | | | | | | | RS1050286, A, YVN |
| | | | | | | | 12P0322, G, YVO |
| | | | | | | | RS1050286, G, YVN |
| | | | | | | | RS2742113, A, OVN |
| | | | | | | | RS3736233, G, YVO |
| | | | | | | | RS3736233, A, YVN |
| | | | | | | | 12P0322, G, YVN |
| | | | | | | | RS3736232, G, YVO |
| | | | | | | | RS1050286, G, YVO |
| | | | | | | | RS3736233, G, YVN |
| | | | | | | | RS3736232, G, YVN |
| | | | | | | | RS3736232, A, YVN |
| | | | | | | | 12P0322, A, YVO |
| PNPLA2 | 0 | 15 | 808902 | 11 | 0.002 | RS6597979, | RS1138714, A, YVN |
| | | | | | | G, YVN | RS1138714, G, YVO |
| | | | | | | | RS6597979, A, YVN |
| | | | | | | | RS1138714, G, YVN |
| | | | | | | | RS6597979, G, YVO |
| | | | | | | | RS1135628, G, YVN |
| | | | | | | | RS6597979, A, YVO |
| | | | | | | | RS1135628, A, YVN |
| | | | | | | | RS1138714, A, YVO |
| TCF4 | 0 | 16 | 51046093 | 18 | 0.0021 | RS1893430, | RS1893430, A, YVO |
| | | | | | | G, YVO | RS2276195, G, YVO |
| | | | | | | | RS1893430, A, YVN |
| | | | | | | | RS2276195, A, YVO |
| | | | | | | | RS1261076, G, YVO |
| | | | | | | | RS1893430, G, YVN |
| | | | | | | | RS2276195, G, OVN |
| | | | | | | | RS2119292, G, YVO |
| | | | | | | | RS2119292, A, YVO |
| | | | | | | | RS1440476, A, YVO |
| ACP5 | 31 | 17 | 11546477 | 19 | 0.0022 | RS2229531, | RS2305799, A, OVN |
| | | | | | | A, OVN | RS2071484, A, OVN |
| | | | | | | | RS2229531, G, YVO |
| | | | | | | | RS2071484, G, YVO |
| | | | | | | | RS2229531, G, OVN |
| | | | | | | | RS2071484, A, YVO |
| | | | | | | | RS2071484, G, OVN |
| | | | | | | | RS2229531, A, YVO |
| | | | | | | | RS2305799, G, OVN |
| | | | | | | | RS2305799, G, YVO |
| | | | | | | | RS2305799, A, YVO |
| SELP | 0 | 18 | 166289748 | 1 | 0.0028 | RS6133, A, | RS6132, A, YVO |
| | | | | | | YVO | RS6132, G, YVO |
| | | | | | | | RS6133, G, YVO |
| | | | | | | | RS6133, G, OVN |
| | | | | | | | RS6132, G, OVN |
| | | | | | | | RS6136, G, OVN |
| | | | | | | | RS6133, A, OVN |
| | | | | | | | RS6132, A, OVN |
| | | | | | | | RS6136, G, YVO |
| BAX | 0 | 19 | 54149998 | 19 | 0.0032 | RS1805419, | RS1805419, A, YVN |
| | | | | | | G, YVN | RS4645887, G, YVO |
| | | | | | | | RS905238, G, YVO |
| | | | | | | | RS4645887, G, YVN |
| | | | | | | | RS4645887, A, YVO |
| | | | | | | | RS4645887, A, YVN |
| CPNE4 | 0 | 20 | 132736261 | 3 | 0.0036 | RS6802186, | RS6802186, A, YVN |
| | | | | | | G, YVN | RS1463518, A, YVO |
| | | | | | | | RS1870713, A, YVO |
| | | | | | | | RS6802186, G, YVO |
| | | | | | | | RS1463518, G, YVO |
| | | | | | | | RS1870713, G, YVO |
| TAL1 | 0 | 21 | 47393984 | 1 | 0.0043 | 1P0330, G, | 1P0330, A, OVN |
| | | | | | | OVN | 1P0330, A, YVN |
| | | | | | | | 1P0330, G, YVN |
| KLF15 | 0 | 22 | 127544177 | 3 | 0.0049 | RS7622890, | none |
| | | | | | | G, YVN | |
| ABCB1 | 0 | 23 | 86777599 | 7 | 0.0051 | RS1045642, | RS1128503, A, YVN |
| | | | | | | A, YVN | RS1045642, G, YVN |
| LHFPL2 | 147 | 24 | 77816810 | 5 | 0.0051 | RS1561735, | RS1561735, G, OVN |
| | | | | | | A, OVN | RS6872179, A, YVN |
| | | | | | | | RS6872179, A, OVN |
| | | | | | | | RS1561735, A, YVN |
| | | | | | | | RS11948997, A, YVN |
| ITGAX | 94 | 25 | 31274010 | 16 | 0.0055 | RS4264407, | RS4264407, A, YVO |
| | | | | | | G, YVO | RS1140195, A, OVN |
| | | | | | | | RS4264407, G, OVN |
| LOC389142 | 0 | 26 | 119206321 | 3 | 0.0057 | RS1486336, | RS1486336, A, YVO |
| | | | | | | G, YVO | RS1968010, A, OVN |
| PLXNC1 | 88 | 27 | 93044967 | 12 | 0.0058 | RS2305971, | RS1681866, A, YVN |
| | | | | | | G, YVN | RS1681866, G, YVN |
| | | | | | | | RS2305971, A, YVN |
| SLA | 46 | 28 | 134118156 | 8 | 0.0058 | RS2252807, | RS2252807, G, YVO |
| | | | | | | A, YVO | RS1533910, G, OVN |
| | | | | | | | RS1533910, A, OVN |
| ELL | 0 | 29 | 18414475 | 19 | 0.0063 | RS6512269, | RS7252848, G, YVO |
| | | | | | | G, YVO | RS748609, G, YVO |
| | | | | | | | RS6512269, A, YVO |
| | | | | | | | RS748609, A, YVO |
| | | | | | | | RS2303692, G, YVO |
| | | | | | | | RS748609, A, YVN |
| | | | | | | | RS7252848, A, YVO |
| | | | | | | | RS2303692, A, YVO |
| | | | | | | | RS7252848, A, YVN |
| | | | | | | | RS6512269, G, OVN |
| NPY | 0 | 30 | 24097047 | 7 | 0.0065 | RS5574, A, | RS5574, G, YVN |
| | | | | | | YVN | RS9785023, G, YVN |
| | | | | | | | RS9785023, A, YVN |
| | | | | | | | RS5574, A, YVO |
| IGSF11 | 0 | 31 | 120102171 | 3 | 0.0066 | RS1468738, | RS1468738, G, OVN |
| | | | | | | A, OVN | RS2160052, A, OVN |
| | | | | | | | RS4687959, A, OVN |
| | | | | | | | RS2160052, A, YVN |
| | | | | | | | RS2903250, G, OVN |
| | | | | | | | RS2903250, A, OVN |
| | | | | | | | RS4687959, G, YVN |
| | | | | | | | RS4687959, A, YVN |
| ITPK1 | 0 | 32 | 92473012 | 14 | 0.0066 | HCV1259613, | RS2402226, A, YVO |
| | | | | | | G, OVN | RS2402226, A, OVN |
| | | | | | | | RS2402226, G, YVO |
| | | | | | | | HCV1259613, A, OVN |
| | | | | | | | RS875395, G, YVO |
| | | | | | | | RS4905043, A, OVN |
| | | | | | | | RS1740595, G, YVO |
| | | | | | | | RS2402226, G, OVN |
| | | | | | | | RS1740595, A, YVO |
| ASB1 | 174 | 33 | 239117626 | 2 | 0.007 | RS507812, | RS507812, A, YVO |
| | | | | | | G, YVO | RS507812, G, YVN |
| SELB | 0 | 34 | 129355049 | 3 | 0.007 | RS2955103, | RS2955103, A, YVN |
| | | | | | | G, YVN | RS760383, G, YVN |
| | | | | | | | RS2811529, G, OVN |
| | | | | | | | RS2811529, G, YVN |
| | | | | | | | RS2687720, G, OVN |
| LOC131873 | 0 | 35 | 131718695 | 3 | 0.0075 | RS1508520, | RS6439249, G, OVN |
| | | | | | | G, OVN | RS6439249, A, OVN |
| | | | | | | | RS9823913, A, OVN |
| | | | | | | | RS1508520, A, OVN |
| | | | | | | | RS9823913, G, OVN |
| | | | | | | | RS6439249, G, YVO |
| | | | | | | | RS6439249, A, YVO |
| PCCA | 0 | 36 | 99539338 | 13 | 0.0086 | RS9518035, | RS9518035, A, OVN |
| | | | | | | G, OVN | RS1296332, A, OVN |
| | | | | | | | RS9518016, G, YVN |
| | | | | | | | RS9518016, A, YVN |
| HAPIP | 0 | 37 | 125296275 | 3 | 0.0087 | RS2272486, | RS13075202, G, YVN |
| | | | | | | G, OVN | RS7621976, A, YVO |
| | | | | | | | RS2272486, A, OVN |
| | | | | | | | RS13075202, A, OVN |
| | | | | | | | RS13075202, A, YVN |
| | | | | | | | RS333284, G, YVO |
| | | | | | | | RS7621976, G, YVO |
| | | | | | | | RS2272486, G, YVO |
| | | | | | | | RS13075202, G, OVN |
| | | | | | | | RS7621976, G, YVN |
| | | | | | | | RS333284, G, OVN |
| PLAUR | 119 | 38 | 48842449 | 19 | 0.0088 | RS2286960, | RS2286960, A, OVN |
| | | | | | | G, OVN | RS2286960, G, YVN |
| | | | | | | | RS2286960, A, YVN |
| SIDT1 | 0 | 39 | 114734183 | 3 | 0.0099 | RS11929640, | RS11929640, G, YVO |
| | | | | | | A, YVO | RS11929640, G, OVN |
| | | | | | | | RS11929640, A, OVN |
| RPN1 | 0 | 40 | 129821511 | 3 | 0.0106 | RS4857914, | RS2712371, G, YVO |
| | | | | | | G, YVO | RS4857914, A, YVO |
| | | | | | | | RS1127030, G, YVO |
| | | | | | | | RS1697, G, YVO |
| | | | | | | | RS4857914, G, OVN |
| BPAG1 | 63 | 41 | 56430744 | 6 | 0.0128 | RS2613118, | RS2024751, A, YVN |
| | | | | | | A, YVO | RS2024751, A, OVN |
| | | | | | | | RS1014310, A, YVN |
| | | | | | | | RS1014310, A, OVN |
| | | | | | | | RS2613118, G, YVO |
| | | | | | | | RS1014310, G, OVN |
| | | | | | | | RS1024196, G, YVO |
| | | | | | | | RS1024196, A, YVO |
| | | | | | | | RS2613118, G, YVN |
| ROR2 | 0 | 42 | 91564439 | 9 | 0.0139 | RS1881385, | RS10116351, G, YVN |
| | | | | | | A, YVN | RS10116351, A, YVN |
| | | | | | | | RS4744098, G, YVO |
| | | | | | | | RS4744098, A, YVO |
| | | | | | | | RS4744098, A, OVN |
| MMP12 | 71 | 43 | 102238686 | 11 | 0.0144 | RS674546, | RS674546, A, YVO |
| | | | | | | G, YVO | RS674546, G, YVN |
| | | | | | | | RS1277718, G, YVN |
| | | | | | | | RS674546, A, YVN |
| | | | | | | | RS2276109, A, YVO |
| | | | | | | | RS2276109, G, YVO |
| | | | | | | | RS652438, G, YVN |
| GAP43 | 0 | 44 | 116825142 | 3 | 0.0148 | RS2918208, | RS14360, A, YVO |
| | | | | | | G, YVO | RS2918208, A, YVN |
| | | | | | | | RS2918208, A, YVO |
| | | | | | | | RS14360, A, YVN |
| | | | | | | | RS2918208, G, YVN |
| | | | | | | | RS14360, G, YVO |
| FSTL1 | 0 | 45 | 121595817 | 3 | 0.0155 | RS1259333, | RS1147707, A, OVN |
| | | | | | | A, OVN | RS1259333, G, OVN |
| | | | | | | | RS1515577, G, OVN |
| | | | | | | | RS2272515, G, YVN |
| | | | | | | | RS2272515, A, YVN |
| | | | | | | | RS2272515, G, OVN |
| | | | | | | | RS2488, G, OVN |
| | | | | | | | RS1515577, G, YVN |
| | | | | | | | RS1147707, G, OVN |
| | | | | | | | RS2488, G, YVN |
| | | | | | | | RS1057231, G, OVN |
| | | | | | | | RS1147707, A, YVO |
| MAP4 | 152 | 46 | 47868362 | 3 | 0.0158 | RS2166770, | RS2166770, G, YVO |
| | | | | | | A, YVO | RS319689, A, YVO |
| | | | | | | | RS319689, G, YVO |
| | | | | | | | RS6442089, A, YVO |
| | | | | | | | RS319689, G, YVN |
| | | | | | | | RS6442089, G, YVO |
| | | | | | | | RS2166770, G, YVN |
| ZNF217 | 53 | 47 | 51617019 | 20 | 0.016 | RS1326862, | RS1326862, G, YVN |
| | | | | | | A, YVN | RS2766669, A, YVN |
| ALOX5 | 73 | 48 | 45189692 | 10 | 0.0161 | RS3740107, | RS3740107, G, YVN |
| | | | | | | G, YVO | RS3740107, A, YVN |
| | | | | | | | RS2242332, A, YVN |
| | | | | | | | RS3740107, A, YVO |
| | | | | | | | RS892691, G, YVO |
| | | | | | | | RS892691, A, YVO |
| NPHP3 | 0 | 49 | 133759684 | 3 | 0.0163 | RS2369832, | RS2369832, A, OVN |
| | | | | | | G, OVN | |
| GPNMB | 189 | 50 | 23059626 | 7 | 0.0166 | RS199347, | RS199347, A, YVO |
| | | | | | | A, OVN | RS199347, G, OVN |
| | | | | | | | RS199348, G, YVO |
| | | | | | | | RS199355, G, OVN |
| | | | | | | | RS199355, G, YVO |
| SPP1 | 2 | 51 | 89253981 | 4 | 0.0174 | RS12502049, | RS12502049, G, YVN |
| | | | | | | A, YVN | |
| ZNF80 | 0 | 52 | 115437790 | 3 | 0.0188 | RS6438191, | RS3732782, G, YVO |
| | | | | | | G, YVO | RS6438191, A, OVN |
| | | | | | | | RS3732782, A, YVO |
| | | | | | | | RS6438191, A, YVO |
| | | | | | | | RS6438191, G, OVN |
| | | | | | | | RS3732782, A, OVN |
| MGP | 0 | 53 | 14926094 | 12 | 0.0189 | RS1800801, | RS4236, G, OVN |
| | | | | | | G, OVN | RS1800801, A, OVN |
| | | | | | | | RS2430738, G, OVN |
| | | | | | | | RS2430737, G, OVN |
| | | | | | | | RS4236, A, OVN |
| | | | | | | | RS2430738, A, OVN |
| C3ORF15 | 0 | 54 | 0 | 3 | 0.0199 | HCV369572, | HCV369572, G, YVO |
| | | | | | | A, YVO | |
| NEK11 | 0 | 55 | 132228421 | 3 | 0.0208 | RS16835847, | RS16835847, G, YVN |
| | | | | | | G, OVN | RS16835847, A, YVN |
| | | | | | | | RS16835847, A, OVN |
| | | | | | | | RS2033182, A, OVN |
| POLQ | 0 | 56 | 122632973 | 3 | 0.0218 | RS2030531, | RS2030531, G, OVN |
| | | | | | | A, OVN | RS2030531, A, YVO |
| | | | | | | | RS2030531, G, YVO |
| ADFP | 67 | 57 | 19105760 | 9 | 0.022 | RS3824369, | RS3824369, G, YVO |
| | | | | | | G, OVN | RS3824369, A, OVN |
| UBXD1 | 0 | 58 | 4396009 | 19 | 0.0223 | RS932276, | RS741923, G, YVN |
| | | | | | | G, YVN | RS11909, G, YVN |
| 38413 | 0 | 59 | 8389289 | 19 | 0.0224 | RS6603068, | RS6603068, G, YVO |
| | | | | | | G, YVN | RS2913984, A, YVN |
| | | | | | | | RS6603068, A, YVO |
| | | | | | | | RS6603068, A, YVN |
| FLJ46299 | 0 | 60 | 0 | 3 | 0.0229 | RS1014470, | |
| | | | | | | G, OVN | |
| ZBTB20 | 0 | 61 | 115540215 | 3 | 0.0229 | RS1818757, | RS1818757, A, YVO |
| | | | | | | A, OVN | RS1357016, A, YVN |
| | | | | | | | RS1818757, G, OVN |
| HLA- | 8 | 62 | 32817199 | 6 | 0.0229 | RS5018343, | RS2213566, A, OVN |
| DQA2 | | | | | | G, OVN | RS2213566, G, OVN |
| | | | | | | | RS5018343, A, OVN |
| | | | | | | | RS2051600, A, YVO |
| | | | | | | | RS2051600, G, YVO |
| | | | | | | | RS2395252, A, YVO |
| ZXDC | 0 | 63 | 127639143 | 3 | 0.0232 | RS1799404, | RS1799404, G, YVO |
| | | | | | | A, YVO | RS1799404, A, OVN |
| GRN | 69 | 64 | 39778174 | 17 | 0.0237 | RS3815057, | RS3859268, G, YVN |
| | | | | | | G, OVN | RS3815057, G, YVO |
| | | | | | | | RS3815057, A, OVN |
| | | | | | | | RS3815057, A, YVO |
| | | | | | | | RS3859268, A, YVN |
| | | | | | | | RS3859268, G, OVN |
| | | | | | | | RS3785817, A, OVN |
| PSCD1 | 0 | 65 | 74181727 | 17 | 0.0244 | RS3936118, | RS1871935, A, YVN |
| | | | | | | A, YVN | RS1384367, A, YVN |
| | | | | | | | HCV12126963, A, YVO |
| GYS1 | 0 | 66 | 54163195 | 19 | 0.0257 | RS2270938, | RS2270938, A, YVO |
| | | | | | | G, YVO | RS1042265, A, OVN |
| | | | | | | | RS1042265, A, YVN |
| | | | | | | | RS2270938, A, YVN |
| | | | | | | | RS2270938, G, YVN |
| | | | | | | | RS1042265, G, OVN |
| C14ORF132 | 11 | 67 | 95575431 | 14 | 0.0265 | RS2104290, | RS1058102, A, OVN |
| | | | | | | G, YVN | RS2104290, A, YVN |
| | | | | | | | RS1058102, G, YVO |
| | | | | | | | RS1058102, G, OVN |
| CD80 | 0 | 68 | 120725832 | 3 | 0.0266 | HCV387937, | RS1523311, A, YVO |
| | | | | | | A, YVN | RS1523311, G, YVO |
| | | | | | | | HCV387937, G, YVN |
| CDGAP | 0 | 69 | 120495910 | 3 | 0.0267 | RS10934490, | RS10934490, G, YVN |
| | | | | | | A, YVN | |
| LMOD1 | 149 | 70 | 198586920 | 1 | 0.0274 | RS2819366, | RS2819366, A, YVN |
| | | | | | | G, YVN | RS7528681, A, YVN |
| SLC41A3 | 0 | 71 | 127207903 | 3 | 0.0277 | HCV123667, | none |
| | | | | | | A, OVN | |
| HOXD1 | 0 | 72 | 176878814 | 2 | 0.0278 | RS1446575, | RS1446575, A, OVN |
| | | | | | | G, OVN | |
| STAT5A | 12 | 73 | 37693865 | 17 | 0.0284 | RS3198502, | RS3198502, G, OVN |
| | | | | | | A, OVN | |
| OPRM1 | 0 | 74 | 154452590 | 6 | 0.0303 | RS609148, | RS524731, A, YVN |
| | | | | | | A, YVO | RS609148, A, OVN |
| | | | | | | | RS524731, A, YVO |
| | | | | | | | RS524731, G, YVN |
| ITPR2 | 162 | 75 | 26765487 | 12 | 0.0305 | RS2291264, | RS2291264, A, YVO |
| | | | | | | G, YVO | RS1449568, G, YVO |
| | | | | | | | RS2291264, A, OVN |
| | | | | | | | RS1449568, A, YVO |
| HIF1A | 0 | 76 | 61231992 | 14 | 0.0307 | RS3783752, | RS2301113, A, YVN |
| | | | | | | A, YVO | |
| PKD2 | 19 | 77 | 89285999 | 4 | 0.0314 | RS2728110, | RS2728116, A, YVO |
| | | | | | | A, YVO | RS2728116, G, YVO |
| STEAP | 35 | 78 | 89428340 | 7 | 0.032 | RS2961269, | RS2158746, A, YVO |
| | | | | | | A, YVN | RS2158746, A, OVN |
| | | | | | | | RS2158746, G, OVN |
| | | | | | | | RS2158746, G, YVO |
| AGTR1 | 0 | 79 | 149898363 | 3 | 0.0322 | RS3772587, | RS9849625, A, YVO |
| | | | | | | A, YVN | RS389566, A, YVO |
| NDUFB4 | 0 | 80 | 121797818 | 3 | 0.0326 | HCV112367 | none |
| | | | | | | 38, G, YVN | |
| GLRA3 | 0 | 81 | 175938660 | 4 | 0.0336 | RS4695942, | RS4695942, A, OVN |
| | | | | | | G, YVN | RS4695942, A, YVN |
| MEF2A | 0 | 82 | 97923738 | 15 | 0.0338 | HCV11709390, | RS325408, A, YVN |
| | | | | | | A, YVN | |
| STXBP5L | 0 | 83 | 122104941 | 3 | 0.0341 | RS4505627, | RS4505627, A, OVN |
| | | | | | | G, OVN | |
| APOBEC3D | 0 | 84 | 37741952 | 22 | 0.0343 | RS5757425, | RS5757425, A, OVN |
| | | | | | | G, OVN | |
| FMNL1 | 0 | 85 | 40655075 | 17 | 0.0352 | RS1989229, | RS1552458, G, YVO |
| | | | | | | A, OVN | RS1989229, G, OVN |
| | | | | | | | RS1552458, G, OVN |
| | | | | | | | RS1801353, G, OVN |
| | | | | | | | RS1552458, A, OVN |
| PLXND1 | 97 | 86 | 130756716 | 3 | 0.0361 | RS2245285, | RS2245285, A, YVO |
| | | | | | | A, OVN | RS2245278, A, YVO |
| | | | | | | | RS2245285, G, YVO |
| | | | | | | | RS2245285, G, OVN |
| | | | | | | | RS2245278, A, OVN |
| ATP2C1 | 0 | 87 | 132095533 | 3 | 0.0368 | RS2669869, | RS2669869, G, YVN |
| | | | | | | G, OVN | RS2669869, A, YVN |
| RUVBL1 | 0 | 88 | 129282501 | 3 | 0.0378 | RS7632756, | none |
| | | | | | | G, YVN | |
| CASR | 0 | 89 | 123455485 | 3 | 0.0379 | RS12635478, | RS12635478, G, OVN |
| | | | | | | A, OVN | RS13095172, A, OVN |
| | | | | | | | RS13095172, G, OVN |
| | | | | | | | HCV1412358, G, YVO |
| | | | | | | | RS13095172, G, YVO |
| | | | | | | | RS2270917, A, OVN |
| | | | | | | | RS1501899, A, YVO |
| | | | | | | | RS2270917, G, YVO |
| PTPRR | 0 | 90 | 69318129 | 12 | 0.0385 | HCV155408, | none |
| | | | | | | G, YVO | |
| SMPDL3A | 96 | 91 | 123152120 | 6 | 0.0396 | RS1385681, | RS1385681, G, YVN |
| | | | | | | A, YVO | RS1385681, A, YVN |
| APOD | 0 | 92 | unmapped | 3 | 0.0397 | RS13303036, | RS13303036, A, YVO |
| | | | | | | G, YVO | |
| APG3L | 0 | 93 | 113734236 | 3 | 0.0401 | RS2638037, | RS2638037, G, YVO |
| | | | | | | G, OVN | |
| FLJ35880 | 0 | 94 | 131642165 | 3 | 0.0406 | RS322115, | RS819086, G, OVN |
| | | | | | | G, YVN | RS9883988, G, OVN |
| | | | | | | | RS819086, G, YVO |
| | | | | | | | RS9883988, G, YVO |
| | | | | | | | RS322115, A, YVN |
| | | | | | | | RS819086, A, YVO |
| | | | | | | | RS819091, A, YVN |
| | | | | | | | RS9883988, A, YVO |
| | | | | | | | RS9883988, A, OVN |
| TMCC1 | 0 | 95 | 130850232 | 3 | 0.0406 | RS2811343, | none |
| | | | | | | G, YVO | |
| CD96 | 0 | 96 | 112743546 | 3 | 0.041 | RS1553970, | RS1553970, G, YVO |
| | | | | | | A, YVO | |
| C1QB | 118 | 97 | 22725046 | 1 | 0.0419 | RS292007, | RS10580, G, YVO |
| | | | | | | A, YVO | RS292007, A, OVN |
| | | | | | | | RS291988, G, OVN |
| | | | | | | | RS10580, A, YVO |
| CTSD | 30 | 98 | 1730561 | 11 | 0.0419 | RS17571, G, | none |
| | | | | | | YVN | |
| FLI1 | 0 | 99 | 128069239 | 11 | 0.0421 | RS660407, | RS497714, G, OVN |
| | | | | | | G, YVO | |
| MMP9 | 178 | 100 | 44070954 | 20 | 0.0421 | RS13969, G, | RS13969, G, YVN |
| | | | | | | OVN | |
| TCIRG1 | 190 | 101 | 67563059 | 11 | 0.0435 | RS2075609, | RS2075609, G, OVN |
| | | | | | | A, OVN | RS2075609, A, YVN |
| | | | | | | | RS906713, A, OVN |
| | | | | | | | RS11481, A, OVN |
| ITGB5 | 0 | 102 | 125964486 | 3 | 0.0452 | RS3772831, | none |
| | | | | | | G, YVO | |
| FLJ25414 | 0 | 103 | 40687543 | 17 | 0.046 | HCV9267944, | none |
| | | | | | | G, OVN | |
| NR1H3 | 68 | 104 | 47236106 | 11 | 0.0463 | RS3758673, | none |
| | | | | | | A, OVN | |
| HSPBAP1 | 0 | 105 | 123941536 | 3 | 0.0468 | HCV1402346, | HCV1402346, A, YVN |
| | | | | | | G, YVN | |
| APOC1 | 1 | 106 | 50109419 | 19 | 0.0469 | RS1064725, | RS1064725, G, YVN |
| | | | | | | A, YVN | |
| THPO | 0 | 107 | 185572475 | 3 | 0.0475 | RS6142, A, | RS6142, A, YVN |
| | | | | | | YVO | RS6142, G, YVN |
| FTL | 0 | 108 | 54160378 | 19 | 0.0476 | RS918546, | RS918546, A, YVO |
| | | | | | | G, YVO | |
| HADHSC | 124 | 109 | 109268516 | 4 | 0.0479 | RS221330, | RS221330, A, YVN |
| | | | | | | G, YVN | |
| ALOX5AP | 0 | 110 | 30207669 | 13 | 0.0481 | RS3803277, | RS3803277, A, YVN |
| | | | | | | A, OVN | |
| LAIR1 | 39 | 111 | 59557945 | 19 | 0.0493 | RS2287824, | RS2287824, A, OVN |
| | | | | | | G, OVN | RS1985841, G, OVN |
| | | | | | | | RS1985841, G, YVO |
| | | | | | | | RS730592, A, YVN |
| | | | | | | | RS730592, G, YVN |
| UPP1 | 79 | 112 | 47901481 | 7 | 0.0524 | RS6463462, | RS7804178, G, YVN |
| | | | | | | G, OVN | RS6463462, A, OVN |
| | | | | | | | RS7804178, G, OVN |
| LAPTM5 | 7 | 113 | 30874409 | 1 | 0.0527 | RS3795438, | 1P0260, A, YVN |
| | | | | | | G, OVN | |
| CSTA | 0 | 114 | 123526773 | 3 | 0.0528 | RS17589, A, | RS17589, A, OVN |
| | | | | | | YVO | RS17589, G, OVN |
| ADCY5 | 0 | 115 | 124486089 | 3 | 0.053 | RS4678030, | none |
| | | | | | | A, YVO | |
| PHLDB2 | 0 | 116 | 113061333 | 3 | 0.0531 | RS1282980, | RS1282980, A, YVO |
| | | | | | | G, YVO | |
| GM2A | 40 | 117 | 150612837 | 5 | 0.0533 | RS153450, | RS153450, G, YVO |
| | | | | | | A, YVO | RS2277028, A, OVN |
| NUDT16 | 0 | 118 | 132583405 | 3 | 0.0536 | RS11914980, | RS11914980, G, YVO |
| | | | | | | A, YVO | |
| ACSL1 | 0 | 119 | 186051899 | 4 | 0.0547 | RS3792311, | RS3749233, G, OVN |
| | | | | | | A, YVO | RS2280297, G, OVN |
| VAMP5 | 10 | 120 | 85723189 | 2 | 0.056 | RS2289976, | RS12888, G, OVN |
| | | | | | | G, OVN | RS2289976, A, OVN |
| ACP2 | 4 | 121 | 47217429 | 11 | 0.0568 | RS2167079, | RS2167079, G, OVN |
| | | | | | | A, OVN | |
| HLA- | 9 | 122 | 33140772 | 6 | 0.0571 | RS1042174, | RS1042174, G, OVN |
| DPA1 | | | | | | A, OVN | |
| TUBA3 | 0 | 123 | 47864852 | 12 | 0.0575 | RS2016107, | RS7954530, A, OVN |
| | | | | | | A, OVN | RS1039225, G, YVN |
| | | | | | | | RS1874908, A, OVN |
| | | | | | | | RS6580703, A, OVN |
| | | | | | | | RS1039225, A, OVN |
| | | | | | | | RS1056875, A, OVN |
| MMP7 | 92 | 124 | 101896449 | 11 | 0.0578 | RS609887, | RS609887, G, YVN |
| | | | | | | A, YVN | |
| H41 | 0 | 125 | 134775272 | 3 | 0.058 | RS1842155, | none |
| | | | | | | G, YVO | |
| NR1I2 | 0 | 126 | 120982021 | 3 | 0.0587 | RS1523130, | none |
| | | | | | | G, YVO | |
| FGFR2 | 28 | 127 | 122473377 | 10 | 0.06 | RS2071616, | RS1047100, G, YVO |
| | | | | | | G, YVO | |
| GBA | 0 | 128 | 152017317 | 1 | 0.0661 | RS1045253, | RS4043, G, OVN |
| | | | | | | G, OVN | |
| CHAF1A | 0 | 129 | 4353661 | 19 | 0.0667 | RS243375, | none |
| | | | | | | G, YVN | |
| GSK3B | 0 | 130 | 121028215 | 3 | 0.0679 | RS12638973, | RS12638973, A, YVN |
| | | | | | | G, YVN | |
| DOCK2 | 70 | 131 | 168996871 | 5 | 0.068 | RS11740057, | none |
| | | | | | | G, YVO | |
| URB | 0 | 132 | 113806101 | 3 | 0.0697 | RS3843366, | none |
| | | | | | | A, YVN | |
| HCLS1 | 241 | 133 | 122832937 | 3 | 0.0711 | RS2070180, | RS11714406, G, YVN |
| | | | | | | G, YVO | RS11716984, G, YVN |
| CD200R1 | 0 | 134 | 114122746 | 3 | 0.0736 | RS9870568, | none |
| | | | | | | G, YVO | |
| SLCO2B1 | 6 | 135 | 74539809 | 11 | 0.0736 | RS2851109, | none |
| | | | | | | A, YVO | |
| B4GALT4 | 0 | 136 | 120413279 | 3 | 0.0746 | RS4687841, | none |
| | | | | | | A, YVN | |
| PLCXD2 | 0 | 137 | 112876213 | 3 | 0.0777 | RS1877575, | none |
| | | | | | | A, YVN | |
| FABP7 | 0 | 138 | 123142345 | 6 | 0.0816 | HCV31425, | none |
| | | | | | | G, OVN | |
| CAMKK2 | 0 | 139 | 120138217 | 12 | 0.0835 | RS25644, G, | none |
| | | | | | | YVN | |
| FCGR1A | 140 | 140 | 146567361 | 1 | 0.0835 | RS1050204, | none |
| | | | | | | A, YVN | |
| SELL | 0 | 141 | 166391466 | 1 | 0.0839 | RS1051091, | none |
| | | | | | | A, YVN | |
| SELE | 0 | 142 | 166423440 | 1 | 0.085 | RS5356, A, | none |
| | | | | | | YVN | |
| HNRPM | 0 | 143 | 8415651 | 19 | 0.0856 | RS6603076, | RS6603076, G, OVN |
| | | | | | | A, OVN | |
| MGC45840 | 0 | 144 | 819297 | 11 | 0.0869 | RS4075289, | none |
| | | | | | | A, OVN | |
| F5 | 0 | 145 | 166215067 | 1 | 0.0896 | RS4524, G, | none |
| | | | | | | OVN | |
| SMTN | 0 | 146 | 29801858 | 22 | 0.0898 | RS1004243, | RS917208, A, OVN |
| | | | | | | G, OVN | |
| RAI3 | 25 | 147 | 12952451 | 12 | 0.0918 | RS850932, | RS850932, A, YVN |
| | | | | | | G, YVN | |
| HLA- | 86 | 148 | 32515646 | 6 | 0.0925 | HCV2455646, | none |
| DRA | | | | | | G, YVN | |
| CSTB | 20 | 149 | 44018260 | 21 | 0.0944 | RS743466, | none |
| | | | | | | G, YVN | |
| FLJ12592 | 0 | 150 | 0 | 3 | 0.0963 | RS6776500, | none |
| | | | | | | G, YVN | |
| TAGLN3 | 0 | 151 | 113200332 | 3 | 0.0972 | RS774763, | none |
| | | | | | | G, YVN | |

While all candidates listed in Table 2 must be considered very strong candidates, the results of these analyses very strongly implicate several genes in the development of atherosclerosis as measured by CADi. The following is a description of the genes in Table 2: AIM1 L: Absent in melanoma 1-like; PLA2G7: Platelet-activating factor acetylhydrolase precursor (EC 3.1.1.47) (PAF acetylhydrolase) (PAF 2-acylhydrolase) (LDL-associated phospholipase A2) (LDL-PLA(2)) (2-acetyl-1-alkylglycerophosphocholine esterase) (1-alkyl-2-acetylglycerophosphocholine esterase); OR7E29P: olfactory receptor, family 7, subfamily E, member 29 pseudogene; PLN: Cardiac phospholamban (PLB); PTPN6: Protein-tyrosine phosphatase, non-receptor type 6 (EC 3.1.3.48) (Protein-tyrosine phosphatase 1C) (PTP-1C) (Hematopoietic cell protein-tyrosine phosphatase) (SH-PTP1) (Protein-tyrosine phosphatase SHP-1); C1ORF38: ICB-1beta (Clorf38 protein); GATA2: Endothelial transcription factor GATA-2; IL7R: Interleukin-7 receptor alpha chain precursor (IL-7R-alpha) (CDw127) (CD127 antigen); MYLK: Myosin light chain kinase, smooth muscle and non-muscle isozymes (EC 2.7.1.117) (MLCK) [Contains: Telokin (Kinase related protein) (KRP)]; ANPEP: Aminopeptidase N (EC 3.4.11.2) (hAPN) (Alanyl aminopeptidase) (Microsomal aminopeptidase) (Aminopeptidase M) (gp150) (Myeloid plasma membrane glycoprotein CD13); PIK3R4: phosphoinositide-3-kinase, regulatory subunit 4, pISO; RPLP2: 60S acidic ribosomal protein P2; OLR1: OXIDISED LOW DENSITY LIPOPROTEIN (LECTIN-LIKE) RECEPTOR 1; SCAVENGER RECEPTOR CLASS E, MEMBER 1; PNPLA2: patatin-like phospholipase domain containing 2; TCF4: Transcription factor 4 (Immunoglobulin transcription factor 2) (ITF-2) (SL3-3 enhancer factor 2) (SEF-2); ACP5: TARTRATE RESISTANT ACID PHOSPHATASE TYPE 5 PRECURSOR (EC 3.1.3.2) (TR-AP) (TARTRATE-RESISTANT ACID ATPASE) (TRATPASE); SELP: P-selectin precursor (Granule membrane protein 140) (GMP-140) (PADGEM) (CD62P) (Leukocyte-endothelial cell adhesion molecule 3) (LECAM3); BAX: BAX protein, cytoplasmic isoform delta; CPNE4: Copine-4 (Copine IV) (Copine-8); TALI: T-cell acute lymphocytic leukemia-1 protein (TAL-1 protein) (Stem cell protein) (T-cell leukemia/lymphoma-5 protein); KLF15: Krueppel-like factor 15 (Kidney-enriched kruppel-like factor); ABCB1: Multidrug resistance protein 1 (P-glycoprotein 1) (CD243 antigen); LHFPL2: Homo sapiens lipoma HMG1C fusion partner-like 2 (LHFPL2), mRNA; ITGAX: Integrin alpha-X precursor (Leukocyte adhesion glycoprotein p150,95 alpha chain) (Leukocyte adhesion receptor p150,95) (CD11c) (Leu M5); LOC389142: hypothetical LOC389142; PLXNC1: Homo sapiens plexin C1 (PLXNC1), mRNA; SLA: SRC-like-adapter (Src-like-adapter protein 1) (hSLAP); ELL: RNA polymerase II elongation factor ELL (Eleven-nineteen lysine-rich leukemia protein); NPY: Neuropeptide Y precursor [Contains: Neuropeptide Y (Neuropeptide tyrosine) (NPY); C-flanking peptide of NPY (CPON)]; IGSF11: Brain and testis-specific immunoglobin superfamily protein; ITPK1: Homo sapiens inositol 1,3,4-triphosphate 5/6 kinase (ITPK1), mRNA; ASB1: Ankyrin repeat and SOCS box containing protein 1 (ASB-1); SELB: Selenocysteine-specific elongation factor (Elongation factor sec); LOC131873: hypothetical protein LOC131873; PCCA: Propionyl-CoA carboxylase alpha chain, mitochondrial precursor (EC 6.4.1.3) (PCCase alpha subunit) (Propanoyl-CoA:carbon dioxide ligase alpha subunit); HAPIP: Huntingtin-associated protein-interacting protein (Duo protein); PLAUR: Urokinase plasminogen activator surface receptor precursor (uPAR) (U-PAR) (Monocyte activation antigen Mo3) (CD87 antigen); SIDTI: SIDI transmembrane family, member 1; RPN1: Dolichyl-diphosphooligosaccharide—protein glycosyltransferase 67 kDa subunit precursor (EC 2.4.1.119) (Ribophorin I) (RPN-I); BPAG1: Bullous pemphigoid antigen 1 isofomms 1/2/3/4/5/8 (230 kDa bullous pemphigoid antigen) (BPA) (Hemidesmosomal plaque protein) (Dystonia musculorum protein) (Fragment); ROR2: TYROSINE-PROTEIN KINASE TRANSMEMBRANE RECEPTOR ROR2-PRECURSOR (EC 2.7.1.112) (NEUROTROPHIC TYROSINE KINASE, RECEPTOR-RELATED 2); MMP12: MACROPHAGE METALLOELASTASE PRECURSOR (EC 3.4.24.65) (HME) (MATRIX METALLOPROTEINASE-12) (MMP-12) (MACROPHAGE ELASTASE) (ME); GAP43: Neuromodulin (Axonal membrane protein GAP-43) (Growth associated protein 43) (PP46) (Neural phosphoprotein B-50); FSTL11: Follistatin-related protein 1 precursor (Follistatin-like 1); MAP4: Microtubule-associated protein 4 (MAP 4); ZNF217: Zinc finger protein 217; ALOX5: ARACHIDONATE 5-LIPOXYGENASE (EC 1.13.11.34) (5-LIPOXYGENASE) (5-LO); NPHP3: nephronophthisis 3; GPNMB: Putative transmembrane protein NMB precursor (Transmembrane glycoprotein HGFIN); SPP1: Osteopontin precursor (Bone sialoprotein 1) (Urinary stone protein) (Secreted phosphoprotein 1) (SPP-1) (Nephropontin) (Uropontin); ZNF80: Zinc finger protein 80 (ZNFPT17); MGP: Matrix Gla-protein precursor (MGP); C30RF15:; NEK11: NIMA (never in mitosis gene a)—related kinase 11; POLQ: polymerase (DNA directed), theta; ADFP: ADIPOPHILIN (ADIPOSE DIFFERENTIATION-RELATED PROTEIN) (ADRP); UBXD1: UBX domain-containing protein 1; 38413: membrane-associated ring finger (C3HC4) 2; FLJ46299:; ZBTB20: Zinc finger and BTB domain containing protein 20 (Zinc finger protein 288) (Dendritic-derived BTB/POZ zinc finger protein); HLA-DQA2: HLA class II histocompatibility antigen, DQ(6) alpha chain precursor (DX alpha chain) (HLA-DQA1); ZXDC: ZXD family zinc finger C; GRN: Granulins precursor (Acrogranin) (Proepithelin) (PEPI) [Contains: Paragranulin; Granulin 1 (Granulin G); Granulin 2 (Granulin F); Granulin 3 (Granulin B); Granulin 4 (Granulin A); Granulin 5 (Granulin C); Granulin 6 (Granulin D); Granulin 7 (Granulin E)]; PSCD1: CYTOHESIN 1 (SEC7 HOMOLOG B2-1).; GYS1: Glycogen [starch] synthase, muscle (EC 2.4.1.11); C14ORF132: NA; CD80: T lymphocyte activation antigen CD80 precursor (Activation B7-1 antigen) (CTLA-4 counter-receptor B7.1) (B7) (BB1); CDGAP: Cdc42 GTPase-activating protein; LMOD1: Leiomodin 1 (Leiomodin, muscle form) (64 kDa autoantigen D1) (64 kDa autoantigen 1D) (64 kDa autoantigen 1D3) (Thyroid-associated opthalmopathy autoantigen) (Smooth muscle leiomodin) (SM-Lmod); SLC41A3: solute carrier family 41, member 3; HOXD1: Homeobox protein Hox-D1; STAT5A: SIGNAL TRANSDUCER AND ACTIVATOR OF TRANSCRIPTION 5A; OPRM1: Mu-type opioid receptor (MOR-1); ITPR2: INOSITOL 1,4,5-TRISPHOSPHATE RECEPTOR TYPE 2 (TYPE 2 INOSITOL 1,4,5-TRISPHOSPHATE RECEPTOR) (TYPE 2 INSP3 RECEPTOR) (IP3 RECEPTOR ISOFORM 2) (INSP3R2); HIF1A: HYPOXIA-INDUCIBLE FACTOR 1 ALPHA (HIF-1 ALPHA) (ARNT INTERACTING PROTEIN) (MEMBER OF PAS PROTEIN 1) (MOP1) (HIF1 ALPHA); PKD2: Polycystin 2 (Autosomal dominant polycystic kidney disease type II protein) (Polycystwin) (R48321); STEAP: Six transmembrane epithelial antigen of prostate; AGTR1: Type-1 angiotensin II receptor (AT1) (AT1AR); NDUFB4: NADH dehydrogenase (ubiquinone) 1 beta subcomplex; GLRA3: Glycine receptor alpha-3 chain precursor; MEF2A: Myocyte-specific enhancer factor 2A (Serum response factor-like protein 1); STX3BP5L: syntaxin binding protein 5-like; APOBEC3D: NA; FMNL1: FORMIN-LIKE PROTEIN (PROTEIN C17ORF1); PLXND1: Homo sapiens plexin D1 (PLXND1), mRNA; ATP2C1: Calcium-transporting ATPase type 2C, member 1 (ATPase 2Cl) (ATP-dependent Ca(2+) pump PMR1); RUVBL1: RuvB-like 1 (EC 3.6.1.-) (49-kDa TATA box-binding protein-interacting protein) (49 kDa TBP-interacting protein) (TIP49a) (Pontin 52) (Nuclear matrix protein 238) (NMP 238) (54 kDa erythrocyte cytosolic protein) (ECP-54) (TIP60-associated protein 54-alpha) (TAP54-alpha); CASR: Extracellular calcium-sensing receptor precursor (CaSR) (Parathyroid Cell calcium-sensing receptor); PTPRR: PROTEIN-TYROSINE PHOSPHATASE R PRECURSOR (EC 3.1.3.48) (PROTEIN-TYROSINE PHOSPHATASE PCPTP1) (NC-PTPCOM1) (CH-1PTPASE); SMPDL3A: Acid sphingomyelinase-like phosphodiesterase 3a precursor (EC 3.1.4.-) (ASM-like phosphodiesterase 3a); APOD: Apolipoprotein D precursor (Apo-D) (ApoD); APG3L: APG3 autophagy 3-like (S. cerevisiae); FLJ35880: FLJ35880: hypothetical protein FLJ35880; TMCCl: transmembrane and coiled-coil domains 1; CD96: T-cell surface protein tactile precursor (CD96 antigen); C1QB: Complement Clq subcomponent, B chain precursor; CTSD: Cathepsin D precursor (EC 3.4.23.5); FLI1: FRIEND LEUKEMIA INTEGRATION 1 TRANSCRIPTION FACTOR (FLI-1 PROTO-ONCOGENE) (ERGB TRANSCRIPTION FACTOR).; MMP9: 92 kDa type IV collagenase precursor (EC 3.4.24.35) (92 kDa gelatinase) (Matrix metalloproteinase-9) (MMP-9) (Gelatinase B) (GELB); TCIRG1: Vacuolar proton translocating ATPase 116 kDa subunit a isoform 3 (V-ATPase 116-kDa isoform a3) (Osteoclastic proton pump 116 kDa subunit) (OC-116 KDa) (OC116) (T-cell immune regulator 1) (T cell immune response cDNA7 protein) (TIRC7); ITGB5: Integrin beta-5 precursor; FLJ25414: NA; NR1H3: OXYSTEROLS RECEPTOR LXR-ALPHA (LIVER X RECEPTOR ALPHA) (NUCLEAR ORPHAN RECEPTOR LXR-ALPHA).; HSPBAP1: HSPB (eat shock 27 kDa) associated protein 1; APOC1: Apolipoprotein C-1 precursor (Apo-CI); THPO: Thrombopoietin precursor (Megakaryocyte colony stimulating factor) (Myeloproliferative leukemia virus oncogene ligand) (C-mpl ligand) (ML) (Megakaryocyte growth and development factor) (MGDF); FTL: Ferritin light chain (Ferritin L subunit); HADHSC: Short chain 3-hydroxyacyl-CoA dehydrogenase, mitochondrial precursor (EC 1.1.1.35) (HCDH) (Medium and short chain L-3-hydroxyacyl-coenzyme A dehydrogenase); ALOX5AP: 5-lipoxygenase activating protein (FLAP) (MK-886-binding protein); LAIR1: Homo sapiens leukocyte-associated Ig-like receptor 1 (LAIR1), transcript variant a, mRNA; UPP1: Uridine phosphorylase 1 (EC 2.4.2.3) (UrdPase 1) (UPase 1); LAPTM5: Lysosomal-associated multitransmembrane protein (Retinoic acid-inducible E3 protein) (HA 1520); CSTA: cystatin A (stefin A); ADCY5: adenylate cyclase 5; PHLDB2: pleckstrin homology-like domain, family B, member 2; LL5 beta [Homo sapiens]; GM2A: Ganglioside GM2 activator precursor (GM2-AP) (Cerebroside sulfate activator protein) (Shingolipid activator protein 3) (SAP-3); NUDT16: nudix-type motif 16; ACSL1: Long-chain-fatty-acid—CoA ligase 1 (EC 6.2.1.3) (Long-chain acyl-CoA synthetase 1) (LACS 1) (Palmitoyl-CoA ligase 1) (Long-chain fatty acid CoA ligase 2) (Long-chain acyl-CoA synthetase 2) (LACS 2) (Acyl-CoA synthetase 1) (ACS1) (Palmitoyl-CoA ligase 2); VAMP5: Vesicule-associated membrane protein 5 (VAMP-5) (Myobrevin) (HSPC191); ACP2: LYSOSOMAL ACID PHOSPHATASE PRECURSOR (EC 3.1.3.2) (LAP); HLA-DPA1: HLA class II histocompatibility antigen, DP alpha chain precursor (HLA-SB alpha chain) (MHC class II DP3-alpha) (DP(W3)) (DP(W4)); TUBA3: tubulin, alpha 3; MMP7: MATRILYSIN PRECURSOR (EC 3.4.24.23) (PUMP-1 PROTEASE) (UTERINE METALLOPROTEINASE) (MATRIX METALLOPROTEINASE-7) (MMP-7) (MATRIN); H41: hypothetical protein H41; NR12: nuclear receptor subfamily 1, group 1, member 2; FGFR2: FIBROBLAST GROWTH FACTOR RECEPTOR 2 PRECURSOR (EC 2.7.1.112) (FGFR-2) (KERATINOCYTE GROWTH FACTOR RECEPTOR 2).; GBA: Glucosylceramidase precursor (EC 3.2.1.45) (Beta-glucocerebrosidase) (Acid beta-glucosidase) (D-glucosyl-N-acylsphingosine glucohydrolase) (Alglucerase) (Imiglucerase); CHAF1A: Chromatin assembly factor 1 subunit A (CAF-1 subunit A) (Chromatin assembly factor 1 p150 subunit) (CAF-1 150 kDa subunit) (CAF-1p150); GSK3B: glycogen synthase kinase 3 beta; DOCK2: Dedicator of cytokinesis protein 2; URB: steroid sensitive gene 1; HCLS1: Hematopoietic lineage cell specific protein (Hematopoietic cell-specific LYN substrate 1) (LCKBP1); CD200R1: CD200 receptor 1; SLCO2B1: SOLUTE CARRIER FAMILY 21 MEMBER 9 (ORGANIC ANION TRANSPORTER B) (OATP-B) (ORGANIC ANION TRANSPORTER POLYPEPTIDE-RELATED PROTEIN 2) (OATP-RP2) (OATPRP2); B4GALT4: Beta-1,4-galactosyltransferase 4 (EC 2.4.1.-) (b4Gal-T4) [Includes: N-acetyllactosamine synthase (EC 2.4.1.90) (NaI synthetase); Beta-N-acetylglucosaminyl-glycolipid beta-1,4 galactosyltransferase (EC 2.4.1.-)]; PLCXD2: phosphatidylinositol-specific phospholipase C, X domain containing 2; FABP7: Fatty acid-binding protein, brain (B-FABP) (Brain lipid-binding protein) (BLBP) (Mammary derived growth inhibitor related); CAMKK2: Homo sapiens calcium/calmodulin-dependent protein kinase kinase 2, beta (CAMKK2), transcript variant 1, mRNA; FCGR1A: High affinity immunoglobulin gamma Fc receptor I precursor (Fc-gamma R1) (FcRI) (IgG Fc receptor I) (CD64 antigen); SELL: L-selectin precursor (Lymph node homing receptor) (Leukocyte adhesion molecule-1) (LAM-1) (Leukocyte surface antigen Leu-8) (TQ1) (gp90-MEL) (Leukocyte-endothelial cell adhesion molecule 1) (LECAM1) (CD62L); SELE: Homo sapiens selectin E (endothelial adhesion molecule 1) (SELE), mRNA; HNRPM: Heterogeneous nuclear ribonucleoprotein M (hnRNP M); MGC45840: hypothetical protein MGC45840; F5: Coagulation factor V precursor (Activated protein C cofactor); SMTN: Smoothelin; RA13: Homo sapiens retinoic acid induced 3 (RA13), mRNA; HLA-DRA: HLA class II histocompatibility antigen, DR alpha chain precursor (MHC class II antigen DRA); CSTB: Cystatin B (Liver thiol proteinase inhibitor) (CPI-B) (Stefin B); FLJ12592: N/A; TAGLN3: Neuronal protein NP25 (Neuronal protein 22) (NP22).

### Example 2

### Methods for Genotyping of the Cathgen Samples and Statistical Analysis Early Onset CAD Case Control Sample (CATHGEN)

CATHGEN subjects were recruited sequentially through the cardiac catheterization laboratories at Duke University Hospital (Durham, N.C.) with approval from the Duke Institutional Review Board. All subjects undergoing catheterization were offered participation in the study and signed informed consent. Medical history and clinical data were collected and stored in the Duke Information System for Cardiovascular Care database maintained at the Duke Clinical Research Institute [1].

Controls and cases were chosen on the basis of extent of coronary artery disease as measured by the CAD index (CADi). CADi is a numerical summary of coronary angiographic data that incorporates the extent and anatomical distribution of coronary disease [2]. CADi has been shown to be a better predictor of clinical outcome than the extent of CAD [3]. Affected status was determined by the presence of significant CAD defined as a CADi≧32 [4]. For patients older than 55 years of age, a higher CADi threshold (CADi≧74) was used to adjust for the higher baseline extent of CAD in this group. Medical records were reviewed to determine the age-of-onset (AOO) of CAD, i.e. the age at first documented surgical or percutaneous coronary revascularization procedure, myocardial infarction (MI), or cardiac catheterization meeting the above defined CADi thresholds. The CATHGEN cases were stratified into a young affected group (AOO ≦55 years), which provides a consistent comparison group for the GENECARD family study. Controls were defined as subjects ≧60 years of age, with no CAD as demonstrated by coronary angiography and no documented history of cerebrovascular or peripheral vascular

A set of at least 5 SNPs with a minor allele frequency (MAF) of >10% [5] was selected for genotyping in each gene CATHOEN samples using the SNPselector program [6]. Genomic DNA for CATHGEN samples was extracted from whole blood using the PureGene system (Gentra Systems, Minneapolis, Minn.). Genotyping was performed using the ABI 7900HT Taqman SNP genotyping system (Applied Biosystems, Foster City, Calif.), which incorporates a standard PCR-based, dual fluor, allelic discrimination assay in 384 well plate format with a dual laser scanner. Allelic discrimination assays were purchased through Applied Biosystems or, in cases in which the assays were not available, primer and probe sets were designed and purchased through Integrated DNA Technologies (IDT, Coralville, Iowa). A total of 15 quality control samples, composed of 6 reference genotype controls in duplicate, two Centre d'Etude du Polymorphisme Humain (CEPH) pedigree individuals and one no-template sample, were included in each quadrant of the 384 well plate. Genotyping was also performed using the Illumina BeadStation 500G SNP genotyping system (Illumina, San Diego, Calif.). Each Sentrix Array generates 1536 genotypes for 96 individuals; within each individual array experiment four quality control samples were included, two CEPH pedigree individuals and two identical in-plate controls. Results of the CEPH and quality control samples were compared to identify possible sample plating errors and genotype calling inconsistencies. SNPs that showed mismatches on quality control samples were reviewed by an independent genotyping supervisor for potential genotyping errors. All SNPs examined were successfully genotyped for 95% or more of the individuals in the study. Error rate estimates for SNPs meeting the quality control benchmarks were determined to be less than 0.2%.

All SNPs were tested for deviations from Hardy-Weinberg equilibrium (HWE) in the affected and unaffected race stratified groups. No such deviations were observed.

Additionally, linkage disequilibrium between pairs of SNPs was assessed using the Graphical Overview of Linkage Disequilibrium (GOLD) package [7] and displayed using Haploview[8]. Allelic association in CATHGEN was examined using multivariable logistic regression modeling adjusted for race and sex, and also for race, sex, and known CAD risk factors (history of hypertension, history of diabetes mellitus, body mass index, history of dyslipidemia, and smoking history) as covariates. These adjustments could hypothetically allow us to control for competing genetic pathways that are independent risk factors for CAD, therefore allowing us to detect a separate CAD genetic effect. SAS 9.1 (SAS Institute, Cary, N.C.) was used for statistical analysis. The haplo.stats package was used to identify and test for association of haplotypes in CATHGEN. Haplo.stats expands on the likelihood approach to account for ambiguity in case-control studies by using a generalized linear model (GLM) to test for haplotype association which allows for adjustment of non-genetic covariates [9]. This method derives a score statistic to test the null hypothesis of no association of the trait with the genotype. In addition to the global statistic, haplo.stats computes score statistics for the components of the genetic vectors, such as individual haplotypes.

Results from these experiments are shown in Tables 3-5. The SNP represented by SEQ ID NO:188 contains a five-base pair deletion relative to the wild-type sequence. As used herein, the term SNP also includes this polymorphism having the five-nucleotide deletion. “RK” indicates rank in predicting CAD, with the most predictive genes having a lower number; “CH” indicates the chromosome in which the gene locus resides in the human genome.

TABLE 3

| | | | | | | SEQ ID | SEQ ID |
|---|---|---|---|---|---|---|---|
| RK | CH | LOCUS | GENBANK | PROBE | NCB135 | (SNP) | (WT) |
| 15 | 1 | HSPG2 | NM_005529 | RS4654773 | 21,997,568 | 1 | 576 |
| 15 | 1 | HSPG2 | NM_005529 | RS17467346 | 22,005,318 | 2 | 577 |
| 15 | 1 | HSPG2 | NM_005529 | RS11587857 | 22,005,614 | 3 | 578 |
| 15 | 1 | HSPG2 | NM_005529 | RS12081298 | 22,007,531 | 4 | 579 |
| 43 | 1 | CDC42 | NM_001791 | RS2501275 | 22,120,371 | 5 | 580 |
| 43 | 1 | CDC42 | NM_001791 | RS2473322 | 22,135,378 | 6 | 581 |
| 43 | 1 | CDC42 | NM_001791 | RS10917139 | 22,146,844 | 7 | 582 |
| 43 | 1 | CDC42 | NM_001791 | RS2056974 | 22,154,400 | 8 | 583 |
| 71 | 1 | C1QB | NM_000491 | RS291989 | 22,725,205 | 9 | 584 |
| 71 | 1 | C1QB | NM_000491 | RS291988 | 22,725,364 | 10 | 585 |
| 71 | 1 | C1QB | NM_000491 | RS291985 | 22,726,245 | 11 | 586 |
| 71 | 1 | C1QB | NM_000491 | RS12756603 | 22,727,182 | 12 | 587 |
| 71 | 1 | C1QB | NM_000491 | RS291982 | 22,727,712 | 13 | 588 |
| 71 | 1 | C1QB | NM_000491 | RS631090 | 22,731,709 | 14 | 589 |
| 71 | 1 | C1QB | NM_000491 | RS623607 | 22,732,022 | 15 | 590 |
| 71 | 1 | C1QB | NM_000491 | RS10580 | 22,733,264 | 16 | 591 |
| 71 | 1 | C1QB | NM_000491 | RS292007 | 22,736,818 | 17 | 592 |
| 4 | 1 | AIM1L | AK095339 | RS7416513 | 26,332,091 | 18 | 593 |
| 4 | 1 | AIM1L | AK095339 | RS17163868 | 26,332,523 | 19 | 594 |
| 4 | 1 | AIM1L | AK095339 | RS4659371 | 26,341,703 | 20 | 595 |
| 4 | 1 | AIM1L | AK095339 | RS4659431 | 26,342,533 | 21 | 596 |
| 4 | 1 | AIM1L | AK095339 | RS7517559 | 26,346,916 | 22 | 597 |
| 4 | 1 | AIM1L | AK095339 | RS4072445 | 26,348,361 | 23 | 598 |
| 4 | 1 | AIM1L | AK095339 | RS11247920 | 26,349,620 | 24 | 599 |
| 4 | 1 | AIM1L | AK095339 | RS7535656 | 26,357,608 | 25 | 600 |
| 4 | 1 | AIM1L | AK095339 | RS10902742 | 26,360,399 | 26 | 601 |
| 4 | 1 | AIM1L | AK095339 | RS4454539 | 26,364,405 | 27 | 602 |
| 4 | 1 | AIM1L | AK095339 | RS4233461 | 26,365,448 | 28 | 603 |
| 19 | 1 | C1ORF38 | AF044896 | RS11247703 | 27,887,795 | 29 | 604 |
| 19 | 1 | C1ORF38 | AF044896 | RS12048235 | 27,890,026 | 30 | 605 |
| 19 | 1 | C1ORF38 | AF044896 | RS3766398 | 27,893,447 | 31 | 606 |
| 19 | 1 | C1ORF38 | AF044896 | RS3766400 | 27,893,508 | 32 | 607 |
| 19 | 1 | C1ORF38 | AF044896 | RS2236074 | 27,895,526 | 33 | 608 |
| 19 | 1 | C1ORF38 | AF044896 | RS1467465 | 27,895,545 | 34 | 609 |
| 19 | 1 | C1ORF38 | AF044896 | RS1467464 | 27,895,792 | 35 | 610 |
| 19 | 1 | C1ORF38 | AF044896 | RS6564 | 27,897,117 | 36 | 611 |
| 19 | 1 | C1ORF38 | AF044896 | RS6565 | 27,897,299 | 37 | 612 |
| 58 | 1 | LAPTM5 | U51240 | RS3795438 | 30,875,730 | 38 | 613 |
| 58 | 1 | LAPTM5 | U51240 | RS12404920 | 30,876,050 | 39 | 614 |
| 58 | 1 | LAPTM5 | U51240 | 1P0258 | 30,877,135 | 40 | 615 |
| 58 | 1 | LAPTM5 | U51240 | RS1188356 | 30,880,175 | 41 | 616 |
| 58 | 1 | LAPTM5 | U51240 | RS1188360 | 30,881,469 | 42 | 617 |
| 58 | 1 | LAPTM5 | U51240 | RS3748602 | 30,883,462 | 43 | 618 |
| 58 | 1 | LAPTM5 | U51240 | RS3748603 | 30,884,064 | 44 | 619 |
| 58 | 1 | LAPTMS | U51240 | RS1050663 | 30,884,457 | 45 | 620 |
| 58 | 1 | LAPTM5 | U51240 | RS11585511 | 30,886,062 | 46 | 621 |
| 58 | 1 | LAPTM5 | U51240 | RS3790495 | 30,890,608 | 47 | 622 |
| 58 | 1 | LAPTM5 | U51240 | RS3790496 | 30,891,084 | 48 | 623 |
| 58 | 1 | LAPTM5 | U51240 | RS1188349 | 30,892,750 | 49 | 624 |
| 58 | 1 | LAPTM5 | U51240 | RS1188347 | 30,895,433 | 50 | 625 |
| 58 | 1 | LAPTM5 | U51240 | RS3790503 | 30,898,168 | 51 | 626 |
| 58 | 1 | LAPTM5 | U51240 | RS1407882 | 30,899,288 | 52 | 627 |
| 58 | 1 | LAPTM5 | U51240 | RS2273979 | 30,899,761 | 53 | 628 |
| 58 | 1 | LAPTM5 | U51240 | RS11801629 | 30,900,219 | 54 | 629 |
| 45 | 1 | CACNA1E | NM_000721 | RS704326 | 178,491,314 | 55 | 630 |
| 72 | 1 | LAMC1 | NM_002293 | RS4652763 | 179,725,741 | 56 | 631 |
| 72 | 1 | LAMC1 | NM_002293 | RS12144261 | 179,745,805 | 57 | 632 |
| 72 | 1 | LAMC1 | NM_002293 | RS10911229 | 179,782,025 | 58 | 633 |
| 72 | 1 | LAMC1 | NM_002293 | RS2296291 | 179,811,166 | 59 | 634 |
| 72 | 1 | LAMC1 | NM_002293 | RS7556132 | 179,817,412 | 60 | 635 |
| 72 | 1 | LAMC1 | NM_002293 | RS7410919 | 179,826,204 | 61 | 636 |
| 72 | 1 | LAMC1 | NM_002293 | RS20559 | 179,831,217 | 62 | 637 |
| 72 | 1 | LAMC1 | NM_002293 | RS4651146 | 179,837,191 | 63 | 638 |
| 72 | 1 | LAMC1 | NM_002293 | RS3738829 | 179,845,519 | 64 | 639 |
| 72 | 1 | LAMC1 | NM_002293 | RS1547715 | 179,845,609 | 65 | 640 |
| 53 | 1 | CFH | NM_000186 | RS529825 | 193,366,763 | 66 | 641 |
| 53 | 1 | CFH | NM_000186 | RS800292 | 193,373,890 | 67 | 642 |
| 53 | 1 | CFH | NM_000186 | RS1061147 | 193,385,981 | 68 | 643 |
| 53 | 1 | CFH | NM_000186 | RS1061170 | 193,390,894 | 69 | 644 |
| 53 | 1 | CFH | NM_000186 | RS10801555 | 193,391,918 | 70 | 645 |
| 53 | 1 | CFH | NM_000186 | RS2019724 | 193,406,574 | 71 | 646 |
| 53 | 1 | CFH | NM_000186 | RS393955 | 193,424,127 | 72 | 647 |
| 53 | 1 | CFH | NM_000186 | RS1065489 | 193,441,431 | 73 | 648 |
| 53 | 1 | CFH | NM_000186 | RS10801560 | 193,446,257 | 74 | 649 |
| 61 | 1 | LMOD1 | X54162 | RS6427922 | 198,587,069 | 75 | 650 |
| 61 | 1 | LMOD1 | X54162 | RS4987074 | 198,597,289 | 76 | 651 |
| 61 | 1 | LMOD1 | X54162 | RS3738289 | 198,599,726 | 77 | 652 |
| 61 | 1 | LMOD1 | X54162 | RS2820312 | 198,600,914 | 78 | 653 |
| 61 | 1 | LMOD1 | X54162 | RS2820315 | 198,603,921 | 79 | 654 |
| 61 | 1 | LMOD1 | X54162 | RS7528681 | 198,606,369 | 80 | 655 |
| 61 | 1 | LMOD1 | X54162 | RS2644121 | 198,612,941 | 81 | 656 |
| 61 | 1 | LMOD1 | X54162 | RS2819346 | 198,613,744 | 82 | 657 |
| 61 | 1 | LMOD1 | X54162 | RS10800796 | 198,617,854 | 83 | 658 |
| 61 | 1 | LMOD1 | X54162 | RS2360545 | 198,623,599 | 84 | 659 |
| 61 | 1 | LMOD1 | X54162 | RS9787358 | 198,629,327 | 85 | 660 |
| 61 | 1 | LMOD1 | X54162 | RS2819366 | 198,639,638 | 86 | 661 |
| 25 | 2 | CAPG | M94345 | RS11678506 | 85,529,829 | 87 | 662 |
| 25 | 2 | CAPG | M94345 | RS2271627 | 85,533,717 | 88 | 663 |
| 25 | 2 | CAPG | M94345 | RS11690650 | 85,533,975 | 89 | 664 |
| 25 | 2 | CAPG | M94345 | RS11539100 | 85,536,880 | 90 | 665 |
| 25 | 2 | CAPG | M94345 | RS11687035 | 85,537,097 | 91 | 666 |
| 25 | 2 | CAPG | M94345 | RS2271625 | 85,537,171 | 92 | 667 |
| 25 | 2 | CAPG | M94345 | RS11539103 | 85,537,991 | 93 | 668 |
| 25 | 2 | CAPG | M94345 | RS2002444 | 85,540,214 | 94 | 669 |
| 25 | 2 | CAPG | M94345 | RS2229669 | 85,540,403 | 95 | 670 |
| 25 | 2 | CAPG | M94345 | RS2229668 | 85,540,641 | 96 | 671 |
| 25 | 2 | CAPG | M94345 | RS13020378 | 85,544,600 | 97 | 672 |
| 25 | 2 | CAPG | M94345 | RS11696093 | 85,547,853 | 98 | 673 |
| 25 | 2 | CAPG | M94345 | RS3770102 | 85,549,495 | 99 | 674 |
| 25 | 2 | CAPG | M94345 | RS11682055 | 85,549,981 | 100 | 675 |
| 25 | 2 | CAPG | M94345 | RS1877954 | 85,565,957 | 101 | 676 |
| 25 | 2 | CAPG | M94345 | RS1877955 | 85,566,184 | 102 | 677 |
| 42 | 2 | VAMP8 | NM_003761 | RS17508727 | 85,711,434 | 103 | 678 |
| 42 | 2 | VAMP8 | NM_003761 | RS13426038 | 85,715,056 | 104 | 679 |
| 42 | 2 | VAMP8 | NM_003761 | RS3770098 | 85,717,025 | 105 | 680 |
| 42 | 2 | VAMP8 | NM_003761 | RS3731828 | 85,717,924 | 106 | 681 |
| 42 | 2 | VAMP8 | NM_003761 | RS1009 | 85,720,395 | 107 | 682 |
| 42 | 2 | VAMP8 | NM_003761 | RS1010 | 85,720,640 | 108 | 683 |
| 50 | 2 | VAMP5 | N90862 | RS1561198 | 85,721,647 | 109 | 684 |
| 50 | 2 | VAMP5 | N90862 | RS1254901 | 85,722,887 | 110 | 685 |
| 50 | 2 | VAMP5 | N90862 | RS12714147 | 85,725,492 | 111 | 686 |
| 50 | 2 | VAMP5 | N90862 | RS10206961 | 85,726,642 | 112 | 687 |
| 50 | 2 | VAMP5 | N90862 | RS1254900 | 85,727,992 | 113 | 688 |
| 50 | 2 | VAMP5 | N90862 | RS719023 | 85,730,146 | 114 | 689 |
| 50 | 2 | VAMP5 | N90862 | RS2289976 | 85,730,455 | 115 | 690 |
| 50 | 2 | VAMP5 | N90862 | RS14976 | 85,730,544 | 116 | 691 |
| 50 | 2 | VAMP5 | N90862 | RS14242 | 85,732,070 | 117 | 692 |
| 2 | 2 | LOC51255 | NM_016494 | RS2232739 | 85,734,340 | 118 | 693 |
| 2 | 2 | LOC51255 | NM_016494 | RS2232745 | 85,735,290 | 119 | 694 |
| 2 | 2 | LOC51255 | NM_016494 | RS6643 | 85,735,909 | 120 | 695 |
| 66 | 2 | HOXD1 | AW001001 | RS1562315 | 176,870,989 | 121 | 696 |
| 66 | 2 | HOXD1 | AW001001 | RS1446575 | 176,873,308 | 122 | 697 |
| 66 | 2 | HOXD1 | AW001001 | RS13390503 | 176,879,561 | 123 | 698 |
| 66 | 2 | HOXD1 | AW001001 | RS13390932 | 176,879,918 | 124 | 699 |
| 66 | 2 | HOXD1 | AW001001 | RS6710142 | 176,880,276 | 125 | 700 |
| 66 | 2 | HOXD1 | AW001001 | RS6725515 | 176,880,600 | 126 | 701 |
| 66 | 2 | HOXD1 | AW001001 | RS11551009 | 176,880,885 | 127 | 702 |
| 66 | 2 | HOXD1 | AW001001 | RS1374326 | 176,883,823 | 128 | 703 |
| 66 | 2 | HOXD1 | AW001001 | RS1026032 | 176,890,330 | 129 | 704 |
| 36 | 3 | RHOA | NM_001664 | RS8179164 | 49,372,288 | 130 | 705 |
| 36 | 3 | RHOA | NM_001664 | RS974495 | 49,375,486 | 131 | 706 |
| 36 | 3 | RHOA | NM_001664 | RS7621003 | 49,386,408 | 132 | 707 |
| 36 | 3 | RHOA | NM_001664 | RS7631908 | 49,400,711 | 133 | 708 |
| 36 | 3 | RHOA | NM_001664 | RS4855877 | 49,423,531 | 134 | 709 |
| 46 | 3 | FLJ39873 | NM_173799 | RS1316642 | 115,506,753 | 135 | 710 |
| 24 | 3 | IGSF11 | NM_152538 | RS1521299 | 120,093,419 | 136 | 711 |
| 24 | 3 | IGSF11 | NM_152538 | RS4687959 | 120,106,104 | 137 | 712 |
| 24 | 3 | IGSF11 | NM_152538 | RS6782002 | 120,107,321 | 138 | 713 |
| 24 | 3 | IGSF11 | NM_152538 | RS1468738 | 120,114,311 | 139 | 714 |
| 24 | 3 | IGSF11 | NM_152538 | RS2160052 | 120,124,569 | 140 | 715 |
| 24 | 3 | IGSF11 | NM_152538 | RS2192365 | 120,126,099 | 141 | 716 |
| 24 | 3 | IGSF11 | NM_152538 | RS2903250 | 120,131,750 | 142 | 717 |
| 24 | 3 | IGSF11 | NM_152538 | RS9837571 | 120,138,354 | 143 | 718 |
| 24 | 3 | IGSF11 | NM_152538 | RS39688 | 120,225,538 | 144 | 719 |
| 24 | 3 | IGSF11 | NM_152538 | RS35859 | 120,233,743 | 145 | 720 |
| 24 | 3 | IGSF11 | NM_152538 | RS1347448 | 120,305,831 | 146 | 721 |
| 68 | 3 | CD80 | NM_005191 | HCV387937 | 120,727,283 | 147 | 722 |
| 68 | 3 | CD80 | NM_005191 | RS1523311 | 120,730,991 | 148 | 723 |
| 68 | 3 | CD80 | NM_005191 | RS2049502 | 120,737,075 | 149 | 724 |
| 68 | 3 | CD80 | NM_005191 | RS626364 | 120,755,573 | 150 | 725 |
| 54 | 3 | FSTL1 | NM_007085 | RS1621291 | 121,588,392 | 151 | 726 |
| 54 | 3 | FSTL1 | NM_007085 | RS2488 | 121,595,976 | 152 | 727 |
| 54 | 3 | FSTL1 | NM_007085 | RS1057231 | 121,596,093 | 153 | 728 |
| 54 | 3 | FSTL1 | NM_007085 | RS13709 | 121,596,818 | 154 | 729 |
| 54 | 3 | FSTL1 | NM_007085 | RS1700 | 121,597,327 | 155 | 730 |
| 54 | 3 | FSTL1 | NM_007085 | RS1147696 | 121,602,169 | 156 | 731 |
| 54 | 3 | FSTL1 | NM_007085 | RS1147704 | 121,610,461 | 157 | 732 |
| 54 | 3 | FSTL1 | NM_007085 | RS1515577 | 121,611,630 | 158 | 733 |
| 54 | 3 | FSTL1 | NM_007085 | RS13097755 | 121,614,452 | 159 | 734 |
| 54 | 3 | FSTL1 | NM_007085 | RS2272515 | 121,617,573 | 160 | 735 |
| 54 | 3 | FSTL1 | NM_007085 | RS1733306 | 121,638,524 | 161 | 736 |
| 54 | 3 | FSTL1 | NM_007085 | RS1123897 | 121,639,724 | 162 | 737 |
| 54 | 3 | FSTL1 | NM_007085 | RS1123898 | 121,639,772 | 163 | 738 |
| 54 | 3 | FSTL1 | NM_007085 | RS1259333 | 121,646,977 | 164 | 739 |
| 54 | 3 | FSTL1 | NM_007085 | RS1147707 | 121,651,938 | 165 | 740 |
| 54 | 3 | FSTL1 | NM_007085 | RS1147709 | 121,654,410 | 166 | 741 |
| 49 | 3 | NDUFB4 | NM_004547 | RS17140284 | 121,797,081 | 167 | 742 |
| 20 | 3 | PARP9 | NM_031458 | RS3817040 | 123,737,459 | 168 | 743 |
| 20 | 3 | PARP9 | NM_031458 | RS7631465 | 123,754,360 | 169 | 744 |
| 16 | 3 | MYLK | NM_053027 | RS9422 | 124,815,030 | 170 | 745 |
| 16 | 3 | MYLK | NM_053027 | RS860224 | 124,820,104 | 171 | 746 |
| 16 | 3 | MYLK | NM_053027 | RS820447 | 124,830,869 | 172 | 747 |
| 16 | 3 | MYLK | NM_053027 | RS820463 | 124,839,727 | 173 | 748 |
| 16 | 3 | MYLK | NM_053027 | RS1254392 | 124,850,703 | 174 | 749 |
| 16 | 3 | MYLK | NM_053027 | RS820325 | 124,868,367 | 175 | 750 |
| 16 | 3 | MYLK | NM_053027 | RS820371 | 124,887,401 | 176 | 751 |
| 16 | 3 | MYLK | NM_053027 | RS11717814 | 124,891,241 | 177 | 752 |
| 16 | 3 | MYLK | NM_053027 | RS40305 | 124,894,279 | 178 | 753 |
| 16 | 3 | MYLK | NM_053027 | RS820335 | 124,898,204 | 179 | 754 |
| 16 | 3 | MYLK | NM_053027 | RS820336 | 124,898,471 | 180 | 755 |
| 16 | 3 | MYLK | NM_053027 | RS3732487 | 124,902,263 | 181 | 756 |
| 16 | 3 | MYLK | NM_053027 | RS3732485 | 124,902,472 | 182 | 757 |
| 16 | 3 | MYLK | NM_053027 | RS7641248 | 124,909,674 | 183 | 758 |
| 16 | 3 | MYLK | NM_053027 | RS820329 | 124,927,474 | 184 | 759 |
| 16 | 3 | MYLK | NM_053027 | RS4678047 | 124,935,528 | 185 | 760 |
| 16 | 3 | MYLK | NM_053027 | RS3796164 | 124,935,751 | 186 | 761 |
| 16 | 3 | MYLK | NM_053027 | RS9840993 | 124,940,583 | 187 | 762 |
| 16 | 3 | MYLK | NM_053027 | RS3085179 | 124,941,793 | 188 | 763 |
| 16 | 3 | MYLK | NM_053027 | RS11718105 | 124,946,398 | 189 | 764 |
| 16 | 3 | MYLK | NM_053027 | RS11707609 | 124,986,114 | 190 | 765 |
| 16 | 3 | MYLK | NM_053027 | RS7639329 | 124,993,625 | 191 | 766 |
| 16 | 3 | MYLK | NM_053027 | RS28497577 | 124,995,317 | 192 | 767 |
| 16 | 3 | MYLK | NM_053027 | RS9846863 | 124,996,168 | 193 | 768 |
| 16 | 3 | MYLK | NM_053027 | RS4678060 | 124,998,930 | 194 | 769 |
| 16 | 3 | MYLK | NM_053027 | RS11714297 | 125,002,269 | 195 | 770 |
| 16 | 3 | MYLK | NM_053027 | RS9816400 | 125,006,336 | 196 | 771 |
| 16 | 3 | MYLK | NM_053027 | RS2124508 | 125,009,601 | 197 | 772 |
| 16 | 3 | MYLK | NM_053027 | RS10934651 | 125,015,899 | 198 | 773 |
| 16 | 3 | MYLK | NM_053027 | RS16834774 | 125,017,283 | 199 | 774 |
| 16 | 3 | MYLK | NM_053027 | RS13094938 | 125,017,560 | 200 | 775 |
| 16 | 3 | MYLK | NM_053027 | RS9289225 | 125,018,733 | 201 | 776 |
| 16 | 3 | MYLK | NM_053027 | RS7652269 | 125,018,872 | 202 | 777 |
| 16 | 3 | MYLK | NM_053027 | RS3911406 | 125,021,533 | 203 | 778 |
| 16 | 3 | MYLK | NM_053027 | RS9829784 | 125,022,826 | 204 | 779 |
| 16 | 3 | MYLK | NM_053027 | HCV1602689 | 125,024,094 | 205 | 780 |
| 16 | 3 | MYLK | NM_053027 | RS2682215 | 125,027,266 | 206 | 781 |
| 16 | 3 | MYLK | NM_053027 | RS2605417 | 125,032,085 | 207 | 782 |
| 16 | 3 | MYLK | NM_053027 | RS2700358 | 125,039,169 | 208 | 783 |
| 16 | 3 | MYLK | NM_053027 | RS2682239 | 125,042,419 | 209 | 784 |
| 16 | 3 | MYLK | NM_053027 | RS7628376 | 125,045,246 | 210 | 785 |
| 16 | 3 | MYLK | NM_053027 | RS4461370 | 125,048,862 | 211 | 786 |
| 16 | 3 | MYLK | NM_053027 | RS1343700 | 125,054,444 | 212 | 787 |
| 16 | 3 | MYLK | NM_053027 | RS16834817 | 125,060,723 | 213 | 788 |
| 16 | 3 | MYLK | NM_053027 | RS12495918 | 125,065,904 | 214 | 789 |
| 16 | 3 | MYLK | NM_053027 | RS2682218 | 125,066,569 | 215 | 790 |
| 16 | 3 | MYLK | NM_053027 | RS4118366 | 125,066,921 | 216 | 791 |
| 16 | 3 | MYLK | NM_053027 | RS16834826 | 125,067,178 | 217 | 792 |
| 16 | 3 | MYLK | NM_053027 | RS13096686 | 125,072,942 | 218 | 793 |
| 16 | 3 | MYLK | NM_053027 | RS2700408 | 125,078,122 | 219 | 794 |
| 16 | 3 | MYLK | NM_053027 | RS2682229 | 125,084,440 | 220 | 795 |
| 16 | 3 | MYLK | NM_053027 | RS2700410 | 125,085,087 | 221 | 796 |
| 16 | 3 | MYLK | NM_053027 | RS1920221 | 125,089,642 | 222 | 797 |
| 6 | 3 | OR7E29P | NG_004130 | RS2979310 | 126,871,199 | 223 | 798 |
| 23 | 3 | KLF15 | NM_014079 | RS7622890 | 127,540,380 | 224 | 799 |
| 23 | 3 | KLF15 | NM_014079 | RS938390 | 127,541,247 | 225 | 800 |
| 23 | 3 | KLF15 | NM_014079 | RS938389 | 127,541,460 | 226 | 801 |
| 23 | 3 | KLF15 | NM_014079 | RS7615776 | 127,543,315 | 227 | 802 |
| 23 | 3 | KLF15 | NM_014079 | RS9838915 | 127,548,918 | 228 | 803 |
| 23 | 3 | KLF15 | NM_014079 | RS9850626 | 127,551,477 | 229 | 804 |
| 23 | 3 | KLF15 | NM_014079 | RS6764427 | 127,552,824 | 230 | 805 |
| 23 | 3 | KLF15 | NM_014079 | RS1358087 | 127,561,588 | 231 | 806 |
| 23 | 3 | KLF15 | NM_014079 | RS7636709 | 127,562,692 | 232 | 807 |
| 63 | 3 | GATA2 | ABC002557 | RS2713594 | 129,679,198 | 233 | 808 |
| 63 | 3 | GATA2 | ABC002557 | RS2713579 | 129,680,802 | 234 | 809 |
| 63 | 3 | GATA2 | ABC002557 | 3P0457 | 129,681,678 | 235 | 810 |
| 63 | 3 | GATA2 | ABC002557 | 3P0456 | 129,681,863 | 236 | 811 |
| 63 | 3 | GATA2 | ABC002557 | 3P0448 | 129,682,014 | 237 | 812 |
| 63 | 3 | GATA2 | ABC002557 | RS3803 | 129,682,078 | 238 | 813 |
| 63 | 3 | GATA2 | ABC002557 | 3P0450 | 129,682,150 | 239 | 814 |
| 63 | 3 | GATA2 | ABC002557 | RS10934857 | 129,682,360 | 240 | 815 |
| 63 | 3 | GATA2 | ABC002557 | 3P0455 | | 241 | 816 |
| 63 | 3 | GATA2 | ABC002557 | RS2713604 | 129,683,157 | 242 | 817 |
| 63 | 3 | GATA2 | ABC002557 | RS2713603 | 129,683,232 | 243 | 818 |
| 63 | 3 | GATA2 | ABC002557 | RS2659689 | 129,685,704 | 244 | 819 |
| 63 | 3 | GATA2 | ABC002557 | RS2659691 | 129,686,398 | 245 | 820 |
| 63 | 3 | GATA2 | ABC002557 | RS2713601 | 129,686,434 | 246 | 821 |
| 63 | 3 | GATA2 | ABC002557 | RS2335052 | 129,687,649 | 247 | 822 |
| 63 | 3 | GATA2 | ABC002557 | RS1573858 | 129,688,558 | 248 | 823 |
| 63 | 3 | GATA2 | ABC002557 | RS1806462 | 129,689,316 | 249 | 824 |
| 63 | 3 | GATA2 | ABC002557 | RS2953120 | 129,692,180 | 250 | 825 |
| 63 | 3 | GATA2 | ABC002557 | RS2860228 | 129,692,365 | 251 | 826 |
| 63 | 3 | GATA2 | ABC002557 | RS9851497 | 129,695,224 | 252 | 827 |
| 63 | 3 | GATA2 | ABC002557 | RS6439129 | 129,695,471 | 253 | 828 |
| 52 | 3 | PLXND1 | NM_015103 | RS2625967 | 130,749,957 | 254 | 829 |
| 52 | 3 | PLXND1 | NM_015103 | RS2285359 | 130,764,416 | 255 | 830 |
| 52 | 3 | PLXND1 | NM_015103 | RS2245285 | 130,769,111 | 256 | 831 |
| 52 | 3 | PLXND1 | NM_015103 | RS2245278 | 130,769,333 | 257 | 832 |
| 52 | 3 | PLXND1 | NM_015103 | RS2285366 | 130,772,785 | 258 | 833 |
| 52 | 3 | PLXND1 | NM_015103 | RS2285368 | 130,774,197 | 259 | 834 |
| 52 | 3 | PLXND1 | NM_015103 | RS2244708 | 130,774,449 | 260 | 835 |
| 52 | 3 | PLXND1 | NM_015103 | RS2255703 | 130,775,954 | 261 | 836 |
| 52 | 3 | PLXND1 | NM_015103 | RS1110168 | 130,779,921 | 262 | 837 |
| 52 | 3 | PLXND1 | NM_015103 | RS10934885 | 130,781,692 | 263 | 838 |
| 52 | 3 | PLXND1 | NM_015103 | RS2285370 | 130,785,153 | 264 | 839 |
| 52 | 3 | PLXND1 | NM_015103 | RS2285371 | 130,785,770 | 265 | 840 |
| 52 | 3 | PLXND1 | NM_015103 | RS2285372 | 130,787,495 | 266 | 841 |
| 52 | 3 | PLXND1 | NM_015103 | RS2301572 | 130,788,158 | 267 | 842 |
| 52 | 3 | PLXND1 | NM_015103 | RS2285373 | 130,790,907 | 268 | 843 |
| 52 | 3 | PLXND1 | NM_015103 | RS4688807 | 130,791,961 | 269 | 844 |
| 22 | 3 | ATP2C1 | NM_001001485 | RS852216 | 132,094,968 | 270 | 845 |
| 22 | 3 | ATP2C1 | NM_001001485 | RS2669869 | 132,100,165 | 271 | 846 |
| 22 | 3 | ATP2C1 | NM_001001485 | RS712984 | 132,131,496 | 272 | 847 |
| 22 | 3 | ATP2C1 | NM_001001485 | RS852214 | 132,144,013 | 273 | 848 |
| 22 | 3 | ATP2C1 | NM_001001485 | RS2685193 | 132,159,002 | 274 | 849 |
| 22 | 3 | ATP2C1 | NM_001001485 | RS218481 | 132,204,901 | 275 | 850 |
| 22 | 3 | ATP2C1 | NM_001001485 | RS190067 | 132,213,062 | 276 | 851 |
| 41 | 3 | BFSP2 | NM_003571 | RS517255 | 134,600,752 | 277 | 852 |
| 41 | 3 | BFSP2 | NM_003571 | RS4854585 | 134,619,982 | 278 | 853 |
| 41 | 3 | BFSP2 | NM_003571 | RS2276737 | 134,650,061 | 279 | 854 |
| 41 | 3 | BFSP2 | NM_003571 | RS1881918 | 134,653,982 | 280 | 855 |
| 41 | 3 | BFSP2 | NM_003571 | RS2737717 | 134,668,532 | 281 | 856 |
| 41 | 3 | BFSP2 | NM_003571 | RS6439410 | 134,676,110 | 282 | 857 |
| 47 | 3 | AGTR1 | D13814 | RS2638362 | 149,903,214 | 283 | 858 |
| 47 | 3 | AGTR1 | D13814 | RS10935724 | 149,903,951 | 284 | 859 |
| 47 | 3 | AGTR1 | D13814 | RS931490 | 149,913,465 | 285 | 860 |
| 47 | 3 | AGTR1 | D13814 | RS2640543 | 149,915,067 | 286 | 861 |
| 47 | 3 | AGTR1 | D13814 | RS718858 | 149,918,210 | 287 | 862 |
| 47 | 3 | AGTR1 | D13814 | RS909383 | 149,918,904 | 288 | 863 |
| 47 | 3 | AGTR1 | D13814 | RS3772620 | 149,919,006 | 289 | 864 |
| 47 | 3 | AGTR1 | D13814 | RS389566 | 149,929,080 | 290 | 865 |
| 47 | 3 | AGTR1 | D13814 | RS385338 | 149,931,854 | 291 | 866 |
| 47 | 3 | AGTR1 | D13814 | RS275649 | 149,936,024 | 292 | 867 |
| 47 | 3 | AGTR1 | D13814 | RS1800766 | 149,940,340 | 293 | 868 |
| 47 | 3 | AGTR1 | D13814 | RS5182 | 149,942,093 | 294 | 869 |
| 47 | 3 | AGTR1 | D13814 | RS5188 | 149,942,917 | 295 | 870 |
| 47 | 3 | AGTR1 | D13814 | RS275645 | 149,947,152 | 296 | 871 |
| 47 | 3 | AGTR1 | D13814 | RS9849625 | 150,022,852 | 297 | 872 |
| 47 | 3 | AGTR1 | D13814 | RS3772587 | 150,059,614 | 298 | 873 |
| 33 | 4 | PPARGC1A | NM_013261 | RS3774923 | 23,471,333 | 299 | 874 |
| 33 | 4 | PPARGC1A | NM_013261 | RS3736265 | 23,490,976 | 300 | 875 |
| 33 | 4 | PPARGC1A | NM_013261 | RS8192678 | 23,491,931 | 301 | 876 |
| 33 | 4 | PPARGC1A | NM_013261 | RS2290604 | 23,506,507 | 302 | 877 |
| 75 | 4 | HADHSC | X96752 | RS221330 | 109,278,971 | 303 | 878 |
| 75 | 4 | HADHSC | X96752 | RS3775974 | 109,283,987 | 304 | 879 |
| 75 | 4 | HADHSC | X96752 | RS141066 | 109,289,155 | 305 | 880 |
| 75 | 4 | HADHSC | X96752 | RS763432 | 109,289,241 | 306 | 881 |
| 75 | 4 | HADHSC | X96752 | RS1051519 | 109,298,336 | 307 | 882 |
| 75 | 4 | HADHSC | X96752 | RS732940 | 109,302,674 | 308 | 883 |
| 75 | 4 | HADHSC | X96752 | RS732941 | 109,302,708 | 309 | 884 |
| 75 | 4 | HADHSC | X96752 | RS3796939 | 109,305,695 | 310 | 885 |
| 75 | 4 | HADHSC | X96752 | RS221347 | 109,313,226 | 311 | 886 |
| 59 | 4 | GLRA3 | U93917 | RS4695942 | 175,942,562 | 312 | 887 |
| 59 | 4 | GLRA3 | U93917 | RS10021195 | 175,953,446 | 313 | 888 |
| 59 | 4 | GLRA3 | U93917 | RS7438094 | 175,981,922 | 314 | 889 |
| 59 | 4 | GLRA3 | U93917 | RS2046485 | 176,034,349 | 315 | 890 |
| 11 | 5 | IL7R | NM_002185 | RS1389832 | 35,894,478 | 316 | 891 |
| 11 | 5 | IL7R | NM_002185 | RS1494558 | 35,896,825 | 317 | 892 |
| 11 | 5 | IL7R | NM_002185 | RS1494555 | 35,906,947 | 318 | 893 |
| 11 | 5 | IL7R | NM_902185 | RS7737000 | 35,907,030 | 319 | 894 |
| 11 | 5 | IL7R | NM_002185 | RS6897932 | 35,910,332 | 320 | 895 |
| 11 | 5 | IL7R | NM_002185 | RS987107 | 35,910,984 | 321 | 896 |
| 11 | 5 | IL7R | NM_002185 | RS987106 | 35,911,350 | 322 | 897 |
| 11 | 5 | IL7R | NM_002185 | RS3194051 | 35,912,031 | 323 | 898 |
| 40 | 5 | LHFPL2 | D86961 | RS1050674 | 77,818,845 | 324 | 899 |
| 40 | 5 | LHFPL2 | D86961 | RS2114978 | 77,851,010 | 325 | 900 |
| 40 | 5 | LHFPL2 | D86961 | RS6872179 | 77,865,568 | 326 | 901 |
| 40 | 5 | LHFPL2 | D86961 | RS11948997 | 77,878,660 | 327 | 902 |
| 40 | 5 | LHFPL2 | D86961 | RS1561735 | 77,901,984 | 328 | 903 |
| 21 | 5 | KIAA0194 | BC005880 | RS4705411 | 149,411,218 | 329 | 904 |
| 73 | 5 | SGCD | NM_000337 | RS10064593 | 155,688,772 | 330 | 905 |
| 73 | 5 | SGCD | NM_000337 | RS4705006 | 155,692,041 | 331 | 906 |
| 73 | 5 | SGCD | NM_000337 | RS7722282 | 155,730,412 | 332 | 907 |
| 73 | 5 | SGCD | NM_000337 | RS6556574 | 155,747,541 | 333 | 908 |
| 73 | 5 | SGCD | NM_000337 | RS4704798 | 155,749,323 | 334 | 909 |
| 73 | 5 | SGCD | NM_000337 | RS4705013 | 155,765,029 | 335 | 910 |
| 73 | 5 | SGCD | NM_000337 | RS11135202 | 155,783,889 | 336 | 911 |
| 73 | 5 | SGCD | NM_000337 | RS2055611 | 155,796,281 | 337 | 912 |
| 73 | 5 | SGCD | NM_000337 | RS4704804 | 155,840,065 | 338 | 913 |
| 73 | 5 | SGCD | NM_000337 | RS256825 | 155,867,548 | 339 | 914 |
| 73 | 5 | SGCD | NM_000337 | RS4705019 | 155,886,086 | 340 | 915 |
| 73 | 5 | SGCD | NM_000337 | RS6556750 | 155,990,742 | 341 | 916 |
| 73 | 5 | SGCD | NM_000337 | RS6871079 | 155,994,305 | 342 | 917 |
| 73 | 5 | SGCD | NM_000337 | RS32054 | 156,008,460 | 343 | 918 |
| 73 | 5 | SGCD | NM_000337 | RS6890150 | 156,050,193 | 344 | 919 |
| 73 | 5 | SGCD | NM_000337 | RS961272 | 156,113,944 | 345 | 920 |
| 57 | 5 | DOCK2 | NM_004946 | RS264869 | 168,999,444 | 346 | 921 |
| 57 | 5 | DOCK2 | NM_004946 | RS264834 | 169,015,068 | 347 | 922 |
| 57 | 5 | DOCK2 | NM_004946 | RS2244445 | 169,034,177 | 348 | 923 |
| 57 | 5 | DOCK2 | NM_004946 | RS2112703 | 169,059,675 | 349 | 924 |
| 57 | 5 | DOCK2 | NM_004946 | RS2279318 | 169,063,452 | 350 | 925 |
| 57 | 5 | DOCK2 | NM_004946 | RS10038749 | 169,081,158 | 351 | 926 |
| 57 | 5 | DOCK2 | NM_004946 | RS262865 | 169,094,611 | 352 | 927 |
| 57 | 5 | DOCK2 | NM_004946 | RS1680567 | 169,145,733 | 353 | 928 |
| 57 | 5 | DOCK2 | NM_004946 | RS688881 | 169,186,359 | 354 | 929 |
| 57 | 5 | DOCK2 | NM_004946 | RS261623 | 169,200,362 | 355 | 930 |
| 57 | 5 | DOCK2 | NM_004946 | RS2291229 | 169,220,956 | 356 | 931 |
| 57 | 5 | DOCK2 | NM_004946 | RS11740057 | 169,237,503 | 357 | 932 |
| 57 | 5 | DOCK2 | NM_004946 | RS155022 | 169,273,854 | 358 | 933 |
| 57 | 5 | DOCK2 | NM_004946 | RS259894 | 169,291,461 | 359 | 934 |
| 57 | 5 | DOCK2 | NM_004946 | RS1422694 | 169,319,665 | 360 | 935 |
| 57 | 5 | DOCK2 | NM_004946 | RS4867906 | 169,338,200 | 361 | 936 |
| 57 | 5 | DOCK2 | NM_004946 | RS3763048 | 169,394,125 | 362 | 937 |
| 57 | 5 | DOCK2 | NM_004946 | RS6879798 | 169,439,532 | 363 | 938 |
| 28 | 5 | LCP2 | NM_005565 | RS315717 | 169,617,741 | 364 | 939 |
| 28 | 5 | LCP2 | NM_005565 | RS315745 | 169,630,285 | 365 | 940 |
| 28 | 5 | LCP2 | NM_005565 | RS315721 | 169,647,616 | 366 | 941 |
| 28 | 5 | LCP2 | NM_005565 | RS3761750 | 169,657,817 | 367 | 942 |
| 9 | 6 | TDRD6 | NM_001010870 | RS12528857 | 46,777,895 | 368 | 943 |
| 3 | 6 | PLA2G7 | U24577 | RS1051931 | 46,780,902 | 369 | 944 |
| 3 | 6 | PLA2G7 | U24577 | RS2216465 | 46,783,978 | 370 | 945 |
| 3 | 6 | PLA2G7 | U24577 | RS4498351 | 46,784,742 | 371 | 946 |
| 3 | 6 | PLA2G7 | U24577 | RS1805018 | 46,787,262 | 372 | 947 |
| 3 | 6 | PLA2G7 | U24577 | RS6899519 | 46,789,859 | 373 | 948 |
| 3 | 6 | PLA2G7 | U24577 | RS1362931 | 46,790,038 | 374 | 949 |
| 3 | 6 | PLA2G7 | U24577 | RS1805017 | 46,792,181 | 375 | 950 |
| 3 | 6 | PLA2G7 | U24577 | RS6929105 | 46,793,245 | 376 | 951 |
| 3 | 6 | PLA2G7 | U24577 | RS12195701 | 46,795,378 | 377 | 952 |
| 3 | 6 | PLA2G7 | U24577 | RS3799863 | 46,795,750 | 378 | 953 |
| 3 | 6 | PLA2G7 | U24577 | RS3799862 | 46,795,890 | 379 | 954 |
| 3 | 6 | PLA2G7 | U24577 | RS3799861 | 46,797,488 | 380 | 955 |
| 3 | 6 | PLA2G7 | U24577 | RS12528807 | 46,804,466 | 381 | 956 |
| 3 | 6 | PLA2G7 | U24577 | RS9357514 | 46,804,800 | 382 | 957 |
| 3 | 6 | PLA2G7 | U24577 | RS9381475 | 46,807,251 | 383 | 958 |
| 3 | 6 | PLA2G7 | U24577 | RS1421378 | 46,811,472 | 384 | 959 |
| 3 | 6 | PLA2G7 | U24577 | RS1421379 | 46,813,953 | 385 | 960 |
| 3 | 6 | PLA2G7 | U24577 | RS1862008 | 46,818,238 | 386 | 961 |
| 37 | 6 | AIM1 | AI800499 | RS1159148 | 107,073,878 | 387 | 962 |
| 14 | 6 | C6ORF204 | NM_206921 | RS6929390 | 118,969,838 | 388 | 963 |
| 14 | 6 | C6ORF204 | NM_206921 | RS9489433 | 118,973,699 | 389 | 964 |
| 5 | 6 | PLN | M63603 | RS9489434 | 118,976,196 | 390 | 965 |
| 5 | 6 | PLN | M63603 | RS3752581 | 118,976,423 | 391 | 966 |
| 5 | 6 | PLN | M63603 | RS9489437 | 118,981,038 | 392 | 967 |
| 5 | 6 | PLN | M63603 | RS9481825 | 118,982,785 | 393 | 968 |
| 5 | 6 | PLN | M63603 | RS503031 | 118,983,503 | 394 | 969 |
| 5 | 6 | PLN | M63603 | RS12198461 | 118,987,333 | 395 | 970 |
| 5 | 6 | PLN | M63603 | 6P0326 | 118,988,353 | 396 | 971 |
| 5 | 6 | PLN | M63603 | RS1051429 | 118,988,515 | 397 | 972 |
| 14 | 6 | C6ORF204 | NM_206921 | RS1998482 | 118,992,805 | 398 | 973 |
| 14 | 6 | C6ORF204 | NM_206921 | RS763254 | 118,993,308 | 399 | 974 |
| 14 | 6 | C6ORF204 | NM_206921 | RS3734382 | 118,993,654 | 400 | 975 |
| 14 | 6 | C6ORF204 | NM_206921 | RS3734381 | 118,993,996 | 401 | 976 |
| 51 | 6 | OPRM1 | L25119 | RS1799972 | 154,452,810 | 402 | 977 |
| 51 | 6 | OPRM1 | L25119 | RS1799971 | 154,452,911 | 403 | 978 |
| 51 | 6 | OPRM1 | L25119 | RS510769 | 154,454,133 | 404 | 979 |
| 51 | 6 | OPRM1 | L25119 | RS524731 | 154,467,206 | 405 | 980 |
| 51 | 6 | OPRM1 | L25119 | RS3823010 | 154,471,266 | 406 | 981 |
| 51 | 6 | OPRM1 | L25119 | RS495491 | 154,474,656 | 407 | 982 |
| 51 | 6 | OPRM1 | L25119 | RS2075572 | 154,504,118 | 408 | 983 |
| 51 | 6 | OPRM1 | L25119 | RS609148 | 154,523,128 | 409 | 984 |
| 51 | 6 | OPRM1 | L25119 | RS4870268 | 154,564,440 | 410 | 985 |
| 44 | 7 | NPY | NM_000905 | RS16148 | 24,095,578 | 411 | 986 |
| 44 | 7 | NPY | NM_000905 | RS16147 | 24,096,650 | 412 | 987 |
| 44 | 7 | NPY | NM_000905 | RS16143 | 24,097,828 | 413 | 988 |
| 44 | 7 | NPY | NM_000905 | RS16478 | 24,097,848 | 414 | 989 |
| 44 | 7 | NPY | NM_000905 | RS16142 | 24,097,910 | 415 | 990 |
| 44 | 7 | NPY | NM_000905 | RS16141 | 24,097,999 | 416 | 991 |
| 44 | 7 | NPY | NM_000905 | RS16140 | 24,098,048 | 417 | 992 |
| 44 | 7 | NPY | NM_000905 | RS16139 | 24,098,119 | 418 | 993 |
| 44 | 7 | NPY | NM_000905 | RS5572 | 24,098,183 | 419 | 994 |
| 44 | 7 | NPY | NM_000905 | RS9785023 | 24,098,249 | 420 | 995 |
| 44 | 7 | NPY | NM_000905 | RS16138 | 24,098,735 | 421 | 996 |
| 44 | 7 | NPY | NM_000905 | RS1468271 | 24,100,221 | 422 | 997 |
| 44 | 7 | NPY | NM_000905 | RS5574 | 24,102,373 | 423 | 998 |
| 44 | 7 | NPY | NM_000905 | RS16132 | 24,102,760 | 424 | 999 |
| 44 | 7 | NPY | NM_000905 | RS16131 | 24,103,077 | 425 | 1000 |
| 44 | 7 | NPY | NM_000905 | RS16475 | 24,104,726 | 426 | 1001 |
| 44 | 7 | NPY | NM_000905 | RS16126 | 24,104,757 | 427 | 1002 |
| 44 | 7 | NPY | NM_000905 | RS16474 | 24,106,850 | 428 | 1003 |
| 44 | 7 | NPY | NM_000905 | RS16473 | 24,106,891 | 429 | 1004 |
| 44 | 7 | NPY | NM_000905 | RS16120 | 24,107,964 | 430 | 1005 |
| 44 | 7 | NPY | NM_000905 | RS16119 | 24,108,170 | 431 | 1006 |
| 17 | 7 | POR | NM_000941 | RS3898649 | 75,191,543 | 432 | 1007 |
| 17 | 7 | POR | NM_000941 | RS1966363 | 75,221,588 | 433 | 1008 |
| 17 | 7 | POR | NM_000941 | RS2868178 | 75,234,751 | 434 | 1009 |
| 17 | 7 | POR | NM_000941 | RS7804806 | 75,240,333 | 435 | 1010 |
| 17 | 7 | POR | NM_000941 | RS4732513 | 75,252,259 | 436 | 1011 |
| 17 | 7 | POR | NM_000941 | RS10954732 | 75,255,800 | 437 | 1012 |
| 38 | 7 | ABCB1 | M14758 | RS1045642 | 86,783,296 | 438 | 1013 |
| 38 | 7 | ABCB1 | M14758 | RS1128503 | 86,824,252 | 439 | 1014 |
| 38 | 7 | ABCB1 | M14758 | RS9282564 | 86,874,091 | 440 | 1015 |
| 38 | 7 | ABCB1 | M14758 | RS2214102 | 86,874,152 | 441 | 1016 |
| 39 | 9 | ROR2 | M97639 | RS1027268 | 91,450,905 | 442 | 1017 |
| 39 | 9 | ROR2 | M97639 | RS10820899 | 91,561,596 | 443 | 1018 |
| 39 | 9 | ROR2 | M97639 | RS2230578 | 91,565,483 | 444 | 1019 |
| 39 | 9 | ROR2 | M97639 | RS4073735 | 91,567,970 | 445 | 1020 |
| 39 | 9 | ROR2 | M97639 | RS9409456 | 91,574,116 | 446 | 1021 |
| 39 | 9 | ROR2 | M97639 | RS16907720 | 91,579,352 | 447 | 1022 |
| 39 | 9 | ROR2 | M97639 | RS3935601 | 91,588,255 | 448 | 1023 |
| 39 | 9 | ROR2 | M97639 | RS9409461 | 91,610,544 | 449 | 1024 |
| 39 | 9 | ROR2 | M97639 | RS7039620 | 91,615,187 | 450 | 1025 |
| 39 | 9 | ROR2 | M97639 | RS4744098 | 91,623,837 | 451 | 1026 |
| 39 | 9 | ROR2 | M97639 | RS4378021 | 91,626,613 | 452 | 1027 |
| 39 | 9 | ROR2 | M97639 | RS2312732 | 91,662,524 | 453 | 1028 |
| 39 | 9 | ROR2 | M97639 | RS1881385 | 91,676,336 | 454 | 1029 |
| 39 | 9 | ROR2 | M97639 | RS10116351 | 91,731,257 | 455 | 1030 |
| 39 | 9 | ROR2 | M97639 | RS10512219 | 91,735,571 | 456 | 1031 |
| 39 | 9 | ROR2 | M97639 | RS1892263 | 91,767,156 | 457 | 1032 |
| 70 | 11 | TCIRG1 | NM_006019 | RS906713 | 67,570,506 | 458 | 1033 |
| 70 | 11 | TCIRG1 | NM_006019 | RS2075609 | 67,573,512 | 459 | 1034 |
| 70 | 11 | TCIRG1 | NM_006019 | RS11228127 | 67,574,452 | 460 | 1035 |
| 70 | 11 | TCIRG1 | NM_006019 | RS11481 | 67,576,911 | 461 | 1036 |
| 10 | 12 | TNFRSF1A | NM_001065 | RS4149578 | 6,317,698 | 462 | 1037 |
| 10 | 12 | TNFRSF1A | NM_001065 | RS4149577 | 6,317,783 | 463 | 1038 |
| 10 | 12 | TNFRSF1A | NM_001065 | RS4149576 | 6,319,376 | 464 | 1039 |
| 10 | 12 | TNFRSF1A | NM_001065 | RS4149573 | 6,319,645 | 465 | 1040 |
| 10 | 12 | TNFRSF1A | NM_001065 | RS4149570 | 6,321,851 | 466 | 1041 |
| 65 | 12 | PLXNC1 | AF030339 | RS2230754 | 93,045,974 | 467 | 1042 |
| 65 | 12 | PLXNC1 | AF030339 | RS7131826 | 93,048,788 | 468 | 1043 |
| 65 | 12 | PLXNC1 | AF030339 | RS11107420 | 93,057,281 | 469 | 1044 |
| 65 | 12 | PLXNC1 | AF030339 | RS3858609 | 93,067,143 | 470 | 1045 |
| 65 | 12 | PLXNC1 | AF030339 | RS6538486 | 93,078,458 | 471 | 1046 |
| 65 | 12 | PLXNC1 | AF030339 | RS10859685 | 93,097,105 | 472 | 1047 |
| 65 | 12 | PLXNC1 | AF030339 | RS7296806 | 93,099,026 | 473 | 1048 |
| 65 | 12 | PLXNC1 | AF030339 | RS3847813 | 93,101,925 | 474 | 1049 |
| 65 | 12 | PLXNC1 | AF030339 | RS2305971 | 93,105,768 | 475 | 1050 |
| 65 | 12 | PLXNC1 | AF030339 | RS2361355 | 93,132,497 | 476 | 1051 |
| 65 | 12 | PLXNC1 | AF030339 | RS2291326 | 93,151,413 | 477 | 1052 |
| 65 | 12 | PLXNC1 | AF030339 | RS2242498 | 93,152,063 | 478 | 1053 |
| 65 | 12 | PLXNC1 | AF030339 | RS17022311 | 93,155,862 | 479 | 1054 |
| 65 | 12 | PLXNC1 | AF030339 | RS832506 | 93,174,211 | 480 | 1055 |
| 65 | 12 | PLXNC1 | AF030339 | RS1681866 | 93,178,913 | 481 | 1056 |
| 65 | 12 | PLXNC1 | AF030339 | RS3803069 | 93,186,271 | 482 | 1057 |
| 48 | 13 | PCCA | X14608 | RS7325252 | 99,547,355 | 483 | 1058 |
| 48 | 13 | PCCA | X14608 | RS7993067 | 99,566,316 | 484 | 1059 |
| 48 | 13 | PCCA | X14608 | RS1890139 | 99,580,093 | 485 | 1060 |
| 48 | 13 | PCCA | X14608 | RS2152881 | 99,615,996 | 486 | 1061 |
| 48 | 13 | PCCA | X14608 | RS9518016 | 99,626,614 | 487 | 1062 |
| 48 | 13 | PCCA | X14608 | RS9743146 | 99,667,871 | 488 | 1063 |
| 48 | 13 | PCCA | X14608 | RS1112044 | 99,682,492 | 489 | 1064 |
| 48 | 13 | PCCA | X14608 | RS538229 | 99,686,123 | 490 | 1065 |
| 48 | 13 | PCCA | X14608 | RS7991183 | 99,711,884 | 491 | 1066 |
| 48 | 13 | PCCA | X14608 | RS9518035 | 99,716,632 | 492 | 1067 |
| 48 | 13 | PCCA | X14608 | RS9557413 | 99,760,924 | 493 | 1068 |
| 48 | 13 | PCCA | X14608 | RS9554686 | 99,870,943 | 494 | 1069 |
| 48 | 13 | PCCA | X14608 | RS8001633 | 99,904,079 | 495 | 1070 |
| 48 | 13 | PCCA | X14608 | RS1296332 | 99,911,747 | 496 | 1071 |
| 48 | 13 | PCCA | X14608 | RS3783171 | 99,922,321 | 497 | 1072 |
| 26 | 14 | ITPK1 | NM_014216 | RS875395 | 92,471,846 | 498 | 1073 |
| 26 | 14 | ITPK1 | NM_014216 | RS1043542 | 92,476,815 | 499 | 1074 |
| 26 | 14 | ITPK1 | NM_014216 | RS11446 | 92,477,001 | 500 | 1075 |
| 26 | 14 | ITPK1 | NM_014216 | RS10873430 | 92,478,831 | 501 | 1076 |
| 26 | 14 | ITPK1 | NM_014216 | RS2295394 | 92,482,496 | 502 | 1077 |
| 26 | 14 | ITPK1 | NM_014216 | RS2402226 | 92,489,288 | 503 | 1078 |
| 26 | 14 | ITPK1 | NM_014216 | RS3825683 | 92,518,490 | 504 | 1079 |
| 26 | 14 | ITPK1 | NM_014216 | RS4905025 | 92,536,179 | 505 | 1080 |
| 26 | 14 | ITPK1 | NM_014216 | RS1614269 | 92,573,258 | 506 | 1081 |
| 26 | 14 | ITPK1 | NM_014216 | RS1740596 | 92,576,559 | 507 | 1082 |
| 26 | 14 | ITPK1 | NM_014216 | RS1740595 | 92,582,283 | 508 | 1083 |
| 26 | 14 | ITPK1 | NM_014216 | RS2749509 | 92,597,867 | 509 | 1084 |
| 26 | 14 | ITPK1 | NM_014216 | RS882023 | 92,601,767 | 510 | 1085 |
| 26 | 14 | ITPK1 | NM_014216 | RS4905043 | 92,619,762 | 511 | 1086 |
| 26 | 14 | ITPK1 | NM_014216 | HCV1258994 | 92,623,971 | 512 | 1087 |
| 26 | 14 | ITPK1 | NM_014216 | RS941540 | 92,630,797 | 513 | 1088 |
| 26 | 14 | ITPK1 | NM_014216 | RS768356 | 92,646,296 | 514 | 1089 |
| 55 | 14 | C14ORF132 | AA149431 | RS4340260 | 95,617,294 | 515 | 1090 |
| 55 | 14 | C14ORF132 | AA149431 | RS10140364 | 95,621,356 | 516 | 1091 |
| 55 | 14 | C14ORF132 | AA149431 | RS1058102 | 95,627,988 | 517 | 1092 |
| 55 | 14 | C14ORF132 | AA149431 | RS1062710 | 95,629,212 | 518 | 1093 |
| 55 | 14 | C14ORF132 | AA149431 | RS2104290 | 95,638,734 | 519 | 1094 |
| 18 | 15 | ANPEP | M22324 | RS967451 | 88,129,048 | 520 | 1095 |
| 18 | 15 | ANPEP | M22324 | RS10584 | 88,129,555 | 521 | 1096 |
| 18 | 15 | ANPEP | M22324 | RS1992250 | 88,134,984 | 522 | 1097 |
| 18 | 15 | ANPEP | M22324 | RS7168793 | 88,135,244 | 523 | 1098 |
| 18 | 15 | ANPEP | M22324 | RS1439120 | 88,139,197 | 524 | 1099 |
| 18 | 15 | ANPEP | M22324 | RS1439119 | 88,139,250 | 525 | 1100 |
| 18 | 15 | ANPEP | M22324 | RS1439118 | 88,139,516 | 526 | 1101 |
| 18 | 15 | ANPEP | M22324 | RS753362 | 88,141,538 | 527 | 1102 |
| 18 | 15 | ANPEP | M22324 | RS893615 | 88,141,723 | 528 | 1103 |
| 18 | 15 | ANPEP | M22324 | RS2007084 | 88,146,339 | 529 | 1104 |
| 18 | 15 | ANPEP | M22324 | RS2305443 | 88,147,865 | 530 | 1105 |
| 18 | 15 | ANPEP | M22324 | RS25653 | 88,150,562 | 531 | 1106 |
| 8 | 16 | MYH11 | D10667 | RS1050163 | 15,718,524 | 532 | 1107 |
| 8 | 16 | MYH11 | D10667 | RS1050162 | 15,718,563 | 533 | 1108 |
| 8 | 16 | MYH11 | D10667 | RS2075511 | 15,725,642 | 534 | 1109 |
| 8 | 16 | MYH11 | D10667 | RS1050113 | 15,746,535 | 535 | 1110 |
| 8 | 16 | MYH11 | D10667 | RS2272554 | 15,757,705 | 536 | 1111 |
| 8 | 16 | MYH11 | D10667 | RS4781689 | 15,772,973 | 537 | 1112 |
| 8 | 16 | MYH11 | D10667 | RS6498574 | 15,795,766 | 538 | 1113 |
| 8 | 16 | MYH11 | D10667 | RS8044595 | 15,813,631 | 539 | 1114 |
| 8 | 16 | MYH11 | D10667 | RS216152 | 15,823,321 | 540 | 1115 |
| 8 | 16 | MYH11 | D10667 | RS1050111 | 15,824,698 | 541 | 1116 |
| 8 | 16 | MYH11 | D10667 | RS215581 | 15,840,675 | 542 | 1117 |
| 8 | 16 | MYH11 | D10667 | RS215571 | 15,851,834 | 543 | 1118 |
| 62 | 16 | ITGAX | Y00093 | RS1106398 | 31,277,953 | 544 | 1119 |
| 62 | 16 | ITGAX | Y00093 | RS4264407 | 31,278,694 | 545 | 1120 |
| 62 | 16 | ITGAX | Y00093 | RS2070896 | 31,292,055 | 546 | 1121 |
| 62 | 16 | ITGAX | Y00093 | RS2929 | 31,300,809 | 547 | 1122 |
| 62 | 16 | ITGAX | Y00093 | RS1140195 | 31,301,680 | 548 | 1123 |
| 35 | 17 | GRN | NM_002087 | RS3859268 | 39,778,789 | 549 | 1124 |
| 35 | 17 | GRN | NM_002087 | RS2879096 | 39,779,082 | 550 | 1125 |
| 35 | 17 | GRN | NM_002087 | RS3785817 | 39,779,191 | 551 | 1126 |
| 35 | 17 | GRN | NM_002087 | RS4792938 | 39,780,125 | 552 | 1127 |
| 35 | 17 | GRN | NM_002087 | RS9897526 | 39,782,466 | 553 | 1128 |
| 35 | 17 | GRN | NM_002087 | RS25646 | 39,783,156 | 554 | 1129 |
| 35 | 17 | GRN | NM_002087 | RS25647 | 39,785,365 | 555 | 1130 |
| 35 | 17 | GRN | NM_002087 | RS5848 | 39,785,770 | 556 | 1131 |
| 27 | 18 | FVT1 | X63657 | RS6810 | 59,149,381 | 557 | 1132 |
| 27 | 18 | FVT1 | X63657 | RS2850767 | 59,152,094 | 558 | 1133 |
| 27 | 18 | FVT1 | X63657 | RS2236719 | 59,157,272 | 559 | 1134 |
| 27 | 18 | FVTI | X63657 | RS2849372 | 59,164,885 | 560 | 1135 |
| 27 | 18 | FVT1 | X63657 | RS2850756 | 59,168,088 | 561 | 1136 |
| 67 | 19 | HNRPM | NM_005968 | RS6603076 | 8,413,177 | 562 | 1137 |
| 67 | 19 | HNRPM | NM_005968 | RS6603078 | 8,417,325 | 563 | 1138 |
| 7 | 19 | PLAUR | X74039 | RS4760 | 48,844,940 | 564 | 1139 |
| 7 | 19 | PLAUR | X74039 | RS2283628 | 48,854,901 | 565 | 1140 |
| 7 | 19 | PLAUR | X74039 | RS399145 | 48,861,362 | 566 | 1141 |
| 7 | 19 | PLAUR | X74039 | RS2286960 | 48,863,865 | 567 | 1142 |
| 74 | 19 | BAX | NM_138763 | RS1009316 | 54,150,382 | 568 | 1143 |
| 74 | 19 | BAX | NM_138763 | RS1B05419 | 54,150,916 | 569 | 1144 |
| 74 | 19 | BAX | NM_138763 | RS4645887 | 54,151,688 | 570 | 1145 |
| 74 | 19 | BAX | NM_138763 | RS2387583 | 54,153,117 | 571 | 1146 |
| 74 | 19 | BAX | NM_138763 | RS905238 | 54,157,196 | 572 | 1147 |
| 69 | 22 | GTSE1 | NM_016426 | RS6008729 | 45,047,947 | 573 | 1148 |
| 64 | 22 | TRMU | NM_018006 | RS6007886 | 45,058,315 | 574 | 1149 |
| 64 | 22 | TRMU | NM_018006 | RS13585 | 45,073,698 | 575 | 1150 |

TABLE 4

| (SEQ ID | SNP Sequence |
|---|---|
| NO:) | (polymorphism location is indicated in brackets) |
| 1 | 5′- GGACACAACAGGACCCACTG[G]GGAAAACAATGATGACTTGG -3′ |
| 2 | 5′- CCCCTCCACTTTGCTCACCC[A]TCTTCCGGGCCCTGAACCCA -3′ |
| 3 | 5′- TCCTGTGCCGGCTGCAGGTA[T]GGAACAAGTAGGCTAGTGTC -3′ |
| 4 | 5′- AGGAAAGACTGTTGGGCCTC[G]GAAAACATCCCACGTGCTAG -3′ |
| 5 | 5′- GGGACTTGGTTTCATGTCTC[T]ATCTCTCAGTTCTGTTTCCC -3′ |
| 6 | 5′- ATAGAGAGGGTCTGTTAGGT[T]CTTGGGATCTTGTTCTTCAA -3′ |
| 7 | 5′- ATTCCAATTGAAGATTGAAA[G]TGGCCTGTTTGGTAAACTGG -3′ |
| 8 | 5′- TAACTCAAAGCACAAAGTTT[T]GAATTCCTACATTCTAAAGA -3′ |
| 9 | 5′- GTCACCTGCCTCGGAGCCAG[T]TAGGCTGTTTAACAGTGCAG -3′ |
| 10 | 5′- GGAGCTTTGGCATCGCAGAG[A]CTTGAGCTGAGTCTGGCTCT -3′ |
| 11 | 5′- CAGAGCCCCTCCCTCTAAAC[A]CAGTCTTTCAAAGGGATTGT -3′ |
| 12 | 5′- CAATTTCTTGCTGAAAGCCC[T]GAGTTATGCCAGACACTGTG -3′ |
| 13 | 5′- ACCTTTGCCCAGATCCAAAT[G]TTTTTTCTTCATTCGAAGCT -3′ |
| 14 | 5′- ACGGATCTCTTACCATTAAA[T]TCAGGTGGAGAGGGAGTGCC -3′ |
| 15 | 5′- TTTCACAGATGAGGAGGCTG[T]CCTCAGGAAATGTGACTCAG -3′ |
| 16 | 5′- CCAACACCACCCCTTGCCCA[G]CCAATGCACACAGTAGGGCT -3′ |
| 17 | 5′- CCCATATCATGCAGAGGATC[T]GGGATTTCAATCCAGGTCTA -3′ |
| 18 | 5′- TGACGTGTGCAGAGAGACAT[C]TCAGCCTGCCCTGCACTTGT -3′ |
| 19 | 5′- GGCAGCATATTAGAAAATAG[C]TTATGTTACAACAAAAACCC -3′ |
| 20 | 5′- TGCCCCTTCTCACTGGTCTG[C]GGCTGGCAGGGCCATCTTTC -3′ |
| 21 | 5′- GAATCCATCCCAAGGACACC[C]TTTGAAAACATGAAATAACA -3′ |
| 22 | 5′- CAGCGGGGAGGGGAAAGGTC[T]GAAATGAGGGGAGAGACGTG -3′ |
| 23 | 5′- GCTGGGCAGAGCCATTCCTG[A]GCTGGCTGGGTGTGTTTGGG -3′ |
| 24 | 5′- ACAGGCATCAGGGATACAGT[G]GTGAACAAGCATACACAATC -3′ |
| 25 | 5′- AGGTGAAGCTGAGGCCTGAG[C]CCAGAAGGAGAGAAAAGGAA -3′ |
| 26 | 5′- CACTCATTAATCCATTAAAC[C]ATTAATCTATTAATCCATGA -3′ |
| 27 | 5′- GTGTATGCTGTGAAGAAGGC[A]ACCCCCCTTCCTGCCCATCC -3′ |
| 28 | 5′- CTGTCACTATGCCCCTGCCT[T]TCTCAGTGTCTATCTCTGTT -3′ |
| 29 | 5′- GGGATGACAGTGAGAGGAGG[C]CAACAGTAAAAGGAGTCATA -3′ |
| 30 | 5′- GTGTGTCTGTCAGGGAATGT[G]TCCCTCTTCCATTCTCTGTG -3′ |
| 31 | 5′- CCATTCTTGGTGGTGAGCCT[G]GACTCTGAGCCTGGGATGTG -3′ |
| 32 | 5′- GTCTGGCTGCCCCTTGGCCT[C]CACYACAGTCAGGTCCAGCC -3′ |
| 33 | 5′- TTGAGGATTAAAGAGCAGAR[G]TCATGTAGCATCTGGCACAT -3′ |
| 34 | 5′- CGTCATGTAGCATCTGGCAC[G]TGGGGGAACGCAATGGAAGT -3′ |
| 35 | 5′- CAGAGAATATTTCACATGCA[T]GTAGCAAAAACACCAGGGGT -3′ |
| 36 | 5′- AACATGGATTAATGTGGGAA[C]TTGGCTTCAAGAACACAACC -3′ |
| 37 | 5′- ATTATTTCATTTTAAAACCA[T]AGAATAAAAATGACACCTGA -3′ |
| 38 | 5′- AAGCAGATTATGAGGCAGGT[C]CACCCCTCCCAGCACTGGGG -3′ |
| 39 | 5′- CCAGCCCTGTAGTGGACATA[T]TTGCCTTTGCCTATTCAGCA -3′ |
| 40 | 5′- GAACTCGGTGGAGGAGAAGA[G]AAACTCCAAGATGCTCCAGA -3′ |
| 41 | 5′- TGTGGGCTGGACTTAGCAAC[G]CACTTCTAACTAACAGAATG -3′ |
| 42 | 5′- GGTGTCAATTCACTCCCAGC[G]GCACTGACTGAGTGCTGACC -3′ |
| 43 | 5′- ATGTTAGGCGGTCCCACCTG[C]GTTCTGGAGATCTTCACACA -3′ |
| 44 | 5′- GGTGGGCAGAGGCTGGATCC[T]ATGGTGAGGAGTTTCCATTT -3′ |
| 45 | 5′- TTGCCATGGGCCACCTCTAC[C]GAGTGCTCGATGAACAACAA -3′ |
| 46 | 5′- TTTGGCTGGGGCAAGCTTAC[G]TGGTTCGGCAGTAGTACCAG -3′ |
| 47 | 5′- GTGGCCCCAGGAATGCGGGC[G]TCTGGTGGTATCTGGGCTGG -3′ |
| 48 | 5′- ATGCATTGTGGTAGATTCAT[A]CAATGGAGTATACACAGCAA -3′ |
| 49 | 5′- GTGGCAGCTGCCATTTTTCC[G]GTGCCACAAATGGTAGTTAC -3′ |
| 50 | 5′- TTGGGAGGAAGACCACAGAG[G]TGATGTGCCAGTCTCAGAAC -3′ |
| 51 | 5′- AAAATACAGGGTACAGGGAC[A]CTCAAAGAGTGATTTGCTTC -3′ |
| 52 | 5′- GTGAGATGGGGCACAGCAGC[G]GCCGGAAGGTTATTTGTGTG -3′ |
| 53 | 5′- GCAGGGCAGAGAAGGGGAAG[C]TGCTGGCTGCCCTCCTCACT -3′ |
| 54 | 5′- GCTCCTGGATTCACTCCTTT[C]ATCCTCACCTCAATCCTTTG -3′ |
| 55 | 5′- AGTTGGCTTGTATGGACCCC[G]CCGATGACGGACAGTTCCAA -3′ |
| 56 | 5′- AGTGGATTGAGGATGGACAT[G]TGTATCTGGAAGCACCAAAA -3′ |
| 57 | 5′- CTGGGTTCACTGGAAATCAG[T]ATTAAGAATGTACAAGGGAA -3′ |
| 58 | 5′- ATGTAAACTGCCTTTGAAAG[C]CTATAACACAGTTCAGTTGG -3′ |
| 59 | 5′- ACTTAATCTTGCTCAGTTCC[T]CAGTTTACACTTTTGAATGG -3′ |
| 60 | 5′- GCAGCATAGATGAATGTAAT[A]TTGAAACAGGAAGATGTGTT -3′ |
| 61 | 5′- CTTAGCCTGCAATTGCAATC[C]GTATGGGACCATGAAGCAGC -3′ |
| 62 | 5′- TAGCCGTTTACAGAATATCC[G]GAATACCATTGAAGAGACTG -3′ |
| 63 | 5′- GTTTCAGATTTTGATAGGCG[C]GTGAACGATAACAAGACGGC -3′ |
| 64 | 5′- ATGAGGGAGAAATGCCCTTT[T]TGGCAATTGTTGGAGCTGGA -3′ |
| 65 | 5′- AGGAACAGTGCTACTTACTG[G]TGGGTAGACTGGGAGAGGTG -3′ |
| 66 | 5′- TTGGCAATGGGTAAGTCTAT[C]GTACTGTGTAAACTTGGACT -3′ |
| 67 | 5′- GATATAGATCTCTTGGAAAT[G]TAATAATGGTATGCAGGAAG -3′ |
| 68 | 5′- GCAACCCGGGGAAATACAGC[C]AAATGCACAAGTACTGGCTG -3′ |
| 69 | 5′- CTGTACAAACTTTCTTCCAT[A]ATTTTGATTATATCCATTTT -3′ |
| 70 | 5′- CCCTCATTATCTGCCTAAAC[G]ATTTTTTCTCAACTCCTATA -3′ |
| 71 | 5′- CTAGCACTGTACACACCCCA[C]ACTGTGTATGCTATTTGTTG -3′ |
| 72 | 5′- CAAAAGTTATCTCTAACCAA[T]GTACTCAAACAGAGTCTTTA -3′ |
| 73 | 5′- CCTTGTAAATCTCCACCTGA[G]ATTTCTCATGGTGTTGTAGC -3′ |
| 74 | 5′- TCCCATAGGAATTATAAAAT[G]GAAAAGTATGACAAAAATTT -3′ |
| 75 | 5′- AGGCCCTTCAGCTTCACCAC[C]TGCTTCTCTTTAAACAAGTC -3′ |
| 76 | 5′- GATAGAATTTGGCCCAGAGA[G]GTTAACTAATATATCCATGA -3′ |
| 77 | 5′- CTGTTTCTCCTTAAAATGGA[G]AAATGGCCTCTACAGAGTAG -3′ |
| 78 | 5′- GCTTGGTGGGGCCACTGGGC[G]TCTGTTTCTCGGGTGTTTTG -3′ |
| 79 | 5′- CCATTCCCTCGGCGAAGAGC[G]GAGGTTGAAGAAATGCTACT -3′ |
| 80 | 5′- GCAAGCGCCAGAGCCTCTGT[G]TGCTGCATTCGGCAACCACA -3′ |
| 81 | 5′- GGTTCCTGAAGGAGGAGTGG[A]AGTTTGGTAAATGGATGGAG -3′ |
| 82 | 5′- TTACCTGCTAAGGCCTGCAA[A]CTTGAGGATGTCCAGGGCTG -3′ |
| 83 | 5′- CCAGAAGGTTTCTTTGCTCC[C]CTTCCCTACAAAGACAGAGC -3′ |
| 84 | 5′- AATTCACTCCTTTAAAATAC[C]CAATGCAGTGTTTTTAGAAA -3′ |
| 85 | 5′- CCACTCCCTCTCCTGCTCTT[G]TGTGTGTGATCCAAAGGGAA -3′ |
| 86 | 5′- CAGGGACAGCTGAAGCCAAG[C]TCTCCCAAAGCAGCCTTGGC -3′ |
| 87 | 5′- GTCAGGAGCCTGGCCAGGCC[G]CACCCCTTGCTGTCTCAGCA -3′ |
| 88 | 5′- GGAGATTCTGCCTCAGGGCC[G]TGAGAGTCCCATCTTCAAGC -3′ |
| 89 | 5′- GCTCAGCTACCGTTGGTGGC[A]TTTATTAAACTGTGCACCCA -3′ |
| 90 | 5′- AAGGTGGCTGACTCCAGCCC[A]TTTGCCCTTGAACTGCTGAT -3′ |
| 91 | 5′- TGAAGACCTGAAAAGCAAAT[T]CCAGGCAGCCCCACTCCCTC -3′ |
| 92 | 5′- TTCTTTGTAATTTGGAATCC[A]CCTAATTTCCAAATGGGTTC -3′ |
| 93 | 5′- GGGACCTGGCCCTGGCCATC[C]GGGACAGTGAGCGACAGGGC -3′ |
| 94 | 5′- AGGTGGGGACCCGGCTCCAA[A]GGCACCCGGGTCTTCTGCAG -3′ |
| 95 | 5′- ACAGGCCGCTCTCCCAGCAG[C]GTGTTGAGGTGCACAGCCAG -3′ |
| 96 | 5′- TGGCGCAAGAGAACCAGGGC[G]TCTTCTTCTCGGGGGACTCC -3′ |
| 97 | 5′- TGCTGTGCCCACATCCCCTG[C]AACAGGCAGGCCAGCCTGTG -3′ |
| 98 | 5′- TGGTGAGTTATGGACCCYCC[T]ACCTCCACTACTACACTGTA -3′ |
| 99 | 5′- TCAGGGCCTGGGGCAGGCGC[G]GCACAGCCCCCACCGCTGCT -3′ |
| 100 | 5′- GCATGGCATGCGGAAGATGG[T]GAAGAATGTTTTATGGCCTC -3′ |
| 101 | 5′- TCTCAGTAGCTGAGACCTGA[G]AAATTTGGAGAATCACTTTG -3′ |
| 102 | 5′- ACATGAGGCCACTGAGGCAG[C]CCTCTTTCCTTCCCCTTCTC -3′ |
| 103 | 5′- CCTATTCTTAATCCTATTTT[G]CAAATGAAGTGACTTGCCCA -3′ |
| 104 | 5′- GGAATGGGTCAAGAATGTTC[G]TTCCCTTCTGAATGTCCCTG -3′ |
| 105 | 5′- AAGCGGGGAGGAGCTAAATA[C]TATTTTTCTCTCCTTGTTCA -3′ |
| 106 | 5′- AACTTGGAACATCTCCGCAA[C]AAGACAGAGGATCTGGAAGC -3′ |
| 107 | 5′- ACATCGCAGAAGGTGGCTCG[A]AAATTCTGGTGGAAGAACGT -3′ |
| 108 | 5′- TTCCCGAGGCCCTGCTGCCA[T]GTTGTATGCCCCAGAAGGTA -3′ |
| 109 | 5′- TGAGAGTCAGGGTTTGGGAC[C]AGATTGGCAAGTCAGGCTCT -3′ |
| 110 | 5′- TCTCCAGGACCTAGTATGGT[G]CCTGACCGTGGCACTCATAG -3′ |
| 111 | 5′- CTACCTCAGAGTATGTGCCC[A]TTGGATGGTGGCTGTTATTC -3′ |
| 112 | 5′- CTAGTCTCTGAGCTGAGTGC[C]GACTTAGGGAGGCAATGTTA -3′ |
| 113 | 5′- ACAGTGTGGCGTAAGGCAGT[G]TGGCCCTTGTCCTCTTGCTT -3′ |
| 114 | 5′- TTAGGGCAGCTGTGCATTGA[C]TGGGTAGACGCCATTCTGGA -3′ |
| 115 | 5′- TGAGGCCCCCACCTGGCCCT[T]ATCTGCCCCTGACATCTAGA -3′ |
| 116 | 5′- CGCATAATTTCCGTCACCTC[A]TTCGCCTGCTGGTGGCACCG -3′ |
| 117 | 5′- CCCCAACATGTGCACCCCTG[C]ATTTCCTGTCATGCCACAGA -3′ |
| 118 | 5′- CCAGATCTCCATCATTGGCG[T]TAGTCTCTGGTCACCTGACT -3′ |
| 119 | 5′- TTTGTTCTGACTTTACATCC[C]CTTCCCCAGGTCACTTTTCA -3′ |
| 120 | 5′- ATTCCTGTCCCTTGTGCCGC[T]ATGAGCTGCCCACTGATGAC -3′ |
| 121 | 5′- TTTGATACCAAGAACACATT[T]CTGCATGAATCCTCCAGCAA -3′ |
| 122 | 5′- TCTAAAATTAGGGGTTTGAT[T]TAGCTTATCTGGAAGGTGTT -3′ |
| 123 | 5′- GATGCGGTCTGGAAAGCACC[A]GGGTGGCCGTCGGCTGACGC -3′ |
| 124 | 5′- CTCCGTGGAACTTCTCCTGG[T]ACAAATTCTGTTCCTAGGGA -3′ |
| 125 | 5′- GAGGGGAGCCACAGGAATGG[C]CGTGGCCAGAAGCCCTTCTC -3′ |
| 126 | 5′- GGCACCTTTTCCCTGATAAG[A]CACAAATCATAACCAAACAA -3′ |
| 127 | 5′- TTGCACTCCAGTTTTTTTTT[C]TTTAAAAAAGCGGTTTCTAC -3′ |
| 128 | 5′- GAAAAGGCTGTCTGATTATC[G]TGTCATCCAAAAAAAACAGA -3′ |
| 129 | 5′- GAACTAAGAGGAATAAAGGT[A]TTGCTTTATACCTGTCCCTA -3′ |
| 130 | 5′- ACTAACATGTCCTGCCTATT[A]TCTGTCAGCTGCAAGGTACT -3′ |
| 131 | 5′- GCTGACCCAGGGTCCACATG[C]TCTTTTTCTAACTTGTTCAT -3′ |
| 132 | 5′- TGCTTCCCCATTTCTGTCCT[A]AAAGCCCTCTGGCAAGACTG -3′ |
| 133 | 5′- CAGTGATGAACTCCTGGGCT[T]AAGTGACCCACCCGCCTCTG -3′ |
| 134 | 5′- GCGACTTCGACTAAGCAACA[T]TGCATCTATTTTCATGCAAC -3′ |
| 135 | 5′- CCTCAAATGTTAGAGTCAGT[G]CACCAGCTCATAGTTTCCAT -3′ |
| 136 | 5′- CGTTTAATTCTTTCTCATCA[G]TTTCCTAGGGCATTTGCAAT -3′ |
| 137 | 5′- CATCAGAGTTTTATGATTAG[T]AGATATATCTTAACTGACAC -3′ |
| 138 | 5′- AGCAAAACCAAAGAAATCAGC[G]GAAGACCATAAAAACAGACG -3′ |
| 139 | 5′- CTATAAAATTAGTATGCTTA[A]AATTATTAAACATATACAGA -3′ |
| 140 | 5′- TAAACACTTTAATGCAGTGA[T]ACTCAGGTATAAAACTCAGA -3′ |
| 141 | 5′- ATAGAAGACAAAGTTTTCAT[C]CGTCTCATTCAAGTTCACTT -3′ |
| 142 | 5′- AGTGCAGGGCAGGACTGCTG[T]CTGACCCCGGGCCACCTGGA -3′ |
| 143 | 5′- AACCTCTTGGTACATGTTAG[G]GGAAATGAAGCTGGCAACAA -3′ |
| 144 | 5′- TCATCAGATCAAGGACATTA[T]GGAATTAAAGGGCTCTAAGA -3′ |
| 145 | 5′- CCACTGCTATTGGTTATTTA[T]CTAGCATCCATTTCCCTTTA -3′ |
| 146 | 5′- ATCTACCTCTCCTGCCTCAT[C]TATTATTACCCAGCCCCTTC -3′ |
| 147 | 5′- GTCAATTGCAAATGGAGGTG[G]GACCTGAGAAAACAAAGAAA -3′ |
| 148 | 5′- GAGTGTGTAACAACTCACCT[A]CCAAATCGACTAGCCCTTAG -3′ |
| 149 | 5′- CTTGTAAGCCATCTTAAGCC[A]TTATAGGCCTAAGATGTATA -3′ |
| 150 | 5′- CTTGAGACCTGTGTCTCCTC[G]TGTTCACACTGTTCCTGACT -3′ |
| 151 | 5′- GAGGCATGGGTTGAACTGCA[C]TCACATATGTACTTAAAAGA -3′ |
| 152 | 5′- TGTTTCTTGAAGTTTGACTA[T]TTAAAAACATAGGTGTAAAG -3′ |
| 153 | 5′- AGAGTCACGGCATGTGGGAA[G]GTTTCCATGGACACTGGATC -3′ |
| 154 | 5′- AATGAGATCTTATGTCAAGG[C]TTTAATCTTTGGTATTCCAA -3′ |
| 155 | 5′- TCTGGACCTCAGTTTCCTCA[G]TGAGCTGGTAAGAATGCACT -3′ |
| 156 | 5′- AGGTTGATAGCAATGTTTGG[A]AGATATGTCCTAGAAGTGTT -3′ |
| 157 | 5′- GCATGATAACCCTAGCCATC[G]CTAAATATTATAGCTTCCTT -3′ |
| 158 | 5′- CTCCAGTTTCTCCCTTTCTC[A]CCAACTAGGTCCATCCAAAC -3′ |
| 159 | 5′- AACTGTAAGGATCTCTTGCT[G]TATATACTATTGGGGGAACA -3′ |
| 160 | 5′- CCTTAGCTCTTCCTAAAACA[T]ACAATCATAAAGGAAACCGT -3′ |
| 161 | 5′- CTGACAGTAAAGGGAACTCA[T]TATGTCTGAGTCTTTGCTCA -3′ |
| 162 | 5′- AACATTTACAGAAGCGAGAA[T]AAGTTTTGTTTGCTTTTGTT -3′ |
| 163 | 5′- TAAGTTCAATAAATCCCAAA[T]TGCACACTCTGAATTAGGGG -3′ |
| 164 | 5′- AAGATAGCCATCTTTGGGCA[C]AGAGTCATGAAATGTACCCT -3′ |
| 165 | 5′- GCTGGGCCGACGGGGACGAG[G]CGGCGACTGGAGCAGCAGCG -3′ |
| 166 | 5′- CTCTGTCTTGGTCACTGTGC[A]AGGATTGAAGGGAACTATTG -3′ |
| 167 | 5′- ATCGTCTTTTACAATAAGAT[A]CATGCCCCTATGAGTATTTT -3′ |
| 168 | 5′- AAGGAGAAAAACAGTGAACC[G]TAGTTCTTACTGCTCACACT -3′ |
| 169 | 5′- GATTATTTGATTGCCATGAA[T]GAAGCTGAATTACATAATTC -3′ |
| 170 | 5′- AGGGACCTGTCTTCAGAATC[G]AAGAAGCATAATGTCCTTAA -3′ |
| 171 | 5′- TAGAGTCCCTACCATGCACC[G]TGGGCAAGAAGTCAGTTCTG -3′ |
| 172 | 5′- TCGGGTCTCTTACCATGCCC[A]CCCTCCCTTCCTCAGGGAAT -3′ |
| 173 | 5′- AGGACCTTCAGAGACCCCGC[A]TTCTCTGAAACCAGGATGGA -3′ |
| 174 | 5′- CAGGGGCTGCACTCACCATC[A]TCTGACACCTCCACTTCATC -3′ |
| 175 | 5′- GTACACAAGGGTAGGGCAGA[A]GATGGACAGCAGGGCAGAAT -3′ |
| 176 | 5′- AGTTTCTGCAGCACTTTATC[C]TTCCATCTGGCCATGAGGAA -3′ |
| 177 | 5′- CAGGCATTGAAGGTCAGCTT[C]TTCTCCTCCTGGGTGAGTTT -3′ |
| 178 | 5′- GGGCACGACCTACCATCCAC[A]GTGACTTGGCAGGAGCACTC -3′ |
| 179 | 5′- TTACTTCTATCCTTGCTTCT[C]GAACTGGTCATTCCCTGACT -3′ |
| 180 | 5′- AGAACAAGCTGTTAGCAGGA[T]GCCTCTGCTGCTGCGGGGCC -3′ |
| 181 | 5′- TCGGCTGGGATCTCCTTCAG[G]TCGTCTTCCGATAGGGTCTT -3′ |
| 182 | 5′- AGGCCTCAGGGACCCATAGC[G]GTCACTACCACCACCATCAG -3′ |
| 183 | 5′- TTGTCCAGAAATCACTGTGA[T]TGGATACACAAATGCAGCAC -3′ |
| 184 | 5′- CTTGGCTGCTGAATGGTGAG[T]TCCCCCTGCCCCAGCTCTCT -3′ |
| 185 | 5′- GAAGTCTTCTGAAGGACCGG[A]GTCTGCGGGGCCGTTCTGGG -3′ |
| 186 | 5′- TGGTGGCTTTTGTTTCTCTC[A]CAAATGACCTGTGTGGTGGT -3′ |
| 187 | 5′- AGGACGGGTCTCCACTGCTG[A]AGCTGAAAATCTATCCCTGT -3′ |
| 188 | 5′- TTTGTGACCTTGTATGGATG[-]ACTTCTCTGAATCTTATTTC -3′ |
| 189 | 5′- AAAACTCAATAAGATGCCTA[C]ATTTTATGCATCTCCATTAA -3′ |
| 190 | 5′- TTCACCATCCCTCTACTTTC[A]GCTTGCCAAAACTTACAGGA -3′ |
| 191 | 5′- TGGCCAGTGCTCAGCAGATG[C]AAGTTCCAAATCGAGTCACT -3′ |
| 192 | 5′- GCATGGAGTCAACTCTTGAG[G]GATCCACACTGAGGGAGGTT -3′ |
| 193 | 5′- TGAGTCCTGGTCCAGGGCCT[G]CTGGGGACTAGATAAGATGT -3′ |
| 194 | 5′- CAAGCTAGAGACTTGGTATA[T]AGCAGCAGTTACATGAGTGG -3′ |
| 195 | 5′- CAGACTGTGGACATCCGAAT[C]GGCAATGACATGAATTTAAG -3′ |
| 196 | 5′- AGGCACCAGGTCCCATGGCC[T]GTTTCCCCTGAGAAAACATT -3′ |
| 197 | 5′- ATGGAGAGCTGCCAAGCCAA[A]CCTGCCAGGGTCATCAGCTC -3′ |
| 198 | 5′- ATAGCTGTCCTTACTCCTTT[G]CTAGACAGACAGTGTCTTGG -3′ |
| 199 | 5′- GCTTTTTATACCGCTTAACG[T]AAATAATTTAAAAGGCTGTC -3′ |
| 200 | 5′- AGCTGCAATGCCTATGAGCA[A]GACCTGGGTTTGTACATCTT -3′ |
| 201 | 5′- CTAGGATAGCAGAGATATTA[T]TTCAGGATCAGATCTTGACT -3′ |
| 202 | 5′- TCTGGGGAGTCTTTAGCCCC[T]AGCAGAGGCCATTTCTAGCA -3′ |
| 203 | 5′- GAATAAAACTTACGGAGAGC[T]TCTAACTTCATTCAATTTGT -3′ |
| 204 | 5′- ATAATATATTTTAAGCAGGG[C]AGGGTATCCCAAGATCTCAA -3′ |
| 205 | 5′- GTATGGTAAAGAATCCCAGT[G]CTGCATCAATCAGTGGGCAA -3′ |
| 206 | 5′- TTTTCCTTACACCAAGCTTA[T]GTGGGTGGCTGTAGCCACAA -3′ |
| 207 | 5′- GCACCATGGGGGAAATTATC[A]GTATTATTTTTTTGAAATCA -3′ |
| 208 | 5′- TATAGYCAAAGAGTTGTGCA[G]TGATCACCTCAATGAATTTA -3′ |
| 209 | 5′- GTTCTGGGCAACTGCTTTAG[C]CTGAATGCAAAAAACTGGAA -3′ |
| 210 | 5′- AAACAAAAGCCCCACAGCAA[G]AAACAGGAAGGAAGGGGAAC -3′ |
| 211 | 5′- ATAGTGAGGGATGACTGTAT[T]TTCCACTTAAAAATCCCAAG -3′ |
| 212 | 5′- GGAAAATAAAACTGTACCTC[A]TCTCCAGTCTCCCCATATTT -3′ |
| 213 | 5′- TAATGGCTTTCAAAGTGCCT[A]AATTCCATTCTACACTAAAA -3′ |
| 214 | 5′- ACCTCAAAAGAAAAAATAAC[G]TAAACAATATTCAACTCAAG -3′ |
| 215 | 5′- GCTTGGTTCAGGCCCTGGTT[G]CATACCTGGATTTCAAATCT -3′ |
| 216 | 5′- ACCCACAGCTTTCAGCAGTG[C]AGAATATGAATGGAAACTGG -3′ |
| 217 | 5′- GAGTGAGGTAGAGAACAGGT[G]TAATTCACCATAAGTCCTGA -3′ |
| 218 | 5′- ACCTGGTTCTTTGAAAGAAC[C]AATAAAATTCACAAACTGCT -3′ |
| 219 | 5′- TTTTTCTCTTCAGCTGGCCC[A]AATTGGTTTCTGTTAATTTT -3′ |
| 220 | 5′- GAAGAGACTAAGAGAATCAC[A]GAAGAGAGAAGGAGGTCAAG -3′ |
| 221 | 5′- TCTTGAAGGGTTTTAGTTCC[A]TAAGTTCCAGGGAGGGGTCT -3′ |
| 222 | 5′- AAACGTTTAATTCTTCTGTG[G]GTTCTGTTCTAATTTCTGAG -3′ |
| 223 | 5′- AGGCCTAGAATTCTCTGAAA[T]GTCATTTTTCAGTTTCTACA -3′ |
| 224 | 5′- GTAGCCTTGCGCCTCACTCT[T]GTGATGGAGCCGCCTGCTAC -3′ |
| 225 | 5′- ATTGTCATTTTCCTTGTGTT[A]TATTGGTTCAGGCTATCCAA -3′ |
| 226 | 5′- CAAGGCATCTTGGCTCCTAC[G]TAGGGCCTTTTGGCTCCTCT -3′ |
| 227 | 5′- AGATCTCCAAGGTTTTCACC[G]AGAAACACTTGACCCGACTT -3′ |
| 228 | 5′- CCTCAATGCAGAGGGGTCAT[G]AGAGCAGGCTGGGAGCCAGA -3′ |
| 229 | 5′- GTTCCTCCTCAGAAACTGCC[T]TGTATGAGTTTGTATCCTTA -3′ |
| 230 | 5′- CATAGGCGAGGCCCAGCCCA[C]GTGTCCAGAGACATCTGTGA -3′ |
| 231 | 5′- GCTCTTCAAGGTCTGGTGCT[T]TCTTCCACAGTACTGTAGCC -3′ |
| 232 | 5′- AAATGGGTGCTCAGACCCCT[A]TCCTACTTACCTCAAAAGGT -3′ |
| 233 | 5′- TGTCAGCAGCCTGGTATTGG[G]AAGAGTTAAAGGAAAATCTC -3′ |
| 234 | 5′- CAGTTCAGGGGAGGAGCCTC[A]GGACGTCAGTGGCAAAATCA -3′ |
| 235 | 5′- GCATAGGCTTAACTCGCTGA[T]GAGTTAATTGTTTTATTTTT -3′ |
| 236 | 5′- AGGGGAAACGTCTCCCAGAT[C]GCTCCCTTGGCTTTGAGGCC -3′ |
| 237 | 5′- AGCCAAAGCCAGAGTGGCCA[C]GGCCCAGGGAGGGTGAGCTG -3′ |
| 238 | 5′- TTTCAGAGAGGGAAGCCAGA[G]GAGAAGAGGGTGCAGGCTGA -3′ |
| 239 | 5′- CAAGTCCTCCGGTTCTTCCT[C]GGGATTGGCGGGTCCACTTG -3′ |
| 240 | 5′- AGGCTGCCTCCGCACCTGAC[C]GCTGCCCAGGTGGGGTTTCC -3′ |
| 241 | 5′- TGGCTAGGACAGGGTCTCGG[G]CTAGGGAAGTGGTTTCTCTG -3′ |
| 242 | 5′- TTACGGGAAGCCCTTCTGGC[G]CTCACTCAGGGCAGCAGCTT -3′ |
| 243 | 5′- GCCTGGGCAGGAAGAGGGAC[T]AGAGGGTCTCCCACATGGGA -3′ |
| 244 | 5′- ATCGTGTTCCCCAGGAAGTT[G]TTCTTGATTTAGTTTAAACT -3′ |
| 245 | 5′- GAACCACCTTCTGTTGCCAG[T]CTGTACTCCTCATTTAGTTT -3′ |
| 246 | 5′- AAGGTGGGAGCCAGAGTGGG[C]TGCTGTAGGGGTGAGGGAGG -3′ |
| 247 | 5′- GCCATCCAGCGCGGCTGCTC[C]GGCGCCACCTCCATGGCCGG -3′ |
| 248 | 5′- TCCCTGGGCCCGTCGCCCTC[G]GGGCTCCCGCCGGAACTCCT -3′ |
| 249 | 5′- ACACAGACATTGTCGAGGGC[G]GGTCCCTCTTTATTGGCCAG -3′ |
| 250 | 5′- GCCTGGTGAGAGCAGATTTA[C]TCCAATTTATGGGCTGGAAC -3′ |
| 251 | 5′- CACACCGACACACATGGCCA[C]ACAATCAGATGCAACTCGGC -3′ |
| 252 | 5′- CTTGTTCACAGAAGTGGGAG[G]CAGGAGGGGGGGAGAAAGTG -3′ |
| 253 | 5′- AGGACCAGGCGGCTAAGCAG[G]GAGAAGAGCCAGAGGGGCGT -3′ |
| 254 | 5′- CGGGCCATGGACACCGACAC[G]CTGACACAGGTCAAGGAGAA -3′ |
| 255 | 5′- CTGCGGTTCAGCTCCTTGGT[G]AGATCTGTCATGTCTGTCTG -3′ |
| 256 | 5′- GCACGTCGGCTCTTGGTACA[G]AAGACGAACAGGGCTGCGGG -3′ |
| 257 | 5′- TCCCCCGGGGCCCTGAGCAA[C]GCATCAGCGCCAGTGGACTT -3′ |
| 258 | 5′- TTCACCAGGACCTGGAGCTC[G]GAGCCTACATGGAGGTCATT -3′ |
| 259 | 5′- ACGGTCACCACACCTGAGAG[T]GGTCCTGGGGCTGGCCCTGT -3′ |
| 260 | 5′- GCGGCAGCCATCACTCCACA[T]GCACAGGTGACCCAGGTCTT -3′ |
| 261 | 5′- AGGATGTTCTGGGAGCCACC[C]GTAGGCACGGGTGCCAGGGG -3′ |
| 262 | 5′- TGGAATGAGCAACACAGGAA[T]GCTCCAGTTGTCCAGACCAT -3′ |
| 263 | 5′- CGAGACTGGTTGGAAACACA[G]GAGTGCTGCTGGCTGCACCA -3′ |
| 264 | 5′- CCCCCATCCATTCCAGACCA[C]GTGACTGTTGAGATGTCTGT -3′ |
| 265 | 5′- TCGATGTGCGCCAGGAGTAC[C]CAGTGAGTCCTGGGGGAGGC -3′ |
| 266 | 5′- AGTTTGACCCAGCAGACTCC[G]GTTACCTTTACCTGATGACG -3′ |
| 267 | 5′- CCTACCTTGAGAAGCCTCCC[G]TTGACCGTGCCCAGGAAGAC -3′ |
| 268 | 5′- AGGCCTCCAGGAAGTGACCC[C]GAGACAATAACTGTGCAACT -3′ |
| 269 | 5′- GTAACTAAGCACACCCCTTA[C]AGAATTTTGGGAAGTCGCCC -3′ |
| 270 | 5′- TAAGCCAGAGGATGCTGTAG[A]GAGTACTTGTATGCAATAAC -3′ |
| 271 | 5′- CTTGTTGTCATGGTGCGTTG[G]AAGAGTAGCCAGTTGTCTTT -3′ |
| 272 | 5′- ATTAGTATGCAGGTCTTATC[T]ACCATTGGAATTAAGCTGTT -3′ |
| 273 | 5′- ACGTTTTTATCACACATTAA[G]CACTTGCATTAATTTTGGAG -3′ |
| 274 | 5′- GATGAGTTAAATGGGCTAGT[G]TCTAAATTTTAAATTTTTAC -3′ |
| 275 | 5′- GTACATCCCATATTCCCTTT[G]CAAAATCTAGTTTCCTATGT -3′ |
| 276 | 5′- GCTTACCAGAAAACACCCTC[G]TTGTTGTTTTTATTTCTCAG -3′ |
| 277 | 5′- GGACAAGGAGGAGAAGCCCC[A]GGAGGTCACGGGAGTTCACT -3′ |
| 278 | 5′- GAGCAGCCATTTCGAAAGGC[A]GCAGAAGAGGAAATTAACTC -3′ |
| 279 | 5′- GCGAGGGGAAGTCATTTTTT[T]AATAACTAGGCTCTATTTGC -3′ |
| 280 | 5′- CAAGGAAAGACCTGGTGTCC[T]TGTGCTAATTTTAACTCTCT -3′ |
| 281 | 5′- TACAGATGCTCATAGGCATC[C]GAAAAAAAAATACTTTGTTA -3′ |
| 282 | 5′- AACTCCTTTGACAGTATGGA[C]GGCACCTAACGCATCCTTGT -3′ |
| 283 | 5′- GAGGTGTTTTCTTGGCTCTT[A]ACKAACGTTTTTAATAAAGC -3′ |
| 284 | 5′- GCGCCCCCTGGACTTCTGCT[A]GAATTTAGATTTAAATAGAT -3′ |
| 285 | 5′- ACATATTTAGAATGGATGCC[G]GAACAGGAGAAATGGGTGGG -3′ |
| 286 | 5′- ATTCATATGCCACCAGCCAT[C]GGCAGAAATGTAACAGGAAA -3′ |
| 287 | 5′- ATGGCTCTGTAAATGGGATG[C]CTCATGTTCAGGTTTCTGGA -3′ |
| 288 | 5′- ATCTCCAGGTGAACATGGAA[C]GCAGTGAAAACCTGGGGTAT -3′ |
| 289 | 5′- TGATAAGTAGTTAATGATCC[T]GAAATAAACTGTTAGGTGCT -3′ |
| 290 | 5′- AAGTAAAATAGTAGATATTG[C]ATTGCTTCTACATTTACTAC -3′ |
| 291 | 5′- AGAGCCCCTACCCAATTGCT[C]TACTATTTATAGTTCCTCAG -3′ |
| 292 | 5′- ATCTGGGGACCTGCTCCTGG[T]AGAGCAATAGGAWCTGTGTG -3′ |
| 293 | 5′- GAGTCCCAAAATTCAACCCT[C]CCGATAGGGCTGGGCCTGAC -3′ |
| 294 | 5′- CCCTAGCCTGCTTTTGTCCT[G]TTATTTTTTATTTCCACATA -3′ |
| 295 | 5′- AGAGGGAACCCAAATATTAG[G]GTGGGAAGCAAGTCATAAAC -3′ |
| 296 | 5′- TAGGGTTACCAATCCACTAG[A]ATGCAAAACTGTACTTATTA -3′ |
| 297 | 5′- AGGCTTCTTTTTCCATTACA[C]TGTAAGACTTTGGAGGGCAG -3′ |
| 298 | 5′- AGCRGTCAGGTGCGGAGGCA[G]CCTCTCAGCGGTGGGGAACA -3′ |
| 299 | 5′- CAGGACAAACAGTGGATTCA[C]TCAGAACACAATATGCTGGT -3′ |
| 300 | 5′- AAGCCACTACAGACACCGCA[C]GCACCGAAATTCTCCCTTGT -3′ |
| 301 | 5′- ATCACTGTCCCTCAGTTCAC[C]GGTCTTGTCTGCTTCGTCGY -3′ |
| 302 | 5′- AATTCTCAGTCTTAAAAACA[A]GGCATAAAGAAAGCTAAAAT -3′ |
| 303 | 5′- AGAAGATAAGTGTTTAGGGT[G]TTGGATATCCCAGTTACCCT -3′ |
| 304 | 5′- CCTTTTTTTGGATGATCCTA[C]AATTAATACAAGTGTATTCT -3′ |
| 305 | 5′- GCCCTTAGTCACCAACTCCT[T]CTCATCCCACCATGCTGTTG -3′ |
| 306 | 5′- GTAAATTAAAATTTGTTTGG[C]TGATTTGTGCTGTATTTCTA -3′ |
| 307 | 5′- AGCAACACTTCCTCCTTGCA[G]ATTACAAGCATAGCTAATGC -3′ |
| 308 | 5′- CCCTCATTTTCTGTTAGGGA[T]GTATGTGTTTACCAAGCTGT -3′ |
| 309 | 5′- ATGAGGGCTTTACTTTTGCA[G]GAAATACTACAGATGGTGAA -3′ |
| 310 | 5′- TCCCTTCTCAGTAACTAACA[T]TAATCATCTCTCTGGAGGAC -3′ |
| 311 | 5′- CATTCCCTCACACAGTACAG[T]TTAATAAATGTGCATTTTGA -3′ |
| 312 | 5′- CCTGTGTGATGAGGGGCAAA[G]GAAGCTCTTGAGAACCTGCT -3′ |
| 313 | 5′- GTAACGAAGAAAGACCAGAG[T]GTCATCCCTGTGATACAGCA -3′ |
| 314 | 5′- TATGTATCTTGCTTTTGTTT[A]AAACAGTCATCCACATTAGT -3′ |
| 315 | 5′- GATAGGTTGCAAAATTTTGG[C]GTGTTCTTGCATTGCATACA -3′ |
| 316 | 5′- ATTGACGGTGTTATAATTAC[C]ATGGTTTTGAAATTACATAG -3′ |
| 317 | 5′- TGAGGACCCAGATGTCAACA[C]CACCAATCTGGAATTTGAAA -3′ |
| 318 | 5′- CTCCTTTTGACCTGAGTGTC[A]TCTATCGGGAAGGAGCCAAT -3′ |
| 319 | 5′- TATGTAAAAGTTTTAATGCA[C]GATGTAGCTTACCGCCAGGA -3′ |
| 320 | 5′- GATGGATCCTATCTTACTAA[C]CATCAGCATTTTGAGTTTTT -3′ |
| 321 | 5′- AATTAGCTGCCAGAGTTGCT[G]TCAGTAAAGAGAAGAAATAA -3′ |
| 322 | 5′- CTGAAATCAGAGAACATTGA[A]AGATGAAGTGAATGGCAGAG -3′ |
| 323 | 5′- GCCCATCTGAGGATGTAGTC[A]TCACTCCAKAAAGCTTTGGA -3′ |
| 324 | 5′- GTGCAGAYCAGATAATTATA[C]AGAGATGGAATGGGACAACC -3′ |
| 325 | 5′- AATCTGCCTCTGGGGCGGGA[T]CTGTCAGGCTTCAGGAAGGG -3′ |
| 326 | 5′- TCCAGGGAGGAGCTTCGTGC[G]ACCTTCCCGGACCACTCAGG -3′ |
| 327 | 5′- CATCACCTCCAGGTAGCTCC[T]AAAATGTCCCTAGAAAGTGG -3′ |
| 328 | 5′- GGAGCACAGAGTAGCAGTGA[T]GCTGTCCAAGGCAGGGGGGA -3′ |
| 329 | 5′- CATTCAGGCCAGTGGCTGCA[G]GGGAGCAGAAAGATCAGGCT -3′ |
| 330 | 5′- TACAGAGGAAGAAATCCAGG[G]CAGAGGTGGAGGCAGTGAAG -3′ |
| 331 | 5′- CTACCTCATTCATTGACCCC[A]CTATCTGACCTGTACATGTT -3′ |
| 332 | 5′- TTGAGGACAAACAGAACATC[G]GTGAGTAAGTGGAATATTAG -3′ |
| 333 | 5′- TTCTTGTGTTCTTCCCTTTC[C]ATTTCAACTCTTCATCTCAG -3′ |
| 334 | 5′- GGTTTGTGTACCAGGATTGG[G]GACCCCTGATGTATAGTGTA -3′ |
| 335 | 5′- GAAGAGGATAGGTTTTTCTA[C]CTTAAACAAAATCTTCCTTA -3′ |
| 336 | 5′- GTTAGGCATCAGGCAACTAC[C]AAGGAGTATACGAGCATGCA -3′ |
| 337 | 5′- CACAGGGTAAATTTAGCCAC[T]GCAGCAGGAGCATGATATAA -3′ |
| 338 | 5′- GGCATGTGAAATAAGTTGGT[C]TAATTAGAGTGAAGCCCAGG -3′ |
| 339 | 5′- TGGATTGTGTGTGTGGTAAT[A]GGATTATTGTTATATTTAAA -3′ |
| 340 | 5′- CACGAGCATCTTGCTGTCTT[A]AATTAAGAAGTTAACTGGAC -3′ |
| 341 | 5′- TTGAAAGCTGAGTCATTTTC[A]TAATGGGTCAGAAAGACATT -3′ |
| 342 | 5′- TACATGACGCATGTATTTGT[G]AAAACCCACAGATCTATTAA -3′ |
| 343 | 5′- CTGAGAGTGCAGTGAACCTT[T]GTGTCTGTGATGGAAGAGGT -3′ |
| 344 | 5′- GCTTAGATGTGAGAGTTGAT[G]CCATAATAATAAAAGTTATT -3′ |
| 345 | 5′- TTGAACTCTATGTACCAAGT[T]TGAACACATTCCAAATATCC -3′ |
| 346 | 5′- GGTATTTTGCTACAGCAGCC[C]GAGCAAACTAATATATCATC -3′ |
| 347 | 5′- AAAGGCGGTCACCTGCAGGA[A]TAGCCATCTTTGGTCCTTTC -3′ |
| 348 | 5′- CCCCCAGGGGTGGTAACAAC[A]GCACGCAAGCACAGCCATTG -3′ |
| 349 | 5′- CCACACCTGGTGGACAGGAC[C]ACCGTGGTGGCCAGGAAGCT -3′ |
| 350 | 5′- GGTTAAAAAGTTCTCTACCA[C]GGAAGTTGGATAAAAGTAAC -3′ |
| 351 | 5′- AAATCAGAATCGAATTATTG[G]TTTGGGGCTAATTGTATCTG -3′ |
| 352 | 5′- CCTGTCAGTGAAAACAACTA[C]CAAAGCTGGATTTTAAATAT -3′ |
| 353 | 5′- CCATTAGCAGTAGGTCTGAA[T]TAACTTTAATATGCAAGTTA -3′ |
| 354 | 5′- AGAGCCAGCTGGGAGAAACA[T]GCAACATAGTTCTTTGCAAT -3′ |
| 355 | 5′- AGCAGCTGGACCATGATCTC[C]TGGATATGGTGGTAGGTGAA -3′ |
| 356 | 5′- AGACGATGTACTGATGTAAG[G]TTTTGTAAATTTCTAAACTG -3′ |
| 357 | 5′- ACTCTGTCTTTCCAATTCCT[C]AACAGCATGCTTGGATGGGA -3′ |
| 358 | 5′- TCAGAAAGAATGGGGTAAGG[T]GAATTGAGTTTTAGAACATA -3′ |
| 359 | 5′- ACAGTGAAGAAAGAGGAACA[T]AGAGAAGGGCAGGCAGGAGG -3′ |
| 360 | 5′- TTGAAGGTGGATGAGGGAAC[G]GTCAGGTTGAGGAGCATTTT -3′ |
| 361 | 5′- ACACAATACTGGGTTTCTCT[T]CTTCTCTCTCACCATCACAC -3′ |
| 362 | 5′- CCACGCACCAGCAGGTTCAC[G]GTGCAGCTCATGCGGTTGTC -3′ |
| 363 | 5′- GATAGTCTAAATGAATGTCC[C]CCACCCCCGCCTGTAGTTGT -3′ |
| 364 | 5′- GCTGGCTGGGGCAAAGGTCT[C]TGATGCACTGTGCAGAAGTA -3′ |
| 365 | 5′- CTGCTCGGGCCAGAAAATCC[G]GAAACGGGCCCTTACCGATG -3′ |
| 366 | 5′- GTTCTGAAATGAAGACACAT[A]TGGCAGGCAGGTTACAACCC -3′ |
| 367 | 5′- CTCACTCACTCCTTGAGGAC[C]CTCTCATGACAACTGTAAAG -3′ |
| 368 | 5′- TTCAAAAACTATTTTGGTAC[C]TTTCAAATACAGTGTTTAAA -3′ |
| 369 | 5′- TGTTGCTAAGATCAATAGCT[G]CATTTGAATCTATGTCTCCC -3′ |
| 370 | 5′- CAGTTTATTATGGGTTATCT[C]ATTGGAATAAAGAGGTATCA -3′ |
| 371 | 5′- AATCATTATGTCACAAAAAA[T]TATATAAAGATAAATTTTTC -3′ |
| 372 | 5′- AGAGCCAAGACTTGTCCCCT[A]TTTCTGCAGCAGATTGGTCC -3′ |
| 373 | 5′- GTTTCTCAAAAGTTCTAAAC[T]TTACAGAGGATAATTTTAAG -3′ |
| 374 | 5′- CCTTGTCTGGACGAGTTGGG[G]TTCCTCAATAATTGGCTGTG -3′ |
| 375 | 5′- GGATCCAAAGGGTGTCAAGG[C]GATCATTATCTTGGGATGGA -3′ |
| 376 | 5′- AATGAAACTAAATGATCATC[C]TTCAACTCTCCCTTCTCACT -3′ |
| 377 | 5′- AGTTGCTCCCCTCTCTGATC[C]ACATTCGTAAAATGACATAA -3′ |
| 378 | 5′- CAGGTGTCCCTACCTTAAGG[T]CCTCCTCCTTGGGACTTCAG -3′ |
| 379 | 5′- CACACAACTRGCTAAGGAGC[T]CCAGGGCCACAGCTGCTGTT -3′ |
| 380 | 5′- ATAAGCAGGAAAATGAATGC[G]TTAGGAGAGGTTTTATATCT -3′ |
| 381 | 5′- TTATGCATACAACACTCAAC[A]GATCCAGTTACTCTIACTCT -3′ |
| 382 | 5′- CACCCCAGTCACCGTGGCTT[G]CACCTGCACAACAGATTCCT -3′ |
| 383 | 5′- AATTTCCCTGCATTTTGTGA[C]GACTTGTTTTTATTGGTAAC -3′ |
| 384 | 5′- TGCGCATTTTCCGCACTCGG[A]TACACTTTACACTGAACACC -3′ |
| 385 | 5′- GACCCAGAGCAGGAAGCATA[G]TCAAGCCCTCGACTAGATTA -3′ |
| 386 | 5′- CACTTGGAAATCCTAACTCC[A]CAGAACAAAATTTTACAAGC -3′ |
| 387 | 5′- ACACACTGACATTCGAGGCC[A]AAGGAATACTCCTGCCTCTA -3′ |
| 388 | 5′- TTCATTTACAAGCCTGATCA[C]CCTTACATGAACTAATGTTT -3′ |
| 389 | 5′- AACACTGTTGCAGGATCTCT[G]ATAATCACTATGTACACTTC -3′ |
| 390 | 5′- AACTCCCCAGCTAAACACCC[G]TAAGACTTCATACAACACAA -3′ |
| 391 | 5′- TAAATGCTTATCCATTTAGT[A]ACAGGAAAAATGAGACAACT -3′ |
| 392 | 5′- GTATGCTTTCCATCGAAAAA[T]TACTCTATTAAACAGCTTAG -3′ |
| 393 | 5′- TATACAGGAGTCATCCCCTA[C]GTTGACACTGGTAAGTTGTA -3′ |
| 394 | 5′- TCAAGTTTAAGCTGCTATGT[T]CCTTATTTTTAACTTTTGTT -3′ |
| 395 | 5′- ATATAATTTATATTACAATG[G]AAAAGCTTCTTTAATACTAA -3′ |
| 396 | 5′- GATGGGGAGGAAGAGAAGGC[G]TTGGTCTTGCAGTCTTGTCT -3′ |
| 397 | 5′- AATGGTAAGCATCTATTTTG[T]AGTCCACTCTACTGAGCTAA -3′ |
| 398 | 5′- TTTATATATGATATCATCAT[A]AAGCACTTTCTATAAGCTGA -3′ |
| 399 | 5′- AACAATCTGTGAACACTTGT[T]ATATGCTTACTGTAAGTGTG -3′ |
| 400 | 5′- ACTATATGTCATGTCTACAG[T]CTGTCTCCTAAGAGTAGAGG -3′ |
| 401 | 5′- TGCAAACATTGGGAAACCAC[G]GTAGGGGGGAGCAGGACTCT -3′ |
| 402 | 5′- TACCATGGACAGCAGCGCTG[C]CCCCACRAACGCCAGCAATT -3′ |
| 403 | 5′- ACTTGTCCCACTTAGATGGC[A]ACCTGTCCGACCCATGCGGT -3′ |
| 404 | 5′- GCATTTCACATTCACATGTA[G]TATTTGAATATACACATCAA -3′ |
| 405 | 5′- TTGAGTCTCCTTCCAATTAA[C]TCATGGAACATCAGAGCCAT -3′ |
| 406 | 5′- TCTTTTGTGGAAATGTGATG[C]ATTTGTTTATATGCAGACAA -3′ |
| 407 | 5′- ACCAGACTTAGGAGAGATAT[A]TCTCACTGTAGAACCAGTGC -3′ |
| 408 | 5′- CTCTGGTCAAGGCTAAAAAT[C]AATGAGCAAAATGGCAGTAT -3′ |
| 409 | 5′- AGCCAAAGTTCAGTTCTCCA[G]TTCATCTGAGCTCAGGCCCA -3′ |
| 410 | 5′- GGTATCRTGGGTCCTTTCRAGTAC[T]AACCGCCTTAGGCTGGAAGC -3′ |
| 411 | 5′- TTTTACCGAAGGCTGTGTCT[T]GTAAGCACCCCCGAGCAACT -3′ |
| 412 | 5′- CTACTCCGGCACCCAGTGGG[T]TGGTAGTCCTGTrGGCAGGA -3′ |
| 413 | 5′- CCAAGAAGCGCGCGGCGAGA[G]TGCAAGGTGGGGGCCCCGCC -3′ |
| 414 | 5′- CTCTCGCCGCGCGCTTCTTG[G]TCCCTGAGACTTCGAACGAA -3′ |
| 415 | 5′- GAGCAGAGGGGCAGGTCCCG[A]CCGGACGGCGCCCGGAGCCC -3′ |
| 416 | 5′- AGAGCGGATTGGGGGTCGCG[T]GTGGTAGCAGGAGGAGGAGC -3′ |
| 417 | 5′- TGGGGATTCAGAGCACCCAC[G]CGCAGCACCTCCCTCCTCTG -3′ |
| 418 | 5′- GGGTCAGTCCGGACAGCCCC[A]GTCGCTTGTTACCTAGCATC -3′ |
| 419 | 5′- CTGGGTGCGCTGGCCGAGGC[G]TACCCCTCCAAGCCGGACAA -3′ |
| 420 | 5′- GACATGGCCAGATACTACTC[G]GCGCTGCGACACTACATCAA -3′ |
| 421 | 5′- CTGACAATGTCTGTGGCAAC[C]CTGCAGTTTACTCCTTGGTT -3′ |
| 422 | 5′- CAGACACCCACTCCTATGTG[T]GTTTCTGAAAATTACAGGGT -3′ |
| 423 | 5′- TCCAGATATGGAAAACGATC[C]AGCCCAGAGACACTGATTTC -3′ |
| 424 | 5′- ATTTCAATTTAGAGTCAGGG[T]CTCACTCTATGCTCCCCTGA -3′ |
| 425 | 5′- TGGAAAGAGGTGCCCACCAA[T]GTCTAAGTGTTAAACATTGA -3′ |
| 426 | 5′- TATCATGCATTCAAAAGTGT[A]TCCTCCTCAATGAAAAATCT -3′ |
| 427 | 5′- TGAAAAATCTATTACAATAG[T]GAGGATTATTTTCGTTAAAC -3′ |
| 428 | 5′- TATTTCTCAAACATTTTCAG[G]TTTAGAATGGGAATAGGTTT -3′ |
| 429 | 5′- GTGCCTTTAAACCTATTCTA[A]AACCTATTTAAACGTATTTC -3′ |
| 430 | 5′- AGGGCTGCCTGGTAAGCTGA[A]TCAGGGTGCCTGGCTGCCGC -3′ |
| 431 | 5′- AACGCCACTTGTGACTGCTC[G]TTACCTTTCAGTTGTGTCCC -3′ |
| 432 | 5′- ATGTTGGGATTTAACTTTCT[G]TTATATGTCAGACTCACTTA -3′ |
| 433 | 5′- TGTGTGTTTTAAATCTTTGC[G]CTTAAATGTTTTTGATTTCT -3′ |
| 434 | 5′- GAAGCTTCCCTCCGACAGGC[G]GCCCCGCACTAAGGTAGGGA -3′ |
| 435 | 5′- CTAATGGTTGGAAACGCCAG[C]CTTTGGTGAAAACAGAAAGT -3′ |
| 436 | 5′- TTCAAGAATTCAACTGCAGA[T]TGAAAATATTTGGAGAAAAA -3′ |
| 437 | 5′- AACCTAGCCACAGAGCCCGA[T]GCGATGTGTCCTTGTCGAGA -3′ |
| 438 | 5′- GCCTCCTTTGCTGCCCTCAC[A]ATCTCTTCCTGTGACACCAC -3′ |
| 439 | 5′- CTCTGCACCTTCAGGTTCAG[G]CCCTTCAAGATCTACCAGGA -3′ |
| 440 | 5′- ACAAGCTAGTTACCTTTTAT[T]GTTCAGTTTAAAAAAGTTCT -3′ |
| 441 | 5′- CGGTCCCCTTCAAGATCCAT[C]CCGACCTGAAGAGAAACCGC -3′ |
| 442 | 5′- TGCTCTTCAAAAAAACCAGA[C]TGAATATTTTTAAAAGTAAT -3′ |
| 443 | 5′- GTTACTTGTAGGGGGAGGGT[G]GAGGGAAATCTGGGCAAATG -3′ |
| 444 | 5′- GGGCTTCTATCCCCGAACCC[T]GGGCCCTGGTGCCACTCAAG -3′ |
| 445 | 5′- TCCCAYTTAAGAGCTATTCT[C]CTATCCTTCCCTGTAAACAA -3′ |
| 446 | 5′- TGGCAGACACAGGACAGGGA[T]CGCTGCTTATGTCTCCGAGG -3′ |
| 447 | 5′- AACCCATCCTCGTGGTAATC[A]TCCCTGGTAAGAAACACACA -3′ |
| 448 | 5′- CATTTCTAATTACCAGCTTC[C]TACTTGGCACTTTCAATTTT -3′ |
| 449 | 5′- CCACAGCGGCTTCCTGCCAT[C]GATGAGGCTGATTTCTGCCT -3′ |
| 450 | 5′- TGCATCCTCTGCTTCTCCTC[A]AACCGTGCTTCACAGCTGCC -3′ |
| 451 | 5′- GGGGCCAAAGGAATATTTAG[G]TGAAGGGGGAGAGAGGCCAC -3′ |
| 452 | 5′- ACTTTGTGTGTACATGTGGA[A]GGAAGTATTTGACATTTTGA -3′ |
| 453 | 5′- ACTTGTGTCCCCCAAAATCA[C]ATATTGAAGTTAAAACCTCC -3′ |
| 454 | 5′- TAGCCATGGCAGAAGACATA[T]TCTCTACACCTTATGCATGG -3′ |
| 455 | 5′- GACAGAGAAGGTATGTCCAC[A]CACACTAGACATACTGCATG -3′ |
| 456 | 5′- AGTATTGATCAGTGGCGGGA[T]ACAGTTTGAAGGTAGAGGGA -3′ |
| 457 | 5′- GCTGTATCTTGGGGGAAGTG[C]GTTCTTGAGAGCTGTGTAAG -3′ |
| 458 | 5′- GGCCGTCCTCATCTTCACAC[G]CTGTTCTCCTTCTATGTGGG -3′ |
| 459 | 5′- TAGCAGGTGGCACAACTGGC[A]CTGGGAACCGGGGGTCCCTT -3′ |
| 460 | 5′- GGCCCCCCGTGCAGGGAGGG[C]TTCAGGCTGCGGCAGGTAGG -3′ |
| 461 | 5′- TACTATACAAATAAAAAAAT[A]AAAACCCAACCTCAAGCTGT -3′ |
| 462 | 5′- CGAATGCTGAGAACTTGCCA[C]GCTCTCTCCCCAGGGCCCCA -3′ |
| 463 | 5′- GCCTCCCCCTGTGATCTCTC[A]GTCCTCTCCGCATTCCTGGG -3′ |
| 464 | 5′- TTCCCTTTGTTTTCCCTTTC[C]TCCAGCTCCAGGCCAGGCTT -3′ |
| 465 | 5′- TGCGCTCTGGGCTAGACACT[G]TGATAGGTGCTGGGATTACA -3′ |
| 466 | 5′- TGGAAAACAGATCCAGACAG[G]TTCAGTTATGTGTCTGAGAA -3′ |
| 467 | 5′- CCCTACTACCCCTACAACTA[C]ACGAGCGGCGCTGCCACCGG -3′ |
| 468 | 5′- GCATGCCTTTTCAAAAACAC[A]TTCAAGACCTGAAAATAAAA -3′ |
| 469 | 5′- TACTGCTGTGGCCTGAATCC[G]TGATTAAAGGAAATGCTAAG -3′ |
| 470 | 5′- TACACAAGTCACTGGGTGAC[A]TCTGTAGCTCCACCAACCTG -3′ |
| 471 | 5′- CTCTGTCTAGGTGCATAGAA[T]TGTGTACATATACATACACA -3′ |
| 472 | 5′- AGTCTGCAAATGTGTTTTTT[G]TGTGCTAAATAGCTCAAAGT -3′ |
| 473 | 5′- TAAGTTTGGTTGATGAGTCT[G]TCTCTCTAGACTGCAAGCTC -3′ |
| 474 | 5′- CACAGAAGTGGGCATTCTGA[G]AGGCCTCTAATTTTCCTCTA -3′ |
| 475 | 5′- TTAAAACAGCGACCCCATAC[A]TGCATTAGTTAAAACTTTCT -3′ |
| 476 | 5′- GCAGATTGAGGTAAATTCAT[T]GTTAATGTCATCACAGCAAT -3′ |
| 477 | 5′- CAAAACAGAATCCCAAGAGC[A]ATATTTTAACTCAACAAACA -3′ |
| 478 | 5′- AGAGTTCTTATGGTTCTCTT[C]GGTAGTTTTTCTTTAGCTGG -3′ |
| 479 | 5′- CTTTCATTCTTGTCGTTGGC[G]TCTCTGTTCTGATAAAAAGA -3′ |
| 480 | 5′- GGAGGCAATGTCTGATTTGC[G]TAGGGCTCAGGGGAGAGATG -3′ |
| 481 | 5′- AGGTTCAGCAGAAAAGAACC[T]AGGAAAAAAGTCTAGGAAAG -3′ |
| 482 | 5′- GATGGGCCTTCTGATAAGGA[A]CGCTGCCAAAAGTTCAAATG -3′ |
| 483 | 5′- ATTCCTTCCTTTCCCTGTTT[A]TACATACCTTACAGATACTG -3′ |
| 484 | 5′- TCTGTTTCAGTCTCAAGGAG[G]CTGAAAAGGTGAATTCCTGT -3′ |
| 485 | 5′- CAGTCTTGTGAGAACATTCT[T]GCCATCTGTACTTTGCATTT -3′ |
| 486 | 5′- CCACACCTGGCCTGAACTCT[G]CTTTAAAAACTGCATGCTGA -3′ |
| 487 | 5′- TCATGCATAGATGGTGTAGC[A]TTAGAAAACTCAGGCCTAGC -3′ |
| 488 | 5′- AGGTGGATTTTTTTAAGAAG[C]ATATTCATACAACTGAATAT -3′ |
| 489 | 5′- GCCTGATATTCTTTCCCTAT[G]AAATTGCTTCCTCATCTAGG -3′ |
| 490 | 5′- GAAGAAGCTGTCAGAATTGC[A]AGGGAAATTGGTAAGTCCTT -3′ |
| 491 | 5′- ACTGTGCCCACCCAAGTTTG[T]GTTTTGAAAAGATTGGTCAA -3′ |
| 492 | 5′- ATGGCATACAGCCTGGGTGA[T]ATTTTTAAACATAAGTGAAA -3′ |
| 493 | 5′- GGGAAAATGTTCATTTAAGT[A]TAAAACATGAAATGGTATTC -3′ |
| 494 | 5′- CTTGTTAGTTCAGGTCTCTT[T]CAGATGAGGAAGACAGATTA -3′ |
| 495 | 5′- AAATGGACAACAAAAGTCAC[C]GGAAAAAAGGGAAAAAAAGA -3′ |
| 496 | 5′- TGAGAAATAAGTGATGTCAT[G]CATTTTTGGTTGTGGATCAT -3′ |
| 497 | 5′- TGTGGTTCTCCCTTCACAGT[G]GAATACAAGGGCTTTTATAT -3′ |
| 498 | 5′- TAATAAGTGGTTATGCCAAG[C]GGTCCCTGCAGCTCAGAGGC -3′ |
| 499 | 5′- TCTTTGGGCCTCCACCCCCT[C]GTCTCTAGTGGACATTTGAG -3′ |
| 500 | 5′- AAAGGAAGCTGGGCGTCCTC[C]GGGCCCCCCAACACACGTCC -3′ |
| 501 | 5′- CTAACACAGTTGCGAACATC[G]GCAGAGCCCTCGGGAGCCAC -3′ |
| 502 | 5′- TTGATGATGATGTCGATGCC[G]AAGAGTGACACGCCCAGTGC -3′ |
| 503 | 5′- CTTCACAGCGCCGCAACAAT[C]ATGCATGAGGGAGTGATTCG -3′ |
| 504 | 5′- GGCCACAGCTGGCCAGTCTC[C]TTGTGCTTTGAATCTCCAGC -3′ |
| 505 | 5′- TGCAGCGTGCGGCAGTGCTT[T]GTTCTTCTTTAAGATGAAAT -3′ |
| 506 | 5′- CCTACACAGGAAGCCCCGGA[G]CCACAGCAATTCTCCCTGCC -3′ |
| 507 | 5′- TGTGCTCTGGCCAGGGGCCT[G]GACCTCATTCTGTTGGTGGT -3′ |
| 508 | 5′- TCGCCCAGGCTGACCACAAG[C]TCCAAACAGGACTTTCTTGT -3′ |
| 509 | 5′- TGCCCAAACAGTATCAAAAG[T]GGATGTTTATCACAATACTA -3′ |
| 510 | 5′- TTAGCAACAAAATCCTGAAG[T]CACTTCTAGACCATAACCCA -3′ |
| 511 | 5′- CAGAGGGCAGGGCCCACACC[G]TACCCCACAGAAGCCCAGGA -3′ |
| 512 | 5′- GGGTACAGCCCAGCATGGCC[G]CAGGGGTCCCTGATGGGAAT -3′ |
| 513 | 5′- GACTGCCAGGTGTGGACACA[T]GCTCGTCAAGTGGTGAAGAA -3′ |
| 514 | 5′- CACACGGACGCTTCCTCCTA[T]GTGAAGTTCTGTTTCCTCCC -3′ |
| 515 | 5′- ATGGTCATATTATGCATGCA[C]GTTTTTGATTTCAAGAATGC -3′ |
| 516 | 5′- ATGCGGTGCTCGGTAACTGT[G]CATCCGATGCAGGCCTCACT -3′ |
| 517 | 5′- ACCAGAATTATCACAGCACC[T]TCTCATTCCCAGCGCGTCCT -3′ |
| 518 | 5′- TGATCATGGTCACTGCCCTG[C]GTTCAAATAATGCGAGCTGA -3′ |
| 519 | 5′- AGGACAACATGCCATTTGTC[G]AAACGTTTTAAAGATATGAT -3′ |
| 520 | 5′- GGGGGAAGCTGGGTGCATGC[G]GAGCACCGTGGAGTCTGGGA -3′ |
| 521 | 5′- CCTTGAAGTCACCCGGCCCC[G]ATGCAAGGTGCCCACATGTG -3′ |
| 522 | 5′- TTTGGAAGGAAAACGTGGCG[T]GTGGGCGTATTCTCCAGAAG -3′ |
| 523 | 5′- TCCCAGACCAGACCTTGCCC[A]ATGACGTTGTTGGTAATGCT -3′ |
| 524 | 5′- TGAGATCCCCCGGACAACAC[A]CTCCACCTTCCCATGGAGCT -3′ |
| 525 | 5′- TTGTTTGTGTCTGTCTCAAA[C]CCAAAGGGGTGGCTCAGCCT -3′ |
| 526 | 5′- GAACCTCCCAGGGGGCAGAA[C]AAAAAGTCAACAAGCTGGAA -3′ |
| 527 | 5′- CAAACGTTGCTGAAGTCTCC[C]CGACCTTTATTGTTTTGCCC -3′ |
| 528 | 5′- GTTCCCTGACCAGGAGTCCA[A]TAGGCAATAGTCTATTAACT -3′ |
| 529 | 5′- TTTGCTCATGCACCTGCCTT[G]CCTTTGTCATCACAACAGAA -3′ |
| 530 | 5′- ACCTCCTTCCCCGTGCKCCA[C]GAGGAGCGGGCTGCACCTTG -3′ |
| 531 | 5′- GCTGAAACCCGATTCCTACC[A]GGTGACGCTGAGACCGTACC -3′ |
| 532 | 5′- TCCTGCTCGACCTGCTCCTC[C]AGCTGTGCAATCTTGGCCTC -3′ |
| 533 | 5′- TCCAGCGCCGCGATGGTGGA[C]TTGAACTTGGACTTGACGGC -3′ |
| 534 | 5′- TACGAGGAGAAGGCGGCCGC[G]TATGATAAACTGGAAAAGAC -3′ |
| 535 | 5′- TTCCGCAGCTTGAGGTAGGC[G]GCGCAGTTCCTCTGAATCAC -3′ |
| 536 | 5′- CCTGTGGCTGGTACCTTCCC[A]GCATAATGGATGATGGAGAA -3′ |
| 537 | 5′- ATGATTGCCATGGCCTCCAC[G]GTTTCCTGGAACATCTCATC -3′ |
| 538 | 5′- CCAGAACCACCAACATCTTC[A]GTCTCTGTATTCAATTTTAT -3′ |
| 539 | 5′- TTTTCCCAGCTGTAAAAGGG[A]GCTAATAATAGCTCTTGCGG -3′ |
| 540 | 5′- GATACCTGACTCCAGGAGCC[A]TCACTTTACAACCTGAGATT -3′ |
| 541 | 5′- TTCTTGCCCTTGTACATGTC[G]ACGATCTTCTCCGAGTAGAT -3′ |
| 542 | 5′- ATCATGCTCAGTGAAACAAA[C]CAGAAAGGCCACACGCTCTA -3′ |
| 543 | 5′- ACCTGGTCAACAGCTTCCCT[T]AGGATTTTACTGCCAAGCCA -3′ |
| 544 | 5′- CACCCAGTCTGACCTTCACT[T]TTTTGTTGATGGGGCTGAGC -3′ |
| 545 | 5′- GCTGCTGGGGGTGGGTGCTT[G]GATCCTGGTGAAATGGCCTC -3′ |
| 546 | 5′- AGAATCATCTTCTCCTTTCC[T]TCACCTGATACCCAGCTTGA -3′ |
| 547 | 5′- CCTGTCAGGCCTGACGGGGA[G]GAACCACTGCACCACCGAGA -3′ |
| 548 | 5′- GGCTATGAATATAGTACCTG[A]AAAAATGCCAAGACATGATT -3′ |
| 549 | 5′- CTTTTGGGAATTTCCTCTCC[C]CTTGGCACTCGGAGTTGGGG -3′ |
| 550 | 5′- CAAGCCATGGCAGCGGACAG[C]CTGCTGAGAACACCCAGGAA -3′ |
| 551 | 5′- GACCAGTGAACTTCATCCTT[A]TCTGTCCAGGAGGTGGCCTC -3′ |
| 552 | 5′- TCAGTATAGATGCACCCATC[G]TAAGCCTAACTACATTGTAT -3′ |
| 553 | 5′- GTGAGCGTGCCATCAGCCCA[G]TGGAGGGGCTTAGGTCTGCA -3′ |
| 554 | 5′- GGTGCCATCCAGTGCCCTGA[T]AGTCAGTTCGAATGCCCGGA -3′ |
| 555 | 5′- GGCCCGTAGCCCTCACGTGG[G]TGTGAAGGACGTGGAGTGTG -3′ |
| 556 | 5′- TCAGGCCTCCCTAGCACCTC[C]CCCTAACCAAATTCTCCCTG -3′ |
| 557 | 5′- AGCCATGAGTTTCCACCAGC[A]GCAGAGTGAGTCCTGAGCAC -3′ |
| 558 | 5′- ATTGCAGAGAATGGAAGAAT[T]TGAAGAACTGAGTGACAAGG -3′ |
| 559 | 5′- AGCTACTGGGTAGAATTTTA[C]GTAGTAACTAGGTAGACACT -3′ |
| 560 | 5′- GGATGGCATAGCGAGAATAC[T]AATCTAGGAAGCGACTGGAC -3′ |
| 561 | 5′- GCTTTCCTGCTATCATAGCC[G]ACTTAAGTAGCTGTATTAGG -3′ |
| 562 | 5′- ATGAGGAAGAGAGAGACGAG[G]TGGGGTGACTCATGCCTGAA -3′ |
| 563 | 5′- TTTCTTTGAGACAGGGTCTC[C]CTCTGTTACCCAAGCTGGRA -3′ |
| 564 | 5′- TCATTAGCAGGGTGATGGTG[A]GGCTGAGATGGGCAGGGCCA -3′ |
| 565 | 5′- ATTGCCAACATAGCTGTTCA[T]ACCTAGAACACCTTTTCCTT -3′ |
| 566 | 5′- CACAACCTCGGTAAGGCTGG[T]GATCTTCAAGCCAGTCCGAT -3′ |
| 567 | 5′- GTCCGTTGTCCACGTTCTAC[C]TCCACCCCACTAACTGAACG -3′ |
| 568 | 5′- AGGCCAGGGGTCTGGATGCA[C]ATAGCGTTCCCCTAGCCTCT -3′ |
| 569 | 5′- TGCAGAGGTGTGGGCCCCTG[G]GGACCCAGAAGTCCAGCCAC -3′ |
| 570 | 5′- GGGTGAAGTAAAGTGGGCAG[T]GTGATTTAGCAGAGTGGTCA -3′ |
| 571 | 5′- GGCACCTGTCATAGTCTTGC[C]GAAAGATGACAAGCCCTGGT -3′ |
| 572 | 5′- CGCAGCCCAGGATGATCTGT[A]CGGGACAGAGGCAGCGGCCT -3′ |
| 573 | 5′- TCGGAACAGCGAGTCCTCTG[C]CGTCGAGAGCAGGGAGGGGT -3′ |
| 574 | 5′- TTTGCCCAGTGACGCAGCAT[T]CCAGGCTGAGATTGCAGAAT -3′ |
| 575 | 5′- GCCCCCTCTGCAGGTCCCCT[C]GGTGTACTCTGAGGTGGGAA -3′ |

TABLE 5

| (SEQ ID | WT Sequence |
|---|---|
| NO:) | (polymorphism location is indicated in brackets) |
| 576 | 5′- GGACACAACAGGACCCACTG[A]GGAAAACAATGATGACTTGG -3′ |
| 577 | 5′- CCCCTCCACTTTGCTCACCC[G]TCTTCCGGGCCCTGAACCCA -3′ |
| 578 | 5′- TCCTGTGCCGGCTGCAGGTA[C]GGAACAAGTAGGCTAGTGTC -3′ |
| 579 | 5′- AGGAAAGACTGTTGGGCCTC[A]GAAAACATCCCACGTGCTAG -3′ |
| 580 | 5′- GGGACTTGGTTTCATGTCTC[C]ATCTCTCAGTTCTGTTTCCC -3′ |
| 581 | 5′- ATAGAGAGGGTCTGTTAGGT[G]CTTGGGATCTTGTTCTTCAA -3′ |
| 582 | 5′- ATTCCAATTGAAGATTGAAA[A]TGGCCTGTTTGGTAAACTGG -3′ |
| 583 | 5′- TAACTCAAAGCACAAAGTTT[A]GAATTCCTACATTCTAAAGA -3′ |
| 584 | 5′- GTCACCTGCCTCGGAGCCAG[C]TAGGCTGTTTAACAGTGCAG -3′ |
| 585 | 5′- GGAGCTTTGGCATCGCAGAG[G]CTTGAGCTGAGTCTGGCTCT -3′ |
| 586 | 5′- CAGAGCCCCTCCCTCTAAAC[C]CAGTCTTTCAAAGGGATTGT -3′ |
| 587 | 5′- CAATTTCTTGCTGAAAGCCC[C]GAGTTATGCCAGACACTGTG -3′ |
| 588 | 5′- ACCTTTGCCCAGATCCAAAT[T]TTTTTTCTTCATTCGAAGCT -3′ |
| 589 | 5′- ACGGATCTCTTACCATTAAA[C]TCAGGTGGAGAGGGAGTGCC -3′ |
| 590 | 5′- TTTCACAGATGAGGAGGCTG[A]CCTCAGGAAATGTGACTCAG -3′ |
| 591 | 5′- CCAACACCACCCCTTGCCCA[A]CCAATGCACACAGTAGGGCT -3′ |
| 592 | 5′- CCCATATCATGCAGAGGATC[C]GGGATTTCAATCCAGGTCTA -3′ |
| 593 | 5′- TGACGTGTGCAGAGAGACAT[G]TCAGCCTGCCCTGCACTTGT -3′ |
| 594 | 5′- GGCAGCATATTAGAAAATAG[T]TTATGTTACAACAAAAACCC -3′ |
| 595 | 5′- TGCCCCTTCTCACTGGTCTG[T]GGCTGGCAGGGCCATCTTTC -3′ |
| 596 | 5′- GAATCCATCCCAAGGACACC[A]TTTGAAAACATGAAATAACA -3′ |
| 597 | 5′- CAGCGGGGAGGGGAAAGGTC[C]GAAATGAGGGGAGAGACGTG -3′ |
| 598 | 5′- GCTGGGCAGAGCCATTCCTG[G]GCTGGCTGGGTGTGTTTGGG -3′ |
| 599 | 5′- ACAGGCATCAGGGATACAGT[A]GTGAACAAGCATACACAATC -3′ |
| 600 | 5′- AGGTGAAGCTGAGGCCTGAG[A]CCAGAAGGAGAGAAAAGGAA -3′ |
| 601 | 5′- CACTCATTAATCCATTAAAC[A]ATTAATCTATTAATCCATGA -3′ |
| 602 | 5′- GTGTATGCTGTGAAGAAGGC[C]ACCCCCCTTCCTGCCCATCC -3′ |
| 603 | 5′- CTGTCACTATGCCCCTGCCT[C]TCTCAGTGTCTATCTCTGTT -3′ |
| 604 | 5′- GGGATGACAGTGAGAGGAGG[G]CAACAGTAAAAGGAGTCATA -3′ |
| 605 | 5′- GTGTGTCTGTCAGGGAATGT[A]TCCCTCTTCCATTCTCTGTG -3′ |
| 606 | 5′- CCATTCTTGGTGGTGAGCCT[A]GACTCTGAGCCTGGGATGTG -3′ |
| 607 | 5′- GTCTGGCTGCCCCTTGGCCT[T]CACYACAGTCAGGTCCAGCC -3′ |
| 608 | 5′- TTGAGGATTAAAGAGCAGAR[A]TCATGTAGCATCTGGCACAT -3′ |
| 609 | 5′- CGTCATGTAGCATCTGGCAC[A]TGGGGGAACGCAATGGAAGT -3′ |
| 610 | 5′- CAGAGAATATTTCACATGCA[C]GTAGCAAAAACACCAGGGGT -3′ |
| 611 | 5′- AACATGGATTAATGTGGGAA[T]TTGGCTTCAAGAACACAACC -3′ |
| 612 | 5′- ATTATTTCATTTTAAAACCA[C]AGAATAAAAATGACACCTGA -3′ |
| 613 | 5′- AAGCAGATTATGAGGCAGCT[T]CACCCCTCCCAGCACTGGGG -3′ |
| 614 | 5′- CCAGCCCTGTAGTGGACATA[C]TTGCCTTTGCCTATTCAGCA -3′ |
| 615 | 5′- GAACTCGGTGGAGGAGAAGA[A]AAACTCCAAGATGCTCCAGA -3′ |
| 616 | 5′- TGTGGGCTGGACTTAGCAAC[T]CACTTCTAACTAACAGAATG -3′ |
| 617 | 5′- GGTGTCAATTCACTCCCAGC[A]GCACTGACTGAGTGCTGACC -3′ |
| 618 | 5′- ATGTTAGGCGGTCCCACCTG[A]GTTCTGGAGATCTTCACACA -3′ |
| 619 | 5′- GGTGGGCAGAGGCTGGATCC[C]ATGGTGAGGAGTTTCCATTT -3′ |
| 620 | 5′- TTGCCATGGGCCACCTCTAC[T]GAGTGCTCGATGAACAACAA -3′ |
| 621 | 5′- TTTGGCTGGGGCAAGCTTAC[A]TGGTTCGGCAGTAGTACCAG -3′ |
| 622 | 5′- GTGGCCCCAGGAATGGGGGC[A]TCTGGTGGTATCTGGGCTGG -3′ |
| 623 | 5′- ATGCATTGTGGTAGAYTCAT[T]CAATGGAGTATACACAGCAA -3′ |
| 624 | 5′- GTGGCAGCTGCGATYTYYCC[A]HTGCCACAAATGGTAGTTAC -3′ |
| 625 | 5′- TTGGGAGGAAGACCACAGAG[A]TGATGTGCCAGTCTCAGAAC -3′ |
| 626 | 5′- AAAATACAGGGTACAGGGAC[G]CTCAAAGAGTGATTTGCTTC -3′ |
| 627 | 5′- GTGAGATGGGGCACAGCAGC[A]GCCGGAAGGTTATTTGTGTG -3′ |
| 628 | 5′- GCAGGGCAGAGAAGGGGAAG[T]TGCTGGCTGCCCTCCTCACT -3′ |
| 629 | 5′- GCTCCTGGATTCACTCCTTT[G]ATCCTCACCTCAATCCTTTG -3′ |
| 630 | 5′- AGTTGGCTTGTATGGACCCC[A]CCGATGACGGACAGTTCCAA -3′ |
| 631 | 5′- AGTGGATTGAGGATGGACAT[A]TGTATCTGGAAGCACCAAAA -3′ |
| 632 | 5′- CTGGGTTCACTGGAAATCAG[C]ATTAAGAATGTACAAGGGAA -3′ |
| 633 | 5′- ATGTAAACTGCCTTTGAAAG[G]CTATAACACAGTTCAGTTGG -3′ |
| 634 | 5′- ACTTAATCTTGCTCAGTTCC[C]CAGTTTACACTTTTGAATGG -3′ |
| 635 | 5′- GCAGCATAGATGAATGTAAT[G]TTGAAACAGGAAGATGTGTT -3′ |
| 636 | 5′- CTTAGCCTGCAATTGCAATC[T]GTATGGGACCATGAAGCAGC -3′ |
| 637 | 5′- TAGCCGTTTACAGAATATCC[A]GAATACCATTGAAGAGACTG -3′ |
| 638 | 5′- GTTTCAGATTTTGATAGGCG[T]GTGAACGATAACAAGACGGC -3′ |
| 639 | 5′- ATGAGGGAGAAATGCCCTTT[C]TGGCAATTGTTGGAGCTGGA -3′ |
| 640 | 5′- AGGAACAGTGCTACTTACTG[A]TGGGTAGACTGGGAGAGGTG -3′ |
| 641 | 5′- TTGGCAATGGGTAAGTCTAT[T]GTACTGTGTAAACTTGGACT -3′ |
| 642 | 5′- GATATAGATCTCTTGGAAAT[A]TAATAATGGTATGCAGGAAG -3′ |
| 643 | 5′- GCAACCCGGGGAAATACAGC[A]AAATGCACAAGTACTGGCTG -3′ |
| 644 | 5′- CTGTACAAACTTTCTTCCAT[G]ATTTTGATTATATCCATTTT -3′ |
| 645 | 5′- CCCTCATTATCTGCCTAAAC[A]ATTTTTTCTCAACTCCTATA -3′ |
| 646 | 5′- CTAGCACTGTACACACCCCA[T]ACTGTGTATGCTATTTGTTG -3′ |
| 647 | 5′- CAAAAGTTATCTCTAACCAA[G]GTACTCAAACAGAGTCTTTA -3′ |
| 648 | 5′- CCTTGTAAATCTCCACCTGA[T]ATTTCTCATGGTGTTGTAGC -3′ |
| 649 | 5′- TCCCATAGGAATTATAAAAT[T]GAAAAGTATGACAAAAATTT -3′ |
| 650 | 5′- AGGCCCTTCAGCTTCACCAC[T]TGCTTCTCTTTAAACAAGTC -3′ |
| 651 | 5′- GATAGAATTTGGCCCAGAGA[A]GTTAACTAATATATCCATGA -3′ |
| 652 | 5′- CTGTTTCTCCTTAAAATGGA[A]AAATGGCCTCTACAGAGTAG -3′ |
| 653 | 5′- GCTTGGTGGGGCCACTGGGC[A]TCTGTTTCTCGGGTGTTTTG -3′ |
| 654 | 5′- CCATTCCCTCGGCGAAGAGC[A]GAGGTTGAAGAAATGCTACT -3′ |
| 655 | 5′- GCAAGGGCCAGAGCCTCTGT[A]TGCTGCATTCGGCAACCACA -3′ |
| 656 | 5′- GGTTCCTGAAGGAGGAGTGG[G]AGTTTGGTAAATGGATGGAG -3′ |
| 657 | 5′- TTACCTGCTAAGGCCTGCAA[C]CTTGAGGATGTCCAGGGCTG -3′ |
| 658 | 5′- CCAGAAGGTTTCTTTGCTCC[T]CTTCCCTACAAAGACAGAGC -3′ |
| 659 | 5′- AATTCACTCCTTTAAAATAC[G]CAATGCAGTGTTTTTAGAAA -3′ |
| 660 | 5′- CCACTCCCTCTCCTGCTCTT[A]TGTGTGTGATCCAAAGGGAA -3′ |
| 661 | 5′- CAGGGACAGCTGAAGCCAAG[T]TCTCCCAAAGCAGCCTTGGC -3′ |
| 662 | 5′- GTCAGGAGCCTGGCCAGGCC[A]CACCCCTTGCTGTCTCAGCA -3′ |
| 663 | 5′- GGAGATTCTGCCTCAGGGCC[A]TGAGAGTCCCATCTTCAAGC -3′ |
| 664 | 5′- GCTCAGCTACCGTTGGTGGC[G]TTTATTAAACTGTGCACCCA -3′ |
| 665 | 5′- AAGGTGGCTGACTCCAGCCC[C]TTTGCCCTTGAACTGCTGAT -3′ |
| 666 | 5′- TGAAGACCTGAAAAGCAAAT[G]CCAGGCAGCCCCACTCCCTC -3′ |
| 667 | 5′- TTCTTTGTAATTTGGAATCC[T]CCTAATTTCCAAATGGGTTC -3′ |
| 668 | 5′- GGGACCTGGCCCTGGCCATC[T]GGGACAGTGAGCGACAGGGC -3′ |
| 669 | 5′- AGGTGGGGACCCGGCTCCAA[G]GGCACCCGGGTCTTCTGCAG -3′ |
| 670 | 5′- ACAGGCCGCTCTCCCAGCAG[T]GTGTTGAGGTGCACAGCCAG -3′ |
| 671 | 5′- TGGCGCAAGAGAACCAGGGC[A]TCTTCTTCTCGGGGGACTCC -3′ |
| 672 | 5′- TGCTGTGCCCACATCCCCTG[G]AACAGGCAGCCCAGCCTGTG -3′ |
| 673 | 5′- TGGTGAGTTATGGACCCYCC[C]ACCTCCACTACTACACTGTA -3′ |
| 674 | 5′- TCAGGGCCTGGGGCAGGCGC[T]GCACAGCCCCCACCGCTGCT -3′ |
| 675 | 5′- GCATGGCATGCGGAAGATGG[C]GAAGAATGTTTTATGGCCTC -3′ |
| 676 | 5′- TCTCAGTAGCTGAGACCTGA[A]AAATTTGGAGAATCACTTTG -3′ |
| 677 | 5′- ACATGAGGCCACTGAGGCAG[G]CCTCTTTCCTTCCCCTTCTC -3′ |
| 678 | 5′- CCTATTCTTAATCCTATTTT[A]CAAATGAAGTGACTTGCCCA -3′ |
| 679 | 5′- GGAATGGGTCAAGAATGTTC[C]TTCCCTTCTGAATGTCCCTG -3′ |
| 680 | 5′- AAGCGGGGAGGAGCTAAATA[A]TATTTTTCTCTCCTTGTTCA -3′ |
| 681 | 5′- AACTTGGAACATCTCCGCAA[T]AAGACAGAGGATCTGGAAGC -3′ |
| 682 | 5′- ACATCGCAGAAGGTGGCTCG[G]AAATTCTGGTGGAAGAACGT -3′ |
| 683 | 5′- TTCCCGAGGCCCTGCTGCCA[C]GTTGTATGCCCCAGAAGGTA -3′ |
| 684 | 5′- TGAGAGTCAGGGTTTCGGAC[T]AGATTGGCAAGTCAGGCTCT -3′ |
| 685 | 5′- TCTCCAGGACCTAGTATGGT[A]CCTGACCGTGGCACTCATAG -3′ |
| 686 | 5′- CTACCTCAGAGTATGTGCCC[G]TTGGATGGTGGCTGTTATTC -3′ |
| 687 | 5′- CTAGTCTCTGAGCTGAGTGC[T]GACTTAGGGAGGCAATGTTA -3′ |
| 688 | 5′- ACAGTGTGGCGTAAGGCAGT[A]TGGCCCTTGTCCTCTTGCTT -3′ |
| 689 | 5′- TTAGGGCAGCTGTGCATTGA[T]TGGGTAGACGCCATTCTGGA -3′ |
| 690 | 5′- TGAGGCCCCCACCTGGCCCT[C]ATCTGCCCCTGAGATCTAGA -3′ |
| 691 | 5′- CGCATAATTTCCGTCACCTC[G]TTCGCCTGCTGCTGGCACCG -3′ |
| 692 | 5′- CCCCAACATGTGCACCCCTG[T]ATTTCCTGTCATGCCACAGA -3′ |
| 693 | 5′- CCAGATCTCCATCATTGGCG[G]TAGTCTCTGGTCACCTGACT -3′ |
| 694 | 5′- TTTGTTCTGACTTTACATCC[T]CTTCCCCAGGTCACTTTTCA -3′ |
| 695 | 5′- ATTCCTGTCCCTTGTGCCGC[C]ATGAGCTGCCCACTGATGAC -3′ |
| 696 | 5′- TTTGATACCAAGAACACATT[A]CTGCATGAATCCTCCAGCAA -3′ |
| 697 | 5′- TCTAAAATTAGGGGTTTGAT[G]TAGCTTATCTGGAAGGTGTT -3′ |
| 698 | 5′- GATGCGGTCTGGAAAGCACC[G]GGGTGGCCGTCGGCTGACGC -3′ |
| 699 | 5′- CTCCGTGGAACTTCTCCTGG[C]ACAAATTCTGTTCCTAGGGA -3′ |
| 700 | 5′- GAGGGGAGCCACAGGAATGG[T]CGTGGCCAGAAGCCCTTCTC -3′ |
| 701 | 5′- GGCACCTTTTCCCTGATAAG[C]CACAAATCATAACCAAACAA -3′ |
| 702 | 5′- TTGCACTCCAGTTTTTTTTT[T]TTTAAAAAAGCGGTTTCTAC -3′ |
| 703 | 5′- GAAAAGGCTGTCTGATTATC[A]TGTCATCCAAAAAAAACAGA -3′ |
| 704 | 5′- GAACTAAGAGGAATAAAGGT[G]TTGCTTTATACCTGTCCCTA -3′ |
| 705 | 5′- ACTAACATGTCCTGCCTATT[T]TCTGTCAGCTGCAAGGTACT -3′ |
| 706 | 5′- GCTGACCCAGGGTCCACATG[T]TCTTTTTCTAACTTGTTCAT -3′ |
| 707 | 5′- TGCTTCCCCATTTCTGTCCT[G]AAAGCCCTCTGGCAAGACTG -3′ |
| 708 | 5′- CAGTGATGAACTCCTGGGCT[G]AAGTGACCCACCCGCCTCTG -3′ |
| 709 | 5′- GCGACTTCGACTAAGCAACA[C]TGCATCTATTTTCATGCAAC -3′ |
| 710 | 5′- CCTCAAATGTTAGAGTCAGT[A]CACCAGCTCATAGTTTCCAT -3′ |
| 711 | 5′- CGTTTAATTCTTTCTCATCA[C]TTTCCTAGGGCATTTGCAAT -3′ |
| 712 | 5′- CATCAGAGTTTTATGATTAG[C]AGATATATCTTAACTGACAC -3′ |
| 713 | 5′- AGCAAAACCAAAGAAATCAGC[A]GAAGACCATAAAAACAGACG -3′ |
| 714 | 5′- CTATAAAATTAGTATGCTTA[C]AATTATTAAACATATACAGA -3′ |
| 715 | 5′- TAAACACTTTAATGCAGTGA[C]ACTCAGGTATAAAACTCAGA -3′ |
| 716 | 5′- ATAGAAGACAAAGTTTTCAT[T]CGTCTCATTCAAGTTCACTT -3′ |
| 717 | 5′- AGTGCAGGGCAGGACTGCTG[G]CTGACCCCGGGCCACCTGGA -3′ |
| 718 | 5′- AACCTCTTGGTACATGTTAG[A]GGAAATGAAGCTGGCAACAA -3′ |
| 719 | 5′- TCATCAGATCAAGGACATTA[C]GGAATTAAAGGGCTCTAAGA -3′ |
| 720 | 5′- CCACTGCTATTGGTTATTTA[C]CTAGCATCCATTTCCCTTTA -3′ |
| 721 | 5′- ATCTACCTCTCCTGCCTCAT[A]TATTATTACCCAGCCCCTTC -3′ |
| 722 | 5′- GTCAATTGCAAATGGAGGTG[A]GACCTGAGAAAACAAAGAAA -3′ |
| 723 | 5′- GAGTGTGTAACAACTCACCT[G]CCAAATCGACTAGCCCTTAG -3′ |
| 724 | 5′- CTTGTAAGCCATCTTAAGCC[G]TTATAGGCCTAAGATGTATA -3′ |
| 725 | 5′- CTTGAGACCTGTGTCTCCTC[A]TGTTCACACTGTTCCTGACT -3′ |
| 726 | 5′- GAGGCATGGGTTGAACTGCA[T]TCACATATGTACTTAAAAGA -3′ |
| 727 | 5′- TGTTTCTTGAAGTTTGACTA[C]TTAAAAACATAGGTGTAAAG -3′ |
| 728 | 5′- AGAGTCACGGCATGTGGGAA[T]GTTTCCATGGACACTGGATC -3′ |
| 729 | 5′- AATGAGATCTTATGTCAAGG[A]TTTAATCTTTGGTATTCCAA -3′ |
| 730 | 5′- TCTGGACCTCAGTTTCCTCA[A]TGAGCTGGTAAGAATGCACT -3′ |
| 731 | 5′- AGGTTGATAGCAATGTTTGG[G]AGATATGTCCTAGAAGTGTT -3′ |
| 732 | 5′- GCATGATAACCCGAGCCATC[A]CTAAATATTATAGCTTCCTT -3′ |
| 733 | 5′- CTCCAGTTTCTCCCTTTCTC[G]CCAACTAGGTCCATCCAAAC -3′ |
| 734 | 5′- AACTGTAAGGATCTCTTGCT[T]TATATACTATTGGGGGAACA -3′ |
| 735 | 5′- CCTTAGCTCTTCCTAAAACA[C]ACAATCATAAAGGAAACCGT -3′ |
| 736 | 5′- CTGACAGTAAAGGGAACTCA[G]TATGTCTGAGTCTTTGCTCA -3′ |
| 737 | 5′- AACATTTACAGAAGCGAGAA[A]AAGTTTTGTTTGCTTTTGTT -3′ |
| 738 | 5′- TAAGTTCAATAAATCCCAAA[C]TGCACACTCTGAATTAGGGG -3′ |
| 739 | 5′- AAGATAGCCATCTTTGGGCA[G]AGAGTCATGAAATGTACCCT -3′ |
| 740 | 5′- GCTGGGCCGACGGGGACGAG[A]CGGCGACTGGAGCAGCAGCG -3′ |
| 741 | 5′- CTCTGTCTTGGTCACTGTGC[G]AGGATTGAAGGGAACTATTG -3′ |
| 742 | 5′- ATCGTCTTTTACAATAAGAT[G]CATGCCCCTATGAGTATTTT -3′ |
| 743 | 5′- AAGGAGAAAAACAGTGAACC[A]TAGTTCTTACTGCTCACACT -3′ |
| 744 | 5′- GATTATTTGATTGCCATGAA[C]GAAGCTGAATTACATAATTC -3′ |
| 745 | 5′- AGGGACCTGTCTTCAGAATC[T]AAGAAGCATAATGTCCTTAA -3′ |
| 746 | 5′- TAGAGTCCCTACCATGCACC[C]TGGGCAAGAAGTCAGTTCTG -3′ |
| 747 | 5′- TCGGGTCTCTTACCATGCCC[G]CCCTCCCTTCCTCAGGGAAT -3′ |
| 748 | 5′- AGGACCTTCAGAGACCCCGC[G]TTCTCTGAAACCAGGATGGA -3′ |
| 749 | 5′- CAGGGGCTGCACTCACCATC[G]TCTGACACCTCCACTTCATC -3′ |
| 750 | 5′- GTACACAAGGGTAGGGCAGA[G]GATGGACAGCAGGGCAGAAT -3′ |
| 751 | 5′- AGTTTCTGCAGCACTTTATC[T]TTCCATCTGGCCATGAGGAA -3′ |
| 752 | 5′- CAGGCATTGAAGGTCAGCTT[G]TTCTCCTCCTGGGTGAGTTT -3′ |
| 753 | 5′- GGGCACGACCTACCATCCAC[G]GTGACTTGGCAGGAGCACTC -3′ |
| 754 | 5′- TTACTTCTATCCTTGCTTCT[T]GAACTGGTCATTCCCTGACT -3′ |
| 755 | 5′- AGAACAAGCTGTTAGCAGGA[C]GCCTCTGCTGCTGCGGGGCC -3′ |
| 756 | 5′- TCGGCTGGGATCTCCTTCAG[T]TCGTCTTCCGATAGGGTCTT -3′ |
| 757 | 5′- AGGCCTCAGGGACCCATAGC[A]GTCACTACCACCACCATCAG -3′ |
| 758 | 5′- TTGTCCAGAAATCACTGTGA[C]TGGATACACAAATGCAGCAC -3′ |
| 759 | 5′- CTTGGCTGCTGAATGGTGAG[A]TCCCCCTGCCCCAGCTCTCT -3′ |
| 760 | 5′- GAAGTCTTCTGAAGGACCGG[G]GTCTGCGGGGCCGTTCTGGG -3′ |
| 761 | 5′- TGGTGGCTTTTGTTTCTCTC[G]CAAATGACCTGTGTGGTGGT -3′ |
| 762 | 5′- AGGACGGGTCTCCACTGCTG[G]AGCTGAAAATCTATCCCTGT -3′ |
| 763 | 5′- TTTGTGACCTTGTATGGATG[ACTTA]ACTTCTCTGAATCTTATTTC -3′ |
| 764 | 5′- AAAACTCAATAAGATGCCTA[T]ATTTTATGCATCTCCATTAA -3′ |
| 765 | 5′- TTCACCATCCCTCTACTTTC[G]GCTTGCCAAAACTTACAGGA -3′ |
| 766 | 5′- TGGCCAGTGCTCAGCAGATG[G]AAGTTCCAAATCGAGTCACT -3′ |
| 767 | 5′- GCATGGAGTCAACTCTTGAG[T]GATCCACACTGAGGGAGGTT -3′ |
| 768 | 5′- TGACTCCTGGTCCAGGGCCT[A]CTGGGGACTAGATAAGATGT -3′ |
| 769 | 5′- CAAGCTAGAGACTTGGTATA[C]AGCAGCAGTTACATGAGTGG -3′ |
| 770 | 5′- CAGACTGTGGACATCCGAAT[T]GGCAATGACATGAATTTAAG -3′ |
| 771 | 5′- AGGCACCAGGTCCCATGGCC[G]GTTTCCCCTGAGAAAACATT -3′ |
| 772 | 5′- ATGGAGAGCTGCCAAGCCAA[C]CCTGCCAGGGTCATCAGCTC -3′ |
| 773 | 5′- ATAGCTGTCCTTACTCCTTT[C]CTAGACAGACAGTGTCTTGG -3′ |
| 774 | 5′- GCTTTTTATACCGCTTAACG[A]AAATAATTTAAAAGGCTGTC -3′ |
| 775 | 5′- AGCTGCAATGCCTATGAGCA[G]GACCTGGGTTTGTACATCTT -3′ |
| 776 | 5′- CTAGGATAGCAGAGATATTA[C]TTCAGGATCAGATCTTGACT -3′ |
| 777 | 5′- TCTGGGGAGTCTTTAGCCCC[C]AGCAGAGGCCATTTCTAGCA -3′ |
| 778 | 5′- GAATAAAACTTACGGAGAGC[C]TCTAACTTCATTCAATTTGT -3′ |
| 779 | 5′- ATAATATATTTTAAGCAGGG[T]AGGGTATCCCAAGATCTCAA -3′ |
| 780 | 5′- GTATGGTAAAGAATCCCACT[C]CTGCATCAATCAGTGGGCAA -3′ |
| 781 | 5′- TTTTCCTTACACCAAGCTTA[C]GTGGGTGGCTGTAGCCACAA -3′ |
| 782 | 5′- GCACCATGGGGGAAATTATC[G]GTATTATTTTTTTGAAATCA -3′ |
| 783 | 5′- TATAGYCAAAGAGTTGTGCA[C]TGATCACCTCAATGAATTTA -3′ |
| 784 | 5′- GTTCTGGGCAACTGCTTTAG[T]CTGAATGCAAAAAACTGGAA -3′ |
| 785 | 5′- AAACAAAAGCCCCACAGCAA[A]AAACAGGAAGGAAGGGGAAC -3′ |
| 786 | 5′- ATAGTGAGGGATGACTGTAT[C]TTCCACTTAAAAATCCCAAG -3′ |
| 787 | 5′- GGAAAATAAAACTGTACCTC[G]TCTCCAGTCTCCCCATATTT -3′ |
| 788 | 5′- TAATGGCTTTCAAAGTGCCT[G]AATTCCATTCTACACTAAAA -3′ |
| 789 | 5′- ACCTCAAAAGAAAAAATAAC[A]TAAACAATATTCAACTCAAG -3′ |
| 790 | 5′- GCTTGGTTCAGGCCCTGGTT[A]CATACCTGGATTTCAAATCT -3′ |
| 791 | 5′- ACCCACAGCTTTCAGCAGTG[A]AGAATATGAATGGAAACTGG -3′ |
| 792 | 5′- GAGTGAGGTAGAGAACAGGT[A]TAATTCACCATAAGTCCTGA -3′ |
| 793 | 5′- ACCTGGTTCTTTGAAAGAAC[A]AATAAAATTCACAAACTGCT -3′ |
| 794 | 5′- TTTTTCTCTTCAGCTGGCCC[G]AATTGGTTTCTGTTAATTTT -3′ |
| 795 | 5′- GAAGAGACTAAGAGAATCAC[G]GAAGAGAGAAGGAGGTCAAG -3′ |
| 796 | 5′- TCTTGAAGGGTTTTAGTTCC[G]TAAGTTCCAGGGAGGGGTCT -3′ |
| 797 | 5′- AAACGTTTAATTCTTCTGTG[A]GTTCTGTTCTAATTTCTGAG -3′ |
| 798 | 5′- AGGCCTAGAATTCTCTGAAA[C]GTCATTTTTCAGTTTCTACA -3′ |
| 799 | 5′- GTAGCCTTGCGCCTCACTCT[C]GTGATGGAGCCGCCTGCTAC -3′ |
| 800 | 5′- ATTGTCATTTTCCTTGTGTT[T]TATTGGTTCAGGCTATCCAA -3′ |
| 801 | 5′- CAAGGCATCTTGGCTCCTAC[A]TAGGGCCTTTTGGCTCCTCT -3′ |
| 802 | 5′- AGATCTCCAAGGTTTTCACC[A]AGAAACACTTGACCCGACTT -3′ |
| 803 | 5′- GCTCAATGCAGAGGGGTCAT[A]AGAGCAGGCTGGGAGCCAGA -3′ |
| 804 | 5′- GTTCCTCCTCAGAAACTGCC[G]TGTATGAGTTTGTATCCTTA -3′ |
| 805 | 5′- CATAGGCGAGGCCCAGCCCA[T]GTGTCCAGAGACATCTGTGA -3′ |
| 806 | 5′- GCTCTTCAAGGTCTGGTGCT[C]TCTTCCACAGTACTGTAGCC -3′ |
| 807 | 5′- AAATGGGTGCTCAGACCCCT[G]TCCTACTTACCTCAAAAGGT -3′ |
| 808 | 5′- TGTCAGCAGCCTGGTATTGG[A]AAGAGTTAAAGGAAAATCTC -3′ |
| 809 | 5′- CAGTTCAGGGGAGGAGCCTC[G]GGACGTCAGTGGCAAAATCA -3′ |
| 810 | 5′- GCATAGGCTTAACTCGCTGA[A]GAGTTAATTGTTTTATTTTT -3′ |
| 811 | 5′- AGGGGAAACGTCTCCCAGAT[T]GCTCCCTTGGCTTTGAGGCC -3′ |
| 812 | 5′- AGCCAAAGCCAGAGTGGCCA[T]GGCCCAGGGAGGGTGAGCTG -3′ |
| 813 | 5′- TTTCAGAGAGGGAAGCCAGA[A]GAGAAGAGGGTGCAGGCTGA -3′ |
| 814 | 5′- CAAGTCCTCCGGTTCTTCCT[T]GGGATTGGCGGGTCCACTTG -3′ |
| 815 | 5′- AGGCTGCCTCCGCACCTGAC[T]GCTGCCCAGGTGGGGTTTCC -3′ |
| 816 | 5′- TGGCTAGGACAGGGTCTCGG[A]CTAGGGAAGTGGTTTCTCTG -3′ |
| 817 | 5′- TTACGGGAAGCCCTTCTGGC[A]CTCACTCAGGGCAGCAGCTT -3′ |
| 818 | 5′- GCCTGGGCAGGAAGAGGGAC[G]AGAGGGTCTCCCACATGGGA -3′ |
| 819 | 5′- ATCGTGTTCCCCAGGAAGTT[A]TTCTTGATTTAGTTTAAACT -3′ |
| 820 | 5′- GAACCACCTTCTCTTGCCAG[G]CTGTACTCCTCATTTAGTTT -3′ |
| 821 | 5′- AAGGTGGGAGCCAGAGTGGG[T]TGCTGTAGGGGTGAGGGAGG -3′ |
| 822 | 5′- GCCATCCAGCGCGGCTGCTC[G]GGCGCCACCTCCATGGCCGG -3′ |
| 823 | 5′- TCCCTGGGCCCGTCGCCCTC[T]GGGCTCCCGCCGGAACTCCT -3′ |
| 824 | 5′- ACACAGACATTGTCGAGCGC[C]GGTCCCTCTTTATTGGCCAG -3′ |
| 825 | 5′- GCCTGGTGAGAGCAGATTTA[T]TCCAATTTATGGGCTGGAAC -3′ |
| 826 | 5′- CACACCGACACACATGGCCA[T]ACAATCAGATGCAACTCGGC -3′ |
| 827 | 5′- CTTGTTCACAGAAGTGGGAG[T]CAGGAGGGGGGGAGAAAGTG -3′ |
| 828 | 5′- AGGACCAGGCGGCTAAGCAG[A]GAGAAGAGCCAGAGGGGCGT -3′ |
| 829 | 5′- CGGGCCATGGACACCGACAC[A]CTGACACAGGTCAAGGAGAA -3′ |
| 830 | 5′- CTGCGGTTCAGCTCCTTGGT[C]AGATCTGTCATGTCTGTCTG -3′ |
| 831 | 5′- GCACGTCGGCTCTTGGTACA[A]AAGACGAACAGGGCTGCGGG -3′ |
| 832 | 5′- TCCCCCGGGGCCCTGAGCAA[T]GCATCAGCGCCAGTGGACTT -3′ |
| 833 | 5′- TTCACCAGGACCTGGAGCTC[A]GAGCCTACATGGAGGTCATT -3′ |
| 834 | 5′- ACGGTCACCACACCTGAGAG[C]GGTCCTGGGGCTGGCCCTGT -3′ |
| 835 | 5′- GCGGCAGCCATCACTCCACA[C]GCACAGGTGACCCAGGTCTT -3′ |
| 836 | 5′- AGGATGTTCTGGGAGCCACC[G]GTAGGCACGGGTGCCAGGGG -3′ |
| 837 | 5′- TGGAATGAGCAACACAGGAA[G]GCTCCAGTTGTCCAGACCAT -3′ |
| 838 | 5′- CGAGACTGGTTGGAAACACA[A]GAGTGCTGCTGGCTGCACCA -3′ |
| 839 | 5′- CCCCCATCCATTCCAGACCA[T]GTGACTGTTGAGATGTCTGT -3′ |
| 840 | 5′- TCGATGTGCGCCAGGAGTAC[T]CAGTGAGTCCTGGGGGAGGC -3′ |
| 841 | 5′- AGTTTGACCCAGCAGACTCC[A]GTTACCTTTACCTGATGACG -3′ |
| 842 | 5′- CCTACCTTGAGAAGCCTCCC[A]TTGACCGTGCCCAGGAAGAC -3′ |
| 843 | 5′- AGGCCTCCAGGAAGTGACCC[T]GAGACAATAACTGTGCAACT -3′ |
| 844 | 5′- GTAACTAAGCACACCCCTTA[A]AGAATTTTGGGAAGTCGCCC -3′ |
| 845 | 5′- TAAGCCAGAGGATGCTGTAG[C]GAGTACTTGTATGCAATAAC -3′ |
| 846 | 5′- CTTGTTGTCATGGTGCGTTG[A]AAGAGTAGCCAGTTGTCTTT -3′ |
| 847 | 5′- ATTAGTATGCAGGTCTTATC[C]ACCATTGGAATTAAGCTGTT -3′ |
| 848 | 5′- ACGTTTTTATCACACATTAA[A]CACTTGCATTAATTTTGGAG -3′ |
| 849 | 5′- GATGAGTTAAATGGGCTAGT[A]TCTAAATTTTAAATTTTTAC -3′ |
| 850 | 5′- GTACATCCCATATTCCCTTT[C]CAAAATCTAGTTTCCTATGT -3′ |
| 851 | 5′- GCTTACCAGAAAACACCCTC[T]TTGTTGTTTTTATTTCTCAG -3′ |
| 852 | 5′- GGACAAGGAGGAGAAGCCCC[G]GGAGGTCACGGGAGTTCACT -3′ |
| 853 | 5′- GAGCAGCCATTTCGAAAGGC[G]GCAGAAGAGGAAATTAACTC -3′ |
| 854 | 5′- GCGAGGGGAAGTCATTTTTT[G]AATAACTAGGCTCTATTTGC -3′ |
| 855 | 5′- CAAGGAAAGACCTGGTGTCC[C]TGTGCTAATTTTAACTCTCT -3′ |
| 856 | 5′- TACAGATGCTCATAGGCATC[T]GAAAAAAAAATACTTTGTTA -3′ |
| 857 | 5′- AACTCCTTTGACAGTATGGA[T]GGCACCTAACGCATCCTTGT -3′ |
| 858 | 5′- GAGGTGTTTTCTTGGCTCTT[C]ACKAACGTTTTTAATAAAGC -3′ |
| 859 | 5′- GCGCCCCCTGGAGTTCTGCT[G]GAATTTAGATTTAAATAGAT -3′ |
| 860 | 5′- ACATATTTAGAATGGATGCC[A]GAACAGGAGAAATGGGTGGG -3′ |
| 861 | 5′- ATTCATATGCCACCAGCCAT[T]GGCAGAAATGTAACAGGAAA -3′ |
| 862 | 5′- ATGGCTCTGTAAATGGGATG[T]CTCATGTTCAGGTTTCTGGA -3′ |
| 863 | 5′- ATCTCCAGGTGAACATGGAA[T]GCAGTGAAAACCTGGGGTAT -3′ |
| 864 | 5′- TGATAAGTAGTTAATGATCC[A]GAAATAAACTGTTAGGTGCT -3′ |
| 865 | 5′- AAGTAAAATAGTAGATATTG[G]ATTGCTTCTACATTTACTAC -3′ |
| 866 | 5′- AGAGCCCCTACCCAATTGCT[T]TACTATTTATAGTTCCTCAG -3′ |
| 867 | 5′- ATCTGGGGACCTGCTCCTGG[C]AGAGCAATAGGAWCTGTGTG -3′ |
| 868 | 5′- GAGTCCCAAAATTCAACCCT[T]CCGATAGGGCTGGGCCTGAC -3′ |
| 869 | 5′- CCCTAGCCTGCTTTTGTCCT[A]TTATTTTTTATTTCCACATA -3′ |
| 870 | 5′- AGAGGGAACCCAAATATTAG[A]GTGGGAAGCAAGTCATAAAC -3′ |
| 871 | 5′- TAGGGTTACCAATCCACTAG[G]ATGCAAAACTGTACTTATTA -3′ |
| 872 | 5′- AGGCTTCTTTTTCCATTACA[T]TGTAAGACTTTGGAGGGCAG -3′ |
| 873 | 5′- AGCRGTCAGGTGCGGAGGCA[A]CCTCTCAGCGGTGGGGAACA -3′ |
| 874 | 5′- CAGGACAAACAGTGGATTCA[T]TCAGAACACAATATGCTGGT -3′ |
| 875 | 5′- AAGCCACTACAGACACCGCA[T]GCACCGAAATTCTCCCTTGT -3′ |
| 876 | 5′- ATCACTGTCCCTCAGTTCAC[T]GGTCTTGTCTGCTTCGTCGY -3′ |
| 877 | 5′- AATTCTCAGTCTTAAAAACA[G]GGCATAAAGAAAGCTAAAAT -3′ |
| 878 | 5′- AGAAGATAAGTGTTTAGGGT[A]TTGGATATCCCAGTTACCCT -3′ |
| 879 | 5′- CCTTTTTTTGGATGATCCTA[G]AATTAATACAAGTGTATTCT -3′ |
| 880 | 5′- GCCCTTAGTCACCAACTCCT[A]CTCATCCCACCATGCTGTTG -3′ |
| 881 | 5′- GTAAATTAAAATTTGTTTGG[G]TGATTTGTGCTGTATTTCTA -3′ |
| 882 | 5′- AGCAACACTTCCTCCTTGCA[T]ATTACAAGCATAGCTAATGC -3′ |
| 883 | 5′- CCCTCATTTTCTGTTAGGGA[G]GTATGTGTTTACCAAGCTGT -3′ |
| 884 | 5′- ATGAGGGCTTTACTTTTGCA[A]GAAATACTACAGATGGTGAA -3′ |
| 885 | 5′- TCCCTTCTCAGTAACTAACA[A]TAATCATCTCTCTGGAGGAC -3′ |
| 886 | 5′- CATTCCCTCACACAGTACAG[A]TTAATAAATGTGCATTTTGA -3′ |
| 887 | 5′- CCTGTGTGATGAGGGGCAAA[A]GAAGCTCTTGAGAACCTGCT -3′ |
| 888 | 5′- GTAACGAAGAAAGACCAGAG[C]GTCATCCCTGTGATACAGCA -3′ |
| 889 | 5′- TATGTATCTTGCTTTTGTTT[C]AAACAGTCATCCACATTAGT -3′ |
| 890 | 5′- GATAGGTTGCAAAATTTTGG[T]GTGTTCTTGCATTGCATACA -3′ |
| 891 | 5′- ATTGACGGTGTTATAATTAC[T]ATGGTTTTGAAATTACATAG -3′ |
| 892 | 5′- TGAGGACCCAGATGTCAACA[T]CACCAATCTGGAATTTGAAA -3′ |
| 893 | 5′- CTCCTTTTGACCTGAGTGTC[G]TCTATCGGGAAGGAGCCAAT -3′ |
| 894 | 5′- TATGTAAAAGTTTTAATGCA[T]GATGTAGCTTACCGCCAGGA -3′ |
| 895 | 5′- GATGGATCCTATCTTACTAA[T]CATCAGCATTTTGAGTTTTT -3′ |
| 896 | 5′- AATTAGCTGCCAGAGTTGCT[A]TCAGTAAAGAGAAGAAATAA -3′ |
| 897 | 5′- CTGAAATCAGAGAACATTGA[T]AGATGAAGTGAATGGCAGAG -3′ |
| 898 | 5′- GCCCATCTGAGGATGTAGTC[G]TCACTCCAKAAAGCTTTGGA -3′ |
| 899 | 5′- GTGCAGAYCAGATAATTATA[G]AGAGATGGAATGGGACAACC -3′ |
| 900 | 5′- AATCTGCCTCTGGGGCGGGA[C]CTGTCAGGCTTCAGGAAGGG -3′ |
| 901 | 5′- TCCAGGGAGGAGCTTCGTGC[A]ACCTTCCCGGACCACTCAGG -3′ |
| 902 | 5′- CATCACCTCCAGGTAGCTCC[C]AAAATGTCCCTAGAAAGTGG -3′ |
| 903 | 5′- GGAGCACAGAGTAGCAGTGA[C]GCTGTCCAAGGCAGGGGGGA -3′ |
| 904 | 5′- CATTCAGGCCAGTGGCTGCA[A]GGGAGCAGAAAGATCAGGCT -3′ |
| 905 | 5′- TACAGAGGAAGAAATCCAGG[A]CAGAGGTGGAGGCAGTGAAG -3′ |
| 906 | 5′- CTACCTCATTCATTGACCCC[G]CTATCTGACCTGTACATGTT -3′ |
| 907 | 5′- TTGAGGACAAACAGAACATC[A]GTGAGTAAGTGGAATATTAG -3′ |
| 908 | 5′- TTCTTGTGTTCTTCCCTTTC[T]ATTTCAACTCTTCATCTCAG -3′ |
| 909 | 5′- GGTTTGTGTACCAGGATTGG[A]GACCCCTGATGTATAGTGTA -3′ |
| 910 | 5′- GAAGAGGATAGGTTTTTCTA[T]CTTAAACAAAATCTTCCTTA -3′ |
| 911 | 5′- GTTAGGCATCAGGCAACTAC[A]AAGGAGTATACGAGCATGCA -3′ |
| 912 | 5′- CACAGGGTAAATTTAGCCAC[G]GCAGCAGGAGCATGATATAA -3′ |
| 913 | 5′- GGCATGTGAAATAAGTTGGT[T]TAATTAGAGTGAAGCCCAGG -3′ |
| 914 | 5′- TGGATTGTGTGTGTGGTAAT[G]GGATTATTGTTATATTTAAA -3′ |
| 915 | 5′- CACGAGCATCTTGCTGTCTT[T]AATTAAGAAGTTAACTGGAC -3′ |
| 916 | 5′- TTGAAAGCTGAGTCATTTTC[G]TAATGGGTCAGAAAGACATT -3′ |
| 917 | 5′- TACATGACGCATGTATTTGT[C]AAAACCCACAGATCTATTAA -3′ |
| 918 | 5′- CTGAGAGTGCAGTGAACCTT[C]GTGTCTGTGATGGAAGAGGT -3′ |
| 919 | 5′- GCTTAGATGTGAGAGTTGAT[T]CCATAATAATAAAAGTTATT -3′ |
| 920 | 5′- TTGAACTCTATGTACCAAGT[C]TGAACACATTCCAAATATCC -3′ |
| 921 | 5′- GGTATTTTGCTACAGCAGCC[T]GAGCAAACTAATATATCATC -3′ |
| 922 | 5′- AAAGGCGGTCACCTGCAGGA[G]TAGCCATCTTTGGTCCTTTC -3′ |
| 923 | 5′- CCCCCAGGGGTGGTAACAAC[G]GCACGCAAGCACAGCCATTG -3′ |
| 924 | 5′- CCACACCTGGTGGACAGGAC[A]ACCGTGGTGGCCAGGAAGCT -3′ |
| 925 | 5′- GGTTAAAAAGTTCTCTACCA[G]GGAAGTTGGATAAAAGTAAC -3′ |
| 926 | 5′- AAATCAGAATCGAATTATTG[A]TTTGGGGCTAATTGTATCTG -3′ |
| 927 | 5′- CCTGTCAGTGAAAACAACTA[T]CAAAGCTGGATTTTAAATAT -3′ |
| 928 | 5′- CCATTAGCAGTAGGTCTGAA[A]TAACTTTAATATGCAAGTTA -3′ |
| 929 | 5′- AGAGCCAGCTGGGAGAAACA[C]GCAACATAGTTCTTTGCAAT -3′ |
| 930 | 5′- AGCAGCTGGACCATGATCTC[T]TGGATATGGTGGTAGGTGAA -3′ |
| 931 | 5′- AGACGATGTACTGATGTAAG[C]TTTTGTAAATTTCTAAACTG -3′ |
| 932 | 5′- ACTCTGTCTTTCCAATTCCT[G]AACAGCATGCTTGGATGGGA -3′ |
| 933 | 5′- TCAGAAAGAATGGGGTAAGG[C]GAATTGAGTTTTAGAACATA -3′ |
| 934 | 5′- ACAGTGAAGAAAGAGGAACA[A]AGAGAAGGGCAGGCAGGAGG -3′ |
| 935 | 5′- TTGAAGGTGGATGAGGGAAC[A]GTCAGGTTGAGGAGCATTTT -3′ |
| 936 | 5′- ACACAATACTGGGTTTCTCT[A]CTTCTCTCTCACCATCACAC -3′ |
| 937 | 5′- CCACGCACCAGCAGGTTCAC[A]GTGCAGCTCATGCGGTTGTC -3′ |
| 938 | 5′- GATAGTCTAAATGAATGTCC[G]CCACCCCCGCCTGTAGTTGT -3′ |
| 939 | 5′- GCTGGCTGGGGCAAAGGTCT[T]TGATGCACTGTGCAGAAGTA -3′ |
| 940 | 5′- CTGCTCGGGCCAGAAAATCC[A]GAAACGGGCCCTTACCGATG -3′ |
| 941 | 5′- GTTCTGAAATGAAGACACAT[G]TGGCAGGCAGGTTACAACCC -3′ |
| 942 | 5′- CTCACTCACTCCTTGAGGAC[G]CTCTCATGACAACTGTAAAG -3′ |
| 943 | 5′- TTCAAAAACTATTTTGGTAC[A]TTTCAAATACAGTGTTTAAA -3′ |
| 944 | 5′- TGTTGCTAAGATCAATAGCT[A]CATTTGAATCTATGTCTCCC -3′ |
| 945 | 5′- CAGTTTATTATGGGTTATCT[G]ATTGGAATAAAGAGGTATCA -3′ |
| 946 | 5′- AATCATTATGTCACAAAAAA[A]TATATAAAGATAAATTTTTC -3′ |
| 947 | 5′- AGAGCCAAGACTTGTCCCCT[G]TTTCTGCAGCAGATTGGTCC -3′ |
| 948 | 5′- GTTTCTCAAAAGTTCTAAAC[G]TTACAGAGGATAATTTTAAG -3′ |
| 949 | 5′- CCTTGTCTGGAGGAGTTGGG[T]TTCCTCAATAATTGGCTGTG -3′ |
| 950 | 5′- GGATCCAAAGGGTGTCAAGG[T]GATCATTATCTTGGGATGGA -3′ |
| 951 | 5′- AATGAAACTAAATGATGATC[T]TTCAACTCTCCCTTCTCACT -3′ |
| 952 | 5′- AGTTGCTCCCCTCTCTGATC[T]ACATTCGTAAAATGACATAA -3′ |
| 953 | 5′- CAGGTGTCCCTACCTTAAGG[A]CCTCCTCCTTGGGACTTCAC -3′ |
| 954 | 5′- CACACAACTRGCTAAGGAGC[C]CCAGGGCCACAGCTGCTGTT -3′ |
| 955 | 5′- ATAAGCAGGAAAATGAATGC[A]TTAGGAGAGGTTTTATATCT -3′ |
| 956 | 5′- TTATGCATACAACACTCAAC[C]GATCCAGTTACTCTTACTCT -3′ |
| 957 | 5′- CACCCCAGTCACCGTGGCTT[A]CACCTGCACAACAGATTCCT -3′ |
| 958 | 5′- AATTTCCCTGCATTTTGTGA[T]GACTTGTTTTTATTGGTAAC -3′ |
| 959 | 5′- TGCGCATTTTCCGCACTCCG[G]TACACTTTACACTGAACACC -3′ |
| 960 | 5′- GACCCAGAGCAGGAAGCATA[A]TCAAGCCCTCCACTAGATTA -3′ |
| 961 | 5′- CACTTGGAAATCCTAACTCC[G]CAGAACAAAATTTTACAAGC -3′ |
| 962 | 5′- ACACACTGACATTCGAGGCC[C]AAGGAATACTCCTGCCTCTA -3′ |
| 963 | 5′- TTCATTTACAAGCCTGATCA[G]CCTTACATGAACTAATGTTT -3′ |
| 964 | 5′- AACACTGTTGCAGGATCTCT[A]ATAATCACTATGTACACTTC -3′ |
| 965 | 5′- AACTCCCCAGCTAAACACCC[A]TAAGACTTCATACAACACAA -3′ |
| 966 | 5′- TAAATGCTTATCCATTTAGT[G]ACAGGAAAAATGAGACAACT -3′ |
| 967 | 5′- GTATGCTTTCCATCGAAAAA[G]TACTCTATTAAACAGCTTAG -3′ |
| 968 | 5′- TATACAGGAGTCATCCCCTA[T]GTTGACACTGGTAAGTTGTA -3′ |
| 969 | 5′- TCAAGTTTAAGCTGCTATGT[C]CCTTATTTTTAACTTTTGTT -3′ |
| 970 | 5′- ATATAATTTATATTACAATG[T]AAAAGCTTCTTTAATACTAA -3′ |
| 971 | 5′- GATGGGGAGGAAGAGAAGGC[A]TTGGTCTTGCAGTCTTGTCT -3′ |
| 972 | 5′- AATGGTAAGCATCTATTTTG[C]AGTCCACTCTACTGAGCTAA -3′ |
| 973 | 5′- TTTATATATGATATCATCAT[T]AAGCACTTTCTATAAGCTGA -3′ |
| 974 | 5′- AACAATCTGTGAACACTTGT[C]ATATGCTTACTGTAAGTGTG -3′ |
| 975 | 5′- ACTATATGTCATGTCTACAG[G]CTGTCTCCTAAGAGTAGAGG -3′ |
| 976 | 5′- TGCAAACATTGGGAAACCAC[A]GTAGGGGGGAGCAGGACTCT -3′ |
| 977 | 5′- TACCATGGACAGCAGCGCTG[T]CCCCACRAACGCCAGCAATT -3′ |
| 978 | 5′- ACTTGTCCCACTTAGATGGC[G]ACCTGTCCGACCCATGCGGT -3′ |
| 979 | 5′- GCATTTCACATTCACATGTA[A]TATTTGAATATACACATCAA -3′ |
| 980 | 5′- TTGAGTCTCCTTCCAATTAA[A]TCATGGAACATCAGAGCCAT -3′ |
| 981 | 5′- TCTTTTGTGGAAATGTGATG[T]ATTTGTTTATATGCAGACAA -3′ |
| 982 | 5′- ACCAGACTTAGGAGAGATAT[G]TCTCACTGTAGAACCAGTGC -3′ |
| 983 | 5′- CTCTGGTCAAGGCTAAAAAT[G]AATGAGCAAAATGGCAGTAT -3′ |
| 984 | 5′- AGCCAAAGTTCAGTTCTCCA[A]TTCATCTGAGCTCAGGCCCA -3′ |
| 985 | 5′- GGTATCRTGGGTCCTTTCRAGTAC[C]AACCGCCTTAGGCTGGAAGC -3′ |
| 986 | 5′- TTTTACCGAAGGCTGTGTCT[C]GTAAGCACCCCCGAGCAACT -3′ |
| 987 | 5′- CTACTCCGGCACCCAGTGGG[C]TGGTAGTCCTGTTGGCAGGA -3′ |
| 988 | 5′- CCAAGAAGCGCGCGGCGAGA[A]TGCAAGGTGGGGGCCCCGCC -3′ |
| 989 | 5′- CTCTCGCCGCGCGCTTCTTG[A]TCCCTGAGACTTCGAACGAA -3′ |
| 990 | 5′- GAGCAGAGGGGCAGGTCCCG[G]CCGGACGGCGCCCGGAGCCC -3′ |
| 991 | 5′- AGAGCGGATTGGGGGTCGCG[G]GTGGTAGCAGGAGGAGGAGC -3′ |
| 992 | 5′- TGGGGATTCAGAGCACCCAC[C]CGCAGCACCTCCCTCCTCTG -3′ |
| 993 | 5′- GGGTCAGTCCGGACAGCCCC[G]GTCGCTTGTTACCTAGCATC -3′ |
| 994 | 5′- CTGGGTGCGCTGGCCGAGGC[A]TACCCCTCCAAGCCGGACAA -3′ |
| 995 | 5′- GACATGGCCAGATACTACTC[A]GCGCTGCGACACTACATCAA -3′ |
| 996 | 5′- CTGACAATGTCTGTGGCAAC[G]CTGCAGTTTACTCCTTGGTT -3′ |
| 997 | 5′- CAGACACCCACTCCTATGTG[C]GTTTCTGAAAATTACAGGGT -3′ |
| 998 | 5′- TCCAGATATGGAAAACGATC[T]AGCCCAGAGACACTGATTTC -3′ |
| 999 | 5′- ATTTCAATTTAGAGTCAGGG[A]CTCACTCTATGCTCCCCTGA -3′ |
| 1,000 | 5′- TGGAAAGAGGTGCCCACCAA[C]GTCTAAGTGTTAAACATTGA -3′ |
| 1,001 | 5′- TATCATGCATTCAAAAGTGT[G]TCCTCCTCAATGAAAAATCT -3′ |
| 1,002 | 5′- TGAAAAATCTATTACAATAG[C]GAGGATTATTTTCGTTAAAC -3′ |
| 1,003 | 5′- TATTTCTCAAACATTTTCAG[T]TTTAGAATGGGAATAGGTTT -3′ |
| 1,004 | 5′- GTGCCTTTAAACCTATTCTA[T]AACCTATTTAAACGTATTTC -3′ |
| 1,005 | 5′- AGGGCTGCCTGGTAAGCTGA[G]TCAGGGTGCCTGGCTGCCGC -3′ |
| 1,006 | 5′- AACGCCACTTGTGACTGCTC[A]TTACCTTTCAGTTGTGTCCC -3′ |
| 1,007 | 5′- ATGTTGGGATTTAACTTTCT[A]TTATATGTCAGACTCACTTA -3′ |
| 1,008 | 5′- TGTGTGTTTTAAATCTTTGC[A]CTTAAATGTTTTTGATTTCT -3′ |
| 1,009 | 5′- GAAGCTTCCCTCCGACAGGC[A]GCCCCGCACTAAGGTAGGGA -3′ |
| 1,010 | 5′- CTAATGGTTGGAAACGCCAG[T]CTTTGGTGAAAACAGAAAGT -3′ |
| 1,011 | 5′- TTCAAGAATTCAACTGCAGA[C]TGAAAATATTTGGAGAAAAA -3′ |
| 1,012 | 5′- AACCTAGCCACAGAGCCCGA[C]GCGATGTGTCCTTGTCGAGA -3′ |
| 1,013 | 5′- GCCTCCTTTGCTGCCCTCAC[G]ATCTCTTCCTGTGACACCAC -3′ |
| 1,014 | 5′- CTCTGCACCTTCAGGTTCAG[A]CCCTTCAAGATCTACCAGGA -3′ |
| 1,015 | 5′- ACAAGCTAGTTACCTTTTAT[C]GTTCAGTTTAAAAAAGTTCT -3′ |
| 1,016 | 5′- CGGTCCCCTTCAAGATCCAT[T]CCGACCTGAAGAGAAACCGC -3′ |
| 1,017 | 5′- TGCTCTTCAAAAAAACCAGA[T]TGAATATTTTTAAAAGTAAT -3′ |
| 1,018 | 5′- GTTACTTGTAGGGGGAGGGT[A]GAGGGAAATCTGGGCAAATG -3′ |
| 1,019 | 5′- GGGCTTCTATCCCCGAACCC[C]GGGCCCTGGTGCCACTCAAG -3′ |
| 1,020 | 5′- TCCCAYTTAAGAGCTATTCT[T]CTATCCTTCCCTGTAAACAA -3′ |
| 1,021 | 5′- TGGCAGACACAGGACAGGGA[G]CGCTGCTTATGTCTCCGAGG -3′ |
| 1,022 | 5′- AACCCATCCTCGTGGTAATC[G]TCCCTGGTAAGAAACACACA -3′ |
| 1,023 | 5′- CATTTCTAATTACCAGCTTC[T]TACTTGGCACTTTCAATTTT -3′ |
| 1,024 | 5′- CCACAGCGGCTTCCTGCCAT[T]GATGAGGCTGATTTCTGCCT -3′ |
| 1,025 | 5′- TGCATCCTCTGCTTCTCCTC[G]AACCGTGCTTCACAGCTGCC -3′ |
| 1,026 | 5′- GGGGCCAAAGGAATATTTAG[C]TGAAGGGGGAGAGAGGCCAC -3′ |
| 1,027 | 5′- ACTTTGTGTGTACATGTGGA[G]GGAAGTATTTGACATTTTGA -3′ |
| 1,028 | 5′- ACTTGTGTCCCCCAAAATCA[T]ATATTGAAGTTAAAACCTCC -3′ |
| 1,029 | 5′- TAGCCATGGCAGAAGACATA[C]TCTCTACACCTTATGCATGG -3′ |
| 1,030 | 5′- GACAGAGAAGGTATGTCCAC[G]CACACTAGACATACTGCATG -3′ |
| 1,031 | 5′- AGTATTGATCAGTGGCGGGA[C]ACAGTTTGAAGGTAGAGGGA -3′ |
| 1,032 | 5′- GCTGTATCTTGGGGGAAGTG[T]GTTCTTGAGAGCTGTGTAAG -3′ |
| 1,033 | 5′- GGCCGTCCTCATCTTCACAC[A]CTGTTCTCCTTCTATGTGGG -3′ |
| 1,034 | 5′- TAGCAGGTGGCACAACTGGC[G]CTGGGAACCGGGGGTCCCTT -3′ |
| 1,035 | 5′- GGCCCCCCGTGCAGGGAGGG[T]TTCAGGCTGCGGCAGGTAGG -3′ |
| 1,036 | 5′- TACTATACAAATAAAAAAAT[T]AAAACCCAACCTCAAGCTGT -3′ |
| 1,037 | 5′- CGAATGCTGAGAACTTGCCA[T]GCTCTCTCCCCAGGGCCCCA -3′ |
| 1,038 | 5′- GCCTCCCCCTGTGATCTCTC[G]GTCCTCTCCGCATTCCTGGG -3′ |
| 1,039 | 5′- TTCCCTTTGTTTTCCCTTTC[T]TCCAGCTCCAGGCCAGGCTT -3′ |
| 1,040 | 5′- TGCGCTCTGGGCTAGACACT[C]TGATAGGTGCTGGGATTACA -3′ |
| 1,041 | 5′- TGGAAAACAGATCCAGACAG[T]TTCAGTTATGTGTCTGAGAA -3′ |
| 1,042 | 5′- CCCTACTACCCCTACAACTA[T]ACGAGCGGCGCTGCCACCGG -3′ |
| 1,043 | 5′- GCATGCCTTTTCAAAAACAC[G]TTCAAGACCTGAAAATAAAA -3′ |
| 1,044 | 5′- TACTGCTGTGGCCTGAATCC[A]TGATTAAAGGAAATGCTAAG -3′ |
| 1,045 | 5′- TACACAAGTCACTGGGTGAC[G]TCTGTAGCTCCACCAACCTG -3′ |
| 1,046 | 5′- CTCTGTCTAGGTGCATAGAA[C]TGTGTACATATACATACACA -3′ |
| 1,047 | 5′- AGTCTGCAAATGTGTTTTTT[A]TGTGCTAAATAGCTCAAAGT -3′ |
| 1,048 | 5′- TAAGTTTGGTTGATGAGTCT[A]TCTCTCTAGACTGCAAGCTC -3′ |
| 1,049 | 5′- CACAGAAGTGGGCATTCTGA[A]AGGCCTCTAATTTTCCTCTA -3′ |
| 1,050 | 5′- TTAAAACAGCGACCCCATAC[G]TGCATTAGTTAAAACTTTCT -3′ |
| 1,051 | 5′- GCAGATTGAGGTAAATTCAT[C]GTTAATGTCATCACAGCAAT -3′ |
| 1,052 | 5′- CAAAACAGAATCCCAAGAGC[G]ATATTTTAACTCAACAAACA -3′ |
| 1,053 | 5′- AGAGTTCTTATGGTTCTCTT[T]GGTAGTTTTTCTTTAGCTGG -3′ |
| 1,054 | 5′- CTTTCATTCTTGTCGTTGGC[A]TCTCTGTTCTGATAAAAAGA -3′ |
| 1,055 | 5′- GGAGGCAATGTCTGATTTGC[C]TAGGGCTCAGGGGAGAGATG -3′ |
| 1,056 | 5′- AGGTTCAGCAGAAAAGAACC[C]AGGAAAAAAGTCTAGGAAAG -3′ |
| 1,057 | 5′- GATGGGCCTTCTGATAAGGA[G]CGCTGCCAAAAGTTCAAATG -3′ |
| 1,058 | 5′- ATTCCTTCCTTTCCCTGTTT[G]TACATACCTTACAGATACTG -3′ |
| 1,059 | 5′- TCTGTTTCAGTCTCAAGGAG[C]CTGAAAAGGTGAATTCCTGT -3′ |
| 1,060 | 5′- CAGTCTTGTGAGAACATTCT[C]GCCATCTGTACTTTGCATTT -3′ |
| 1,061 | 5′- CCACACCTGGCCTGAACTCT[T]CTTTAAAAACTGCATGCTGA -3′ |
| 1,062 | 5′- TCATGCATAGATGGTGTAGC[T]TTAGAAAACTCAGGCCTAGC -3′ |
| 1,063 | 5′- AGGTGGATTTTTTTAAGAAG[T]ATATTCATACAACTGAATAT -3′ |
| 1,064 | 5′- GCCTGATATTCTTTCCCTAT[T]AAATTGCTTCCTCATCTAGG -3′ |
| 1,065 | 5′- GAAGAAGCTGTCAGAATTGC[G]AGGGAAATTGGTAAGTCCTT -3′ |
| 1,066 | 5′- ACTGTGCCCACCCAAGTTTG[C]GTTTTGAAAAGATTGGTCAA -3′ |
| 1,067 | 5′- ATGGCATACAGCCTGGGTGA[C]ATTTTTAAACATAAGTGAAA -3′ |
| 1,068 | 5′- GGGAAAATGTTCATTTAAGT[G]TAAAACATGAAATGGTATTC -3′ |
| 1,069 | 5′- CTTGTTAGTTCAGGTCTCTT[C]CAGATGAGGAAGAGAGATTA -3′ |
| 1,070 | 5′- AAATGGACAACAAAAGTCAC[T]GGAAAAAAGGGAAAAAAAGA -3′ |
| 1,071 | 5′- TGAGAAATAAGTGATGTCAT[A]CATTTTTGGTTGTGGATCAT -3′ |
| 1,072 | 5′- TGTGGTTCTCCCTTCACAGT[T]GAATACAAGGGCTTTTATAT -3′ |
| 1,073 | 5′- TAATAAGTGGTTATGCCAAG[G]GGTCCCTGCAGCTCAGAGGC -3′ |
| 1,074 | 5′- TCTTTGGGCCTCCACCCCCT[T]GTCTCTAGTGGACATTTGAG -3′ |
| 1,075 | 5′- AAAGGAAGCTGGGCGTCCTC[T]GGGCCCCCCAACACACGTCC -3′ |
| 1,076 | 5′- CTAACACAGTTGCGAACATC[A]GCAGAGCCGTCGGGAGCCAC -3′ |
| 1,077 | 5′- TTGATGATGATGTCGATGCC[A]AAGAGTGACACGCCCAGTGC -3′ |
| 1,078 | 5′- CTTCACAGCGCCGCAACAAT[T]ATGCATGAGGGAGTGATTCG -3′ |
| 1,079 | 5′- GGCCACAGCTGGCCAGTCTC[T]TTGTGCTTTGAATCTCCAGC -3′ |
| 1,080 | 5′- TGCAGCGTGCGGCAGTGCTT[C]GTTCTTCTTTAAGATGAAAT -3′ |
| 1,081 | 5′- CCTACACAGGAAGCCCCGGA[A]CCACAGCAATTCTCCCTGCC -3′ |
| 1,082 | 5′- TGTGCTCTGGCCAGGGGCCT[T]GACCTCATTCTGTTGGTGGT -3′ |
| 1,083 | 5′- TCGCCCAGGCTGACCACAAG[T]TCCAAACAGGACTTTCTTGT -3′ |
| 1,084 | 5′- TGCCCAAACAGTATCAAAAG[C]GGATGTTTATCACAATACTA -3′ |
| 1,085 | 5′- TTAGCAACAAAATCCTGAAG[C]CACTTCTAGACCATAACCCA -3′ |
| 1,086 | 5′- CAGAGGGCAGGGCCCACACC[A]TACCCCACAGAAGCCCAGGA -3′ |
| 1,087 | 5′- GGGTACAGCCCAGCATGGCC[A]CAGGGGTCCCTGATGGGAAT -3′ |
| 1,088 | 5′- GACTGCCAGGTGTGGACACA[C]GCTCGTCAAGTGGTGAAGAA -3′ |
| 1,089 | 5′- CACACGGACGCTTCCTCCTA[C]GTGAAGTTCTGTTTGCTCCC -3′ |
| 1,090 | 5′- ATGGTCATATTATGCATGCA[T]GTTTTTGATTTCAAGAATGC -3′ |
| 1,091 | 5′- ATGCGGTGCTCGGTAACTGT[T]CATCCGATGCAGGCCTCACT -3′ |
| 1,092 | 5′- ACCAGAATTATCACAGCACC[C]TCTCATTCCCAGCGCGTCCT -3′ |
| 1,093 | 5′- TGATCATGGTCACTGCCCTG[A]GTTCAAATAATGCGAGCTGA -3′ |
| 1,094 | 5′- AGGACAACATGCCATTTGTC[C]AAACGTTTTAAAGATATGAT -3′ |
| 1,095 | 5′- GGGGGAAGCTGGGTGCATGC[A]GAGCACCGTGGAGTCTGGGA -3′ |
| 1,096 | 5′- CCTTGAAGTCACCCGGCCCC[C]ATGCAAGGTGCCCACATGTG -3′ |
| 1,097 | 5′- TTTGGAAGGAAAACGTGGCG[G]GTGGGCGTATTCTCCAGAAG -3′ |
| 1,098 | 5′- TCCCAGACCAGACCTTGCCC[G]ATGACGTTGTTGGTAATGCT -3′ |
| 1,099 | 5′- TGAGATCCCCCGGACAACAC[G]CTCCACCTTCCCATGGAGCT -3′ |
| 1,100 | 5′- TTGTTTGTGTCTGTCTCAAA[T]CCAAAGGGGTGGCTCAGCCT -3′ |
| 1,101 | 5′- GAACCTCCCAGGGGGCAGAA[T]AAAAAGTCAACAAGCTGGAA -3′ |
| 1,102 | 5′- CAAACGTTGCTGAAGTCTCC[G]CGACCTTTATTGTTTTGCCC -3′ |
| 1,103 | 5′- GTTCCCTGACCAGGAGTCCA[G]TAGGCAATAGTCTATTAACT -3′ |
| 1,104 | 5′- TTTGCTCATGCACCTGCCTT[A]CCTTTGTCATCACAACAGAA -3′ |
| 1,105 | 5′- ACCTCCTTCCCCGTGCKCCA[T]GAGGAGCGGGCTGCACCTTG -3′ |
| 1,106 | 5′- GCTGAAACCCGATTCCTACC[G]GGTGACGCTGAGACCGTACC -3′ |
| 1,107 | 5′- TCCTGCTCGACCTGCTCCTC[T]AGCTGTGCAATCTTGGCCTC -3′ |
| 1,108 | 5′- TCCAGCGCCGCGATGGTGGA[T]TTGAACTTGGACTTGACGGC -3′ |
| 1,109 | 5′- TACGAGGAGAAGGCGGCCGC[T]TATGATAAACTGGAAAAGAC -3′ |
| 1,110 | 5′- TTCCGCAGCTTGAGGTAGGC[A]GCGCAGTTCCTCTGAATCAC -3′ |
| 1,111 | 5′- CCTGTGGCTGGTACCTTCCC[G]GCATAATGGATGATGGAGAA -3′ |
| 1,112 | 5′- ATGATTGCCATGGCCTCCAC[A]GTTTCCTGGAACATCTCATC -3′ |
| 1,113 | 5′- CCAGAACCACCAACATCTTC[G]GTCTCTGTATTCAATTTTAT -3′ |
| 1,114 | 5′- TTTTCCCAGCTGTAAAAGGG[G]GCTAATAATAGCTCTTGCGG -3′ |
| 1,115 | 5′- GATACCTGACTCCAGGAGCC[G]TCACTTTACAACCTGAGATT -3′ |
| 1,116 | 5′- TTCTTGCCCTTGTACATGTC[A]ACGATCTTCTCCGAGTAGAT -3′ |
| 1,117 | 5′- ATCATGCTCAGTGAAACAAA[T]CAGAAAGGCCACACGCTCTA -3′ |
| 1,118 | 5′- ACCTGGTCAACAGCTTCCCT[C]AGGATTTTACTGCCAAGCCA -3′ |
| 1,119 | 5′- CACCCAGTCTGACCTTCACT[C]TTTTGTTGATGGGGCTGAGC -3′ |
| 1,120 | 5′- GCTGCTGGGGGTGGGTGCTT[C]GATCCTGGTGAAATGGCCTC -3′ |
| 1,121 | 5′- AGAATCATCTTCTCCTTTCC[C]TCACCTGATACCCAGCTTGA -3′ |
| 1,122 | 5′- CCTGTCAGGCCTGACGGGGA[A]GAACCACTGCACCACCGAGA -3′ |
| 1,123 | 5′- GGCTATGAATATAGTACCTG[G]AAAAATGCCAAGACATGATT -3′ |
| 1,124 | 5′- CTTTTGGGAATTTCCTCTCC[T]CTTGGCACTCGGAGTTGGGG -3′ |
| 1,125 | 5′- CAAGCCATGGCAGCGGACAG[T]CTGCTGAGAACACCCAGGAA -3′ |
| 1,126 | 5′- GACCAGTGAACTTCATCCTT[G]TCTGTCCAGGAGGTGGCCTC -3′ |
| 1,127 | 5′- TCAGTATAGATGCACCCATC[C]TAAGCCTAACTACATTGTAT -3′ |
| 1,128 | 5′- GTGAGCGTGCCATCAGCCCA[A]TGGAGGGGCTTAGGTCTGCA -3′ |
| 1,129 | 5′- GGTGCCATCCAGTGCCCTGA[C]AGTCAGTTCGAATGCCCGGA -3′ |
| 1,130 | 5′- GGCCCGTAGCCCTCACGTGG[C]TGTGAAGGACGTGGAGTGTG -3′ |
| 1,131 | 5′- TCAGGCCTCCCTAGCACCTC[T]CCCTAACCAAATTCTCCCTG -3′ |
| 1,132 | 5′- AGCCATGAGTTTCCACCAGC[G]GCAGAGTGAGTCCTGAGCAC -3′ |
| 1,133 | 5′- ATTGCAGAGAATGGAAGAAT[G]TGAAGAACTGAGTGACAAGG -3′ |
| 1,134 | 5′- AGCTACTGGGTAGAATTTTA[T]GTAGTAACTAGGTAGACACT -3′ |
| 1,135 | 5′- GGATGGCATAGCGAGAATAC[C]AATCTAGGAAGCGACTGGAC -3′ |
| 1,136 | 5′- GCTTTCCTGCTATCATAGCC[T]ACTTAAGTAGCTGTATTAGG -3′ |
| 1,137 | 5′- ATGAGGAAGAGAGAGACGAG[A]TGGGGTGACTCATGCCTGAA -3′ |
| 1,138 | 5′- TTTCTTTGAGACAGGGTCTC[G]CTCTGTTACCCAAGCTGGRA -3′ |
| 1,139 | 5′- TCATTAGCAGGGTGATGGTG[G]GGCTGAGATGGGCAGGGCCA -3′ |
| 1,140 | 5′- ATTGCCAACATAGCTGTTCA[C]ACCTAGAACACCTTTTCCTT -3′ |
| 1,141 | 5′- CACAACCTCGGTAAGGCTGG[C]GATCTTCAAGCCAGTCCGAT -3′ |
| 1,142 | 5′- GTCCGTTGTCCACGTTCTAC[T]TCCACCCCACTAACTGAACG -3′ |
| 1,143 | 5′- AGGCCAGGGGTCTGGATGCA[T]ATAGCGTTCCCCTAGCCTCT -3′ |
| 1,144 | 5′- TGCAGAGGTGTGGGCCCCTG[A]GGACCCAGAAGTCCAGCCAC -3′ |
| 1,145 | 5′- GGGTGAAGTAAAGTGGGCAG[A]GTGATTTAGCAGAGTGGTCA -3′ |
| 1,146 | 5′- GGCACCTGTCATAGTCTTGC[T]GAAAGATGACAACCCCTGGT -3′ |
| 1,147 | 5′- CGCAGCCCAGGATGATCTGT[G]CGGGACAGAGGCAGCGGCCT -3′ |
| 1,148 | 5′- TCGGAACAGCGAGTCCTCTG[G]CGTCGAGAGCAGGGAGGGGT -3′ |
| 1,149 | 5′- TTTGCCCAGTGACGCAGCAT[C]CCAGGCTGAGATTGCAGAAT -3′ |
| 1,150 | 5′- GCCCCCTCTGCAGGTCCCCT[T]GGTGTACTCTGAGGTGGGAA -3′ |

### REFERENCES CITED IN EXAMPLE 2

1. Fortin D F, Califf R M, Pryor D B, Mark D B (1995) The way of the future redux. Am J Cardiol 76: 1177-1182.
2. Smith L R, Harrell F E, Rankin J S, Califf R M, Pryor D B, et al. (1991) Determinants of Early Versus Late Cardiac Death in Patients Undergoing Coronary-Artery Bypass Graft-Surgery. Circulation 84:245-253.
3. Kong D F, Shaw L K, Harrell F E, Muhlbaier L H, Lee K L, et al. (2002) Predicting survival from the coronary arteriogram: an experience-based statistical index of coronary artery disease severity. Journal of the American College of Cardiology 39(Suppl A): 327A.
4. Felker G M, Shaw L K, O'Connor C M (2002) A standardized definition of ischemic cardiomyopathy for use in clinical research. J Am Coll Cardiol 39: 210-218.
5. Carlson C S, Eberle M A, Rieder M J, Yi Q, Kruglyak L, et al. (2004) Selecting a maximally informative set of single-nucleotide polymorphisms for association analyses using linkage disequilibrium. Am J Hum Genet. 74: 106-120.
6. Xu H, Gregory S G, Hauser E R, Stenger J E, Pericak-Vance M A, et al. (2005) SNPselector: a web toot for selecting SNPs for genetic association studies. Bioinformatics 21: 4181-4186.
7. Abecasis G R, Cookson W O (2000) GOLD—graphical overview of linkage disequilibrium. BioInformatics 16: 182-183.
8. Barrett J C, Fry B, Maller J, Daly M J (2005) Haploview: analysis and visualization of LD and haplotype maps. Bioinformatics 21: 263-265.
9. Schaid D J, Rowland C M, Tines D E, Jacobson R M, Poland G A (2002) Score tests for association between traits and haplotypes when linkage phase is ambiguous. Am J Hum Genet. 70: 425-434. The results are shown in Tables 3-5 below.

1. A method of estimating the risk of developing coronary artery disease (CAD) in a subject, the method comprising
(i) providing a nucleic acid sample from the subject;
(ii) detecting the presence of one or more single nucleotide polymorphisms (SNPs) in a CAD-determinative gene in the genomic sample,
wherein the CAD-determinative gene is selected from Table 2 or 3, and wherein the presence of one or more SNPs reflects a higher risk of developing coronary artery disease.

2. The method of claim 1, comprising detecting the presence of two or more single nucleotide polymorphisms (SNPs) from at least two CAD-determinative genes.

3. The method of claim 1, comprising detecting the presence of one or more single nucleotide polymorphisms (SNPs) from at least three genes in the genomic sample, wherein the genes are selected from AIM1L, PLA2G7, OR7E29P, PLN, PTPN6, C1ORF38, GATA2, IL7R, MYLK.

4. The method of claim 1, the CAD-determinative gene is selected from A1M1L, PLA2G7, OR7E29P, PLN, PTPN6, C1ORF38, GATA2, IL7R, MYLK.

5. The method of claim 1, wherein the step of detecting the presence of one or more single nucleotide polymorphisms comprises performing one or more procedures selected from:
(i) chain terminating sequencing;
(ii) restriction digestion;
(iii) allele-specific polymerase reaction;
(iv) single-stranded conformational polymorphism analysis,
(v) genetic bit analysis,
(vi) temperature gradient gel electrophoresis,
(vii) ligase chain reaction,
(viii) ligase/polymerase genetic bit analysis;
(ix) allele specific hybridization;
(x) size analysis; nucleotide sequencing,
(xi) 5′ nuclease digestion; and
(xiii) primer specific extension; oligonucleotide ligation assay.

6. The method of claim 1, wherein the nucleic acid sample is a genomic nucleic acid sample.

7. The method of claim 1, wherein the SNP is selected from any one of tables 1-4.

8. The method of claim 1, wherein the gene is AIM1L.

9. The method of claim 1, wherein the gene is PLA2G7.

10. The method of claim 1, wherein the gene is OR7E29P.

11. The method of claim 1, wherein the gene is PLN.

12. The method of claim 1, wherein the gene is PTPN6.

13. The method of claim 1, wherein the gene is C1ORF38.

14. The method of claim 1, wherein the gene is GATA2.

15. The method of claim 7, wherein the SNP is selected from a SNP listed in Table 4.

16. The method of claim 1, wherein the gene is IL7R.

17. The method of claim 1, wherein the gene is MYLK.

18. The method of claim 1, wherein the polymorphism is detected by
(i) contacting a nucleic acid sample from the individual with a polynucleotide probe which specifically hybridizes to the polymorphism; and
(ii) determining whether hybridization has occurred, thereby indicating the presence of the polymorphism.

19. A method of reducing the likelihood that a subject will develop CAD, or of delaying the onset of CAD in a subject, comprising:
(i) estimating the risk that the subject will develop coronary artery disease (CAD) according to the method of any one of claims 1-18;
(ii) administering to the subject, if the subject is at risk of developing CAD as estimated in step (ii), with a agent chosen from an anti-inflammatory agent, an antithrombotic agent, an anti-platelet agent, a fibrinolytic agent, a lipid-reducing agent, a direct thrombin inhibitor, a glycoprotein lib/IIIa receptor inhibitor, a calcium channel blocker, a beta-adrenergic receptor blocker, a cyclooxygenase-2 inhibitor or an angiotensin system inhibitor.

20. A method of estimating the risk of developing coronary artery disease (CAD) in a subject, the method comprising
(i) providing a nucleic acid sample from the subject;
(ii) detecting the presence of one or more single nucleotide polymorphisms (SNPs),
wherein at least one of the SNPs is a SNP listed in Table 4, and wherein the presence of one or more SNPs reflects a higher risk of developing coronary artery disease.


SEQUENCE LISTING (1150 sequences)

SEQ ID NO: 1 - DNA, 41 bp, Homo sapiens
ggacacaacaggacccactggggaaaacaatgatgacttgg

SEQ ID NO: 2 - DNA, 41 bp, Homo sapiens
cccctccactttgctcacccatcttccgggccctgaaccca

SEQ ID NO: 3 - DNA, 41 bp, Homo sapiens
tcctgtgccggctgcaggtatggaacaagtaggctagtgtc

SEQ ID NO: 4 - DNA, 41 bp, Homo sapiens
aggaaagactgttgggcctcggaaaacatcccacgtgctag

SEQ ID NO: 5 - DNA, 41 bp, Homo sapiens
gggacttggtttcatgtctctatctctcagttctgtttccc

SEQ ID NO: 6 - DNA, 41 bp, Homo sapiens
atagagagggtctgttaggttcttgggatcttgttcttcaa

SEQ ID NO: 7 - DNA, 41 bp, Homo sapiens
attccaattgaagattgaaagtggcctgtttggtaaactgg

SEQ ID NO: 8 - DNA, 41 bp, Homo sapiens
taactcaaagcacaaagttttgaattcctacattctaaaga

SEQ ID NO: 9 - DNA, 41 bp, Homo sapiens
gtcacctgcctcggagccagttaggctgtttaacagtgcag

SEQ ID NO: 10 - DNA, 41 bp, Homo sapiens
ggagctttggcatcgcagagacttgagctgagtctggctct

SEQ ID NO: 11 - DNA, 41 bp, Homo sapiens
cagagcccctccctctaaacacagtctttcaaagggattgt

SEQ ID NO: 12 - DNA, 41 bp, Homo sapiens
caatttcttgctgaaagccctgagttatgccagacactgtg

SEQ ID NO: 13 - DNA, 41 bp, Homo sapiens
acctttgcccagatccaaatgttttttcttcattcgaagct

SEQ ID NO: 14 - DNA, 41 bp, Homo sapiens
acggatctcttaccattaaattcaggtggagagggagtgcc

SEQ ID NO: 15 - DNA, 41 bp, Homo sapiens
tttcacagatgaggaggctgtcctcaggaaatgtgactcag

SEQ ID NO: 16 - DNA, 41 bp, Homo sapiens
ccaacaccaccccttgcccagccaatgcacacagtagggct

SEQ ID NO: 17 - DNA, 41 bp, Homo sapiens
cccatatcatgcagaggatctgggatttcaatccaggtcta

SEQ ID NO: 18 - DNA, 41 bp, Homo sapiens
tgacgtgtgcagagagacatctcagcctgccctgcacttgt

SEQ ID NO: 19 - DNA, 41 bp, Homo sapiens
ggcagcatattagaaaatagcttatgttacaacaaaaaccc

SEQ ID NO: 20 - DNA, 41 bp, Homo sapiens
tgccccttctcactggtctgcggctggcagggccatctttc

SEQ ID NO: 21 - DNA, 41 bp, Homo sapiens
gaatccatcccaaggacaccctttgaaaacatgaaataaca

SEQ ID NO: 22 - DNA, 41 bp, Homo sapiens
cagcggggaggggaaaggtctgaaatgaggggagagacgtg

SEQ ID NO: 23 - DNA, 41 bp, Homo sapiens
gctgggcagagccattcctgagctggctgggtgtgtttggg

SEQ ID NO: 24 - DNA, 41 bp, Homo sapiens
acaggcatcagggatacagtggtgaacaagcatacacaatc

SEQ ID NO: 25 - DNA, 41 bp, Homo sapiens
aggtgaagctgaggcctgagcccagaaggagagaaaaggaa

SEQ ID NO: 26 - DNA, 41 bp, Homo sapiens
cactcattaatccattaaaccattaatctattaatccatga

SEQ ID NO: 27 - DNA, 41 bp, Homo sapiens
gtgtatgctgtgaagaaggcaaccccccttcctgcccatcc

SEQ ID NO: 28 - DNA, 41 bp, Homo sapiens
ctgtcactatgcccctgcctttctcagtgtctatctctgtt

SEQ ID NO: 29 - DNA, 41 bp, Homo sapiens
gggatgacagtgagaggaggccaacagtaaaaggagtcata

SEQ ID NO: 30 - DNA, 41 bp, Homo sapiens
gtgtgtctgtcagggaatgtgtccctcttccattctctgtg

SEQ ID NO: 31 - DNA, 41 bp, Homo sapiens
ccattcttggtggtgagcctggactctgagcctgggatgtg

SEQ ID NO: 32 - DNA, 41 bp, Homo sapiens
gtctggctgccccttggcctccacyacagtcaggtccagcc

SEQ ID NO: 33 - DNA, 41 bp, Homo sapiens
ttgaggattaaagagcagargtcatgtagcatctggcacat

SEQ ID NO: 34 - DNA, 41 bp, Homo sapiens
cgtcatgtagcatctggcacgtgggggaacgcaatggaagt

SEQ ID NO: 35 - DNA, 41 bp, Homo sapiens
cagagaatatttcacatgcatgtagcaaaaacaccaggggt

SEQ ID NO: 36 - DNA, 41 bp, Homo sapiens
aacatggattaatgtgggaacttggcttcaagaacacaacc

SEQ ID NO: 37 - DNA, 41 bp, Homo sapiens
attatttcattttaaaaccatagaataaaaatgacacctga

SEQ ID NO: 38 - DNA, 41 bp, Homo sapiens
aagcagattatgaggcagctccacccctcccagcactgggg

SEQ ID NO: 39 - DNA, 41 bp, Homo sapiens
ccagccctgtagtggacatatttgcctttgcctattcagca

SEQ ID NO: 40 - DNA, 41 bp, Homo sapiens
gaactcggtggaggagaagagaaactccaagatgctccaga

SEQ ID NO: 41 - DNA, 41 bp, Homo sapiens
tgtgggctggacttagcaacgcacttctaactaacagaatg

SEQ ID NO: 42 - DNA, 41 bp, Homo sapiens
ggtgtcaattcactcccagcggcactgactgagtgctgacc

SEQ ID NO: 43 - DNA, 41 bp, Homo sapiens
atgttaggcggtcccacctgcgttctggagatcttcacaca

SEQ ID NO: 44 - DNA, 41 bp, Homo sapiens
ggtgggcagaggctggatcctatggtgaggagtttccattt

SEQ ID NO: 45 - DNA, 41 bp, Homo sapiens
ttgccatgggccacctctaccgagtgctcgatgaacaacaa

SEQ ID NO: 46 - DNA, 41 bp, Homo sapiens
tttggctggggcaagcttacgtggttcggcagtagtaccag

SEQ ID NO: 47 - DNA, 41 bp, Homo sapiens
gtggccccaggaatgggggcgtctggtggtatctgggctgg

SEQ ID NO: 48 - DNA, 41 bp, Homo sapiens
atgcattgtggtagattcatacaatggagtatacacagcaa

SEQ ID NO: 49 - DNA, 41 bp, Homo sapiens
gtggcagctgccatttttccggtgccacaaatggtagttac

SEQ ID NO: 50 - DNA, 41 bp, Homo sapiens
ttgggaggaagaccacagaggtgatgtgccagtctcagaac

SEQ ID NO: 51 - DNA, 41 bp, Homo sapiens
aaaatacagggtacagggacactcaaagagtgatttgcttc

SEQ ID NO: 52 - DNA, 41 bp, Homo sapiens
gtgagatggggcacagcagcggccggaaggttatttgtgtg

SEQ ID NO: 53 - DNA, 41 bp, Homo sapiens
gcagggcagagaaggggaagctgctggctgccctcctcact

SEQ ID NO: 54 - DNA, 41 bp, Homo sapiens
gctcctggattcactcctttcatcctcacctcaatcctttg

SEQ ID NO: 55 - DNA, 41 bp, Homo sapiens
agttggcttgtatggaccccgccgatgacggacagttccaa

SEQ ID NO: 56 - DNA, 41 bp, Homo sapiens
agtggattgaggatggacatgtgtatctggaagcaccaaaa

SEQ ID NO: 57 - DNA, 41 bp, Homo sapiens
ctgggttcactggaaatcagtattaagaatgtacaagggaa

SEQ ID NO: 58 - DNA, 41 bp, Homo sapiens
atgtaaactgcctttgaaagcctataacacagttcagttgg

SEQ ID NO: 59 - DNA, 41 bp, Homo sapiens
acttaatcttgctcagttcctcagtttacacttttgaatgg

SEQ ID NO: 60 - DNA, 41 bp, Homo sapiens
gcagcatagatgaatgtaatattgaaacaggaagatgtgtt

SEQ ID NO: 61 - DNA, 41 bp, Homo sapiens
cttagcctgcaattgcaatccgtatgggaccatgaagcagc

SEQ ID NO: 62 - DNA, 41 bp, Homo sapiens
tagccgtttacagaatatccggaataccattgaagagactg

SEQ ID NO: 63 - DNA, 41 bp, Homo sapiens
gtttcagattttgataggcgcgtgaacgataacaagacggc

SEQ ID NO: 64 - DNA, 41 bp, Homo sapiens
atgagggagaaatgccctttttggcaattgttggagctgga

SEQ ID NO: 65 - DNA, 41 bp, Homo sapiens
aggaacagtgctacttactggtgggtagactgggagaggtg

SEQ ID NO: 66 - DNA, 41 bp, Homo sapiens
ttggcaatgggtaagtctatcgtactgtgtaaacttggact

SEQ ID NO: 67 - DNA, 41 bp, Homo sapiens
gatatagatctcttggaaatgtaataatggtatgcaggaag

SEQ ID NO: 68 - DNA, 41 bp, Homo sapiens
gcaacccggggaaatacagccaaatgcacaagtactggctg

SEQ ID NO: 69 - DNA, 41 bp, Homo sapiens
ctgtacaaactttcttccataattttgattatatccatttt

SEQ ID NO: 70 - DNA, 41 bp, Homo sapiens
ccctcattatctgcctaaacgattttttctcaactcctata

SEQ ID NO: 71 - DNA, 41 bp, Homo sapiens
ctagcactgtacacaccccacactgtgtatgctatttgttg

SEQ ID NO: 72 - DNA, 41 bp, Homo sapiens
caaaagttatctctaaccaatgtactcaaacagagtcttta

SEQ ID NO: 73 - DNA, 41 bp, Homo sapiens
ccttgtaaatctccacctgagatttctcatggtgttgtagc

SEQ ID NO: 74 - DNA, 41 bp, Homo sapiens
tcccataggaattataaaatggaaaagtatgacaaaaattt

SEQ ID NO: 75 - DNA, 41 bp, Homo sapiens
aggcccttcagcttcaccacctgcttctctttaaacaagtc

SEQ ID NO: 76 - DNA, 41 bp, Homo sapiens
gatagaatttggcccagagaggttaactaatatatccatga

SEQ ID NO: 77 - DNA, 41 bp, Homo sapiens
ctgtttctccttaaaatggagaaatggcctctacagagtag

SEQ ID NO: 78 - DNA, 41 bp, Homo sapiens
gcttggtggggccactgggcgtctgtttctcgggtgttttg

SEQ ID NO: 79 - DNA, 41 bp, Homo sapiens
ccattccctcggcgaagagcggaggttgaagaaatgctact

SEQ ID NO: 80 - DNA, 41 bp, Homo sapiens
gcaagggccagagcctctgtgtgctgcattcggcaaccaca

SEQ ID NO: 81 - DNA, 41 bp, Homo sapiens
ggttcctgaaggaggagtggaagtttggtaaatggatggag

SEQ ID NO: 82 - DNA, 41 bp, Homo sapiens
ttacctgctaaggcctgcaaacttgaggatgtccagggctg

SEQ ID NO: 83 - DNA, 41 bp, Homo sapiens
ccagaaggtttctttgctccccttccctacaaagacagagc

SEQ ID NO: 84 - DNA, 41 bp, Homo sapiens
aattcactcctttaaaatacccaatgcagtgtttttagaaa

SEQ ID NO: 85 - DNA, 41 bp, Homo sapiens
ccactccctctcctgctcttgtgtgtgtgatccaaagggaa

SEQ ID NO: 86 - DNA, 41 bp, Homo sapiens
cagggacagctgaagccaagctctcccaaagcagccttggc

SEQ ID NO: 87 - DNA, 41 bp, Homo sapiens
gtcaggagcctggccaggccgcaccccttgctgtctcagca

SEQ ID NO: 88 - DNA, 41 bp, Homo sapiens
ggagattctgcctcagggccgtgagagtcccatcttcaagc

SEQ ID NO: 89 - DNA, 41 bp, Homo sapiens
gctcagctaccgttggtggcatttattaaactgtgcaccca

SEQ ID NO: 90 - DNA, 41 bp, Homo sapiens
aaggtggctgactccagcccatttgcccttgaactgctgat

SEQ ID NO: 91 - DNA, 41 bp, Homo sapiens
tgaagacctgaaaagcaaattccaggcagccccactccctc

SEQ ID NO: 92 - DNA, 41 bp, Homo sapiens
ttctttgtaatttggaatccacctaatttccaaatgggttc

SEQ ID NO: 93 - DNA, 41 bp, Homo sapiens
gggacctggccctggccatccgggacagtgagcgacagggc

SEQ ID NO: 94 - DNA, 41 bp, Homo sapiens
aggtggggacccggctccaaaggcacccgggtcttctgcag

SEQ ID NO: 95 - DNA, 41 bp, Homo sapiens
acaggccgctctcccagcagcgtgttgaggtgcacagccag

SEQ ID NO: 96 - DNA, 41 bp, Homo sapiens
tggcgcaagagaaccagggcgtcttcttctcgggggactcc

SEQ ID NO: 97 - DNA, 41 bp, Homo sapiens
tgctgtgcccacatcccctgcaacaggcagcccagcctgtg

SEQ ID NO: 98 - DNA, 41 bp, Homo sapiens
tggtgagttatggacccycctacctccactactacactgta

SEQ ID NO: 99 - DNA, 41 bp, Homo sapiens
tcagggcctggggcaggcgcggcacagcccccaccgctgct

SEQ ID NO: 100 - DNA, 41 bp, Homo sapiens
gcatggcatgcggaagatggtgaagaatgttttatggcctc

SEQ ID NO: 101 - DNA, 41 bp, Homo sapiens
tctcagtagctgagacctgagaaatttggagaatcactttg

SEQ ID NO: 102 - DNA, 41 bp, Homo sapiens
acatgaggccactgaggcagccctctttccttccccttctc

SEQ ID NO: 103 - DNA, 41 bp, Homo sapiens
cctattcttaatcctattttgcaaatgaagtgacttgccca

SEQ ID NO: 104 - DNA, 41 bp, Homo sapiens
ggaatgggtcaagaatgttcgttcccttctgaatgtccctg

SEQ ID NO: 105 - DNA, 41 bp, Homo sapiens
aagcggggaggagctaaatactatttttctctccttgttca

SEQ ID NO: 106 - DNA, 41 bp, Homo sapiens
aacttggaacatctccgcaacaagacagaggatctggaagc

SEQ ID NO: 107 - DNA, 41 bp, Homo sapiens
acatcgcagaaggtggctcgaaaattctggtggaagaacgt

SEQ ID NO: 108 - DNA, 41 bp, Homo sapiens
ttcccgaggccctgctgccatgttgtatgccccagaaggta

SEQ ID NO: 109 - DNA, 41 bp, Homo sapiens
tgagagtcagggtttgggaccagattggcaagtcaggctct

SEQ ID NO: 110 - DNA, 41 bp, Homo sapiens
tctccaggacctagtatggtgcctgaccgtggcactcatag

SEQ ID NO: 111 - DNA, 41 bp, Homo sapiens
ctacctcagagtatgtgcccattggatggtggctgttattc

SEQ ID NO: 112 - DNA, 41 bp, Homo sapiens
ctagtctctgagctgagtgccgacttagggaggcaatgtta

SEQ ID NO: 113 - DNA, 41 bp, Homo sapiens
acagtgtggcgtaaggcagtgtggcccttgtcctcttgctt

SEQ ID NO: 114 - DNA, 41 bp, Homo sapiens
ttagggcagctgtgcattgactgggtagacgccattctgga

SEQ ID NO: 115 - DNA, 41 bp, Homo sapiens
tgaggcccccacctggcccttatctgcccctgagatctaga

SEQ ID NO: 116 - DNA, 41 bp, Homo sapiens
cgcataatttccgtcacctcattcgcctgctgctggcaccg

SEQ ID NO: 117 - DNA, 41 bp, Homo sapiens
ccccaacatgtgcacccctgcatttcctgtcatgccacaga

SEQ ID NO: 118 - DNA, 41 bp, Homo sapiens
ccagatctccatcattggcgttagtctctggtcacctgact

SEQ ID NO: 119 - DNA, 41 bp, Homo sapiens
tttgttctgactttacatccccttccccaggtcacttttca

SEQ ID NO: 120 - DNA, 41 bp, Homo sapiens
attcctgtcccttgtgccgctatgagctgcccactgatgac

SEQ ID NO: 121 - DNA, 41 bp, Homo sapiens
tttgataccaagaacacatttctgcatgaatcctccagcaa

SEQ ID NO: 122 - DNA, 41 bp, Homo sapiens
tctaaaattaggggtttgatttagcttatctggaaggtgtt

SEQ ID NO: 123 - DNA, 41 bp, Homo sapiens
gatgcggtctggaaagcaccagggtggccgtcggctgacgc

SEQ ID NO: 124 - DNA, 41 bp, Homo sapiens
ctccgtggaacttctcctggtacaaattctgttcctaggga

SEQ ID NO: 125 - DNA, 41 bp, Homo sapiens
gaggggagccacaggaatggccgtggccagaagcccttctc

SEQ ID NO: 126 - DNA, 41 bp, Homo sapiens
ggcaccttttccctgataagacacaaatcataaccaaacaa

SEQ ID NO: 127 - DNA, 41 bp, Homo sapiens
ttgcactccagtttttttttctttaaaaaagcggtttctac

SEQ ID NO: 128 - DNA, 41 bp, Homo sapiens
gaaaaggctgtctgattatcgtgtcatccaaaaaaaacaga

SEQ ID NO: 129 - DNA, 41 bp, Homo sapiens
gaactaagaggaataaaggtattgctttatacctgtcccta

SEQ ID NO: 130 - DNA, 41 bp, Homo sapiens
actaacatgtcctgcctattatctgtcagctgcaaggtact

SEQ ID NO: 131 - DNA, 41 bp, Homo sapiens
gctgacccagggtccacatgctctttttctaacttgttcat

SEQ ID NO: 132 - DNA, 41 bp, Homo sapiens
tgcttccccatttctgtcctaaaagccctctggcaagactg

SEQ ID NO: 133 - DNA, 41 bp, Homo sapiens
cagtgatgaactcctgggcttaagtgacccacccgcctctg

SEQ ID NO: 134 - DNA, 41 bp, Homo sapiens
gcgacttcgactaagcaacattgcatctattttcatgcaac

SEQ ID NO: 135 - DNA, 41 bp, Homo sapiens
cctcaaatgttagagtcagtgcaccagctcatagtttccat

SEQ ID NO: 136 - DNA, 41 bp, Homo sapiens
cgtttaattctttctcatcagtttcctagggcatttgcaat

SEQ ID NO: 137 - DNA, 41 bp, Homo sapiens
catcagagttttatgattagtagatatatcttaactgacac

SEQ ID NO: 138 - DNA, 42 bp, Homo sapiens
agcaaaaccaaagaaatcagcggaagaccataaaaacagacg

SEQ ID NO: 139 - DNA, 41 bp, Homo sapiens
ctataaaattagtatgcttaaaattattaaacatatacaga

SEQ ID NO: 140 - DNA, 41 bp, Homo sapiens
taaacactttaatgcagtgatactcaggtataaaactcaga

SEQ ID NO: 141 - DNA, 41 bp, Homo sapiens
atagaagacaaagttttcatccgtctcattcaagttcactt

SEQ ID NO: 142 - DNA, 41 bp, Homo sapiens
agtgcagggcaggactgctgtctgaccccgggccacctgga

SEQ ID NO: 143 - DNA, 41 bp, Homo sapiens
aacctcttggtacatgttaggggaaatgaagctggcaacaa

SEQ ID NO: 144 - DNA, 41 bp, Homo sapiens
tcatcagatcaaggacattatggaattaaagggctctaaga

SEQ ID NO: 145 - DNA, 41 bp, Homo sapiens
ccactgctattggttatttatctagcatccatttcccttta

SEQ ID NO: 146 - DNA, 41 bp, Homo sapiens
atctacctctcctgcctcatctattattacccagccccttc

SEQ ID NO: 147 - DNA, 41 bp, Homo sapiens
gtcaattgcaaatggaggtgggacctgagaaaacaaagaaa

SEQ ID NO: 148 - DNA, 41 bp, Homo sapiens
gagtgtgtaacaactcacctaccaaatcgactagcccttag

SEQ ID NO: 149 - DNA, 41 bp, Homo sapiens
cttgtaagccatcttaagccattataggcctaagatgtata

SEQ ID NO: 150 - DNA, 41 bp, Homo sapiens
cttgagacctgtgtctcctcgtgttcacactgttcctgact

SEQ ID NO: 151 - DNA, 41 bp, Homo sapiens
gaggcatgggttgaactgcactcacatatgtacttaaaaga

SEQ ID NO: 152 - DNA, 41 bp, Homo sapiens
tgtttcttgaagtttgactatttaaaaacataggtgtaaag

SEQ ID NO: 153 - DNA, 41 bp, Homo sapiens
agagtcacggcatgtgggaaggtttccatggacactggatc

SEQ ID NO: 154 - DNA, 41 bp, Homo sapiens
aatgagatcttatgtcaaggctttaatctttggtattccaa

SEQ ID NO: 155 - DNA, 41 bp, Homo sapiens
tctggacctcagtttcctcagtgagctggtaagaatgcact

SEQ ID NO: 156 - DNA, 41 bp, Homo sapiens
aggttgatagcaatgtttggaagatatgtcctagaagtgtt

SEQ ID NO: 157 - DNA, 41 bp, Homo sapiens
gcatgataacccgagccatcgctaaatattatagcttcctt

SEQ ID NO: 158 - DNA, 41 bp, Homo sapiens
ctccagtttctccctttctcaccaactaggtccatccaaac

SEQ ID NO: 159 - DNA, 41 bp, Homo sapiens
aactgtaaggatctcttgctgtatatactattgggggaaca

SEQ ID NO: 160 - DNA, 41 bp, Homo sapiens
ccttagctcttcctaaaacatacaatcataaaggaaaccgt

SEQ ID NO: 161 - DNA, 41 bp, Homo sapiens
ctgacagtaaagggaactcattatgtctgagtctttgctca

SEQ ID NO: 162 - DNA, 41 bp, Homo sapiens
aacatttacagaagcgagaataagttttgtttgcttttgtt

SEQ ID NO: 163 - DNA, 41 bp, Homo sapiens
taagttcaataaatcccaaattgcacactctgaattagggg

SEQ ID NO: 164 - DNA, 41 bp, Homo sapiens
aagatagccatctttgggcacagagtcatgaaatgtaccct

SEQ ID NO: 165 - DNA, 41 bp, Homo sapiens
gctgggccgacggggacgaggcggcgactggagcagcagcg

SEQ ID NO: 166 - DNA, 41 bp, Homo sapiens
ctctgtcttggtcactgtgcaaggattgaagggaactattg

SEQ ID NO: 167 - DNA, 41 bp, Homo sapiens
atcgtcttttacaataagatacatgcccctatgagtatttt

SEQ ID NO: 168 - DNA, 41 bp, Homo sapiens
aaggagaaaaacagtgaaccgtagttcttactgctcacact

SEQ ID NO: 169 - DNA, 41 bp, Homo sapiens
gattatttgattgccatgaatgaagctgaattacataattc

SEQ ID NO: 170 - DNA, 41 bp, Homo sapiens
agggacctgtcttcagaatcgaagaagcataatgtccttaa

SEQ ID NO: 171 - DNA, 41 bp, Homo sapiens
tagagtccctaccatgcaccgtgggcaagaagtcagttctg

SEQ ID NO: 172 - DNA, 41 bp, Homo sapiens
tcgggtctcttaccatgcccaccctcccttcctcagggaat

SEQ ID NO: 173 - DNA, 41 bp, Homo sapiens
aggaccttcagagaccccgcattctctgaaaccaggatgga

SEQ ID NO: 174 - DNA, 41 bp, Homo sapiens
caggggctgcactcaccatcatctgacacctccacttcatc

SEQ ID NO: 175 - DNA, 41 bp, Homo sapiens
gtacacaagggtagggcagaagatggacagcagggcagaat

SEQ ID NO: 176 - DNA, 41 bp, Homo sapiens
agtttctgcagcactttatccttccatctggccatgaggaa

SEQ ID NO: 177 - DNA, 41 bp, Homo sapiens
caggcattgaaggtcagcttcttctcctcctgggtgagttt

SEQ ID NO: 178 - DNA, 41 bp, Homo sapiens
gggcacgacctaccatccacagtgacttggcaggagcactc

SEQ ID NO: 179 - DNA, 41 bp, Homo sapiens
ttacttctatccttgcttctcgaactggtcattccctgact

SEQ ID NO: 180 - DNA, 41 bp, Homo sapiens
agaacaagctgttagcaggatgcctctgctgctgcggggcc

SEQ ID NO: 181 - DNA, 41 bp, Homo sapiens
tcggctgggatctccttcaggtcgtcttccgatagggtctt

SEQ ID NO: 182 - DNA, 41 bp, Homo sapiens
aggcctcagggacccatagcggtcactaccaccaccatcag

SEQ ID NO: 183 - DNA, 41 bp, Homo sapiens
ttgtccagaaatcactgtgattggatacacaaatgcagcac

SEQ ID NO: 184 - DNA, 41 bp, Homo sapiens
cttggctgctgaatggtgagttccccctgccccagctctct

SEQ ID NO: 185 - DNA, 41 bp, Homo sapiens
gaagtcttctgaaggaccggagtctgcggggccgttctggg

SEQ ID NO: 186 - DNA, 41 bp, Homo sapiens
tggtggcttttgtttctctcacaaatgacctgtgtggtggt

SEQ ID NO: 187 - DNA, 41 bp, Homo sapiens
aggacgggtctccactgctgaagctgaaaatctatccctgt

SEQ ID NO: 188 - DNA, 40 bp, Homo sapiens
tttgtgaccttgtatggatgacttctctgaatcttatttc

SEQ ID NO: 189 - DNA, 41 bp, Homo sapiens
aaaactcaataagatgcctacattttatgcatctccattaa

SEQ ID NO: 190 - DNA, 41 bp, Homo sapiens
ttcaccatccctctactttcagcttgccaaaacttacagga

SEQ ID NO: 191 - DNA, 41 bp, Homo sapiens
tggccagtgctcagcagatgcaagttccaaatcgagtcact

SEQ ID NO: 192 - DNA, 41 bp, Homo sapiens
gcatggagtcaactcttgagggatccacactgagggaggtt

SEQ ID NO: 193 - DNA, 41 bp, Homo sapiens
tgactcctggtccagggcctgctggggactagataagatgt

SEQ ID NO: 194 - DNA, 41 bp, Homo sapiens
caagctagagacttggtatatagcagcagttacatgagtgg

SEQ ID NO: 195 - DNA, 41 bp, Homo sapiens
cagactgtggacatccgaatcggcaatgacatgaatttaag

SEQ ID NO: 196 - DNA, 41 bp, Homo sapiens
aggcaccaggtcccatggcctgtttcccctgagaaaacatt

SEQ ID NO: 197 - DNA, 41 bp, Homo sapiens
atggagagctgccaagccaaacctgccagggtcatcagctc

SEQ ID NO: 198 - DNA, 41 bp, Homo sapiens
atagctgtccttactcctttgctagacagacagtgtcttgg

SEQ ID NO: 199 - DNA, 41 bp, Homo sapiens
gctttttataccgcttaacgtaaataatttaaaaggctgtc

SEQ ID NO: 200 - DNA, 41 bp, Homo sapiens
agctgcaatgcctatgagcaagacctgggtttgtacatctt

SEQ ID NO: 201 - DNA, 41 bp, Homo sapiens
ctaggatagcagagatattatttcaggatcagatcttgact

SEQ ID NO: 202 - DNA, 41 bp, Homo sapiens
tctggggagtctttagcccctagcagaggccatttctagca

SEQ ID NO: 203 - DNA, 41 bp, Homo sapiens
gaataaaacttacggagagcttctaacttcattcaatttgt

SEQ ID NO: 204 - DNA, 41 bp, Homo sapiens
ataatatattttaagcagggcagggtatcccaagatctcaa

SEQ ID NO: 205 - DNA, 41 bp, Homo sapiens
gtatggtaaagaatcccactgctgcatcaatcagtgggcaa

SEQ ID NO: 206 - DNA, 41 bp, Homo sapiens
ttttccttacaccaagcttatgtgggtggctgtagccacaa

SEQ ID NO: 207 - DNA, 41 bp, Homo sapiens
gcaccatgggggaaattatcagtattatttttttgaaatca

SEQ ID NO: 208 - DNA, 41 bp, Homo sapiens
tatagycaaagagttgtgcagtgatcacctcaatgaattta

SEQ ID NO: 209 - DNA, 41 bp, Homo sapiens
gttctgggcaactgctttagcctgaatgcaaaaaactggaa

SEQ ID NO: 210 - DNA, 41 bp, Homo sapiens
aaacaaaagccccacagcaagaaacaggaaggaaggggaac

SEQ ID NO: 211 - DNA, 41 bp, Homo sapiens
atagtgagggatgactgtattttccacttaaaaatcccaag

SEQ ID NO: 212 - DNA, 41 bp, Homo sapiens
ggaaaataaaactgtacctcatctccagtctccccatattt

SEQ ID NO: 213 - DNA, 41 bp, Homo sapiens
taatggctttcaaagtgcctaaattccattctacactaaaa

SEQ ID NO: 214 - DNA, 41 bp, Homo sapiens
acctcaaaagaaaaaataacgtaaacaatattcaactcaag

SEQ ID NO: 215 - DNA, 41 bp, Homo sapiens
gcttggttcaggccctggttgcatacctggatttcaaatct

SEQ ID NO: 216 - DNA, 41 bp, Homo sapiens
acccacagctttcagcagtgcagaatatgaatggaaactgg

SEQ ID NO: 217 - DNA, 41 bp, Homo sapiens
gagtgaggtagagaacaggtgtaattcaccataagtcctga

SEQ ID NO: 218 - DNA, 41 bp, Homo sapiens
acctggttctttgaaagaaccaataaaattcacaaactgct

SEQ ID NO: 219 - DNA, 41 bp, Homo sapiens
tttttctcttcagctggcccaaattggtttctgttaatttt

SEQ ID NO: 220 - DNA, 41 bp, Homo sapiens
gaagagactaagagaatcacagaagagagaaggaggtcaag

SEQ ID NO: 221 - DNA, 41 bp, Homo sapiens
tcttgaagggttttagttccataagttccagggaggggtct

SEQ ID NO: 222 - DNA, 41 bp, Homo sapiens
aaacgtttaattcttctgtgggttctgttctaatttctgag

SEQ ID NO: 223 - DNA, 41 bp, Homo sapiens
aggcctagaattctctgaaatgtcatttttcagtttctaca

SEQ ID NO: 224 - DNA, 41 bp, Homo sapiens
gtagccttgcgcctcactcttgtgatggagccgcctgctac

SEQ ID NO: 225 - DNA, 41 bp, Homo sapiens
attgtcattttccttgtgttatattggttcaggctatccaa

SEQ ID NO: 226 - DNA, 41 bp, Homo sapiens
caaggcatcttggctcctacgtagggccttttggctcctct

SEQ ID NO: 227 - DNA, 41 bp, Homo sapiens
agatctccaaggttttcaccgagaaacacttgacccgactt

SEQ ID NO: 228 - DNA, 41 bp, Homo sapiens
cctcaatgcagaggggtcatgagagcaggctgggagccaga

SEQ ID NO: 229 - DNA, 41 bp, Homo sapiens
gttcctcctcagaaactgccttgtatgagtttgtatcctta

SEQ ID NO: 230 - DNA, 41 bp, Homo sapiens
cataggcgaggcccagcccacgtgtccagagacatctgtga

SEQ ID NO: 231 - DNA, 41 bp, Homo sapiens
gctcttcaaggtctggtgctttcttccacagtactgtagcc

SEQ ID NO: 232 - DNA, 41 bp, Homo sapiens
aaatgggtgctcagacccctatcctacttacctcaaaaggt

SEQ ID NO: 233 - DNA, 41 bp, Homo sapiens
tgtcagcagcctggtattgggaagagttaaaggaaaatctc

SEQ ID NO: 234 - DNA, 41 bp, Homo sapiens
cagttcaggggaggagcctcaggacgtcagtggcaaaatca

SEQ ID NO: 235 - DNA, 41 bp, Homo sapiens
gcataggcttaactcgctgatgagttaattgttttattttt

SEQ ID NO: 236 - DNA, 41 bp, Homo sapiens
aggggaaacgtctcccagatcgctcccttggctttgaggcc

SEQ ID NO: 237 - DNA, 41 bp, Homo sapiens
agccaaagccagagtggccacggcccagggagggtgagctg

SEQ ID NO: 238 - DNA, 41 bp, Homo sapiens
tttcagagagggaagccagaggagaagagggtgcaggctga

SEQ ID NO: 239 - DNA, 41 bp, Homo sapiens
caagtcctccggttcttcctcgggattggcgggtccacttg

SEQ ID NO: 240 - DNA, 41 bp, Homo sapiens
aggctgcctccgcacctgaccgctgcccaggtggggtttcc

SEQ ID NO: 241 - DNA, 41 bp, Homo sapiens
tggctaggacagggtctcgggctagggaagtggtttctctg

SEQ ID NO: 242 - DNA, 41 bp, Homo sapiens
ttacgggaagcccttctggcgctcactcagggcagcagctt

SEQ ID NO: 243 - DNA, 41 bp, Homo sapiens
gcctgggcaggaagagggactagagggtctcccacatggga

SEQ ID NO: 244 - DNA, 41 bp, Homo sapiens
atcgtgttccccaggaagttgttcttgatttagtttaaact

SEQ ID NO: 245 - DNA, 41 bp, Homo sapiens
gaaccaccttctcttgccagtctgtactcctcatttagttt

SEQ ID NO: 246 - DNA, 41 bp, Homo sapiens
aaggtgggagccagagtgggctgctgtaggggtgagggagg

SEQ ID NO: 247 - DNA, 41 bp, Homo sapiens
gccatccagcgcggctgctccggcgccacctccatggccgg

SEQ ID NO: 248 - DNA, 41 bp, Homo sapiens
tccctgggcccgtcgccctcggggctcccgccggaactcct

SEQ ID NO: 249 - DNA, 41 bp, Homo sapiens
acacagacattgtcgagcgcgggtccctctttattggccag

SEQ ID NO: 250 - DNA, 41 bp, Homo sapiens
gcctggtgagagcagatttactccaatttatgggctggaac

SEQ ID NO: 251 - DNA, 41 bp, Homo sapiens
cacaccgacacacatggccacacaatcagatgcaactcggc

SEQ ID NO: 252 - DNA, 41 bp, Homo sapiens
cttgttcacagaagtgggaggcaggagggggggagaaagtg

SEQ ID NO: 253 - DNA, 41 bp, Homo sapiens
aggaccaggcggctaagcagggagaagagccagaggggcgt

SEQ ID NO: 254 - DNA, 41 bp, Homo sapiens
cgggccatggacaccgacacgctgacacaggtcaaggagaa

SEQ ID NO: 255 - DNA, 41 bp, Homo sapiens
ctgcggttcagctccttggtgagatctgtcatgtctgtctg

SEQ ID NO: 256 - DNA, 41 bp, Homo sapiens
gcacgtcggctcttggtacagaagacgaacagggctgcggg

SEQ ID NO: 257 - DNA, 41 bp, Homo sapiens
tcccccggggccctgagcaacgcatcagcgccagtggactt

SEQ ID NO: 258 - DNA, 41 bp, Homo sapiens
ttcaccaggacctggagctcggagcctacatggaggtcatt

SEQ ID NO: 259 - DNA, 41 bp, Homo sapiens
acggtcaccacacctgagagtggtcctggggctggccctgt

SEQ ID NO: 260 - DNA, 41 bp, Homo sapiens
gcggcagccatcactccacatgcacaggtgacccaggtctt

SEQ ID NO: 261 - DNA, 41 bp, Homo sapiens
aggatgttctgggagccacccgtaggcacgggtgccagggg

SEQ ID NO: 262 - DNA, 41 bp, Homo sapiens
tggaatgagcaacacaggaatgctccagttgtccagaccat

SEQ ID NO: 263 - DNA, 41 bp, Homo sapiens
cgagactggttggaaacacaggagtgctgctggctgcacca

SEQ ID NO: 264 - DNA, 41 bp, Homo sapiens
cccccatccattccagaccacgtgactgttgagatgtctgt

SEQ ID NO: 265 - DNA, 41 bp, Homo sapiens
tcgatgtgcgccaggagtacccagtgagtcctgggggaggc

SEQ ID NO: 266 - DNA, 41 bp, Homo sapiens
agtttgacccagcagactccggttacctttacctgatgacg

SEQ ID NO: 267 - DNA, 41 bp, Homo sapiens
cctaccttgagaagcctcccgttgaccgtgcccaggaagac

SEQ ID NO: 268 - DNA, 41 bp, Homo sapiens
aggcctccaggaagtgaccccgagacaataactgtgcaact

SEQ ID NO: 269 - DNA, 41 bp, Homo sapiens
gtaactaagcacaccccttacagaattttgggaagtcgccc

SEQ ID NO: 270 - DNA, 41 bp, Homo sapiens
taagccagaggatgctgtagagagtacttgtatgcaataac

SEQ ID NO: 271 - DNA, 41 bp, Homo sapiens
cttgttgtcatggtgcgttggaagagtagccagttgtcttt

SEQ ID NO: 272 - DNA, 41 bp, Homo sapiens
attagtatgcaggtcttatctaccattggaattaagctgtt

SEQ ID NO: 273 - DNA, 41 bp, Homo sapiens
acgtttttatcacacattaagcacttgcattaattttggag

SEQ ID NO: 274 - DNA, 41 bp, Homo sapiens
gatgagttaaatgggctagtgtctaaattttaaatttttac

SEQ ID NO: 275 - DNA, 41 bp, Homo sapiens
gtacatcccatattccctttgcaaaatctagtttcctatgt

SEQ ID NO: 276 - DNA, 41 bp, Homo sapiens
gcttaccagaaaacaccctcgttgttgtttttatttctcag

SEQ ID NO: 277 - DNA, 41 bp, Homo sapiens
ggacaaggaggagaagccccaggaggtcacgggagttcact

SEQ ID NO: 278 - DNA, 41 bp, Homo sapiens
gagcagccatttcgaaaggcagcagaagaggaaattaactc

SEQ ID NO: 279 - DNA, 41 bp, Homo sapiens
gcgaggggaagtcatttttttaataactaggctctatttgc

SEQ ID NO: 280 - DNA, 41 bp, Homo sapiens
caaggaaagacctggtgtccttgtgctaattttaactctct

SEQ ID NO: 281 - DNA, 41 bp, Homo sapiens
tacagatgctcataggcatccgaaaaaaaaatactttgtta

SEQ ID NO: 282 - DNA, 41 bp, Homo sapiens
aactcctttgacagtatggacggcacctaacgcatccttgt

SEQ ID NO: 283 - DNA, 41 bp, Homo sapiens
gaggtgttttcttggctcttaackaacgtttttaataaagc

SEQ ID NO: 284 - DNA, 41 bp, Homo sapiens
gcgccccctggacttctgctagaatttagatttaaatagat

SEQ ID NO: 285 - DNA, 41 bp, Homo sapiens
acatatttagaatggatgccggaacaggagaaatgggtggg

SEQ ID NO: 286 - DNA, 41 bp, Homo sapiens
attcatatgccaccagccatcggcagaaatgtaacaggaaa

SEQ ID NO: 287 - DNA, 41 bp, Homo sapiens
atggctctgtaaatgggatgcctcatgttcaggtttctgga

SEQ ID NO: 288 - DNA, 41 bp, Homo sapiens
atctccaggtgaacatggaacgcagtgaaaacctggggtat

SEQ ID NO: 289 - DNA, 41 bp, Homo sapiens
tgataagtagttaatgatcctgaaataaactgttaggtgct

SEQ ID NO: 290 - DNA, 41 bp, Homo sapiens
aagtaaaatagtagatattgcattgcttctacatttactac

SEQ ID NO: 291 - DNA, 41 bp, Homo sapiens
agagcccctacccaattgctctactatttatagttcctcag

SEQ ID NO: 292 - DNA, 41 bp, Homo sapiens
atctggggacctgctcctggtagagcaataggawctgtgtg

SEQ ID NO: 293 - DNA, 41 bp, Homo sapiens
gagtcccaaaattcaaccctcccgatagggctgggcctgac

SEQ ID NO: 294 - DNA, 41 bp, Homo sapiens
ccctagcctgcttttgtcctgttattttttatttccacata

SEQ ID NO: 295 - DNA, 41 bp, Homo sapiens
agagggaacccaaatattagggtgggaagcaagtcataaac

SEQ ID NO: 296 - DNA, 41 bp, Homo sapiens
tagggttaccaatccactagaatgcaaaactgtacttatta

SEQ ID NO: 297 - DNA, 41 bp, Homo sapiens
aggcttctttttccattacactgtaagactttggagggcag

SEQ ID NO: 298 - DNA, 41 bp, Homo sapiens
agcrgtcaggtgcggaggcagcctctcagcggtggggaaca

SEQ ID NO: 299 - DNA, 41 bp, Homo sapiens
caggacaaacagtggattcactcagaacacaatatgctggt

SEQ ID NO: 300 - DNA, 41 bp, Homo sapiens
aagccactacagacaccgcacgcaccgaaattctcccttgt

SEQ ID NO: 301 - DNA, 41 bp, Homo sapiens
atcactgtccctcagttcaccggtcttgtctgcttcgtcgy

SEQ ID NO: 302 - DNA, 41 bp, Homo sapiens
aattctcagtcttaaaaacaaggcataaagaaagctaaaat

SEQ ID NO: 303 - DNA, 41 bp, Homo sapiens
agaagataagtgtttagggtgttggatatcccagttaccct

SEQ ID NO: 304 - DNA, 41 bp, Homo sapiens
cctttttttggatgatcctacaattaatacaagtgtattct

SEQ ID NO: 305 - DNA, 41 bp, Homo sapiens
gcccttagtcaccaactccttctcatcccaccatgctgttg

SEQ ID NO: 306 - DNA, 41 bp, Homo sapiens
gtaaattaaaatttgtttggctgatttgtgctgtatttcta

SEQ ID NO: 307 - DNA, 41 bp, Homo sapiens
agcaacacttcctccttgcagattacaagcatagctaatgc

SEQ ID NO: 308 - DNA, 41 bp, Homo sapiens
ccctcattttctgttagggatgtatgtgtttaccaagctgt

SEQ ID NO: 309 - DNA, 41 bp, Homo sapiens
atgagggctttacttttgcaggaaatactacagatggtgaa

SEQ ID NO: 310 - DNA, 41 bp, Homo sapiens
tcccttctcagtaactaacattaatcatctctctggaggac

SEQ ID NO: 311 - DNA, 41 bp, Homo sapiens
cattccctcacacagtacagtttaataaatgtgcattttga

SEQ ID NO: 312 - DNA, 41 bp, Homo sapiens
cctgtgtgatgaggggcaaaggaagctcttgagaacctgct

SEQ ID NO: 313 - DNA, 41 bp, Homo sapiens
gtaacgaagaaagaccagagtgtcatccctgtgatacagca

SEQ ID NO: 314 - DNA, 41 bp, Homo sapiens
tatgtatcttgcttttgtttaaaacagtcatccacattagt

SEQ ID NO: 315 - DNA, 41 bp, Homo sapiens
gataggttgcaaaattttggcgtgttcttgcattgcataca

SEQ ID NO: 316 - DNA, 41 bp, Homo sapiens
attgacggtgttataattaccatggttttgaaattacatag

SEQ ID NO: 317 - DNA, 41 bp, Homo sapiens
tgaggacccagatgtcaacaccaccaatctggaatttgaaa

SEQ ID NO: 318 - DNA, 41 bp, Homo sapiens
ctccttttgacctgagtgtcatctatcgggaaggagccaat

SEQ ID NO: 319 - DNA, 41 bp, Homo sapiens
tatgtaaaagttttaatgcacgatgtagcttaccgccagga

SEQ ID NO: 320 - DNA, 41 bp, Homo sapiens
gatggatcctatcttactaaccatcagcattttgagttttt

SEQ ID NO: 321 - DNA, 41 bp, Homo sapiens
aattagctgccagagttgctgtcagtaaagagaagaaataa

SEQ ID NO: 322 - DNA, 41 bp, Homo sapiens
ctgaaatcagagaacattgaaagatgaagtgaatggcagag

SEQ ID NO: 323 - DNA, 41 bp, Homo sapiens
gcccatctgaggatgtagtcatcactccakaaagctttgga

SEQ ID NO: 324 - DNA, 41 bp, Homo sapiens
gtgcagaycagataattatacagagatggaatgggacaacc

SEQ ID NO: 325 - DNA, 41 bp, Homo sapiens
aatctgcctctggggcgggatctgtcaggcttcaggaaggg

SEQ ID NO: 326 - DNA, 41 bp, Homo sapiens
tccagggaggagcttcgtgcgaccttcccggaccactcagg

SEQ ID NO: 327 - DNA, 41 bp, Homo sapiens
catcacctccaggtagctcctaaaatgtccctagaaagtgg

SEQ ID NO: 328 - DNA, 41 bp, Homo sapiens
ggagcacagagtagcagtgatgctgtccaaggcagggggga

SEQ ID NO: 329 - DNA, 41 bp, Homo sapiens
cattcaggccagtggctgcaggggagcagaaagatcaggct

SEQ ID NO: 330 - DNA, 41 bp, Homo sapiens
tacagaggaagaaatccagggcagaggtggaggcagtgaag

SEQ ID NO: 331 - DNA, 41 bp, Homo sapiens
ctacctcattcattgaccccactatctgacctgtacatgtt

SEQ ID NO: 332 - DNA, 41 bp, Homo sapiens
ttgaggacaaacagaacatcggtgagtaagtggaatattag

SEQ ID NO: 333 - DNA, 41 bp, Homo sapiens
ttcttgtgttcttccctttccatttcaactcttcatctcag

SEQ ID NO: 334 - DNA, 41 bp, Homo sapiens
ggtttgtgtaccaggattggggacccctgatgtatagtgta

SEQ ID NO: 335 - DNA, 41 bp, Homo sapiens
gaagaggataggtttttctaccttaaacaaaatcttcctta

SEQ ID NO: 336 - DNA, 41 bp, Homo sapiens
gttaggcatcaggcaactaccaaggagtatacgagcatgca

SEQ ID NO: 337 - DNA, 41 bp, Homo sapiens
cacagggtaaatttagccactgcagcaggagcatgatataa

SEQ ID NO: 338 - DNA, 41 bp, Homo sapiens
ggcatgtgaaataagttggtctaattagagtgaagcccagg

SEQ ID NO: 339 - DNA, 41 bp, Homo sapiens
tggattgtgtgtgtggtaataggattattgttatatttaaa

SEQ ID NO: 340 - DNA, 41 bp, Homo sapiens
cacgagcatcttgctgtcttaaattaagaagttaactggac

SEQ ID NO: 341 - DNA, 41 bp, Homo sapiens
ttgaaagctgagtcattttcataatgggtcagaaagacatt

SEQ ID NO: 342 - DNA, 41 bp, Homo sapiens
tacatgacgcatgtatttgtgaaaacccacagatctattaa

SEQ ID NO: 343 - DNA, 41 bp, Homo sapiens
ctgagagtgcagtgaacctttgtgtctgtgatggaagaggt

SEQ ID NO: 344 - DNA, 41 bp, Homo sapiens
gcttagatgtgagagttgatgccataataataaaagttatt

SEQ ID NO: 345 - DNA, 41 bp, Homo sapiens
ttgaactctatgtaccaagtttgaacacattccaaatatcc

SEQ ID NO: 346 - DNA, 41 bp, Homo sapiens
ggtattttgctacagcagcccgagcaaactaatatatcatc

SEQ ID NO: 347 - DNA, 41 bp, Homo sapiens
aaaggcggtcacctgcaggaatagccatctttggtcctttc

SEQ ID NO: 348 - DNA, 41 bp, Homo sapiens
cccccaggggtggtaacaacagcacgcaagcacagccattg

SEQ ID NO: 349 - DNA, 41 bp, Homo sapiens
ccacacctggtggacaggaccaccgtggtggccaggaagct

SEQ ID NO: 350 - DNA, 41 bp, Homo sapiens
ggttaaaaagttctctaccacggaagttggataaaagtaac

SEQ ID NO: 351 - DNA, 41 bp, Homo sapiens
aaatcagaatcgaattattggtttggggctaattgtatctg

SEQ ID NO: 352 - DNA, 41 bp, Homo sapiens
cctgtcagtgaaaacaactaccaaagctggattttaaatat

SEQ ID NO: 353 - DNA, 41 bp, Homo sapiens
ccattagcagtaggtctgaattaactttaatatgcaagtta

SEQ ID NO: 354 - DNA, 41 bp, Homo sapiens
agagccagctgggagaaacatgcaacatagttctttgcaat

SEQ ID NO: 355 - DNA, 41 bp, Homo sapiens
agcagctggaccatgatctcctggatatggtggtaggtgaa

SEQ ID NO: 356 - DNA, 41 bp, Homo sapiens
agacgatgtactgatgtaaggttttgtaaatttctaaactg

SEQ ID NO: 357 - DNA, 41 bp, Homo sapiens
actctgtctttccaattcctcaacagcatgcttggatggga

SEQ ID NO: 358 - DNA, 41 bp, Homo sapiens
tcagaaagaatggggtaaggtgaattgagttttagaacata

SEQ ID NO: 359 - DNA, 41 bp, Homo sapiens
acagtgaagaaagaggaacatagagaagggcaggcaggagg

SEQ ID NO: 360 - DNA, 41 bp, Homo sapiens
ttgaaggtggatgagggaacggtcaggttgaggagcatttt

SEQ ID NO: 361 - DNA, 41 bp, Homo sapiens
acacaatactgggtttctcttcttctctctcaccatcacac

SEQ ID NO: 362 - DNA, 41 bp, Homo sapiens
ccacgcaccagcaggttcacggtgcagctcatgcggttgtc

SEQ ID NO: 363 - DNA, 41 bp, Homo sapiens
gatagtctaaatgaatgtcccccacccccgcctgtagttgt

SEQ ID NO: 364 - DNA, 41 bp, Homo sapiens
gctggctggggcaaaggtctctgatgcactgtgcagaagta

SEQ ID NO: 365 - DNA, 41 bp, Homo sapiens
ctgctcgggccagaaaatccggaaacgggcccttaccgatg

SEQ ID NO: 366 - DNA, 41 bp, Homo sapiens
gttctgaaatgaagacacatatggcaggcaggttacaaccc

SEQ ID NO: 367 - DNA, 41 bp, Homo sapiens
ctcactcactccttgaggaccctctcatgacaactgtaaag

SEQ ID NO: 368 - DNA, 41 bp, Homo sapiens
ttcaaaaactattttggtacctttcaaatacagtgtttaaa

SEQ ID NO: 369 - DNA, 41 bp, Homo sapiens
tgttgctaagatcaatagctgcatttgaatctatgtctccc

SEQ ID NO: 370 - DNA, 41 bp, Homo sapiens
cagtttattatgggttatctcattggaataaagaggtatca

SEQ ID NO: 371 - DNA, 41 bp, Homo sapiens
aatcattatgtcacaaaaaattatataaagataaatttttc

SEQ ID NO: 372 - DNA, 41 bp, Homo sapiens
agagccaagacttgtcccctatttctgcagcagattggtcc

SEQ ID NO: 373 - DNA, 41 bp, Homo sapiens
gtttctcaaaagttctaaactttacagaggataattttaag

SEQ ID NO: 374 - DNA, 41 bp, Homo sapiens
ccttgtctggaggagttggggttcctcaataattggctgtg

SEQ ID NO: 375 - DNA, 41 bp, Homo sapiens
ggatccaaagggtgtcaaggcgatcattatcttgggatgga

SEQ ID NO: 376 - DNA, 41 bp, Homo sapiens
aatgaaactaaatgatcatccttcaactctcccttctcact

SEQ ID NO: 377 - DNA, 41 bp, Homo sapiens
agttgctcccctctctgatccacattcgtaaaatgacataa

SEQ ID NO: 378 - DNA, 41 bp, Homo sapiens
caggtgtccctaccttaaggtcctcctccttgggacttcac

SEQ ID NO: 379 - DNA, 41 bp, Homo sapiens
cacacaactrgctaaggagctccagggccacagctgctgtt

SEQ ID NO: 380 - DNA, 41 bp, Homo sapiens
ataagcaggaaaatgaatgcgttaggagaggttttatatct

SEQ ID NO: 381 - DNA, 41 bp, Homo sapiens
ttatgcatacaacactcaacagatccagttactcttactct

SEQ ID NO: 382 - DNA, 41 bp, Homo sapiens
caccccagtcaccgtggcttgcacctgcacaacagattcct

SEQ ID NO: 383 - DNA, 41 bp, Homo sapiens
aatttccctgcattttgtgacgacttgtttttattggtaac

SEQ ID NO: 384 - DNA, 41 bp, Homo sapiens
tgcgcattttccgcactccgatacactttacactgaacacc

SEQ ID NO: 385 - DNA, 41 bp, Homo sapiens
gacccagagcaggaagcatagtcaagccctccactagatta

SEQ ID NO: 386 - DNA, 41 bp, Homo sapiens
cacttggaaatcctaactccacagaacaaaattttacaagc

SEQ ID NO: 387 - DNA, 41 bp, Homo sapiens
acacactgacattcgaggccaaaggaatactcctgcctcta

SEQ ID NO: 388 - DNA, 41 bp, Homo sapiens
ttcatttacaagcctgatcacccttacatgaactaatgttt

SEQ ID NO: 389 - DNA, 41 bp, Homo sapiens
aacactgttgcaggatctctgataatcactatgtacacttc

SEQ ID NO: 390 - DNA, 41 bp, Homo sapiens
aactccccagctaaacacccgtaagacttcatacaacacaa

SEQ ID NO: 391 - DNA, 41 bp, Homo sapiens
taaatgcttatccatttagtaacaggaaaaatgagacaact

SEQ ID NO: 392 - DNA, 41 bp, Homo sapiens
gtatgctttccatcgaaaaattactctattaaacagcttag

SEQ ID NO: 393 - DNA, 41 bp, Homo sapiens
tatacaggagtcatcccctacgttgacactggtaagttgta

SEQ ID NO: 394 - DNA, 41 bp, Homo sapiens
tcaagtttaagctgctatgttccttatttttaacttttgtt

SEQ ID NO: 395 - DNA, 41 bp, Homo sapiens
atataatttatattacaatggaaaagcttctttaatactaa

SEQ ID NO: 396 - DNA, 41 bp, Homo sapiens
gatggggaggaagagaaggcgttggtcttgcagtcttgtct

SEQ ID NO: 397 - DNA, 41 bp, Homo sapiens
aatggtaagcatctattttgtagtccactctactgagctaa

SEQ ID NO: 398 - DNA, 41 bp, Homo sapiens
tttatatatgatatcatcataaagcactttctataagctga

SEQ ID NO: 399 - DNA, 41 bp, Homo sapiens
aacaatctgtgaacacttgttatatgcttactgtaagtgtg

SEQ ID NO: 400 - DNA, 41 bp, Homo sapiens
actatatgtcatgtctacagtctgtctcctaagagtagagg

SEQ ID NO: 401 - DNA, 41 bp, Homo sapiens
tgcaaacattgggaaaccacggtaggggggagcaggactct

SEQ ID NO: 402 - DNA, 41 bp, Homo sapiens
taccatggacagcagcgctgcccccacraacgccagcaatt

SEQ ID NO: 403 - DNA, 41 bp, Homo sapiens
acttgtcccacttagatggcaacctgtccgacccatgcggt

SEQ ID NO: 404 - DNA, 41 bp, Homo sapiens
gcatttcacattcacatgtagtatttgaatatacacatcaa

SEQ ID NO: 405 - DNA, 41 bp, Homo sapiens
ttgagtctccttccaattaactcatggaacatcagagccat

SEQ ID NO: 406 - DNA, 41 bp, Homo sapiens
tcttttgtggaaatgtgatgcatttgtttatatgcagacaa

SEQ ID NO: 407 - DNA, 41 bp, Homo sapiens
accagacttaggagagatatatctcactgtagaaccagtgc

SEQ ID NO: 408 - DNA, 41 bp, Homo sapiens
ctctggtcaaggctaaaaatcaatgagcaaaatggcagtat

SEQ ID NO: 409 - DNA, 41 bp, Homo sapiens
agccaaagttcagttctccagttcatctgagctcaggccca

SEQ ID NO: 410 - DNA, 45 bp, Homo sapiens
ggtatcrtgggtcctttcragtactaaccgccttaggctggaagc

SEQ ID NO: 411 - DNA, 41 bp, Homo sapiens
ttttaccgaaggctgtgtcttgtaagcacccccgagcaact

SEQ ID NO: 412 - DNA, 41 bp, Homo sapiens
ctactccggcacccagtgggttggtagtcctgttggcagga

SEQ ID NO: 413 - DNA, 41 bp, Homo sapiens
ccaagaagcgcgcggcgagagtgcaaggtgggggccccgcc

SEQ ID NO: 414 - DNA, 41 bp, Homo sapiens
ctctcgccgcgcgcttcttggtccctgagacttcgaacgaa

SEQ ID NO: 415 - DNA, 41 bp, Homo sapiens
gagcagaggggcaggtcccgaccggacggcgcccggagccc

SEQ ID NO: 416 - DNA, 41 bp, Homo sapiens
agagcggattgggggtcgcgtgtggtagcaggaggaggagc

SEQ ID NO: 417 - DNA, 41 bp, Homo sapiens
tggggattcagagcacccacgcgcagcacctccctcctctg

SEQ ID NO: 418 - DNA, 41 bp, Homo sapiens
gggtcagtccggacagccccagtcgcttgttacctagcatc

SEQ ID NO: 419 - DNA, 41 bp, Homo sapiens
ctgggtgcgctggccgaggcgtacccctccaagccggacaa

SEQ ID NO: 420 - DNA, 41 bp, Homo sapiens
gacatggccagatactactcggcgctgcgacactacatcaa

SEQ ID NO: 421 - DNA, 41 bp, Homo sapiens
ctgacaatgtctgtggcaaccctgcagtttactccttggtt

SEQ ID NO: 422 - DNA, 41 bp, Homo sapiens
cagacacccactcctatgtgtgtttctgaaaattacagggt

SEQ ID NO: 423 - DNA, 41 bp, Homo sapiens
tccagatatggaaaacgatccagcccagagacactgatttc

SEQ ID NO: 424 - DNA, 41 bp, Homo sapiens
atttcaatttagagtcagggtctcactctatgctcccctga

SEQ ID NO: 425 - DNA, 41 bp, Homo sapiens
tggaaagaggtgcccaccaatgtctaagtgttaaacattga

SEQ ID NO: 426 - DNA, 41 bp, Homo sapiens
tatcatgcattcaaaagtgtatcctcctcaatgaaaaatct

SEQ ID NO: 427 - DNA, 41 bp, Homo sapiens
tgaaaaatctattacaatagtgaggattattttcgttaaac

SEQ ID NO: 428 - DNA, 41 bp, Homo sapiens
tatttctcaaacattttcaggtttagaatgggaataggttt

SEQ ID NO: 429 - DNA, 41 bp, Homo sapiens
gtgcctttaaacctattctaaaacctatttaaacgtatttc

SEQ ID NO: 430 - DNA, 41 bp, Homo sapiens
agggctgcctggtaagctgaatcagggtgcctggctgccgc

SEQ ID NO: 431 - DNA, 41 bp, Homo sapiens
aacgccacttgtgactgctcgttacctttcagttgtgtccc

SEQ ID NO: 432 - DNA, 41 bp, Homo sapiens
atgttgggatttaactttctgttatatgtcagactcactta

SEQ ID NO: 433 - DNA, 41 bp, Homo sapiens
tgtgtgttttaaatctttgcgcttaaatgtttttgatttct

SEQ ID NO: 434 - DNA, 41 bp, Homo sapiens
gaagcttccctccgacaggcggccccgcactaaggtaggga

SEQ ID NO: 435 - DNA, 41 bp, Homo sapiens
ctaatggttggaaacgccagcctttggtgaaaacagaaagt

SEQ ID NO: 436 - DNA, 41 bp, Homo sapiens
ttcaagaattcaactgcagattgaaaatatttggagaaaaa

SEQ ID NO: 437 - DNA, 41 bp, Homo sapiens
aacctagccacagagcccgatgcgatgtgtccttgtcgaga

SEQ ID NO: 438 - DNA, 41 bp, Homo sapiens
gcctcctttgctgccctcacaatctcttcctgtgacaccac

SEQ ID NO: 439 - DNA, 41 bp, Homo sapiens
ctctgcaccttcaggttcaggcccttcaagatctaccagga

SEQ ID NO: 440 - DNA, 41 bp, Homo sapiens
acaagctagttaccttttattgttcagtttaaaaaagttct

SEQ ID NO: 441 - DNA, 41 bp, Homo sapiens
cggtccccttcaagatccatcccgacctgaagagaaaccgc

SEQ ID NO: 442 - DNA, 41 bp, Homo sapiens
tgctcttcaaaaaaaccagactgaatatttttaaaagtaat

SEQ ID NO: 443 - DNA, 41 bp, Homo sapiens
gttacttgtagggggagggtggagggaaatctgggcaaatg

SEQ ID NO: 444 - DNA, 41 bp, Homo sapiens
gggcttctatccccgaaccctgggccctggtgccactcaag

SEQ ID NO: 445 - DNA, 41 bp, Homo sapiens
tcccayttaagagctattctcctatccttccctgtaaacaa

SEQ ID NO: 446 - DNA, 41 bp, Homo sapiens
tggcagacacaggacagggatcgctgcttatgtctccgagg

SEQ ID NO: 447 - DNA, 41 bp, Homo sapiens
aacccatcctcgtggtaatcatccctggtaagaaacacaca

SEQ ID NO: 448 - DNA, 41 bp, Homo sapiens
catttctaattaccagcttcctacttggcactttcaatttt

SEQ ID NO: 449 - DNA, 41 bp, Homo sapiens
ccacagcggcttcctgccatcgatgaggctgatttctgcct

SEQ ID NO: 450 - DNA, 41 bp, Homo sapiens
tgcatcctctgcttctcctcaaaccgtgcttcacagctgcc

SEQ ID NO: 451 - DNA, 41 bp, Homo sapiens
ggggccaaaggaatatttaggtgaagggggagagaggccac

SEQ ID NO: 452 - DNA, 41 bp, Homo sapiens
actttgtgtgtacatgtggaaggaagtatttgacattttga

SEQ ID NO: 453 - DNA, 41 bp, Homo sapiens
acttgtgtcccccaaaatcacatattgaagttaaaacctcc

SEQ ID NO: 454 - DNA, 41 bp, Homo sapiens
tagccatggcagaagacatattctctacaccttatgcatgg

SEQ ID NO: 455 - DNA, 41 bp, Homo sapiens
gacagagaaggtatgtccacacacactagacatactgcatg

SEQ ID NO: 456 - DNA, 41 bp, Homo sapiens
agtattgatcagtggcgggatacagtttgaaggtagaggga

SEQ ID NO: 457 - DNA, 41 bp, Homo sapiens
gctgtatcttgggggaagtgcgttcttgagagctgtgtaag

SEQ ID NO: 458 - DNA, 41 bp, Homo sapiens
ggccgtcctcatcttcacacgctgttctccttctatgtggg

SEQ ID NO: 459 - DNA, 41 bp, Homo sapiens
tagcaggtggcacaactggcactgggaaccgggggtccctt

SEQ ID NO: 460 - DNA, 41 bp, Homo sapiens
ggccccccgtgcagggagggcttcaggctgcggcaggtagg

SEQ ID NO: 461 - DNA, 41 bp, Homo sapiens
tactatacaaataaaaaaataaaaacccaacctcaagctgt

SEQ ID NO: 462 - DNA, 41 bp, Homo sapiens
cgaatgctgagaacttgccacgctctctccccagggcccca

SEQ ID NO: 463 - DNA, 41 bp, Homo sapiens
gcctccccctgtgatctctcagtcctctccgcattcctggg

SEQ ID NO: 464 - DNA, 41 bp, Homo sapiens
ttccctttgttttccctttcctccagctccaggccaggctt

SEQ ID NO: 465 - DNA, 41 bp, Homo sapiens
tgcgctctgggctagacactgtgataggtgctgggattaca

SEQ ID NO: 466 - DNA, 41 bp, Homo sapiens
tggaaaacagatccagacaggttcagttatgtgtctgagaa

SEQ ID NO: 467 - DNA, 41 bp, Homo sapiens
ccctactacccctacaactacacgagcggcgctgccaccgg

SEQ ID NO: 468 - DNA, 41 bp, Homo sapiens
gcatgccttttcaaaaacacattcaagacctgaaaataaaa

SEQ ID NO: 469 - DNA, 41 bp, Homo sapiens
tactgctgtggcctgaatccgtgattaaaggaaatgctaag

SEQ ID NO: 470 - DNA, 41 bp, Homo sapiens
tacacaagtcactgggtgacatctgtagctccaccaacctg

SEQ ID NO: 471 - DNA, 41 bp, Homo sapiens
ctctgtctaggtgcatagaattgtgtacatatacatacaca

SEQ ID NO: 472 - DNA, 41 bp, Homo sapiens
agtctgcaaatgtgttttttgtgtgctaaatagctcaaagt

SEQ ID NO: 473 - DNA, 41 bp, Homo sapiens
taagtttggttgatgagtctgtctctctagactgcaagctc

SEQ ID NO: 474 - DNA, 41 bp, Homo sapiens
cacagaagtgggcattctgagaggcctctaattttcctcta

SEQ ID NO: 475 - DNA, 41 bp, Homo sapiens
ttaaaacagcgaccccatacatgcattagttaaaactttct

SEQ ID NO: 476 - DNA, 41 bp, Homo sapiens
gcagattgaggtaaattcattgttaatgtcatcacagcaat

SEQ ID NO: 477 - DNA, 41 bp, Homo sapiens
caaaacagaatcccaagagcaatattttaactcaacaaaca

SEQ ID NO: 478 - DNA, 41 bp, Homo sapiens
agagttcttatggttctcttcggtagtttttctttagctgg

SEQ ID NO: 479 - DNA, 41 bp, Homo sapiens
ctttcattcttgtcgttggcgtctctgttctgataaaaaga

SEQ ID NO: 480 - DNA, 41 bp, Homo sapiens
ggaggcaatgtctgatttgcgtagggctcaggggagagatg

SEQ ID NO: 481 - DNA, 41 bp, Homo sapiens
aggttcagcagaaaagaacctaggaaaaaagtctaggaaag

SEQ ID NO: 482 - DNA, 41 bp, Homo sapiens
gatgggccttctgataaggaacgctgccaaaagttcaaatg

SEQ ID NO: 483 - DNA, 41 bp, Homo sapiens
attccttcctttccctgtttatacataccttacagatactg

SEQ ID NO: 484 - DNA, 41 bp, Homo sapiens
tctgtttcagtctcaaggaggctgaaaaggtgaattcctgt

SEQ ID NO: 485 - DNA, 41 bp, Homo sapiens
cagtcttgtgagaacattcttgccatctgtactttgcattt

SEQ ID NO: 486 - DNA, 41 bp, Homo sapiens
ccacacctggcctgaactctgctttaaaaactgcatgctga

SEQ ID NO: 487 - DNA, 41 bp, Homo sapiens
tcatgcatagatggtgtagcattagaaaactcaggcctagc

SEQ ID NO: 488 - DNA, 41 bp, Homo sapiens
aggtggatttttttaagaagcatattcatacaactgaatat

SEQ ID NO: 489 - DNA, 41 bp, Homo sapiens
gcctgatattctttccctatgaaattgcttcctcatctagg

SEQ ID NO: 490 - DNA, 41 bp, Homo sapiens
gaagaagctgtcagaattgcaagggaaattggtaagtcctt

SEQ ID NO: 491 - DNA, 41 bp, Homo sapiens
actgtgcccacccaagtttgtgttttgaaaagattggtcaa

SEQ ID NO: 492 - DNA, 41 bp, Homo sapiens
atggcatacagcctgggtgatatttttaaacataagtgaaa

SEQ ID NO: 493 - DNA, 41 bp, Homo sapiens
gggaaaatgttcatttaagtataaaacatgaaatggtattc

SEQ ID NO: 494 - DNA, 41 bp, Homo sapiens
cttgttagttcaggtctctttcagatgaggaagagagatta

SEQ ID NO: 495 - DNA, 41 bp, Homo sapiens
aaatggacaacaaaagtcaccggaaaaaagggaaaaaaaga

SEQ ID NO: 496 - DNA, 41 bp, Homo sapiens
tgagaaataagtgatgtcatgcatttttggttgtggatcat

SEQ ID NO: 497 - DNA, 41 bp, Homo sapiens
tgtggttctcccttcacagtggaatacaagggcttttatat

SEQ ID NO: 498 - DNA, 41 bp, Homo sapiens
taataagtggttatgccaagcggtccctgcagctcagaggc

SEQ ID NO: 499 - DNA, 41 bp, Homo sapiens
tctttgggcctccaccccctcgtctctagtggacatttgag

SEQ ID NO: 500 - DNA, 41 bp, Homo sapiens
aaaggaagctgggcgtcctccgggccccccaacacacgtcc

SEQ ID NO: 501 - DNA, 41 bp, Homo sapiens
ctaacacagttgcgaacatcggcagagccctcgggagccac

SEQ ID NO: 502 - DNA, 41 bp, Homo sapiens
ttgatgatgatgtcgatgccgaagagtgacacgcccagtgc

SEQ ID NO: 503 - DNA, 41 bp, Homo sapiens
cttcacagcgccgcaacaatcatgcatgagggagtgattcg

SEQ ID NO: 504 - DNA, 41 bp, Homo sapiens
ggccacagctggccagtctccttgtgctttgaatctccagc

SEQ ID NO: 505 - DNA, 41 bp, Homo sapiens
tgcagcgtgcggcagtgctttgttcttctttaagatgaaat

SEQ ID NO: 506 - DNA, 41 bp, Homo sapiens
cctacacaggaagccccggagccacagcaattctccctgcc

SEQ ID NO: 507 - DNA, 41 bp, Homo sapiens
tgtgctctggccaggggcctggacctcattctgttggtggt

SEQ ID NO: 508 - DNA, 41 bp, Homo sapiens
tcgcccaggctgaccacaagctccaaacaggactttcttgt

SEQ ID NO: 509 - DNA, 41 bp, Homo sapiens
tgcccaaacagtatcaaaagtggatgtttatcacaatacta

SEQ ID NO: 510 - DNA, 41 bp, Homo sapiens
ttagcaacaaaatcctgaagtcacttctagaccataaccca

SEQ ID NO: 511 - DNA, 41 bp, Homo sapiens
cagagggcagggcccacaccgtaccccacagaagcccagga

SEQ ID NO: 512 - DNA, 41 bp, Homo sapiens
gggtacagcccagcatggccgcaggggtccctgatgggaat

SEQ ID NO: 513 - DNA, 41 bp, Homo sapiens
gactgccaggtgtggacacatgctcgtcaagtggtgaagaa

SEQ ID NO: 514 - DNA, 41 bp, Homo sapiens
cacacggacgcttcctcctatgtgaagttctgtttgctccc

SEQ ID NO: 515 - DNA, 41 bp, Homo sapiens
atggtcatattatgcatgcacgtttttgatttcaagaatgc

SEQ ID NO: 516 - DNA, 41 bp, Homo sapiens
atgcggtgctcggtaactgtgcatccgatgcaggcctcact

SEQ ID NO: 517 - DNA, 41 bp, Homo sapiens
accagaattatcacagcaccttctcattcccagcgcgtcct

SEQ ID NO: 518 - DNA, 41 bp, Homo sapiens
tgatcatggtcactgccctgcgttcaaataatgcgagctga

SEQ ID NO: 519 - DNA, 41 bp, Homo sapiens
aggacaacatgccatttgtcgaaacgttttaaagatatgat

SEQ ID NO: 520 - DNA, 41 bp, Homo sapiens
gggggaagctgggtgcatgcggagcaccgtggagtctggga

SEQ ID NO: 521 - DNA, 41 bp, Homo sapiens
ccttgaagtcacccggccccgatgcaaggtgcccacatgtg

SEQ ID NO: 522 - DNA, 41 bp, Homo sapiens
tttggaaggaaaacgtggcgtgtgggcgtattctccagaag

SEQ ID NO: 523 - DNA, 41 bp, Homo sapiens
tcccagaccagaccttgcccaatgacgttgttggtaatgct

SEQ ID NO: 524 - DNA, 41 bp, Homo sapiens
tgagatcccccggacaacacactccaccttcccatggagct

SEQ ID NO: 525 - DNA, 41 bp, Homo sapiens
ttgtttgtgtctgtctcaaacccaaaggggtggctcagcct

SEQ ID NO: 526 - DNA, 41 bp, Homo sapiens
gaacctcccagggggcagaacaaaaagtcaacaagctggaa

SEQ ID NO: 527 - DNA, 41 bp, Homo sapiens
caaacgttgctgaagtctccccgacctttattgttttgccc

SEQ ID NO: 528 - DNA, 41 bp, Homo sapiens
gttccctgaccaggagtccaataggcaatagtctattaact

SEQ ID NO: 529 - DNA, 41 bp, Homo sapiens
tttgctcatgcacctgccttgcctttgtcatcacaacagaa

SEQ ID NO: 530 - DNA, 41 bp, Homo sapiens
acctccttccccgtgckccacgaggagcgggctgcaccttg

SEQ ID NO: 531 - DNA, 41 bp, Homo sapiens
gctgaaacccgattcctaccaggtgacgctgagaccgtacc

SEQ ID NO: 532 - DNA, 41 bp, Homo sapiens
tcctgctcgacctgctcctccagctgtgcaatcttggcctc

SEQ ID NO: 533 - DNA, 41 bp, Homo sapiens
tccagcgccgcgatggtggacttgaacttggacttgacggc

SEQ ID NO: 534 - DNA, 41 bp, Homo sapiens
tacgaggagaaggcggccgcgtatgataaactggaaaagac

SEQ ID NO: 535 - DNA, 41 bp, Homo sapiens
ttccgcagcttgaggtaggcggcgcagttcctctgaatcac

SEQ ID NO: 536 - DNA, 41 bp, Homo sapiens
cctgtggctggtaccttcccagcataatggatgatggagaa

SEQ ID NO: 537 - DNA, 41 bp, Homo sapiens
atgattgccatggcctccacggtttcctggaacatctcatc

SEQ ID NO: 538 - DNA, 41 bp, Homo sapiens
ccagaaccaccaacatcttcagtctctgtattcaattttat

SEQ ID NO: 539 - DNA, 41 bp, Homo sapiens
ttttcccagctgtaaaagggagctaataatagctcttgcgg

SEQ ID NO: 540 - DNA, 41 bp, Homo sapiens
gatacctgactccaggagccatcactttacaacctgagatt

SEQ ID NO: 541 - DNA, 41 bp, Homo sapiens
ttcttgcccttgtacatgtcgacgatcttctccgagtagat

SEQ ID NO: 542 - DNA, 41 bp, Homo sapiens
atcatgctcagtgaaacaaaccagaaaggccacacgctcta

SEQ ID NO: 543 - DNA, 41 bp, Homo sapiens
acctggtcaacagcttcccttaggattttactgccaagcca

SEQ ID NO: 544 - DNA, 41 bp, Homo sapiens
cacccagtctgaccttcacttttttgttgatggggctgagc

SEQ ID NO: 545 - DNA, 41 bp, Homo sapiens
gctgctgggggtgggtgcttggatcctggtgaaatggcctc

SEQ ID NO: 546 - DNA, 41 bp, Homo sapiens
agaatcatcttctcctttccttcacctgatacccagcttga

SEQ ID NO: 547 - DNA, 41 bp, Homo sapiens
cctgtcaggcctgacggggaggaaccactgcaccaccgaga

SEQ ID NO: 548 - DNA, 41 bp, Homo sapiens
ggctatgaatatagtacctgaaaaaatgccaagacatgatt

SEQ ID NO: 549 - DNA, 41 bp, Homo sapiens
cttttgggaatttcctctccccttggcactcggagttgggg

SEQ ID NO: 550 - DNA, 41 bp, Homo sapiens
caagccatggcagcggacagcctgctgagaacacccaggaa

SEQ ID NO: 551 - DNA, 41 bp, Homo sapiens
gaccagtgaacttcatccttatctgtccaggaggtggcctc

SEQ ID NO: 552 - DNA, 41 bp, Homo sapiens
tcagtatagatgcacccatcgtaagcctaactacattgtat

SEQ ID NO: 553 - DNA, 41 bp, Homo sapiens
gtgagcgtgccatcagcccagtggaggggcttaggtctgca

SEQ ID NO: 554 - DNA, 41 bp, Homo sapiens
ggtgccatccagtgccctgatagtcagttcgaatgcccgga

SEQ ID NO: 555 - DNA, 41 bp, Homo sapiens
ggcccgtagccctcacgtgggtgtgaaggacgtggagtgtg

SEQ ID NO: 556 - DNA, 41 bp, Homo sapiens
tcaggcctccctagcacctccccctaaccaaattctccctg

SEQ ID NO: 557 - DNA, 41 bp, Homo sapiens
agccatgagtttccaccagcagcagagtgagtcctgagcac

SEQ ID NO: 558 - DNA, 41 bp, Homo sapiens
attgcagagaatggaagaatttgaagaactgagtgacaagg

SEQ ID NO: 559 - DNA, 41 bp, Homo sapiens
agctactgggtagaattttacgtagtaactaggtagacact

SEQ ID NO: 560 - DNA, 41 bp, Homo sapiens
ggatggcatagcgagaatactaatctaggaagcgactggac

SEQ ID NO: 561 - DNA, 41 bp, Homo sapiens
gctttcctgctatcatagccgacttaagtagctgtattagg

SEQ ID NO: 562 - DNA, 41 bp, Homo sapiens
atgaggaagagagagacgaggtggggtgactcatgcctgaa

SEQ ID NO: 563 - DNA, 41 bp, Homo sapiens
tttctttgagacagggtctccctctgttacccaagctggra

SEQ ID NO: 564 - DNA, 41 bp, Homo sapiens
tcattagcagggtgatggtgaggctgagatgggcagggcca

SEQ ID NO: 565 - DNA, 41 bp, Homo sapiens
attgccaacatagctgttcatacctagaacaccttttcctt

SEQ ID NO: 566 - DNA, 41 bp, Homo sapiens
cacaacctcggtaaggctggtgatcttcaagccagtccgat

SEQ ID NO: 567 - DNA, 41 bp, Homo sapiens
gtccgttgtccacgttctacctccaccccactaactgaacg

SEQ ID NO: 568 - DNA, 41 bp, Homo sapiens
aggccaggggtctggatgcacatagcgttcccctagcctct

SEQ ID NO: 569 - DNA, 41 bp, Homo sapiens
tgcagaggtgtgggcccctggggacccagaagtccagccac

SEQ ID NO: 570 - DNA, 41 bp, Homo sapiens
gggtgaagtaaagtgggcagtgtgatttagcagagtggtca

SEQ ID NO: 571 - DNA, 41 bp, Homo sapiens
ggcacctgtcatagtcttgccgaaagatgacaagccctggt

SEQ ID NO: 572 - DNA, 41 bp, Homo sapiens
cgcagcccaggatgatctgtacgggacagaggcagcggcct

SEQ ID NO: 573 - DNA, 41 bp, Homo sapiens
tcggaacagcgagtcctctgccgtcgagagcagggaggggt

SEQ ID NO: 574 - DNA, 41 bp, Homo sapiens
tttgcccagtgacgcagcattccaggctgagattgcagaat

SEQ ID NO: 575 - DNA, 41 bp, Homo sapiens
gccccctctgcaggtcccctcggtgtactctgaggtgggaa

SEQ ID NO: 576 - DNA, 41 bp, Homo sapiens
ggacacaacaggacccactgaggaaaacaatgatgacttgg

SEQ ID NO: 577 - DNA, 41 bp, Homo sapiens
cccctccactttgctcacccgtcttccgggccctgaaccca

SEQ ID NO: 578 - DNA, 41 bp, Homo sapiens
tcctgtgccggctgcaggtacggaacaagtaggctagtgtc

SEQ ID NO: 579 - DNA, 41 bp, Homo sapiens
aggaaagactgttgggcctcagaaaacatcccacgtgctag

SEQ ID NO: 580 - DNA, 41 bp, Homo sapiens
gggacttggtttcatgtctccatctctcagttctgtttccc

SEQ ID NO: 581 - DNA, 41 bp, Homo sapiens
atagagagggtctgttaggtgcttgggatcttgttcttcaa

SEQ ID NO: 582 - DNA, 41 bp, Homo sapiens
attccaattgaagattgaaaatggcctgtttggtaaactgg

SEQ ID NO: 583 - DNA, 41 bp, Homo sapiens
taactcaaagcacaaagtttagaattcctacattctaaaga

SEQ ID NO: 584 - DNA, 41 bp, Homo sapiens
gtcacctgcctcggagccagctaggctgtttaacagtgcag

SEQ ID NO: 585 - DNA, 41 bp, Homo sapiens
ggagctttggcatcgcagaggcttgagctgagtctggctct

SEQ ID NO: 586 - DNA, 41 bp, Homo sapiens
cagagcccctccctctaaacccagtctttcaaagggattgt

SEQ ID NO: 587 - DNA, 41 bp, Homo sapiens
caatttcttgctgaaagccccgagttatgccagacactgtg

SEQ ID NO: 588 - DNA, 41 bp, Homo sapiens
acctttgcccagatccaaattttttttcttcattcgaagct

SEQ ID NO: 589 - DNA, 41 bp, Homo sapiens
acggatctcttaccattaaactcaggtggagagggagtgcc

SEQ ID NO: 590 - DNA, 41 bp, Homo sapiens
tttcacagatgaggaggctgacctcaggaaatgtgactcag

SEQ ID NO: 591 - DNA, 41 bp, Homo sapiens
ccaacaccaccccttgcccaaccaatgcacacagtagggct

SEQ ID NO: 592 - DNA, 41 bp, Homo sapiens
cccatatcatgcagaggatccgggatttcaatccaggtcta

SEQ ID NO: 593 - DNA, 41 bp, Homo sapiens
tgacgtgtgcagagagacatgtcagcctgccctgcacttgt

SEQ ID NO: 594 - DNA, 41 bp, Homo sapiens
ggcagcatattagaaaatagtttatgttacaacaaaaaccc

SEQ ID NO: 595 - DNA, 41 bp, Homo sapiens
tgccccttctcactggtctgtggctggcagggccatctttc

SEQ ID NO: 596 - DNA, 41 bp, Homo sapiens
gaatccatcccaaggacaccatttgaaaacatgaaataaca

SEQ ID NO: 597 - DNA, 41 bp, Homo sapiens
cagcggggaggggaaaggtccgaaatgaggggagagacgtg

SEQ ID NO: 598 - DNA, 41 bp, Homo sapiens
gctgggcagagccattcctgggctggctgggtgtgtttggg

SEQ ID NO: 599 - DNA, 41 bp, Homo sapiens
acaggcatcagggatacagtagtgaacaagcatacacaatc

SEQ ID NO: 600 - DNA, 41 bp, Homo sapiens
aggtgaagctgaggcctgagaccagaaggagagaaaaggaa

SEQ ID NO: 601 - DNA, 41 bp, Homo sapiens
cactcattaatccattaaacaattaatctattaatccatga

SEQ ID NO: 602 - DNA, 41 bp, Homo sapiens
gtgtatgctgtgaagaaggccaccccccttcctgcccatcc

SEQ ID NO: 603 - DNA, 41 bp, Homo sapiens
ctgtcactatgcccctgcctctctcagtgtctatctctgtt

SEQ ID NO: 604 - DNA, 41 bp, Homo sapiens
gggatgacagtgagaggagggcaacagtaaaaggagtcata

SEQ ID NO: 605 - DNA, 41 bp, Homo sapiens
gtgtgtctgtcagggaatgtatccctcttccattctctgtg

SEQ ID NO: 606 - DNA, 41 bp, Homo sapiens
ccattcttggtggtgagcctagactctgagcctgggatgtg

SEQ ID NO: 607 - DNA, 41 bp, Homo sapiens
gtctggctgccccttggccttcacyacagtcaggtccagcc

SEQ ID NO: 608 - DNA, 41 bp, Homo sapiens
ttgaggattaaagagcagaratcatgtagcatctggcacat

SEQ ID NO: 609 - DNA, 41 bp, Homo sapiens
cgtcatgtagcatctggcacatgggggaacgcaatggaagt

SEQ ID NO: 610 - DNA, 41 bp, Homo sapiens
cagagaatatttcacatgcacgtagcaaaaacaccaggggt

SEQ ID NO: 611 - DNA, 41 bp, Homo sapiens
aacatggattaatgtgggaatttggcttcaagaacacaacc

SEQ ID NO: 612 - DNA, 41 bp, Homo sapiens
attatttcattttaaaaccacagaataaaaatgacacctga

SEQ ID NO: 613 - DNA, 41 bp, Homo sapiens
aagcagattatgaggcagcttcacccctcccagcactgggg

SEQ ID NO: 614 - DNA, 41 bp, Homo sapiens
ccagccctgtagtggacatacttgcctttgcctattcagca

SEQ ID NO: 615 - DNA, 41 bp, Homo sapiens
gaactcggtggaggagaagaaaaactccaagatgctccaga

SEQ ID NO: 616 - DNA, 41 bp, Homo sapiens
tgtgggctggacttagcaactcacttctaactaacagaatg

SEQ ID NO: 617 - DNA, 41 bp, Homo sapiens
ggtgtcaattcactcccagcagcactgactgagtgctgacc

SEQ ID NO: 618 - DNA, 41 bp, Homo sapiens
atgttaggcggtcccacctgagttctggagatcttcacaca

SEQ ID NO: 619 - DNA, 41 bp, Homo sapiens
ggtgggcagaggctggatcccatggtgaggagtttccattt

SEQ ID NO: 620 - DNA, 41 bp, Homo sapiens
ttgccatgggccacctctactgagtgctcgatgaacaacaa

SEQ ID NO: 621 - DNA, 41 bp, Homo sapiens
tttggctggggcaagcttacatggttcggcagtagtaccag

SEQ ID NO: 622 - DNA, 41 bp, Homo sapiens
gtggccccaggaatgggggcatctggtggtatctgggctgg

SEQ ID NO: 623 - DNA, 41 bp, Homo sapiens
atgcattgtggtagattcattcaatggagtatacacagcaa

SEQ ID NO: 624 - DNA, 41 bp, Homo sapiens
gtggcagctgccatttttccagtgccacaaatggtagttac

SEQ ID NO: 625 - DNA, 41 bp, Homo sapiens
ttgggaggaagaccacagagatgatgtgccagtctcagaac

SEQ ID NO: 626 - DNA, 41 bp, Homo sapiens
aaaatacagggtacagggacgctcaaagagtgatttgcttc

SEQ ID NO: 627 - DNA, 41 bp, Homo sapiens
gtgagatggggcacagcagcagccggaaggttatttgtgtg

SEQ ID NO: 628 - DNA, 41 bp, Homo sapiens
gcagggcagagaaggggaagttgctggctgccctcctcact

SEQ ID NO: 629 - DNA, 41 bp, Homo sapiens
gctcctggattcactcctttgatcctcacctcaatcctttg

SEQ ID NO: 630 - DNA, 41 bp, Homo sapiens
agttggcttgtatggaccccaccgatgacggacagttccaa

SEQ ID NO: 631 - DNA, 41 bp, Homo sapiens
agtggattgaggatggacatatgtatctggaagcaccaaaa

SEQ ID NO: 632 - DNA, 41 bp, Homo sapiens
ctgggttcactggaaatcagcattaagaatgtacaagggaa

SEQ ID NO: 633 - DNA, 41 bp, Homo sapiens
atgtaaactgcctttgaaaggctataacacagttcagttgg

SEQ ID NO: 634 - DNA, 41 bp, Homo sapiens
acttaatcttgctcagttccccagtttacacttttgaatgg

SEQ ID NO: 635 - DNA, 41 bp, Homo sapiens
gcagcatagatgaatgtaatgttgaaacaggaagatgtgtt

SEQ ID NO: 636 - DNA, 41 bp, Homo sapiens
cttagcctgcaattgcaatctgtatgggaccatgaagcagc

SEQ ID NO: 637 - DNA, 41 bp, Homo sapiens
tagccgtttacagaatatccagaataccattgaagagactg

SEQ ID NO: 638 - DNA, 41 bp, Homo sapiens
gtttcagattttgataggcgtgtgaacgataacaagacggc

SEQ ID NO: 639 - DNA, 41 bp, Homo sapiens
atgagggagaaatgccctttctggcaattgttggagctgga

SEQ ID NO: 640 - DNA, 41 bp, Homo sapiens
aggaacagtgctacttactgatgggtagactgggagaggtg

SEQ ID NO: 641 - DNA, 41 bp, Homo sapiens
ttggcaatgggtaagtctattgtactgtgtaaacttggact

SEQ ID NO: 642 - DNA, 41 bp, Homo sapiens
gatatagatctcttggaaatataataatggtatgcaggaag

SEQ ID NO: 643 - DNA, 41 bp, Homo sapiens
gcaacccggggaaatacagcaaaatgcacaagtactggctg

SEQ ID NO: 644 - DNA, 41 bp, Homo sapiens
ctgtacaaactttcttccatgattttgattatatccatttt

SEQ ID NO: 645 - DNA, 41 bp, Homo sapiens
ccctcattatctgcctaaacaattttttctcaactcctata

SEQ ID NO: 646 - DNA, 41 bp, Homo sapiens
ctagcactgtacacaccccatactgtgtatgctatttgttg

SEQ ID NO: 647 - DNA, 41 bp, Homo sapiens
caaaagttatctctaaccaaggtactcaaacagagtcttta

SEQ ID NO: 648 - DNA, 41 bp, Homo sapiens
ccttgtaaatctccacctgatatttctcatggtgttgtagc

SEQ ID NO: 649 - DNA, 41 bp, Homo sapiens
tcccataggaattataaaattgaaaagtatgacaaaaattt

SEQ ID NO: 650 - DNA, 41 bp, Homo sapiens
aggcccttcagcttcaccacttgcttctctttaaacaagtc

SEQ ID NO: 651 - DNA, 41 bp, Homo sapiens
gatagaatttggcccagagaagttaactaatatatccatga

SEQ ID NO: 652 - DNA, 41 bp, Homo sapiens
ctgtttctccttaaaatggaaaaatggcctctacagagtag

SEQ ID NO: 653 - DNA, 41 bp, Homo sapiens
gcttggtggggccactgggcatctgtttctcgggtgttttg

SEQ ID NO: 654 - DNA, 41 bp, Homo sapiens
ccattccctcggcgaagagcagaggttgaagaaatgctact

SEQ ID NO: 655 - DNA, 41 bp, Homo sapiens
gcaagggccagagcctctgtatgctgcattcggcaaccaca

SEQ ID NO: 656 - DNA, 41 bp, Homo sapiens
ggttcctgaaggaggagtgggagtttggtaaatggatggag

SEQ ID NO: 657 - DNA, 41 bp, Homo sapiens
ttacctgctaaggcctgcaaccttgaggatgtccagggctg

SEQ ID NO: 658 - DNA, 41 bp, Homo sapiens
ccagaaggtttctttgctcctcttccctacaaagacagagc

SEQ ID NO: 659 - DNA, 41 bp, Homo sapiens
aattcactcctttaaaatacgcaatgcagtgtttttagaaa

SEQ ID NO: 660 - DNA, 41 bp, Homo sapiens
ccactccctctcctgctcttatgtgtgtgatccaaagggaa

SEQ ID NO: 661 - DNA, 41 bp, Homo sapiens
cagggacagctgaagccaagttctcccaaagcagccttggc

SEQ ID NO: 662 - DNA, 41 bp, Homo sapiens
gtcaggagcctggccaggccacaccccttgctgtctcagca

SEQ ID NO: 663 - DNA, 41 bp, Homo sapiens
ggagattctgcctcagggccatgagagtcccatcttcaagc

SEQ ID NO: 664 - DNA, 41 bp, Homo sapiens
gctcagctaccgttggtggcgtttattaaactgtgcaccca

SEQ ID NO: 665 - DNA, 41 bp, Homo sapiens
aaggtggctgactccagcccctttgcccttgaactgctgat

SEQ ID NO: 666 - DNA, 41 bp, Homo sapiens
tgaagacctgaaaagcaaatgccaggcagccccactccctc

SEQ ID NO: 667 - DNA, 41 bp, Homo sapiens
ttctttgtaatttggaatcctcctaatttccaaatgggttc

SEQ ID NO: 668 - DNA, 41 bp, Homo sapiens
gggacctggccctggccatctgggacagtgagcgacagggc

SEQ ID NO: 669 - DNA, 41 bp, Homo sapiens
aggtggggacccggctccaagggcacccgggtcttctgcag

SEQ ID NO: 670 - DNA, 41 bp, Homo sapiens
acaggccgctctcccagcagtgtgttgaggtgcacagccag

SEQ ID NO: 671 - DNA, 41 bp, Homo sapiens
tggcgcaagagaaccagggcatcttcttctcgggggactcc

SEQ ID NO: 672 - DNA, 41 bp, Homo sapiens
tgctgtgcccacatcccctggaacaggcagcccagcctgtg

SEQ ID NO: 673 - DNA, 41 bp, Homo sapiens
tggtgagttatggacccycccacctccactactacactgta

SEQ ID NO: 674 - DNA, 41 bp, Homo sapiens
tcagggcctggggcaggcgctgcacagcccccaccgctgct

SEQ ID NO: 675 - DNA, 41 bp, Homo sapiens
gcatggcatgcggaagatggcgaagaatgttttatggcctc

SEQ ID NO: 676 - DNA, 41 bp, Homo sapiens
tctcagtagctgagacctgaaaaatttggagaatcactttg

SEQ ID NO: 677 - DNA, 41 bp, Homo sapiens
acatgaggccactgaggcaggcctctttccttccccttctc

SEQ ID NO: 678 - DNA, 41 bp, Homo sapiens
cctattcttaatcctattttacaaatgaagtgacttgccca

SEQ ID NO: 679 - DNA, 41 bp, Homo sapiens
ggaatgggtcaagaatgttccttcccttctgaatgtccctg

SEQ ID NO: 680 - DNA, 41 bp, Homo sapiens
aagcggggaggagctaaataatatttttctctccttgttca

SEQ ID NO: 681 - DNA, 41 bp, Homo sapiens
aacttggaacatctccgcaataagacagaggatctggaagc

SEQ ID NO: 682 - DNA, 41 bp, Homo sapiens
acatcgcagaaggtggctcggaaattctggtggaagaacgt

SEQ ID NO: 683 - DNA, 41 bp, Homo sapiens
ttcccgaggccctgctgccacgttgtatgccccagaaggta

SEQ ID NO: 684 - DNA, 41 bp, Homo sapiens
tgagagtcagggtttgggactagattggcaagtcaggctct

SEQ ID NO: 685 - DNA, 41 bp, Homo sapiens
tctccaggacctagtatggtacctgaccgtggcactcatag

SEQ ID NO: 686 - DNA, 41 bp, Homo sapiens
ctacctcagagtatgtgcccgttggatggtggctgttattc

SEQ ID NO: 687 - DNA, 41 bp, Homo sapiens
ctagtctctgagctgagtgctgacttagggaggcaatgtta

SEQ ID NO: 688 - DNA, 41 bp, Homo sapiens
acagtgtggcgtaaggcagtatggcccttgtcctcttgctt

SEQ ID NO: 689 - DNA, 41 bp, Homo sapiens
ttagggcagctgtgcattgattgggtagacgccattctgga

SEQ ID NO: 690 - DNA, 41 bp, Homo sapiens
tgaggcccccacctggccctcatctgcccctgagatctaga

SEQ ID NO: 691 - DNA, 41 bp, Homo sapiens
cgcataatttccgtcacctcgttcgcctgctgctggcaccg

SEQ ID NO: 692 - DNA, 41 bp, Homo sapiens
ccccaacatgtgcacccctgtatttcctgtcatgccacaga

SEQ ID NO: 693 - DNA, 41 bp, Homo sapiens
ccagatctccatcattggcggtagtctctggtcacctgact

SEQ ID NO: 694 - DNA, 41 bp, Homo sapiens
tttgttctgactttacatcctcttccccaggtcacttttca

SEQ ID NO: 695 - DNA, 41 bp, Homo sapiens
attcctgtcccttgtgccgccatgagctgcccactgatgac

SEQ ID NO: 696 - DNA, 41 bp, Homo sapiens
tttgataccaagaacacattactgcatgaatcctccagcaa

SEQ ID NO: 697 - DNA, 41 bp, Homo sapiens
tctaaaattaggggtttgatgtagcttatctggaaggtgtt

SEQ ID NO: 698 - DNA, 41 bp, Homo sapiens
gatgcggtctggaaagcaccggggtggccgtcggctgacgc

SEQ ID NO: 699 - DNA, 41 bp, Homo sapiens
ctccgtggaacttctcctggcacaaattctgttcctaggga

SEQ ID NO: 700 - DNA, 41 bp, Homo sapiens
gaggggagccacaggaatggtcgtggccagaagcccttctc

SEQ ID NO: 701 - DNA, 41 bp, Homo sapiens
ggcaccttttccctgataagccacaaatcataaccaaacaa

SEQ ID NO: 702 - DNA, 41 bp, Homo sapiens
ttgcactccagtttttttttttttaaaaaagcggtttctac

SEQ ID NO: 703 - DNA, 41 bp, Homo sapiens
gaaaaggctgtctgattatcatgtcatccaaaaaaaacaga

SEQ ID NO: 704 - DNA, 41 bp, Homo sapiens
gaactaagaggaataaaggtgttgctttatacctgtcccta

SEQ ID NO: 705 - DNA, 41 bp, Homo sapiens
actaacatgtcctgcctattttctgtcagctgcaaggtact

SEQ ID NO: 706 - DNA, 41 bp, Homo sapiens
gctgacccagggtccacatgttctttttctaacttgttcat

SEQ ID NO: 707 - DNA, 41 bp, Homo sapiens
tgcttccccatttctgtcctgaaagccctctggcaagactg

SEQ ID NO: 708 - DNA, 41 bp, Homo sapiens
cagtgatgaactcctgggctgaagtgacccacccgcctctg

SEQ ID NO: 709 - DNA, 41 bp, Homo sapiens
gcgacttcgactaagcaacactgcatctattttcatgcaac

SEQ ID NO: 710 - DNA, 41 bp, Homo sapiens
cctcaaatgttagagtcagtacaccagctcatagtttccat

SEQ ID NO: 711 - DNA, 41 bp, Homo sapiens
cgtttaattctttctcatcactttcctagggcatttgcaat

SEQ ID NO: 712 - DNA, 41 bp, Homo sapiens
catcagagttttatgattagcagatatatcttaactgacac

SEQ ID NO: 713 - DNA, 42 bp, Homo sapiens
agcaaaaccaaagaaatcagcagaagaccataaaaacagacg

SEQ ID NO: 714 - DNA, 41 bp, Homo sapiens
ctataaaattagtatgcttacaattattaaacatatacaga

SEQ ID NO: 715 - DNA, 41 bp, Homo sapiens
taaacactttaatgcagtgacactcaggtataaaactcaga

SEQ ID NO: 716 - DNA, 41 bp, Homo sapiens
atagaagacaaagttttcattcgtctcattcaagttcactt

SEQ ID NO: 717 - DNA, 41 bp, Homo sapiens
agtgcagggcaggactgctggctgaccccgggccacctgga

SEQ ID NO: 718 - DNA, 41 bp, Homo sapiens
aacctcttggtacatgttagaggaaatgaagctggcaacaa

SEQ ID NO: 719 - DNA, 41 bp, Homo sapiens
tcatcagatcaaggacattacggaattaaagggctctaaga

SEQ ID NO: 720 - DNA, 41 bp, Homo sapiens
ccactgctattggttatttacctagcatccatttcccttta

SEQ ID NO: 721 - DNA, 41 bp, Homo sapiens
atctacctctcctgcctcatatattattacccagccccttc

SEQ ID NO: 722 - DNA, 41 bp, Homo sapiens
gtcaattgcaaatggaggtgagacctgagaaaacaaagaaa

SEQ ID NO: 723 - DNA, 41 bp, Homo sapiens
gagtgtgtaacaactcacctgccaaatcgactagcccttag

SEQ ID NO: 724 - DNA, 41 bp, Homo sapiens
cttgtaagccatcttaagccgttataggcctaagatgtata

SEQ ID NO: 725 - DNA, 41 bp, Homo sapiens
cttgagacctgtgtctcctcatgttcacactgttcctgact

SEQ ID NO: 726 - DNA, 41 bp, Homo sapiens
gaggcatgggttgaactgcattcacatatgtacttaaaaga

SEQ ID NO: 727 - DNA, 41 bp, Homo sapiens
tgtttcttgaagtttgactacttaaaaacataggtgtaaag

SEQ ID NO: 728 - DNA, 41 bp, Homo sapiens
agagtcacggcatgtgggaatgtttccatggacactggatc

SEQ ID NO: 729 - DNA, 41 bp, Homo sapiens
aatgagatcttatgtcaaggatttaatctttggtattccaa

SEQ ID NO: 730 - DNA, 41 bp, Homo sapiens
tctggacctcagtttcctcaatgagctggtaagaatgcact

SEQ ID NO: 731 - DNA, 41 bp, Homo sapiens
aggttgatagcaatgtttgggagatatgtcctagaagtgtt

SEQ ID NO: 732 - DNA, 41 bp, Homo sapiens
gcatgataacccgagccatcactaaatattatagcttcctt

SEQ ID NO: 733 - DNA, 41 bp, Homo sapiens
ctccagtttctccctttctcgccaactaggtccatccaaac

SEQ ID NO: 734 - DNA, 41 bp, Homo sapiens
aactgtaaggatctcttgctttatatactattgggggaaca

SEQ ID NO: 735 - DNA, 41 bp, Homo sapiens
ccttagctcttcctaaaacacacaatcataaaggaaaccgt

SEQ ID NO: 736 - DNA, 41 bp, Homo sapiens
ctgacagtaaagggaactcagtatgtctgagtctttgctca

SEQ ID NO: 737 - DNA, 41 bp, Homo sapiens
aacatttacagaagcgagaaaaagttttgtttgcttttgtt

SEQ ID NO: 738 - DNA, 41 bp, Homo sapiens
taagttcaataaatcccaaactgcacactctgaattagggg

SEQ ID NO: 739 - DNA, 41 bp, Homo sapiens
aagatagccatctttgggcagagagtcatgaaatgtaccct

SEQ ID NO: 740 - DNA, 41 bp, Homo sapiens
gctgggccgacggggacgagacggcgactggagcagcagcg

SEQ ID NO: 741 - DNA, 41 bp, Homo sapiens
ctctgtcttggtcactgtgcgaggattgaagggaactattg

SEQ ID NO: 742 - DNA, 41 bp, Homo sapiens
atcgtcttttacaataagatgcatgcccctatgagtatttt

SEQ ID NO: 743 - DNA, 41 bp, Homo sapiens
aaggagaaaaacagtgaaccatagttcttactgctcacact

SEQ ID NO: 744 - DNA, 41 bp, Homo sapiens
gattatttgattgccatgaacgaagctgaattacataattc

SEQ ID NO: 745 - DNA, 41 bp, Homo sapiens
agggacctgtcttcagaatctaagaagcataatgtccttaa

SEQ ID NO: 746 - DNA, 41 bp, Homo sapiens
tagagtccctaccatgcaccctgggcaagaagtcagttctg

SEQ ID NO: 747 - DNA, 41 bp, Homo sapiens
tcgggtctcttaccatgcccgccctcccttcctcagggaat

SEQ ID NO: 748 - DNA, 41 bp, Homo sapiens
aggaccttcagagaccccgcgttctctgaaaccaggatgga

SEQ ID NO: 749 - DNA, 41 bp, Homo sapiens
caggggctgcactcaccatcgtctgacacctccacttcatc

SEQ ID NO: 750 - DNA, 41 bp, Homo sapiens
gtacacaagggtagggcagaggatggacagcagggcagaat

SEQ ID NO: 751 - DNA, 41 bp, Homo sapiens
agtttctgcagcactttatctttccatctggccatgaggaa

SEQ ID NO: 752 - DNA, 41 bp, Homo sapiens
caggcattgaaggtcagcttgttctcctcctgggtgagttt

SEQ ID NO: 753 - DNA, 41 bp, Homo sapiens
gggcacgacctaccatccacggtgacttggcaggagcactc

SEQ ID NO: 754 - DNA, 41 bp, Homo sapiens
ttacttctatccttgcttcttgaactggtcattccctgact

SEQ ID NO: 755 - DNA, 41 bp, Homo sapiens
agaacaagctgttagcaggacgcctctgctgctgcggggcc

SEQ ID NO: 756 - DNA, 41 bp, Homo sapiens
tcggctgggatctccttcagttcgtcttccgatagggtctt

SEQ ID NO: 757 - DNA, 41 bp, Homo sapiens
aggcctcagggacccatagcagtcactaccaccaccatcag

SEQ ID NO: 758 - DNA, 41 bp, Homo sapiens
ttgtccagaaatcactgtgactggatacacaaatgcagcac

SEQ ID NO: 759 - DNA, 41 bp, Homo sapiens
cttggctgctgaatggtgagatccccctgccccagctctct

SEQ ID NO: 760 - DNA, 41 bp, Homo sapiens
gaagtcttctgaaggaccggggtctgcggggccgttctggg

SEQ ID NO: 761 - DNA, 41 bp, Homo sapiens
tggtggcttttgtttctctcgcaaatgacctgtgtggtggt

SEQ ID NO: 762 - DNA, 41 bp, Homo sapiens
aggacgggtctccactgctggagctgaaaatctatccctgt

SEQ ID NO: 763 - DNA, 45 bp, Homo sapiens
tttgtgaccttgtatggatgacttaacttctctgaatcttatttc

SEQ ID NO: 764 - DNA, 41 bp, Homo sapiens
aaaactcaataagatgcctatattttatgcatctccattaa

SEQ ID NO: 765 - DNA, 41 bp, Homo sapiens
ttcaccatccctctactttcggcttgccaaaacttacagga

SEQ ID NO: 766 - DNA, 41 bp, Homo sapiens
tggccagtgctcagcagatggaagttccaaatcgagtcact

SEQ ID NO: 767 - DNA, 41 bp, Homo sapiens
gcatggagtcaactcttgagtgatccacactgagggaggtt

SEQ ID NO: 768 - DNA, 41 bp, Homo sapiens
tgactcctggtccagggcctactggggactagataagatgt

SEQ ID NO: 769 - DNA, 41 bp, Homo sapiens
caagctagagacttggtatacagcagcagttacatgagtgg

SEQ ID NO: 770 - DNA, 41 bp, Homo sapiens
cagactgtggacatccgaattggcaatgacatgaatttaag

SEQ ID NO: 771 - DNA, 41 bp, Homo sapiens
aggcaccaggtcccatggccggtttcccctgagaaaacatt

SEQ ID NO: 772 - DNA, 41 bp, Homo sapiens
atggagagctgccaagccaaccctgccagggtcatcagctc

SEQ ID NO: 773 - DNA, 41 bp, Homo sapiens
atagctgtccttactcctttcctagacagacagtgtcttgg

SEQ ID NO: 774 - DNA, 41 bp, Homo sapiens
gctttttataccgcttaacgaaaataatttaaaaggctgtc

SEQ ID NO: 775 - DNA, 41 bp, Homo sapiens
agctgcaatgcctatgagcaggacctgggtttgtacatctt

SEQ ID NO: 776 - DNA, 41 bp, Homo sapiens
ctaggatagcagagatattacttcaggatcagatcttgact

SEQ ID NO: 777 - DNA, 41 bp, Homo sapiens
tctggggagtctttagcccccagcagaggccatttctagca

SEQ ID NO: 778 - DNA, 41 bp, Homo sapiens
gaataaaacttacggagagcctctaacttcattcaatttgt

SEQ ID NO: 779 - DNA, 41 bp, Homo sapiens
ataatatattttaagcagggtagggtatcccaagatctcaa

SEQ ID NO: 780 - DNA, 41 bp, Homo sapiens
gtatggtaaagaatcccactcctgcatcaatcagtgggcaa

SEQ ID NO: 781 - DNA, 41 bp, Homo sapiens
ttttccttacaccaagcttacgtgggtggctgtagccacaa

SEQ ID NO: 782 - DNA, 41 bp, Homo sapiens
gcaccatgggggaaattatcggtattatttttttgaaatca

SEQ ID NO: 783 - DNA, 41 bp, Homo sapiens
tatagycaaagagttgtgcactgatcacctcaatgaattta

SEQ ID NO: 784 - DNA, 41 bp, Homo sapiens
gttctgggcaactgctttagtctgaatgcaaaaaactggaa

SEQ ID NO: 785 - DNA, 41 bp, Homo sapiens
aaacaaaagccccacagcaaaaaacaggaaggaaggggaac

SEQ ID NO: 786 - DNA, 41 bp, Homo sapiens
atagtgagggatgactgtatcttccacttaaaaatcccaag

SEQ ID NO: 787 - DNA, 41 bp, Homo sapiens
ggaaaataaaactgtacctcgtctccagtctccccatattt

SEQ ID NO: 788 - DNA, 41 bp, Homo sapiens
taatggctttcaaagtgcctgaattccattctacactaaaa

SEQ ID NO: 789 - DNA, 41 bp, Homo sapiens
acctcaaaagaaaaaataacataaacaatattcaactcaag

SEQ ID NO: 790 - DNA, 41 bp, Homo sapiens
gcttggttcaggccctggttacatacctggatttcaaatct

SEQ ID NO: 791 - DNA, 41 bp, Homo sapiens
acccacagctttcagcagtgaagaatatgaatggaaactgg

SEQ ID NO: 792 - DNA, 41 bp, Homo sapiens
gagtgaggtagagaacaggtataattcaccataagtcctga

SEQ ID NO: 793 - DNA, 41 bp, Homo sapiens
acctggttctttgaaagaacaaataaaattcacaaactgct

SEQ ID NO: 794 - DNA, 41 bp, Homo sapiens
tttttctcttcagctggcccgaattggtttctgttaatttt

SEQ ID NO: 795 - DNA, 41 bp, Homo sapiens
gaagagactaagagaatcacggaagagagaaggaggtcaag

SEQ ID NO: 796 - DNA, 41 bp, Homo sapiens
tcttgaagggttttagttccgtaagttccagggaggggtct

SEQ ID NO: 797 - DNA, 41 bp, Homo sapiens
aaacgtttaattcttctgtgagttctgttctaatttctgag

SEQ ID NO: 798 - DNA, 41 bp, Homo sapiens
aggcctagaattctctgaaacgtcatttttcagtttctaca

SEQ ID NO: 799 - DNA, 41 bp, Homo sapiens
gtagccttgcgcctcactctcgtgatggagccgcctgctac

SEQ ID NO: 800 - DNA, 41 bp, Homo sapiens
attgtcattttccttgtgttttattggttcaggctatccaa

SEQ ID NO: 801 - DNA, 41 bp, Homo sapiens
caaggcatcttggctcctacatagggccttttggctcctct

SEQ ID NO: 802 - DNA, 41 bp, Homo sapiens
agatctccaaggttttcaccaagaaacacttgacccgactt

SEQ ID NO: 803 - DNA, 41 bp, Homo sapiens
cctcaatgcagaggggtcataagagcaggctgggagccaga

SEQ ID NO: 804 - DNA, 41 bp, Homo sapiens
gttcctcctcagaaactgccgtgtatgagtttgtatcctta

SEQ ID NO: 805 - DNA, 41 bp, Homo sapiens
cataggcgaggcccagcccatgtgtccagagacatctgtga

SEQ ID NO: 806 - DNA, 41 bp, Homo sapiens
gctcttcaaggtctggtgctctcttccacagtactgtagcc

SEQ ID NO: 807 - DNA, 41 bp, Homo sapiens
aaatgggtgctcagacccctgtcctacttacctcaaaaggt

SEQ ID NO: 808 - DNA, 41 bp, Homo sapiens
tgtcagcagcctggtattggaaagagttaaaggaaaatctc

SEQ ID NO: 809 - DNA, 41 bp, Homo sapiens
cagttcaggggaggagcctcgggacgtcagtggcaaaatca

SEQ ID NO: 810 - DNA, 41 bp, Homo sapiens
gcataggcttaactcgctgaagagttaattgttttattttt

SEQ ID NO: 811 - DNA, 41 bp, Homo sapiens
aggggaaacgtctcccagattgctcccttggctttgaggcc

SEQ ID NO: 812 - DNA, 41 bp, Homo sapiens
agccaaagccagagtggccatggcccagggagggtgagctg

SEQ ID NO: 813 - DNA, 41 bp, Homo sapiens
tttcagagagggaagccagaagagaagagggtgcaggctga

SEQ ID NO: 814 - DNA, 41 bp, Homo sapiens
caagtcctccggttcttccttgggattggcgggtccacttg

SEQ ID NO: 815 - DNA, 41 bp, Homo sapiens
aggctgcctccgcacctgactgctgcccaggtggggtttcc

SEQ ID NO: 816 - DNA, 41 bp, Homo sapiens
tggctaggacagggtctcggactagggaagtggtttctctg

SEQ ID NO: 817 - DNA, 41 bp, Homo sapiens
ttacgggaagcccttctggcactcactcagggcagcagctt

SEQ ID NO: 818 - DNA, 41 bp, Homo sapiens
gcctgggcaggaagagggacgagagggtctcccacatggga

SEQ ID NO: 819 - DNA, 41 bp, Homo sapiens
atcgtgttccccaggaagttattcttgatttagtttaaact

SEQ ID NO: 820 - DNA, 41 bp, Homo sapiens
gaaccaccttctcttgccaggctgtactcctcatttagttt

SEQ ID NO: 821 - DNA, 41 bp, Homo sapiens
aaggtgggagccagagtgggttgctgtaggggtgagggagg

SEQ ID NO: 822 - DNA, 41 bp, Homo sapiens
gccatccagcgcggctgctcgggcgccacctccatggccgg

SEQ ID NO: 823 - DNA, 41 bp, Homo sapiens
tccctgggcccgtcgccctctgggctcccgccggaactcct

SEQ ID NO: 824 - DNA, 41 bp, Homo sapiens
acacagacattgtcgagcgccggtccctctttattggccag

SEQ ID NO: 825 - DNA, 41 bp, Homo sapiens
gcctggtgagagcagatttattccaatttatgggctggaac

SEQ ID NO: 826 - DNA, 41 bp, Homo sapiens
cacaccgacacacatggccatacaatcagatgcaactcggc

SEQ ID NO: 827 - DNA, 41 bp, Homo sapiens
cttgttcacagaagtgggagtcaggagggggggagaaagtg

SEQ ID NO: 828 - DNA, 41 bp, Homo sapiens
aggaccaggcggctaagcagagagaagagccagaggggcgt

SEQ ID NO: 829 - DNA, 41 bp, Homo sapiens
cgggccatggacaccgacacactgacacaggtcaaggagaa

SEQ ID NO: 830 - DNA, 41 bp, Homo sapiens
ctgcggttcagctccttggtcagatctgtcatgtctgtctg

SEQ ID NO: 831 - DNA, 41 bp, Homo sapiens
gcacgtcggctcttggtacaaaagacgaacagggctgcggg

SEQ ID NO: 832 - DNA, 41 bp, Homo sapiens
tcccccggggccctgagcaatgcatcagcgccagtggactt

SEQ ID NO: 833 - DNA, 41 bp, Homo sapiens
ttcaccaggacctggagctcagagcctacatggaggtcatt

SEQ ID NO: 834 - DNA, 41 bp, Homo sapiens
acggtcaccacacctgagagcggtcctggggctggccctgt

SEQ ID NO: 835 - DNA, 41 bp, Homo sapiens
gcggcagccatcactccacacgcacaggtgacccaggtctt

SEQ ID NO: 836 - DNA, 41 bp, Homo sapiens
aggatgttctgggagccaccggtaggcacgggtgccagggg

SEQ ID NO: 837 - DNA, 41 bp, Homo sapiens
tggaatgagcaacacaggaaggctccagttgtccagaccat

SEQ ID NO: 838 - DNA, 41 bp, Homo sapiens
cgagactggttggaaacacaagagtgctgctggctgcacca

SEQ ID NO: 839 - DNA, 41 bp, Homo sapiens
cccccatccattccagaccatgtgactgttgagatgtctgt

SEQ ID NO: 840 - DNA, 41 bp, Homo sapiens
tcgatgtgcgccaggagtactcagtgagtcctgggggaggc

SEQ ID NO: 841 - DNA, 41 bp, Homo sapiens
agtttgacccagcagactccagttacctttacctgatgacg

SEQ ID NO: 842 - DNA, 41 bp, Homo sapiens
cctaccttgagaagcctcccattgaccgtgcccaggaagac

SEQ ID NO: 843 - DNA, 41 bp, Homo sapiens
aggcctccaggaagtgaccctgagacaataactgtgcaact

SEQ ID NO: 844 - DNA, 41 bp, Homo sapiens
gtaactaagcacaccccttaaagaattttgggaagtcgccc

SEQ ID NO: 845 - DNA, 41 bp, Homo sapiens
taagccagaggatgctgtagcgagtacttgtatgcaataac

SEQ ID NO: 846 - DNA, 41 bp, Homo sapiens
cttgttgtcatggtgcgttgaaagagtagccagttgtcttt

SEQ ID NO: 847 - DNA, 41 bp, Homo sapiens
attagtatgcaggtcttatccaccattggaattaagctgtt

SEQ ID NO: 848 - DNA, 41 bp, Homo sapiens
acgtttttatcacacattaaacacttgcattaattttggag

SEQ ID NO: 849 - DNA, 41 bp, Homo sapiens
gatgagttaaatgggctagtatctaaattttaaatttttac

SEQ ID NO: 850 - DNA, 41 bp, Homo sapiens
gtacatcccatattccctttccaaaatctagtttcctatgt

SEQ ID NO: 851 - DNA, 41 bp, Homo sapiens
gcttaccagaaaacaccctctttgttgtttttatttctcag

SEQ ID NO: 852 - DNA, 41 bp, Homo sapiens
ggacaaggaggagaagccccgggaggtcacgggagttcact

SEQ ID NO: 853 - DNA, 41 bp, Homo sapiens
gagcagccatttcgaaaggcggcagaagaggaaattaactc

SEQ ID NO: 854 - DNA, 41 bp, Homo sapiens
gcgaggggaagtcattttttgaataactaggctctatttgc

SEQ ID NO: 855 - DNA, 41 bp, Homo sapiens
caaggaaagacctggtgtccctgtgctaattttaactctct

SEQ ID NO: 856 - DNA, 41 bp, Homo sapiens
tacagatgctcataggcatctgaaaaaaaaatactttgtta

SEQ ID NO: 857 - DNA, 41 bp, Homo sapiens
aactcctttgacagtatggatggcacctaacgcatccttgt

SEQ ID NO: 858 - DNA, 41 bp, Homo sapiens
gaggtgttttcttggctcttcackaacgtttttaataaagc

SEQ ID NO: 859 - DNA, 41 bp, Homo sapiens
gcgccccctggacttctgctggaatttagatttaaatagat

SEQ ID NO: 860 - DNA, 41 bp, Homo sapiens
acatatttagaatggatgccagaacaggagaaatgggtggg

SEQ ID NO: 861 - DNA, 41 bp, Homo sapiens
attcatatgccaccagccattggcagaaatgtaacaggaaa

SEQ ID NO: 862 - DNA, 41 bp, Homo sapiens
atggctctgtaaatgggatgtctcatgttcaggtttctgga

SEQ ID NO: 863 - DNA, 41 bp, Homo sapiens
atctccaggtgaacatggaatgcagtgaaaacctggggtat

SEQ ID NO: 864 - DNA, 41 bp, Homo sapiens
tgataagtagttaatgatccagaaataaactgttaggtgct

SEQ ID NO: 865 - DNA, 41 bp, Homo sapiens
aagtaaaatagtagatattggattgcttctacatttactac

SEQ ID NO: 866 - DNA, 41 bp, Homo sapiens
agagcccctacccaattgctttactatttatagttcctcag

SEQ ID NO: 867 - DNA, 41 bp, Homo sapiens
atctggggacctgctcctggcagagcaataggawctgtgtg

SEQ ID NO: 868 - DNA, 41 bp, Homo sapiens
gagtcccaaaattcaacccttccgatagggctgggcctgac

SEQ ID NO: 869 - DNA, 41 bp, Homo sapiens
ccctagcctgcttttgtcctattattttttatttccacata

SEQ ID NO: 870 - DNA, 41 bp, Homo sapiens
agagggaacccaaatattagagtgggaagcaagtcataaac

SEQ ID NO: 871 - DNA, 41 bp, Homo sapiens
tagggttaccaatccactaggatgcaaaactgtacttatta

SEQ ID NO: 872 - DNA, 41 bp, Homo sapiens
aggcttctttttccattacattgtaagactttggagggcag

SEQ ID NO: 873 - DNA, 41 bp, Homo sapiens
agcrgtcaggtgcggaggcaacctctcagcggtggggaaca

SEQ ID NO: 874 - DNA, 41 bp, Homo sapiens
caggacaaacagtggattcattcagaacacaatatgctggt

SEQ ID NO: 875 - DNA, 41 bp, Homo sapiens
aagccactacagacaccgcatgcaccgaaattctcccttgt

SEQ ID NO: 876 - DNA, 41 bp, Homo sapiens
atcactgtccctcagttcactggtcttgtctgcttcgtcgy

SEQ ID NO: 877 - DNA, 41 bp, Homo sapiens
aattctcagtcttaaaaacagggcataaagaaagctaaaat

SEQ ID NO: 878 - DNA, 41 bp, Homo sapiens
agaagataagtgtttagggtattggatatcccagttaccct

SEQ ID NO: 879 - DNA, 41 bp, Homo sapiens
cctttttttggatgatcctagaattaatacaagtgtattct

SEQ ID NO: 880 - DNA, 41 bp, Homo sapiens
gcccttagtcaccaactcctactcatcccaccatgctgttg

SEQ ID NO: 881 - DNA, 41 bp, Homo sapiens
gtaaattaaaatttgtttgggtgatttgtgctgtatttcta

SEQ ID NO: 882 - DNA, 41 bp, Homo sapiens
agcaacacttcctccttgcatattacaagcatagctaatgc

SEQ ID NO: 883 - DNA, 41 bp, Homo sapiens
ccctcattttctgttagggaggtatgtgtttaccaagctgt

SEQ ID NO: 884 - DNA, 41 bp, Homo sapiens
atgagggctttacttttgcaagaaatactacagatggtgaa

SEQ ID NO: 885 - DNA, 41 bp, Homo sapiens
tcccttctcagtaactaacaataatcatctctctggaggac

SEQ ID NO: 886 - DNA, 41 bp, Homo sapiens
cattccctcacacagtacagattaataaatgtgcattttga

SEQ ID NO: 887 - DNA, 41 bp, Homo sapiens
cctgtgtgatgaggggcaaaagaagctcttgagaacctgct

SEQ ID NO: 888 - DNA, 41 bp, Homo sapiens
gtaacgaagaaagaccagagcgtcatccctgtgatacagca

SEQ ID NO: 889 - DNA, 41 bp, Homo sapiens
tatgtatcttgcttttgtttcaaacagtcatccacattagt

SEQ ID NO: 890 - DNA, 41 bp, Homo sapiens
gataggttgcaaaattttggtgtgttcttgcattgcataca

SEQ ID NO: 891 - DNA, 41 bp, Homo sapiens
attgacggtgttataattactatggttttgaaattacatag

SEQ ID NO: 892 - DNA, 41 bp, Homo sapiens
tgaggacccagatgtcaacatcaccaatctggaatttgaaa

SEQ ID NO: 893 - DNA, 41 bp, Homo sapiens
ctccttttgacctgagtgtcgtctatcgggaaggagccaat

SEQ ID NO: 894 - DNA, 41 bp, Homo sapiens
tatgtaaaagttttaatgcatgatgtagcttaccgccagga

SEQ ID NO: 895 - DNA, 41 bp, Homo sapiens
gatggatcctatcttactaatcatcagcattttgagttttt

SEQ ID NO: 896 - DNA, 41 bp, Homo sapiens
aattagctgccagagttgctatcagtaaagagaagaaataa

SEQ ID NO: 897 - DNA, 41 bp, Homo sapiens
ctgaaatcagagaacattgatagatgaagtgaatggcagag

SEQ ID NO: 898 - DNA, 41 bp, Homo sapiens
gcccatctgaggatgtagtcgtcactccakaaagctttgga

SEQ ID NO: 899 - DNA, 41 bp, Homo sapiens
gtgcagaycagataattatagagagatggaatgggacaacc

SEQ ID NO: 900 - DNA, 41 bp, Homo sapiens
aatctgcctctggggcgggacctgtcaggcttcaggaaggg

SEQ ID NO: 901 - DNA, 41 bp, Homo sapiens
tccagggaggagcttcgtgcaaccttcccggaccactcagg

SEQ ID NO: 902 - DNA, 41 bp, Homo sapiens
catcacctccaggtagctcccaaaatgtccctagaaagtgg

SEQ ID NO: 903 - DNA, 41 bp, Homo sapiens
ggagcacagagtagcagtgacgctgtccaaggcagggggga

SEQ ID NO: 904 - DNA, 41 bp, Homo sapiens
cattcaggccagtggctgcaagggagcagaaagatcaggct

SEQ ID NO: 905 - DNA, 41 bp, Homo sapiens
tacagaggaagaaatccaggacagaggtggaggcagtgaag

SEQ ID NO: 906 - DNA, 41 bp, Homo sapiens
ctacctcattcattgaccccgctatctgacctgtacatgtt

SEQ ID NO: 907 - DNA, 41 bp, Homo sapiens
ttgaggacaaacagaacatcagtgagtaagtggaatattag

SEQ ID NO: 908 - DNA, 41 bp, Homo sapiens
ttcttgtgttcttccctttctatttcaactcttcatctcag

SEQ ID NO: 909 - DNA, 41 bp, Homo sapiens
ggtttgtgtaccaggattggagacccctgatgtatagtgta

SEQ ID NO: 910 - DNA, 41 bp, Homo sapiens
gaagaggataggtttttctatcttaaacaaaatcttcctta

SEQ ID NO: 911 - DNA, 41 bp, Homo sapiens
gttaggcatcaggcaactacaaaggagtatacgagcatgca

SEQ ID NO: 912 - DNA, 41 bp, Homo sapiens
cacagggtaaatttagccacggcagcaggagcatgatataa

SEQ ID NO: 913 - DNA, 41 bp, Homo sapiens
ggcatgtgaaataagttggtttaattagagtgaagcccagg

SEQ ID NO: 914 - DNA, 41 bp, Homo sapiens
tggattgtgtgtgtggtaatgggattattgttatatttaaa

SEQ ID NO: 915 - DNA, 41 bp, Homo sapiens
cacgagcatcttgctgtctttaattaagaagttaactggac

SEQ ID NO: 916 - DNA, 41 bp, Homo sapiens
ttgaaagctgagtcattttcgtaatgggtcagaaagacatt

SEQ ID NO: 917 - DNA, 41 bp, Homo sapiens
tacatgacgcatgtatttgtcaaaacccacagatctattaa

SEQ ID NO: 918 - DNA, 41 bp, Homo sapiens
ctgagagtgcagtgaaccttcgtgtctgtgatggaagaggt

SEQ ID NO: 919 - DNA, 41 bp, Homo sapiens
gcttagatgtgagagttgattccataataataaaagttatt

SEQ ID NO: 920 - DNA, 41 bp, Homo sapiens
ttgaactctatgtaccaagtctgaacacattccaaatatcc

SEQ ID NO: 921 - DNA, 41 bp, Homo sapiens
ggtattttgctacagcagcctgagcaaactaatatatcatc

SEQ ID NO: 922 - DNA, 41 bp, Homo sapiens
aaaggcggtcacctgcaggagtagccatctttggtcctttc

SEQ ID NO: 923 - DNA, 41 bp, Homo sapiens
cccccaggggtggtaacaacggcacgcaagcacagccattg

SEQ ID NO: 924 - DNA, 41 bp, Homo sapiens
ccacacctggtggacaggacaaccgtggtggccaggaagct

SEQ ID NO: 925 - DNA, 41 bp, Homo sapiens
ggttaaaaagttctctaccagggaagttggataaaagtaac

SEQ ID NO: 926 - DNA, 41 bp, Homo sapiens
aaatcagaatcgaattattgatttggggctaattgtatctg

SEQ ID NO: 927 - DNA, 41 bp, Homo sapiens
cctgtcagtgaaaacaactatcaaagctggattttaaatat

SEQ ID NO: 928 - DNA, 41 bp, Homo sapiens
ccattagcagtaggtctgaaataactttaatatgcaagtta

SEQ ID NO: 929 - DNA, 41 bp, Homo sapiens
agagccagctgggagaaacacgcaacatagttctttgcaat

SEQ ID NO: 930 - DNA, 41 bp, Homo sapiens
agcagctggaccatgatctcttggatatggtggtaggtgaa

SEQ ID NO: 931 - DNA, 41 bp, Homo sapiens
agacgatgtactgatgtaagcttttgtaaatttctaaactg

SEQ ID NO: 932 - DNA, 41 bp, Homo sapiens
actctgtctttccaattcctgaacagcatgcttggatggga

SEQ ID NO: 933 - DNA, 41 bp, Homo sapiens
tcagaaagaatggggtaaggcgaattgagttttagaacata

SEQ ID NO: 934 - DNA, 41 bp, Homo sapiens
acagtgaagaaagaggaacaaagagaagggcaggcaggagg

SEQ ID NO: 935 - DNA, 41 bp, Homo sapiens
ttgaaggtggatgagggaacagtcaggttgaggagcatttt

SEQ ID NO: 936 - DNA, 41 bp, Homo sapiens
acacaatactgggtttctctacttctctctcaccatcacac

SEQ ID NO: 937 - DNA, 41 bp, Homo sapiens
ccacgcaccagcaggttcacagtgcagctcatgcggttgtc

SEQ ID NO: 938 - DNA, 41 bp, Homo sapiens
gatagtctaaatgaatgtccgccacccccgcctgtagttgt

SEQ ID NO: 939 - DNA, 41 bp, Homo sapiens
gctggctggggcaaaggtctttgatgcactgtgcagaagta

SEQ ID NO: 940 - DNA, 41 bp, Homo sapiens
ctgctcgggccagaaaatccagaaacgggcccttaccgatg

SEQ ID NO: 941 - DNA, 41 bp, Homo sapiens
gttctgaaatgaagacacatgtggcaggcaggttacaaccc

SEQ ID NO: 942 - DNA, 41 bp, Homo sapiens
ctcactcactccttgaggacgctctcatgacaactgtaaag

SEQ ID NO: 943 - DNA, 41 bp, Homo sapiens
ttcaaaaactattttggtacatttcaaatacagtgtttaaa

SEQ ID NO: 944 - DNA, 41 bp, Homo sapiens
tgttgctaagatcaatagctacatttgaatctatgtctccc

SEQ ID NO: 945 - DNA, 41 bp, Homo sapiens
cagtttattatgggttatctgattggaataaagaggtatca

SEQ ID NO: 946 - DNA, 41 bp, Homo sapiens
aatcattatgtcacaaaaaaatatataaagataaatttttc

SEQ ID NO: 947 - DNA, 41 bp, Homo sapiens
agagccaagacttgtcccctgtttctgcagcagattggtcc

SEQ ID NO: 948 - DNA, 41 bp, Homo sapiens
gtttctcaaaagttctaaacgttacagaggataattttaag

SEQ ID NO: 949 - DNA, 41 bp, Homo sapiens
ccttgtctggaggagttgggtttcctcaataattggctgtg

SEQ ID NO: 950 - DNA, 41 bp, Homo sapiens
ggatccaaagggtgtcaaggtgatcattatcttgggatgga

SEQ ID NO: 951 - DNA, 41 bp, Homo sapiens
aatgaaactaaatgatcatctttcaactctcccttctcact

SEQ ID NO: 952 - DNA, 41 bp, Homo sapiens
agttgctcccctctctgatctacattcgtaaaatgacataa

SEQ ID NO: 953 - DNA, 41 bp, Homo sapiens
caggtgtccctaccttaaggacctcctccttgggacttcac

SEQ ID NO: 954 - DNA, 41 bp, Homo sapiens
cacacaactrgctaaggagccccagggccacagctgctgtt

SEQ ID NO: 955 - DNA, 41 bp, Homo sapiens
ataagcaggaaaatgaatgcattaggagaggttttatatct

SEQ ID NO: 956 - DNA, 41 bp, Homo sapiens
ttatgcatacaacactcaaccgatccagttactcttactct

SEQ ID NO: 957 - DNA, 41 bp, Homo sapiens
caccccagtcaccgtggcttacacctgcacaacagattcct

SEQ ID NO: 958 - DNA, 41 bp, Homo sapiens
aatttccctgcattttgtgatgacttgtttttattggtaac

SEQ ID NO: 959 - DNA, 41 bp, Homo sapiens
tgcgcattttccgcactccggtacactttacactgaacacc

SEQ ID NO: 960 - DNA, 41 bp, Homo sapiens
gacccagagcaggaagcataatcaagccctccactagatta

SEQ ID NO: 961 - DNA, 41 bp, Homo sapiens
cacttggaaatcctaactccgcagaacaaaattttacaagc

SEQ ID NO: 962 - DNA, 41 bp, Homo sapiens
acacactgacattcgaggcccaaggaatactcctgcctcta

SEQ ID NO: 963 - DNA, 41 bp, Homo sapiens
ttcatttacaagcctgatcagccttacatgaactaatgttt

SEQ ID NO: 964 - DNA, 41 bp, Homo sapiens
aacactgttgcaggatctctaataatcactatgtacacttc

SEQ ID NO: 965 - DNA, 41 bp, Homo sapiens
aactccccagctaaacacccataagacttcatacaacacaa

SEQ ID NO: 966 - DNA, 41 bp, Homo sapiens
taaatgcttatccatttagtgacaggaaaaatgagacaact

SEQ ID NO: 967 - DNA, 41 bp, Homo sapiens
gtatgctttccatcgaaaaagtactctattaaacagcttag

SEQ ID NO: 968 - DNA, 41 bp, Homo sapiens
tatacaggagtcatcccctatgttgacactggtaagttgta

SEQ ID NO: 969 - DNA, 41 bp, Homo sapiens
tcaagtttaagctgctatgtcccttatttttaacttttgtt

SEQ ID NO: 970 - DNA, 41 bp, Homo sapiens
atataatttatattacaatgtaaaagcttctttaatactaa

SEQ ID NO: 971 - DNA, 41 bp, Homo sapiens
gatggggaggaagagaaggcattggtcttgcagtcttgtct

SEQ ID NO: 972 - DNA, 41 bp, Homo sapiens
aatggtaagcatctattttgcagtccactctactgagctaa

SEQ ID NO: 973 - DNA, 41 bp, Homo sapiens
tttatatatgatatcatcattaagcactttctataagctga

SEQ ID NO: 974 - DNA, 41 bp, Homo sapiens
aacaatctgtgaacacttgtcatatgcttactgtaagtgtg

SEQ ID NO: 975 - DNA, 41 bp, Homo sapiens
actatatgtcatgtctacaggctgtctcctaagagtagagg

SEQ ID NO: 976 - DNA, 41 bp, Homo sapiens
tgcaaacattgggaaaccacagtaggggggagcaggactct

SEQ ID NO: 977 - DNA, 41 bp, Homo sapiens
taccatggacagcagcgctgtccccacraacgccagcaatt

SEQ ID NO: 978 - DNA, 41 bp, Homo sapiens
acttgtcccacttagatggcgacctgtccgacccatgcggt

SEQ ID NO: 979 - DNA, 41 bp, Homo sapiens
gcatttcacattcacatgtaatatttgaatatacacatcaa

SEQ ID NO: 980 - DNA, 41 bp, Homo sapiens
ttgagtctccttccaattaaatcatggaacatcagagccat

SEQ ID NO: 981 - DNA, 41 bp, Homo sapiens
tcttttgtggaaatgtgatgtatttgtttatatgcagacaa

SEQ ID NO: 982 - DNA, 41 bp, Homo sapiens
accagacttaggagagatatgtctcactgtagaaccagtgc

SEQ ID NO: 983 - DNA, 41 bp, Homo sapiens
ctctggtcaaggctaaaaatgaatgagcaaaatggcagtat

SEQ ID NO: 984 - DNA, 41 bp, Homo sapiens
agccaaagttcagttctccaattcatctgagctcaggccca

SEQ ID NO: 985 - DNA, 45 bp, Homo sapiens
ggtatcrtgggtcctttcragtaccaaccgccttaggctggaagc

SEQ ID NO: 986 - DNA, 41 bp, Homo sapiens
ttttaccgaaggctgtgtctcgtaagcacccccgagcaact

SEQ ID NO: 987 - DNA, 41 bp, Homo sapiens
ctactccggcacccagtgggctggtagtcctgttggcagga

SEQ ID NO: 988 - DNA, 41 bp, Homo sapiens
ccaagaagcgcgcggcgagaatgcaaggtgggggccccgcc

SEQ ID NO: 989 - DNA, 41 bp, Homo sapiens
ctctcgccgcgcgcttcttgatccctgagacttcgaacgaa

SEQ ID NO: 990 - DNA, 41 bp, Homo sapiens
gagcagaggggcaggtcccggccggacggcgcccggagccc

SEQ ID NO: 991 - DNA, 41 bp, Homo sapiens
agagcggattgggggtcgcgggtggtagcaggaggaggagc

SEQ ID NO: 992 - DNA, 41 bp, Homo sapiens
tggggattcagagcacccacccgcagcacctccctcctctg

SEQ ID NO: 993 - DNA, 41 bp, Homo sapiens
gggtcagtccggacagccccggtcgcttgttacctagcatc

SEQ ID NO: 994 - DNA, 41 bp, Homo sapiens
ctgggtgcgctggccgaggcatacccctccaagccggacaa

SEQ ID NO: 995 - DNA, 41 bp, Homo sapiens
gacatggccagatactactcagcgctgcgacactacatcaa

SEQ ID NO: 996 - DNA, 41 bp, Homo sapiens
ctgacaatgtctgtggcaacgctgcagtttactccttggtt

SEQ ID NO: 997 - DNA, 41 bp, Homo sapiens
cagacacccactcctatgtgcgtttctgaaaattacagggt

SEQ ID NO: 998 - DNA, 41 bp, Homo sapiens
tccagatatggaaaacgatctagcccagagacactgatttc

SEQ ID NO: 999 - DNA, 41 bp, Homo sapiens
atttcaatttagagtcagggactcactctatgctcccctga

SEQ ID NO: 1000 - DNA, 41 bp, Homo sapiens
tggaaagaggtgcccaccaacgtctaagtgttaaacattga

SEQ ID NO: 1001 - DNA, 41 bp, Homo sapiens
tatcatgcattcaaaagtgtgtcctcctcaatgaaaaatct

SEQ ID NO: 1002 - DNA, 41 bp, Homo sapiens
tgaaaaatctattacaatagcgaggattattttcgttaaac

SEQ ID NO: 1003 - DNA, 41 bp, Homo sapiens
tatttctcaaacattttcagttttagaatgggaataggttt

SEQ ID NO: 1004 - DNA, 41 bp, Homo sapiens
gtgcctttaaacctattctataacctatttaaacgtatttc

SEQ ID NO: 1005 - DNA, 41 bp, Homo sapiens
agggctgcctggtaagctgagtcagggtgcctggctgccgc

SEQ ID NO: 1006 - DNA, 41 bp, Homo sapiens
aacgccacttgtgactgctcattacctttcagttgtgtccc

SEQ ID NO: 1007 - DNA, 41 bp, Homo sapiens
atgttgggatttaactttctattatatgtcagactcactta

SEQ ID NO: 1008 - DNA, 41 bp, Homo sapiens
tgtgtgttttaaatctttgcacttaaatgtttttgatttct

SEQ ID NO: 1009 - DNA, 41 bp, Homo sapiens
gaagcttccctccgacaggcagccccgcactaaggtaggga

SEQ ID NO: 1010 - DNA, 41 bp, Homo sapiens
ctaatggttggaaacgccagtctttggtgaaaacagaaagt

SEQ ID NO: 1011 - DNA, 41 bp, Homo sapiens
ttcaagaattcaactgcagactgaaaatatttggagaaaaa

SEQ ID NO: 1012 - DNA, 41 bp, Homo sapiens
aacctagccacagagcccgacgcgatgtgtccttgtcgaga

SEQ ID NO: 1013 - DNA, 41 bp, Homo sapiens
gcctcctttgctgccctcacgatctcttcctgtgacaccac

SEQ ID NO: 1014 - DNA, 41 bp, Homo sapiens
ctctgcaccttcaggttcagacccttcaagatctaccagga

SEQ ID NO: 1015 - DNA, 41 bp, Homo sapiens
acaagctagttaccttttatcgttcagtttaaaaaagttct

SEQ ID NO: 1016 - DNA, 41 bp, Homo sapiens
cggtccccttcaagatccattccgacctgaagagaaaccgc

SEQ ID NO: 1017 - DNA, 41 bp, Homo sapiens
tgctcttcaaaaaaaccagattgaatatttttaaaagtaat

SEQ ID NO: 1018 - DNA, 41 bp, Homo sapiens
gttacttgtagggggagggtagagggaaatctgggcaaatg

SEQ ID NO: 1019 - DNA, 41 bp, Homo sapiens
gggcttctatccccgaaccccgggccctggtgccactcaag

SEQ ID NO: 1020 - DNA, 41 bp, Homo sapiens
tcccayttaagagctattcttctatccttccctgtaaacaa

SEQ ID NO: 1021 - DNA, 41 bp, Homo sapiens
tggcagacacaggacagggagcgctgcttatgtctccgagg

SEQ ID NO: 1022 - DNA, 41 bp, Homo sapiens
aacccatcctcgtggtaatcgtccctggtaagaaacacaca

SEQ ID NO: 1023 - DNA, 41 bp, Homo sapiens
catttctaattaccagcttcttacttggcactttcaatttt

SEQ ID NO: 1024 - DNA, 41 bp, Homo sapiens
ccacagcggcttcctgccattgatgaggctgatttctgcct

SEQ ID NO: 1025 - DNA, 41 bp, Homo sapiens
tgcatcctctgcttctcctcgaaccgtgcttcacagctgcc

SEQ ID NO: 1026 - DNA, 41 bp, Homo sapiens
ggggccaaaggaatatttagctgaagggggagagaggccac

SEQ ID NO: 1027 - DNA, 41 bp, Homo sapiens
actttgtgtgtacatgtggagggaagtatttgacattttga

SEQ ID NO: 1028 - DNA, 41 bp, Homo sapiens
acttgtgtcccccaaaatcatatattgaagttaaaacctcc

SEQ ID NO: 1029 - DNA, 41 bp, Homo sapiens
tagccatggcagaagacatactctctacaccttatgcatgg

SEQ ID NO: 1030 - DNA, 41 bp, Homo sapiens
gacagagaaggtatgtccacgcacactagacatactgcatg

SEQ ID NO: 1031 - DNA, 41 bp, Homo sapiens
agtattgatcagtggcgggacacagtttgaaggtagaggga

SEQ ID NO: 1032 - DNA, 41 bp, Homo sapiens
gctgtatcttgggggaagtgtgttcttgagagctgtgtaag

SEQ ID NO: 1033 - DNA, 41 bp, Homo sapiens
ggccgtcctcatcttcacacactgttctccttctatgtggg

SEQ ID NO: 1034 - DNA, 41 bp, Homo sapiens
tagcaggtggcacaactggcgctgggaaccgggggtccctt

SEQ ID NO: 1035 - DNA, 41 bp, Homo sapiens
ggccccccgtgcagggagggtttcaggctgcggcaggtagg

SEQ ID NO: 1036 - DNA, 41 bp, Homo sapiens
tactatacaaataaaaaaattaaaacccaacctcaagctgt

SEQ ID NO: 1037 - DNA, 41 bp, Homo sapiens
cgaatgctgagaacttgccatgctctctccccagggcccca

SEQ ID NO: 1038 - DNA, 41 bp, Homo sapiens
gcctccccctgtgatctctcggtcctctccgcattcctggg

SEQ ID NO: 1039 - DNA, 41 bp, Homo sapiens
ttccctttgttttccctttcttccagctccaggccaggctt

SEQ ID NO: 1040 - DNA, 41 bp, Homo sapiens
tgcgctctgggctagacactctgataggtgctgggattaca

SEQ ID NO: 1041 - DNA, 41 bp, Homo sapiens
tggaaaacagatccagacagtttcagttatgtgtctgagaa

SEQ ID NO: 1042 - DNA, 41 bp, Homo sapiens
ccctactacccctacaactatacgagcggcgctgccaccgg

SEQ ID NO: 1043 - DNA, 41 bp, Homo sapiens
gcatgccttttcaaaaacacgttcaagacctgaaaataaaa

SEQ ID NO: 1044 - DNA, 41 bp, Homo sapiens
tactgctgtggcctgaatccatgattaaaggaaatgctaag

SEQ ID NO: 1045 - DNA, 41 bp, Homo sapiens
tacacaagtcactgggtgacgtctgtagctccaccaacctg

SEQ ID NO: 1046 - DNA, 41 bp, Homo sapiens
ctctgtctaggtgcatagaactgtgtacatatacatacaca

SEQ ID NO: 1047 - DNA, 41 bp, Homo sapiens
agtctgcaaatgtgttttttatgtgctaaatagctcaaagt

SEQ ID NO: 1048 - DNA, 41 bp, Homo sapiens
taagtttggttgatgagtctatctctctagactgcaagctc

SEQ ID NO: 1049 - DNA, 41 bp, Homo sapiens
cacagaagtgggcattctgaaaggcctctaattttcctcta

SEQ ID NO: 1050 - DNA, 41 bp, Homo sapiens
ttaaaacagcgaccccatacgtgcattagttaaaactttct

SEQ ID NO: 1051 - DNA, 41 bp, Homo sapiens
gcagattgaggtaaattcatcgttaatgtcatcacagcaat

SEQ ID NO: 1052 - DNA, 41 bp, Homo sapiens
caaaacagaatcccaagagcgatattttaactcaacaaaca

SEQ ID NO: 1053 - DNA, 41 bp, Homo sapiens
agagttcttatggttctctttggtagtttttctttagctgg

SEQ ID NO: 1054 - DNA, 41 bp, Homo sapiens
ctttcattcttgtcgttggcatctctgttctgataaaaaga

SEQ ID NO: 1055 - DNA, 41 bp, Homo sapiens
ggaggcaatgtctgatttgcctagggctcaggggagagatg

SEQ ID NO: 1056 - DNA, 41 bp, Homo sapiens
aggttcagcagaaaagaacccaggaaaaaagtctaggaaag

SEQ ID NO: 1057 - DNA, 41 bp, Homo sapiens
gatgggccttctgataaggagcgctgccaaaagttcaaatg

SEQ ID NO: 1058 - DNA, 41 bp, Homo sapiens
attccttcctttccctgtttgtacataccttacagatactg

SEQ ID NO: 1059 - DNA, 41 bp, Homo sapiens
tctgtttcagtctcaaggagcctgaaaaggtgaattcctgt

SEQ ID NO: 1060 - DNA, 41 bp, Homo sapiens
cagtcttgtgagaacattctcgccatctgtactttgcattt

SEQ ID NO: 1061 - DNA, 41 bp, Homo sapiens
ccacacctggcctgaactcttctttaaaaactgcatgctga

SEQ ID NO: 1062 - DNA, 41 bp, Homo sapiens
tcatgcatagatggtgtagctttagaaaactcaggcctagc

SEQ ID NO: 1063 - DNA, 41 bp, Homo sapiens
aggtggatttttttaagaagtatattcatacaactgaatat

SEQ ID NO: 1064 - DNA, 41 bp, Homo sapiens
gcctgatattctttccctattaaattgcttcctcatctagg

SEQ ID NO: 1065 - DNA, 41 bp, Homo sapiens
gaagaagctgtcagaattgcgagggaaattggtaagtcctt

SEQ ID NO: 1066 - DNA, 41 bp, Homo sapiens
actgtgcccacccaagtttgcgttttgaaaagattggtcaa

SEQ ID NO: 1067 - DNA, 41 bp, Homo sapiens
atggcatacagcctgggtgacatttttaaacataagtgaaa

SEQ ID NO: 1068 - DNA, 41 bp, Homo sapiens
gggaaaatgttcatttaagtgtaaaacatgaaatggtattc

SEQ ID NO: 1069 - DNA, 41 bp, Homo sapiens
cttgttagttcaggtctcttccagatgaggaagagagatta

SEQ ID NO: 1070 - DNA, 41 bp, Homo sapiens
aaatggacaacaaaagtcactggaaaaaagggaaaaaaaga

SEQ ID NO: 1071 - DNA, 41 bp, Homo sapiens
tgagaaataagtgatgtcatacatttttggttgtggatcat

SEQ ID NO: 1072 - DNA, 41 bp, Homo sapiens
tgtggttctcccttcacagttgaatacaagggcttttatat

SEQ ID NO: 1073 - DNA, 41 bp, Homo sapiens
taataagtggttatgccaaggggtccctgcagctcagaggc

SEQ ID NO: 1074 - DNA, 41 bp, Homo sapiens
tctttgggcctccacccccttgtctctagtggacatttgag

SEQ ID NO: 1075 - DNA, 41 bp, Homo sapiens
aaaggaagctgggcgtcctctgggccccccaacacacgtcc

SEQ ID NO: 1076 - DNA, 41 bp, Homo sapiens
ctaacacagttgcgaacatcagcagagccctcgggagccac

SEQ ID NO: 1077 - DNA, 41 bp, Homo sapiens
ttgatgatgatgtcgatgccaaagagtgacacgcccagtgc

SEQ ID NO: 1078 - DNA, 41 bp, Homo sapiens
cttcacagcgccgcaacaattatgcatgagggagtgattcg

SEQ ID NO: 1079 - DNA, 41 bp, Homo sapiens
ggccacagctggccagtctctttgtgctttgaatctccagc

SEQ ID NO: 1080 - DNA, 41 bp, Homo sapiens
tgcagcgtgcggcagtgcttcgttcttctttaagatgaaat

SEQ ID NO: 1081 - DNA, 41 bp, Homo sapiens
cctacacaggaagccccggaaccacagcaattctccctgcc

SEQ ID NO: 1082 - DNA, 41 bp, Homo sapiens
tgtgctctggccaggggccttgacctcattctgttggtggt

SEQ ID NO: 1083 - DNA, 41 bp, Homo sapiens
tcgcccaggctgaccacaagttccaaacaggactttcttgt

SEQ ID NO: 1084 - DNA, 41 bp, Homo sapiens
tgcccaaacagtatcaaaagcggatgtttatcacaatacta

SEQ ID NO: 1085 - DNA, 41 bp, Homo sapiens
ttagcaacaaaatcctgaagccacttctagaccataaccca

SEQ ID NO: 1086 - DNA, 41 bp, Homo sapiens
cagagggcagggcccacaccataccccacagaagcccagga

SEQ ID NO: 1087 - DNA, 41 bp, Homo sapiens
gggtacagcccagcatggccacaggggtccctgatgggaat

SEQ ID NO: 1088 - DNA, 41 bp, Homo sapiens
gactgccaggtgtggacacacgctcgtcaagtggtgaagaa

SEQ ID NO: 1089 - DNA, 41 bp, Homo sapiens
cacacggacgcttcctcctacgtgaagttctgtttgctccc

SEQ ID NO: 1090 - DNA, 41 bp, Homo sapiens
atggtcatattatgcatgcatgtttttgatttcaagaatgc

SEQ ID NO: 1091 - DNA, 41 bp, Homo sapiens
atgcggtgctcggtaactgttcatccgatgcaggcctcact

SEQ ID NO: 1092 - DNA, 41 bp, Homo sapiens
accagaattatcacagcaccctctcattcccagcgcgtcct

SEQ ID NO: 1093 - DNA, 41 bp, Homo sapiens
tgatcatggtcactgccctgagttcaaataatgcgagctga

SEQ ID NO: 1094 - DNA, 41 bp, Homo sapiens
aggacaacatgccatttgtccaaacgttttaaagatatgat

SEQ ID NO: 1095 - DNA, 41 bp, Homo sapiens
gggggaagctgggtgcatgcagagcaccgtggagtctggga

SEQ ID NO: 1096 - DNA, 41 bp, Homo sapiens
ccttgaagtcacccggcccccatgcaaggtgcccacatgtg

SEQ ID NO: 1097 - DNA, 41 bp, Homo sapiens
tttggaaggaaaacgtggcgggtgggcgtattctccagaag

SEQ ID NO: 1098 - DNA, 41 bp, Homo sapiens
tcccagaccagaccttgcccgatgacgttgttggtaatgct

SEQ ID NO: 1099 - DNA, 41 bp, Homo sapiens
tgagatcccccggacaacacgctccaccttcccatggagct

SEQ ID NO: 1100 - DNA, 41 bp, Homo sapiens
ttgtttgtgtctgtctcaaatccaaaggggtggctcagcct

SEQ ID NO: 1101 - DNA, 41 bp, Homo sapiens
gaacctcccagggggcagaataaaaagtcaacaagctggaa

SEQ ID NO: 1102 - DNA, 41 bp, Homo sapiens
caaacgttgctgaagtctccgcgacctttattgttttgccc

SEQ ID NO: 1103 - DNA, 41 bp, Homo sapiens
gttccctgaccaggagtccagtaggcaatagtctattaact

SEQ ID NO: 1104 - DNA, 41 bp, Homo sapiens
tttgctcatgcacctgccttacctttgtcatcacaacagaa

SEQ ID NO: 1105 - DNA, 41 bp, Homo sapiens
acctccttccccgtgckccatgaggagcgggctgcaccttg

SEQ ID NO: 1106 - DNA, 41 bp, Homo sapiens
gctgaaacccgattcctaccgggtgacgctgagaccgtacc

SEQ ID NO: 1107 - DNA, 41 bp, Homo sapiens
tcctgctcgacctgctcctctagctgtgcaatcttggcctc

SEQ ID NO: 1108 - DNA, 41 bp, Homo sapiens
tccagcgccgcgatggtggatttgaacttggacttgacggc

SEQ ID NO: 1109 - DNA, 41 bp, Homo sapiens
tacgaggagaaggcggccgcttatgataaactggaaaagac

SEQ ID NO: 1110 - DNA, 41 bp, Homo sapiens
ttccgcagcttgaggtaggcagcgcagttcctctgaatcac

SEQ ID NO: 1111 - DNA, 41 bp, Homo sapiens
cctgtggctggtaccttcccggcataatggatgatggagaa

SEQ ID NO: 1112 - DNA, 41 bp, Homo sapiens
atgattgccatggcctccacagtttcctggaacatctcatc

SEQ ID NO: 1113 - DNA, 41 bp, Homo sapiens
ccagaaccaccaacatcttcggtctctgtattcaattttat

SEQ ID NO: 1114 - DNA, 41 bp, Homo sapiens
ttttcccagctgtaaaagggggctaataatagctcttgcgg

SEQ ID NO: 1115 - DNA, 41 bp, Homo sapiens
gatacctgactccaggagccgtcactttacaacctgagatt

SEQ ID NO: 1116 - DNA, 41 bp, Homo sapiens
ttcttgcccttgtacatgtcaacgatcttctccgagtagat

SEQ ID NO: 1117 - DNA, 41 bp, Homo sapiens
atcatgctcagtgaaacaaatcagaaaggccacacgctcta

SEQ ID NO: 1118 - DNA, 41 bp, Homo sapiens
acctggtcaacagcttccctcaggattttactgccaagcca

SEQ ID NO: 1119 - DNA, 41 bp, Homo sapiens
cacccagtctgaccttcactcttttgttgatggggctgagc

SEQ ID NO: 1120 - DNA, 41 bp, Homo sapiens
gctgctgggggtgggtgcttcgatcctggtgaaatggcctc

SEQ ID NO: 1121 - DNA, 41 bp, Homo sapiens
agaatcatcttctcctttccctcacctgatacccagcttga

SEQ ID NO: 1122 - DNA, 41 bp, Homo sapiens
cctgtcaggcctgacggggaagaaccactgcaccaccgaga

SEQ ID NO: 1123 - DNA, 41 bp, Homo sapiens
ggctatgaatatagtacctggaaaaatgccaagacatgatt

SEQ ID NO: 1124 - DNA, 41 bp, Homo sapiens
cttttgggaatttcctctcctcttggcactcggagttgggg

SEQ ID NO: 1125 - DNA, 41 bp, Homo sapiens
caagccatggcagcggacagtctgctgagaacacccaggaa

SEQ ID NO: 1126 - DNA, 41 bp, Homo sapiens
gaccagtgaacttcatccttgtctgtccaggaggtggcctc

SEQ ID NO: 1127 - DNA, 41 bp, Homo sapiens
tcagtatagatgcacccatcctaagcctaactacattgtat

SEQ ID NO: 1128 - DNA, 41 bp, Homo sapiens
gtgagcgtgccatcagcccaatggaggggcttaggtctgca

SEQ ID NO: 1129 - DNA, 41 bp, Homo sapiens
ggtgccatccagtgccctgacagtcagttcgaatgcccgga

SEQ ID NO: 1130 - DNA, 41 bp, Homo sapiens
ggcccgtagccctcacgtggctgtgaaggacgtggagtgtg

SEQ ID NO: 1131 - DNA, 41 bp, Homo sapiens
tcaggcctccctagcacctctccctaaccaaattctccctg

SEQ ID NO: 1132 - DNA, 41 bp, Homo sapiens
agccatgagtttccaccagcggcagagtgagtcctgagcac

SEQ ID NO: 1133 - DNA, 41 bp, Homo sapiens
attgcagagaatggaagaatgtgaagaactgagtgacaagg

SEQ ID NO: 1134 - DNA, 41 bp, Homo sapiens
agctactgggtagaattttatgtagtaactaggtagacact

SEQ ID NO: 1135 - DNA, 41 bp, Homo sapiens
ggatggcatagcgagaataccaatctaggaagcgactggac

SEQ ID NO: 1136 - DNA, 41 bp, Homo sapiens
gctttcctgctatcatagcctacttaagtagctgtattagg

SEQ ID NO: 1137 - DNA, 41 bp, Homo sapiens
atgaggaagagagagacgagatggggtgactcatgcctgaa

SEQ ID NO: 1138 - DNA, 41 bp, Homo sapiens
tttctttgagacagggtctcgctctgttacccaagctggra

SEQ ID NO: 1139 - DNA, 41 bp, Homo sapiens
tcattagcagggtgatggtggggctgagatgggcagggcca

SEQ ID NO: 1140 - DNA, 41 bp, Homo sapiens
attgccaacatagctgttcacacctagaacaccttttcctt

SEQ ID NO: 1141 - DNA, 41 bp, Homo sapiens
cacaacctcggtaaggctggcgatcttcaagccagtccgat

SEQ ID NO: 1142 - DNA, 41 bp, Homo sapiens
gtccgttgtccacgttctacttccaccccactaactgaacg

SEQ ID NO: 1143 - DNA, 41 bp, Homo sapiens
aggccaggggtctggatgcatatagcgttcccctagcctct

SEQ ID NO: 1144 - DNA, 41 bp, Homo sapiens
tgcagaggtgtgggcccctgaggacccagaagtccagccac

SEQ ID NO: 1145 - DNA, 41 bp, Homo sapiens
gggtgaagtaaagtgggcagagtgatttagcagagtggtca

SEQ ID NO: 1146 - DNA, 41 bp, Homo sapiens
ggcacctgtcatagtcttgctgaaagatgacaagccctggt

SEQ ID NO: 1147 - DNA, 41 bp, Homo sapiens
cgcagcccaggatgatctgtgcgggacagaggcagcggcct

SEQ ID NO: 1148 - DNA, 41 bp, Homo sapiens
tcggaacagcgagtcctctggcgtcgagagcagggaggggt

SEQ ID NO: 1149 - DNA, 41 bp, Homo sapiens
tttgcccagtgacgcagcatcccaggctgagattgcagaat

SEQ ID NO: 1150 - DNA, 41 bp, Homo sapiens
gccccctctgcaggtccccttggtgtactctgaggtgggaa